(19) United States
(12) Patent Application Publication
(10) Pub. No.: US 2024/0252693 A1
(43) Pub. Date: Aug. 1, 2024

(54) FOLATE RECEPTOR-TARGETED RADIOTHERAPEUTIC AGENTS AND THEIR USE

(71) Applicant: Novartis AG, Basel (CH)

(72) Inventors: Christopher P. LEAMON, West Lafayette, IN (US); Iontcho R. VLAHOV, West Lafayette, IN (US); Joseph A. REDDY, Davis, CA (US); Hari Krishna R. SANTHAPURAM, West Lafayette, IN (US)

(21) Appl. No.: 18/555,224

(22) PCT Filed: Apr. 13, 2022
(86) PCT No.: PCT/IB2022/053493

§ 371 Date: Oct. 12, 2023

Related U.S. Application Data
(60) Provisional application No. 63/175,883, filed on Apr. 16, 2021.

(51) Int. Cl. A61K 51/04; A61K 31/519; A61K 45/06; A61P 35/00; C07D 475/04

(52) U.S. Cl. CPC A61K 51/0459; A61K 31/519; A61K 45/06; A61P 35/00; C07D 475/04; A61K 2121/00; A61K 2123/00

(57) ABSTRACT

The present disclosure relates to folate receptor-targeted radiotherapeutic compounds and their use. The present disclosure relates to folate receptor-targeted radiolabeled imaging conjugates and their use. The present disclosure also relates to pharmaceutical compositions of the compounds and conjugates described herein, methods of making and methods of using the same.

### CROSS-REFERENCE TO RELATED APPLICATIONS

This application claims the benefit of and priority to U.S. Patent Application No. 63/175,883, filed Apr. 16, 2021, which is herein incorporated by reference in its entirety, for all purposes.

### TECHNICAL FIELD

The present disclosure relates to folate receptor-targeted radiotherapeutic compounds and their use, for example, in radionuclide therapy, in imaging, diagnostic, and theragnostic methods. The present disclosure also relates to pharmaceutical compositions of the compounds described herein, methods of making, and methods of using the same.

### BACKGROUND OF THE INVENTION

The mammalian immune system provides a means for the recognition and elimination of pathogenic cells, such as tumor cells, and other invading foreign pathogens. While the immune system normally provides a strong line of defense, there are many instances where pathogenic cells, such as cancer cells, and other infectious agents evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they lack sufficient selectivity to preferentially destroy pathogenic cells, and therefore, may also harm normal host cells, such as cells of the hematopoietic system, and other non-pathogenic cells. The adverse side effects of these anticancer drugs highlight the need for the development of new therapies selective for pathogenic cell populations and with reduced host toxicity.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diptheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991).

Another approach for targeting populations of pathogenic cells, such as cancer cells or foreign pathogens, in a host is to enhance the host immune response against the pathogenic cells to avoid the need for administration of compounds that may also exhibit independent host toxicity. One reported strategy for immunotherapy is to bind antibodies, for example, genetically engineered multimeric antibodies, to the surface of tumor cells to display the constant region of the antibodies on the cell surface and thereby induce tumor cell killing by various immune-system mediated processes (De Vita, V. T., Biologic Therapy of Cancer, 2d ed. Philadelphia, Lippincott, 1995; Soulillou, J. P., U.S. Pat. No. 5,672,486). However, these approaches have been complicated by the difficulties in defining tumor-specific antigens.

Another approach that has been the subject of recent interest is the delivery of radioisotopes of certain metals to a patient. Such an approach has been applied to functional nanoparticles, antibodies, and small molecule conjugates (see for example Teo M Y, Morris M J, “Prostate-Specific Membrane Antigen-Directed Therapy for Metastatic Castration-Resistant Prostate Cancer,” Cancer J. 2016; 22(5):347-352; Jeon J., “Review of Therapeutic Applications of Radiolabeled Functional Nanomaterials,” Int J Mol Sci. 2019; 20(9):2323); and Steiner, M, Neri, D., “Antibody-Radionuclide Conjugates for Cancer Therapy: Historical Considerations and New Trends,” Clin Cancer Res Oct. 15, 2011 (17) (20) 6406-6416. These approaches have provided mixed success within the large variety of known cancers and known cancer drivers. Given the diversity of cancer types and cancer drivers, there is continued interest and a significant unmet need for new approaches to radiotherapeutic agents.

Folate plays important roles in nucleotide biosynthesis and cell division, intracellular activities which occur in both malignant and certain normal cells. The folate receptor has a high affinity for folate, which, upon binding the folate receptor, impacts the cell cycle in dividing cells. Folate receptors have been implicated in a variety of frequent tumor types, for example, ovarian, brain, lung, renal and colorectal cancers, which have been shown to demonstrate high folate receptor expression. In contrast, folate receptor expression in normal tissues is limited with the notable exception of kidney. Although folate receptor (FR) targeting is an attractive strategy for new therapies, FR targeted radionuclide therapy has not yet been possible. The primary reason is the generally high accumulation of FR-targeting radioconjugates in the kidney and associated potential for damage of renal tissue. There is a great need for the development of FR-targeting radioconjugates, particularly FR-targeting radioconjugates with reduced kidney uptake, FR-targeted radionuclide therapy with these radioconjugates, and methods to diagnose and image FR positive cancers.

### SUMMARY OF THE INVENTION

The present disclosure includes FR-targeting radioconjugates for FR-targeted radionuclide therapy, diagnosis and imaging of FR positive cancers. The present disclosure further includes pharmaceutical compositions and combinations with these FR-targeting compounds. When used for treatment the FR targeting compound typically includes a radioelement, for example, a radioelement such as ²²⁵Ac or ¹⁷⁷Lu complexed by a chelating group in the compound. When used for diagnosis or imaging, the FR targeting compound typically includes a radioelement suitable for imaging, which can also be a radioelement, or chelated Si-¹⁸F, B-¹⁸F, or Al-¹⁸F, or a radiolabeled prosthetic group.

Various embodiments of the invention are described herein.

Within certain aspects, provided herein is a compound of formula (I),

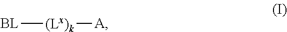

or a pharmaceutically acceptable salt thereof, wherein
BL is a folate receptor binding ligand,
A is a chelating group Ch which can comprise a metal, a radioelement, Si-¹⁸F, B-¹⁸F, or Al-¹⁸F, or A is a radiolabeled prosthetic group PG,
k is 1, 2, 3, 4, 5, 6, 7, 8, 9, or 10, and
each L^{X} is independently selected from AA, L¹, L² or L³, wherein
each AA is independently an amino acid residue;
each L¹, L² and L³ are independently as provided herein in embodiments of the present disclosure.

In another aspect, the invention provides a pharmaceutical composition comprising a compound of the present disclosure, for example, of formula (I), or a pharmaceutically acceptable salt thereof, and one or more pharmaceutically acceptable carriers.

In another aspect, the invention provides a combination, in particular a pharmaceutical combination, comprising a compound of the present disclosure, for example, of formula (I), or a pharmaceutically acceptable salt thereof, and one or more therapeutically active agents.

In another aspect, the invention provides a method of treating a folate receptor (FR) expressing tumor or cell, the method comprising contacting the one or more FR expressing tumor or cell with an effective amount of a compound of the present disclosure, for example, of formula (I), or pharmaceutically acceptable salt thereof, or with an effective amount of a pharmaceutical composition of the present disclosure, wherein the compound comprises a radiolabeled prosthetic group or a chelating group which chelates a radioelement.

In another aspect, the invention provides a method of a proliferative disease in a subject in need thereof, comprising administering to the subject a therapeutically effective amount of a compound of the present disclosure, or a pharmaceutically acceptable salt thereof, or a therapeutically effective amount of a pharmaceutical composition of the present disclosure, wherein the compound comprises a radiolabeled prosthetic group or a chelating group which chelates a radioelement.

In another aspect, the invention provides a method for imaging FR expressing cells in a subject (e.g., abnormal cell growth or tumors associated with FR expressing cancer) in a subject, comprising administering to the subject an effective amount of a compound of the present disclosure, or a pharmaceutically acceptable salt thereof, or an effective amount of a pharmaceutical composition of the present disclosure, in an amount effective for imaging the abnormal cell growth, wherein the compound comprises a metal, radioelement or radiohalogen.

### BRIEF DESCRIPTION OF THE DRAWINGS

FIG. 1 is a chart showing the relative affinity of test compounds to folate receptor positive KB cells at various concentrations after 1 hour of incubation time: (●) folic acid; (▴) Compound 37; (▪) Compound 34.

FIG. 2 is a chart showing the binding affinity of Compound 37 and Compound 34 in folate receptor positive KB cells at various concentrations: (♦) [¹⁷⁷Lu]-Compound 37 (Kd=8.99 nM); (▪) [¹⁷⁷Lu]-Compound 34 (Kd=7.21 nM).

FIG. 3 is a chart showing the results of an in-vivo biodistribution analysis of a 200 nmol/kg dose of [¹⁷⁷Lu]-Compound 45, [¹⁷⁷Lu]-Compound 17, and [¹⁷⁷Lu]-Compound 68, and a 300 nmol/kg dose of [¹⁷⁷Lu]-Compound 37 and [¹⁷⁷Lu]-Compound 34 in female nu nu mice bearing folate receptor positive M109 tumors, 24 hours post-injection.

FIG. 4 is a chart providing the tumor to kidney ratios corresponding to the results of the biodistribution analysis shown in FIG. 3 for compounds [¹⁷⁷Lu]-Compound 45, [¹⁷⁷Lu]-Compound 17, [¹⁷⁷Lu]-Compound 68, [¹⁷⁷Lu]-Compound 37 and [¹⁷⁷Lu]-Compound 34.

FIG. 5 is a chart showing the anti-tumor activity of [¹⁷⁷Lu]-Compound 37 and [¹⁷⁷Lu]-Compound 34 at 300 nmol/3.7 GBq/kg in female nu nu mice bearing MDA-MB-231 tumors (n=5); four partial responses (PRs) and one complete response (CR) were found for [¹⁷⁷Lu]-Compound 37 and five PRs were found for [¹⁷⁷Lu]-Compound 34; (▪) control; (▴) [¹⁷⁷Lu]-Compound 37; (●) [¹⁷⁷Lu]-Compound 34.

FIG. 6 is a chart showing the average weight of mice from the study in FIG. 5. The results show treatment was well tolerated; mice in both of the treated groups did not lose any significant weight immediately after dosing and beyond; (▪) control; (▴) [¹⁷⁷Lu]-Compound 37; (●) [¹⁷⁷Lu]-Compound 34.

FIG. 7 is a chart showing the anti-tumor activity of [²²⁵Ac]-Compound 5 at 100 nmol/30 mCi/kg in mice bearing MDA-MB-231 tumors. The results show treatment with [²²⁵Ac]-Compound 5 provided 50% complete response and 50% partial response. (▪) control; (●) [²²⁵Ac]-Compound 5.

FIG. 8 is a chart showing the anti-tumor activity of [²²⁵Ac]-Compound 5 at 100 nmol/30 mCi/kg in mice bearing KB tumors. The results show treatment with [²²⁵Ac]-Compound 5 provided 80% partial response and 20% stable disease. (▪) control; (●) [²²⁵Ac]-Compound 5.

FIG. 9 is a chart showing the results of an in-vivo biodistribution analysis of a 600 nmol/kg dose of [¹⁷⁷Lu]-Compound 37 and [¹⁷⁷Lu]-Compound 34 in female Athymic Nude-Foxn1^{nu} mice bearing folate receptor positive IGROV-1 tumors, 4 and 24 hours post-injection; particularly, the percentage injected dose per gram of tissue (% ID/g tissue) at 4 h and 24 h post injection for various tissue samples (mean±SD).

FIG. 10 is a chart showing the results of an in-vivo biodistribution analysis, particularly, the percentage injected dose per gram of tissue (% ID/g) at different time points (30 min, 1 h, 4 h, 24 h, 48 h, and 72 h) post injection of a mass dose of 100 nmol/kg BW of [¹⁷⁷Lu]-Compound 34 in female Athymic Nude-Foxn1^{nu} mice.

FIG. 11 is a chart showing the results of an in-vivo biodistribution analysis, particularly, the percentage injected dose per gram of tissue (% ID/g) at different time points (30 min, 1 h, 4 h, 24 h, 48 h, and 72 h) post injection of a mass dose of 200 nmol/kg BW of [¹⁷⁷Lu]-Compound 34 in female Athymic Nude-Foxn1^{nu} mice.

FIG. 12 is a chart showing the results of an in-vivo biodistribution analysis, particularly, the percentage injected dose per gram of tissue (% ID/g) at different time points (30 min, 1 h, 4 h, 24 h, 48 h, and 72 h) post injection of a mass dose of 200 nmol/kg BW of [¹⁷⁷Lu]-Compound 34 in female Athymic Nude-Foxn1^{nu} mice.

### DETAILED DESCRIPTION OF THE INVENTION

The present disclosure includes FR-targeting radioconjugates (also referred to as “FR-targeting compounds” or “compounds”), compositions thereof, and combinations thereof, for therapy, diagnosis and imaging of a proliferative disease such as FR expressing cancers.

Within certain aspects, provided herein is a compound of formula (I),

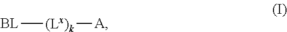

or a pharmaceutically acceptable salt thereof, wherein
BL is a folate receptor binding ligand,
A is a chelating group Ch which can comprise a metal, a radioelement, Si-¹⁸F, B-¹⁸F, or Al-¹⁸F, or A is a radiolabeled prosthetic group PG,
k is 1, 2, 3, 4, 5, 6, 7, 8, 9, or 10, and
each L^{X} is independently AA, L¹, L² or L³, wherein
each AA is independently an amino acid residue;
each L¹ is independently of the formula

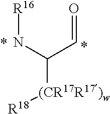

wherein
R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′}.
each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl, or a sugar;
w is 1, 2, 3, 4 or 5;
p is 1, 2, 3, 4 or 5;
q is 1, 2, 3, 4 or 5;
each L² is independently of the formula

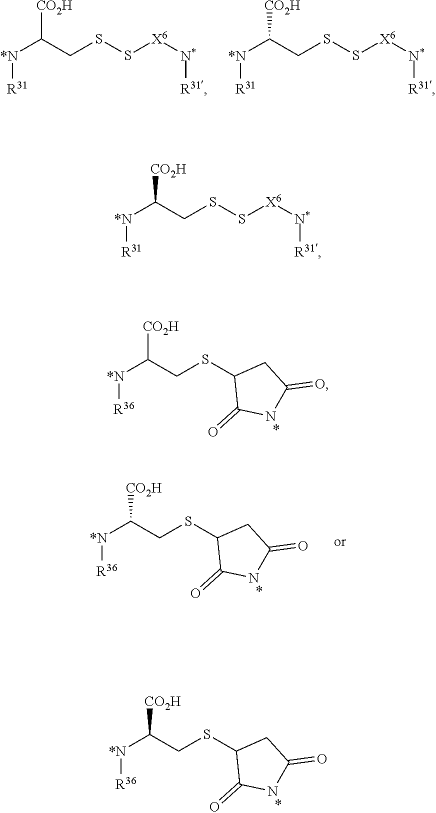

wherein
each R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′} —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
X⁶ is C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R³⁵, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} are independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
R³⁶ is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each L³ is independently C₁-C₆ alkylene, —OC₁-C₆ alkylene, —SC₁-C₆ alkylene, C₃-C₆ cycloalkylene, —C(O)C₃-C₆ cycloalkylene-, —C(O)C₃-C₆ cycloalkylene-(CR³⁹R^{39′})ᵣ—, —C(O)C₃-C₆ cycloalkylene-(CR³⁹R^{39′})ᵣNR³⁷—, 3- to 7-membered heterocycloalkylene, C₆-C₁₀ aryl, 5- to 7-membered heteroaryl, —NR³⁶(CR^{36′}R^{36″})ᵣ—S-(succinimid-1-yl)-, —(CR^{36′}R^{36″})ᵣC(O)NR³⁷—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ—, —C(O)O(CR³⁹R^{39′})ᵣ—, —NR³⁷C(O)(CR³⁹R^{39′})ᵣ—, —(CR³⁹R^{39′})ᵣC(O)NR³⁷—, —NR³⁷C(O)(CR^{39′}R^{39″})ᵣS—, —NR³⁷(CR³⁹R^{39′})ᵣ—, —(CR³⁹R^{39′})ᵣNR³⁸—, —NR³⁷(CR³⁹R^{39′})ᵣNR³⁸—, —NR³⁷(CR³⁹R^{39′})ᵣS—, —NR³⁷(CR³⁹R^{39′}CR³⁹R^{39′})ᵣ—, —NR³⁷(CR³⁹R^{39′}CR³⁹R^{39′}O)ᵣₚ—(CR³⁶R^{36′})ₜC(O)—, —C(O)(CR³⁶R^{36′})ₜ—(OCR³⁹R^{39′}CR³⁹R^{39′})ₚ—NR³⁷—, (CR³⁹R^{39′}CR³⁹R^{39′}O)ᵣ—(CR³⁶R^{36′})ₜC(O)—, —C(O)(CR³⁶R^{36′})ₜ(OCR³⁹R^{39′}CR³⁹R^{39′}CR³⁹R^{39′})ᵣ—, —C(O)(CR³⁶R^{36′})ₜ(OCR³⁹R^{39′}CR³⁹R^{39′}CR³⁹R^{39′})ᵣNR³⁷—, —C(O)(CR³⁶R^{36′})ᵣ—O—(C₆-C₁₀ aryl)- (CR^{36″}R^{36′″})ₜNR³⁷—, —NR³⁷(CR³⁶R^{36′})ᵣ—(C₆-C₁₀ aryl)-O—(CR^{36″}R^{36′″})ₜC(O)—, —C(O)—(CR³⁶R^{36′})ᵣ—NR³⁷—C(O)—(C₆-C₁₀ aryl)-NR^{37′}—, —NR³⁷—(C₆-C₁₀ aryl)-C(O)— NR^{37′}—(CR³⁶R^{36′})ᵣ—C(O)—, —NR³⁷(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NR³⁷—, —NR³⁷(CR³⁶R^{36′})ᵣ—(C₆-C₁₀ aryl)-O—(CR^{36″}R^{36′″})ₜ—NR^{37′}—, or —NR^{37′}—(CR^{36″}R^{36′″})ₜ—O—(C₆-C₁₀ aryl)-(CR³⁶R^{36′})ᵣ—NR³⁷—, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
wherein
each R³⁶, R^{36′}, R^{36″} and R^{36′″} is independently selected from the group consisting of H, C₁-C₆ alkyl, C₂-C₆ alkenyl, C₂-C₆ alkynyl, C₃-C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰R^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR⁴⁰ and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each r independently is 1, 2, 3, 4, 5, 6, 7, 8, 9, or 10;
each rp independently is an integer from 1 to 80; and
each t independently is 1, 2, 3, 4, 5, 6, 7, 8, 9, or 10;
each * represents a covalent bond;
wherein for k larger than 3, at least 2 of the L^{x} in formula (I) are independently selected from

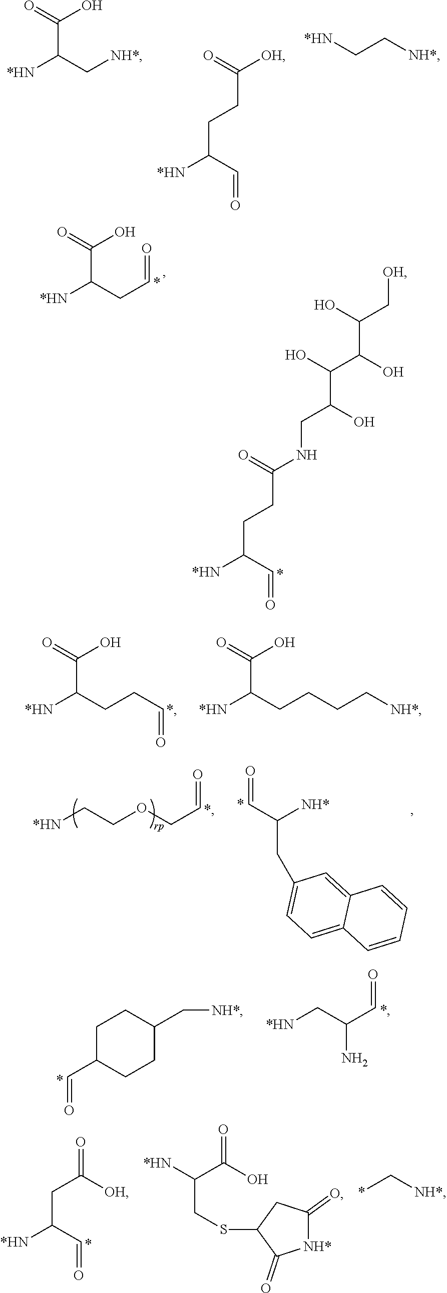

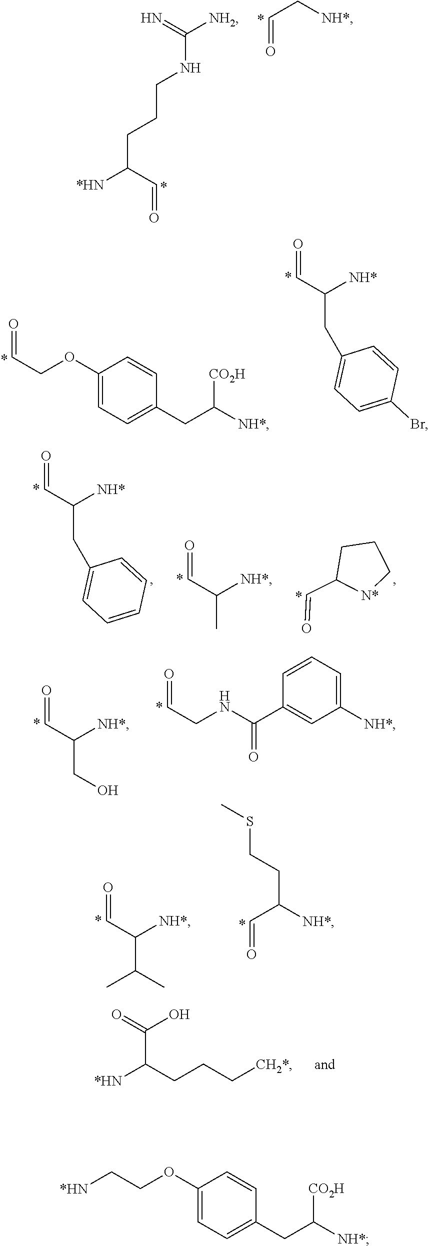

and
provided that the compound is not

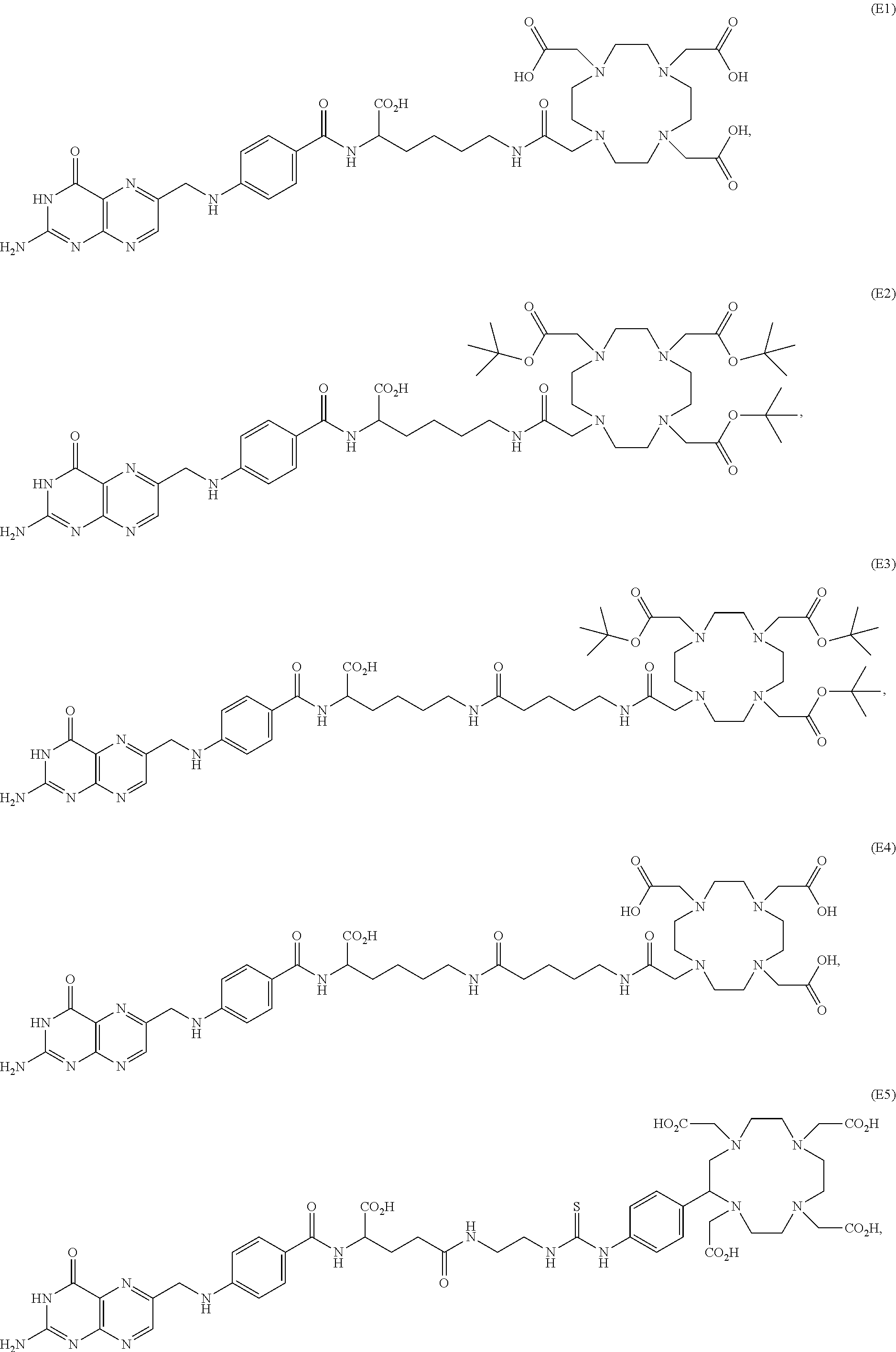

a tautomer of (E1)-(E5), a compound of (E1)-(E5) in which a metal or radioelement is chelated, or a pharmaceutical salt of (E1)-(E5) thereof.

Unless specified otherwise, the term “compounds of the present disclosure” or “compound of the present disclosure” refers to compounds of formula (I), subformulae thereof, and exemplified compounds, and salts thereof, as well as all stereoisomers (including diastereoisomers and enantiomers), rotamers, tautomers and isotopically labeled compounds (including deuterium substitutions), as well as inherently formed moieties.

The present disclosure provides the following exemplary embodiments:
Embodiment 1: A compound of formula (I)

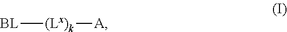

or a pharmaceutically acceptable salt thereof, wherein
BL is a folate receptor binding ligand,
A is a chelating group Ch which can comprise a metal, a radioelement, Si-¹⁸F, B-¹⁸F, or Al-¹⁸F, or A is a radiolabeled prosthetic group PG,
k is 1, 2, 3, 4, 5, 6, 7, 8, 9, or 10, and
each L^{X} is independently AA, L¹, L² or L³, wherein
each AA is independently an amino acid residue;
each L¹ is independently of the formula

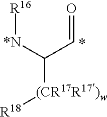

wherein
R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′} —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl, or a sugar;
w is 1, 2, 3, 4 or 5;
p is 1, 2, 3, 4 or 5;
q is 1, 2, 3, 4 or 5;
each L² is independently of the formula

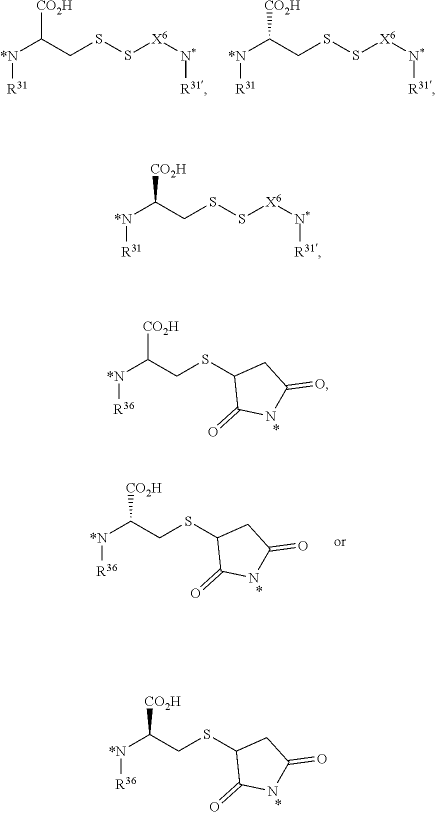

wherein
each R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′} —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
X⁶ is C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R³⁵, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} are independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
R³⁶ is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each L³ is independently C₁-C₆ alkylene, —OC₁-C₆ alkylene, —SC₁-C₆ alkylene, C₃-C₆ cycloalkylene, —C(O)C₃-C₆ cycloalkylene-, —C(O)C₃-C₆ cycloalkylene-(CR³⁹R^{39′})ᵣ—, —C(O)C₃-C₆ cycloalkylene-(CR³⁹R^{39′})ᵣNR³⁷—, 3- to 7-membered heterocycloalkylene, C₆-C₁₀ aryl, 5- to 7-membered heteroaryl, —NR³⁶(CR^{36′}R^{36″})ᵣ—S-(succinimid-1-yl)-, —(CR^{36′}R^{36″})ᵣC(O)NR³⁷—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ—, —C(O)O(CR³⁹R^{39′})ᵣ—, —NR³⁷C(O)(CR³⁹R^{39′})ᵣ—, —(CR³⁹R^{39′})ᵣC(O)NR³⁷—, —NR³⁷C(O)(CR^{39′}R^{39″})ᵣS—, —NR³⁷(CR³⁹R^{39′})ᵣ—, —(CR³⁹R^{39′})ᵣNR³⁸—, —NR³⁷(CR³⁹R^{39′})ᵣNR³⁸—, —NR³⁷(CR³⁹R^{39′})ᵣS—, —NR³⁷(CR³⁹R^{39′}CR³⁹R^{39′}O)ᵣ—, —NR³⁷(CR³⁹R^{39′}CR³⁹R^{39′}O)ᵣₚ—(CR³⁶R^{36′})ₜC(O)—, —C(O)(CR³⁶R^{36′})ₜ—(OCR³⁹R^{39′}CR³⁹R^{39′})ₚ—NR³⁷—, (CR³⁹R^{39′}CR³⁹R^{39′}O)ᵣ—(CR³⁶R^{36′})ₜC(O)—, —C(O)(CR³⁶R^{36′})ₜ(OCR³⁹R^{39′}CR³⁹R^{39′}CR³⁹R^{39′})ᵣ—, —C(O)(CR³⁶R^{36′})ₜ(OCR³⁹R^{39′}CR³⁹R^{39′}CR³⁹R^{39′})ᵣNR³⁷—, —C(O)(CR³⁶R^{36′})ᵣ—O—(C₆-C₁₀ aryl)- (CR^{36″}R^{36′″})ₜNR³⁷—, —NR³⁷(CR³⁶R^{36′})ᵣ—(C₆-C₁₀ aryl)-O—(CR^{36″}R^{36′″})ₜC(O)—, —C(O)—(CR³⁶R^{36′})ᵣ—NR³⁷—C(O)—(C₆-C₁₀ aryl)-NR^{37′}—, —NR³⁷—(C₆-C₁₀ aryl)-C(O)— NR^{37′}—(CR³⁶R^{36′})ᵣ—C(O)—, —NR³⁷(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NR³⁷—, —NR³⁷(CR³⁶R^{36′})ᵣ—(C₆-C₁₀ aryl)-O—(CR^{36″}R^{36′″})ₜ—NR^{37′}—, or —NR^{37′}—(CR^{36″}R^{36′″})ₜ—O—(C₆-C₁₀ aryl)-(CR³⁶R^{36′})ᵣ—NR³⁷—, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
wherein
each R³⁶, R^{36′}, R^{36″} and R^{36′″} is independently selected from the group consisting of H, C₁-C₆ alkyl, C₂-C₆ alkenyl, C₂-C₆ alkynyl, C₃-C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰R^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR⁴⁰ and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each r independently is 1, 2, 3, 4, 5, 6, 7, 8, 9, or 10;
each rp independently is an integer from 1 to 80; and
each t independently is 1, 2, 3, 4, 5, 6, 7, 8, 9, or 10;
each * represents a covalent bond;
wherein for k larger than 3, at least 2 of the L^{x} in formula (I) are independently selected from

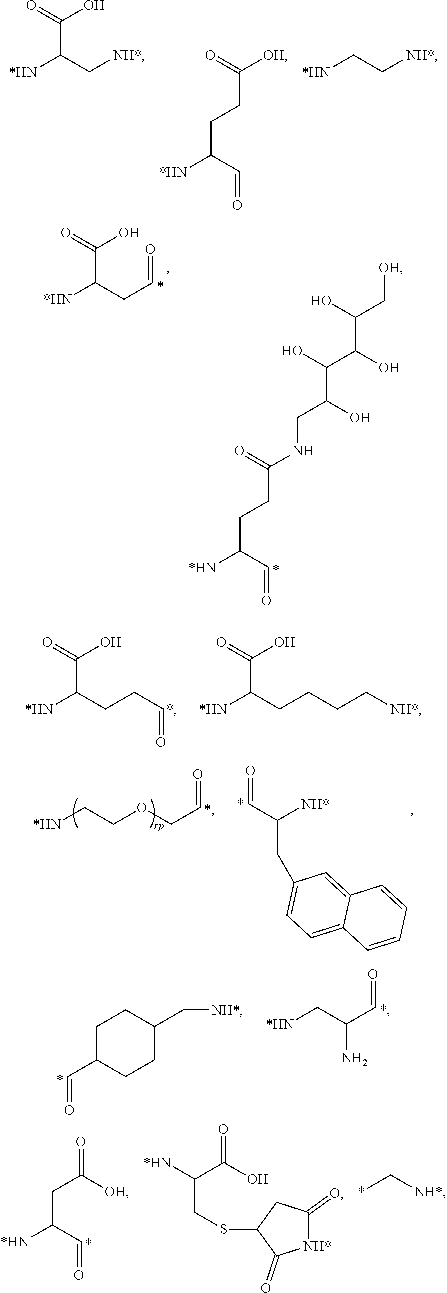

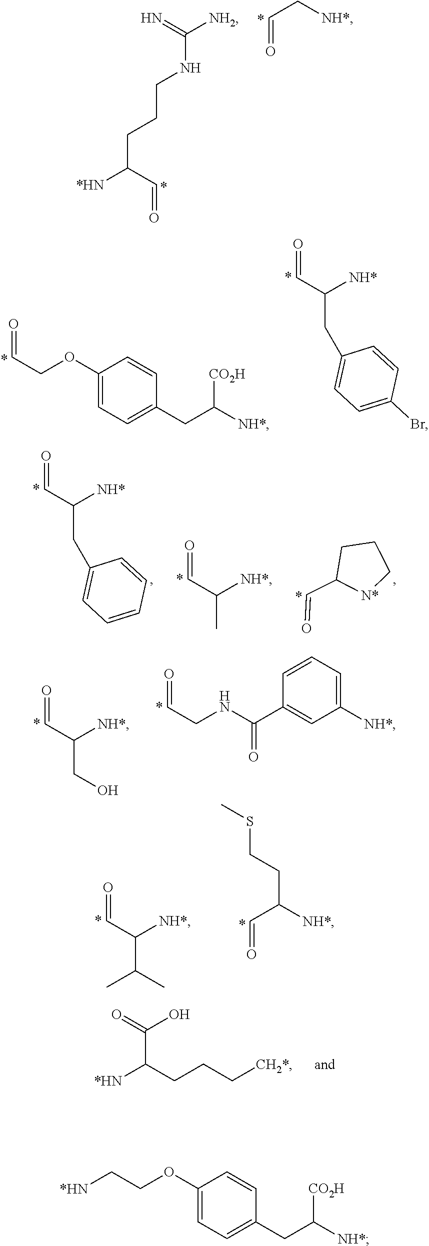

and
provided that the compound is not

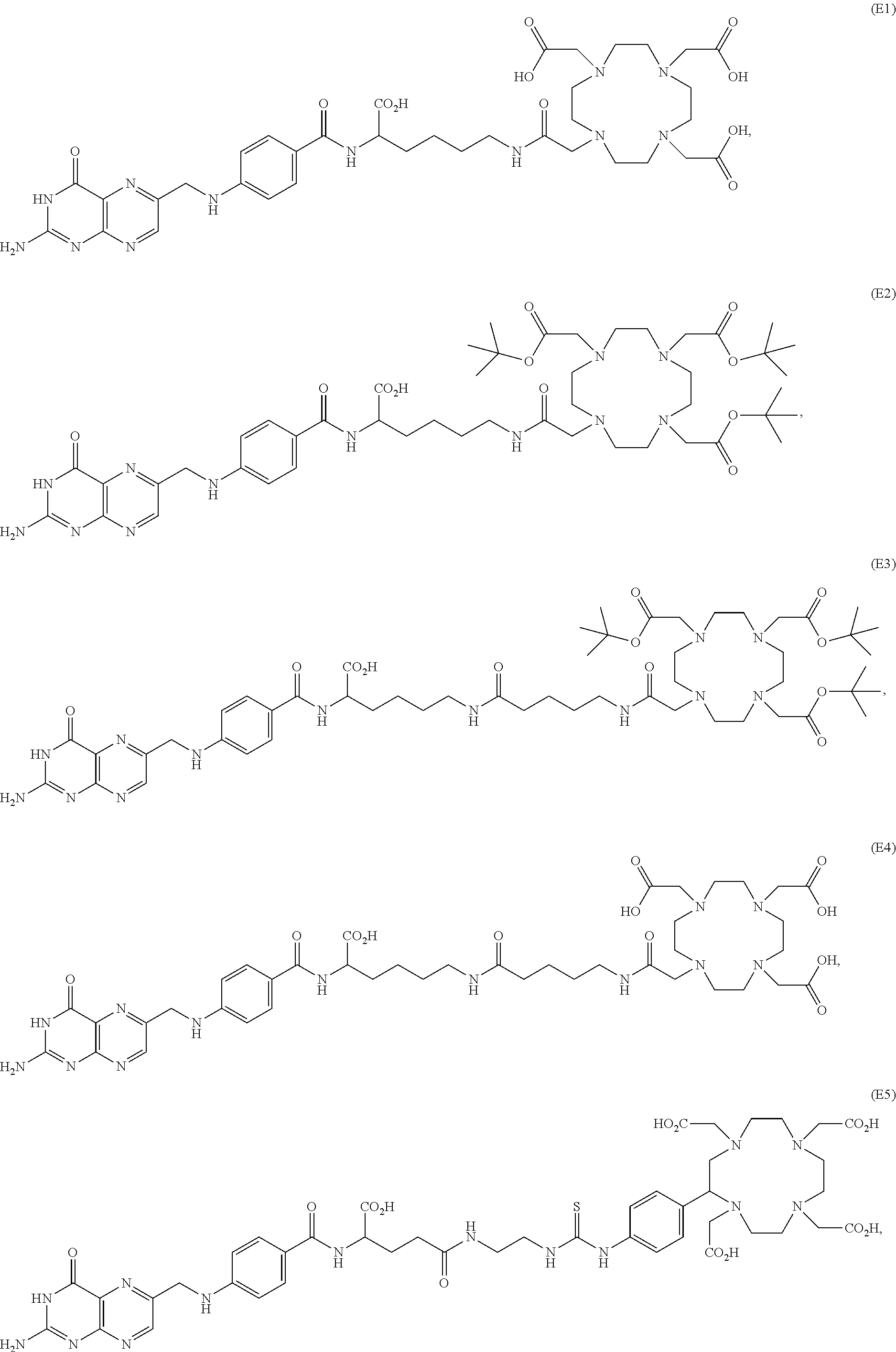

a tautomer of (E1)-(E5), a compound of (E1)-(E5) in which a metal or radioelement is chelated, or a pharmaceutical salt thereof.
Embodiment 2: The compound of Embodiment 1, or a pharmaceutically acceptable salt thereof, wherein
each L¹ is independently of the formula

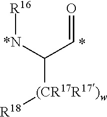

wherein
R¹⁶ is H,
each R¹⁷ and R^{17′} is independently H, C₁-C₆ alkyl, or —C(O)OH,
each R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen; each L² is independently of the formula

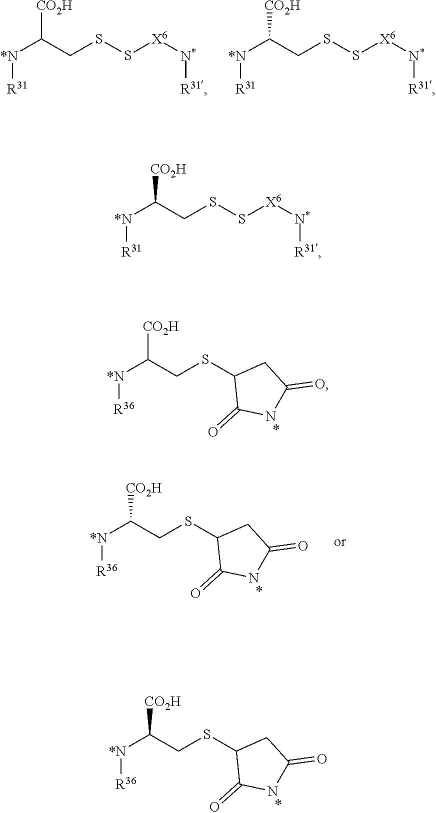

wherein
R³¹ and R^{31′} are H,
R³⁶ is H; and
each L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—;
   wherein
   each R³⁶, R^{36′}, R^{36″}, R^{36′″}, R³⁹ and R^{39′} is independently H or —COOH;
   each r independently is 1, 2, 3, 4, 5; and
   each t independently is 1, 2, 3, 4, 5.
Embodiment 3: The compound of Embodiment 1 or 2, or a pharmaceutically acceptable salt thereof, wherein BL-(L^{x})ₖ-Ch is BL-L³-Ch, BL-L¹-L³-Ch, BL-L³-L³-L¹-L¹-L¹-L³-Ch, BL-L³-L¹-Ch, BL-L³-L³-L³-Ch, BL-L³-L³-L¹-L³-L³-Ch, BL-L³-L¹-L³-Ch, BL-L³-L³-AA-L¹-L²-L³-Ch, BL-L³-L³-L¹-L¹-L¹-L²-Ch, BL-L³-L³-L³-L¹-AA-Ch, BL-L³-L³-AA-Ch, BL-L³-L³-Ch, BL-L³-L¹-AA-Ch, BL-L³-L³-L³-L¹-Ch, BL-L³-L³-L³-L¹-L¹-Ch, BL-L³-L¹-L¹-L¹-L¹-AA-AA-AA-AA-Ch, BL-L³-AA-Ch, BL-L³-L¹-L¹-L¹-AA-AA-AA-AA-Ch, BL-L³-L³-L³-AA-Ch, or BL-L³-L³-L³-L³-Ch, wherein each AA independently is an amino acid residue.
Embodiment 4: The compound of any one of Embodiments 1 to 3, or a pharmaceutically acceptable salt thereof, wherein BL comprises one amino acid residue covalently attached to a pteryl group or derivative thereof and BL-(L^{x})ₖ-Ch is BL-L³-Ch, BL-L¹-L¹-L¹-L³-Ch, BL-L¹-Ch, BL-L³-L³-Ch, BL-L¹-L³-L³-Ch, BL-L¹-L³-Ch, BL-L³-L³-AA-L¹-L²-L³-Ch, BL-L¹-L¹-L¹-L²-Ch, BL-L³-L³-L¹-AA-Ch, BL-L³-AA-Ch, BL-L¹-AA-Ch, BL-L³-L³-L¹-Ch, BL-L³-L¹-Ch, BL-L³-L³-L¹-L¹-Ch, BL-L³-L¹-L¹-Ch, BL-L¹-L¹-L¹-L¹-AA-AA-AA-AA-Ch, BL-AA-Ch, BL-L¹-L¹-L¹-AA-AA-AA-AA-Ch, or BL-L¹-L¹-L¹-L¹-L²-L³-L¹-L¹-Ch, wherein each AA independently is an amino acid residue.
Embodiment 5: The compound of any one of Embodiments 1 to 4, or a pharmaceutically acceptable salt thereof, wherein when k is larger than 4, at least 3 of the L^{x} in formula (I) are independently selected from

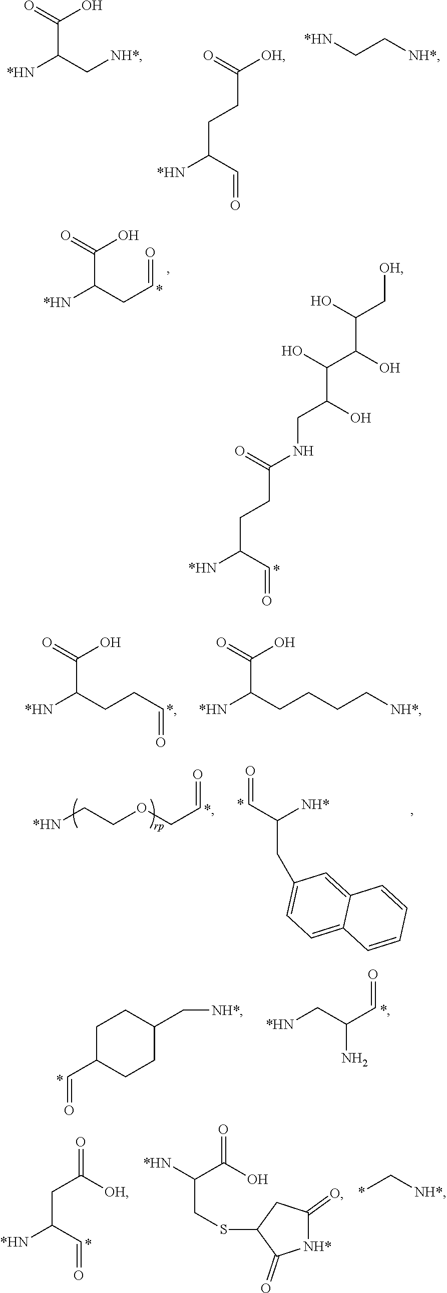

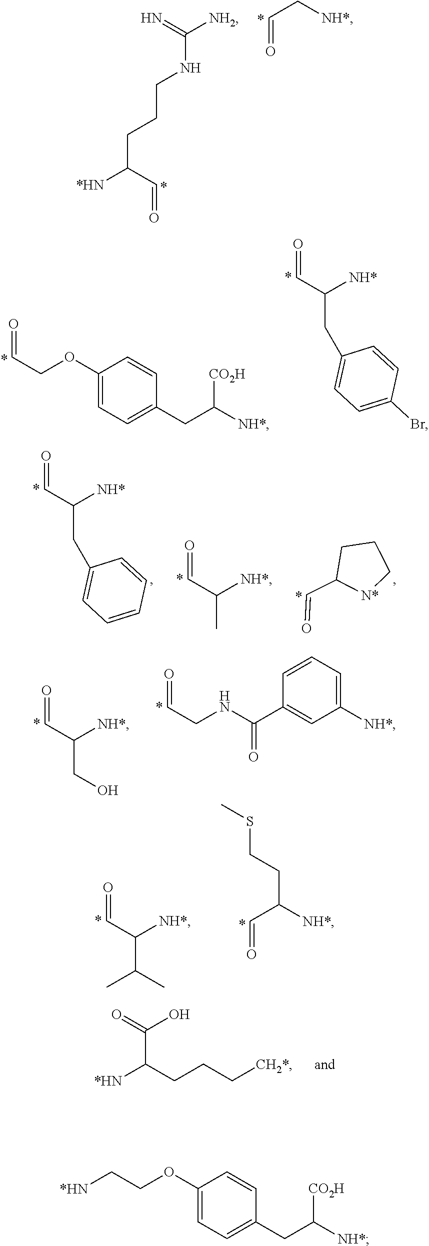

Embodiment 6: The compound of any one of Embodiments 1 to 4, or a pharmaceutically acceptable salt thereof, wherein when k is larger than 4, at least 3 of the L^{x} in formula (I) are independently selected from

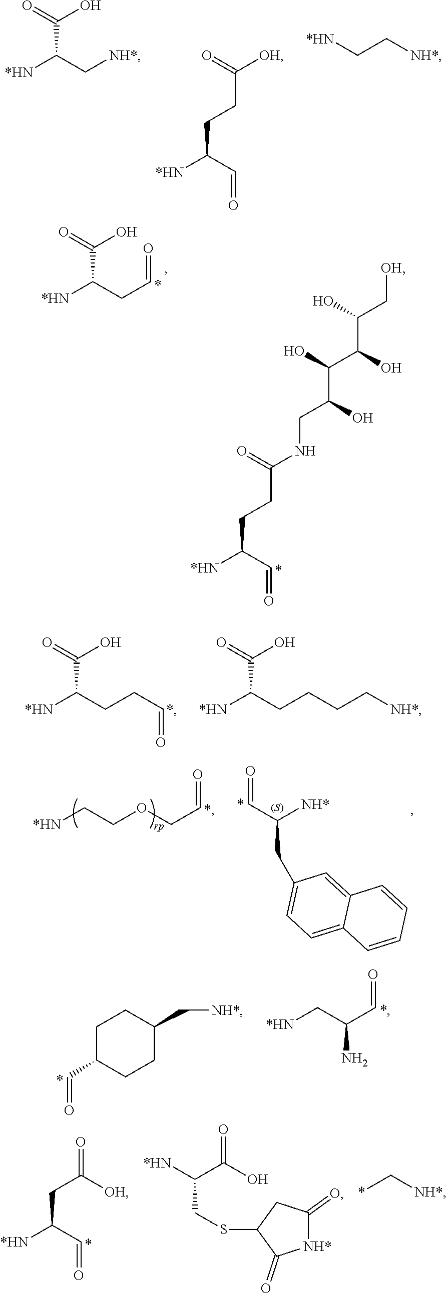

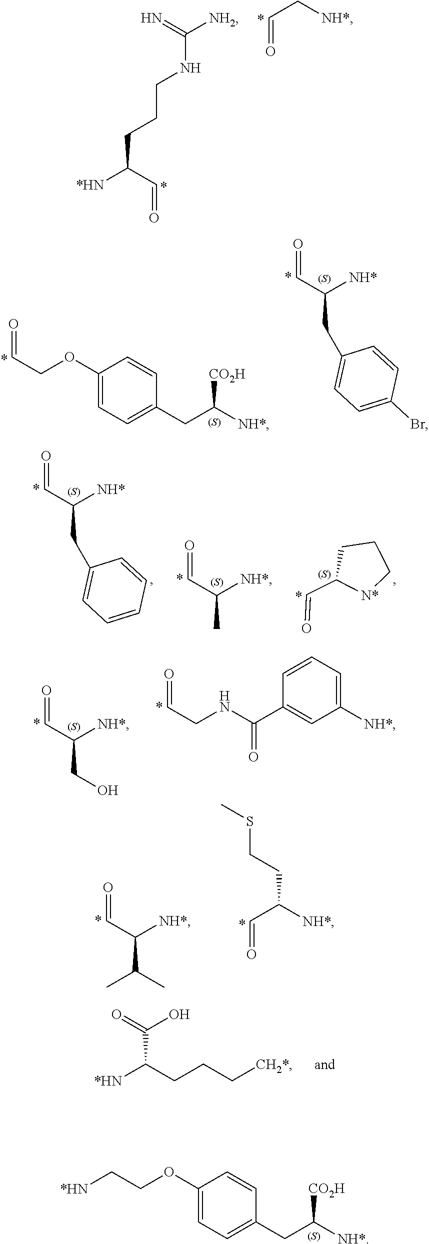

Embodiment 7: The compound of any one of Embodiments 1 to 6, or a pharmaceutically acceptable salt thereof, wherein at least one L^{x} is

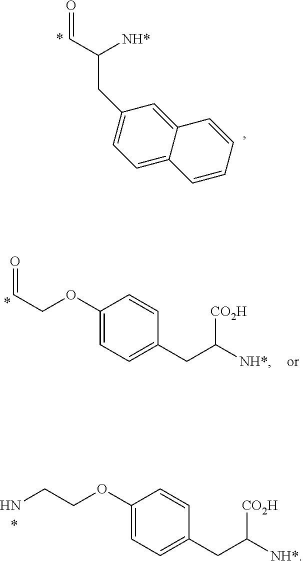

Embodiment 8: The compound of any one of Embodiments 1 to 6, or a pharmaceutically acceptable salt thereof, wherein BL-(L^{x})ₖ-Ch is of the formula BL-L^{x}-L^{a}-L^{x}-Ch, BL-L^{x}-L^{x}-L^{a}-L^{x}-Ch, BL-L^{x}-L^{x}-L^{a}-Ch, or BL-L^{x}-L^{x}-L^{a}-L^{a}-Ch wherein L^{a} is

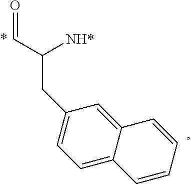

and each L^{x} independently is AA, L¹, or L³.
Embodiment 9: The compound of any one of Embodiments 1 to 6, or a pharmaceutically acceptable salt thereof, wherein BL-(L^{x})ₖ-Ch is of the formula BL-L^{a}-L^{x}-Ch, BL-L^{x}-L^{a}-L^{x}-Ch, BL-L^{x}-L^{a}-Ch, or BL-L^{x}-L^{a}-L^{a}-Ch, wherein L^{a} is

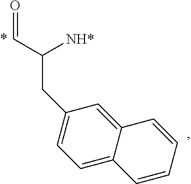

and each L^{x} independently is AA, L¹, or L.
Embodiment 10: The compound of Embodiment 8 or 9, or pharmaceutically acceptable salt thereof, wherein L^{a} is

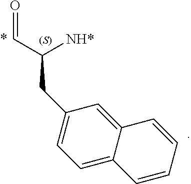

Embodiment 11: The compound of any one of Embodiments 1 to 6, or a pharmaceutically acceptable salt thereof, wherein at least one L^{x} is

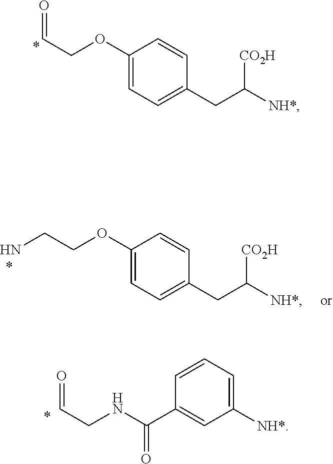

Embodiment 12: The compound of any one of Embodiments 1 to 6, or a pharmaceutically acceptable salt thereof, wherein at least one L^{x} is

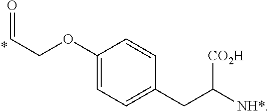

Embodiment 13: The compound of any one of Embodiments 1 to 6, or a pharmaceutically acceptable salt thereof, wherein BL-(L^{x})ₖ-Ch is of the formula BL-L^{x}-L^{b}-L^{x}-Ch, BL-L^{x}-L^{b}-Ch, or BL-L^{x}-L^{b}-L^{b}-Ch, wherein L^{b} is

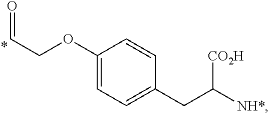

and each L^{x} independently is AA, L¹, or L³.
Embodiment 14: The compound of any one of Embodiments 1 to 6, or a pharmaceutically acceptable salt thereof, wherein BL-(L^{x})ₖ-Ch is of the formula BL-L^{b}-L^{x}-Ch, BL-L^{b}-Ch, or BL-L^{b}-L^{b}-Ch, wherein L^{b} is

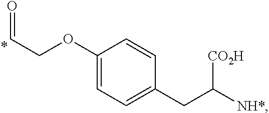

and each L^{x} independently is AA, L¹, or L³.
Embodiment 15: The compound of Embodiment 13 or 14, or pharmaceutically acceptable salt thereof, wherein L^{b} is

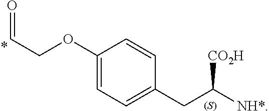

Embodiment 16: The compound of any one of the preceding Embodiments, or a pharmaceutically acceptable salt thereof, wherein BL comprises a pteryl group or a derivative thereof.
Embodiment 17: The compound of any one of the preceding Embodiments, or a pharmaceutically acceptable salt thereof, wherein BL is of the formula

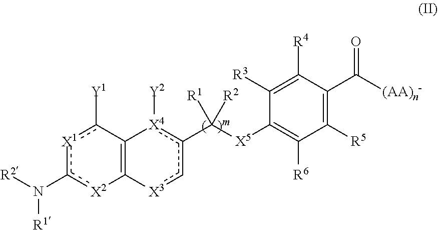

wherein
R¹ and R² in each instance are independently selected from the group consisting of H, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂-C₆ alkynyl;
X¹ is —N(R¹¹)—, ═N—, —N═, —C(R¹¹)═ or ═C(R¹¹)—;
X² is —N(R^{11″})— or ═N—;
X³ is —N(R^{11″})—, —N═ or —C(R^{11′})═;
X⁴ is —N═ or —C═;
X⁵ is —N(R¹²)— or —C(R¹²)(R^{12′})—;
Y¹ is H, —OR¹³ or —SR¹³ when X¹ is —N═ or —C(R¹¹)═, or Y¹ is ═O when X¹ is —NR¹¹—, ═N— or ═C(R¹¹)—;
Y² is H, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;
R^{1′}, R^{2′}, R¹¹, R^{11′}, R^{11″}, R¹², R^{12′}, R¹³, R¹⁴ and R^{14′} are each independently selected from the group consisting of H, C₁-C₆ alkyl, C₂-C₆ alkenyl, C₂-C₆ alkynyl, —C(O)R¹⁵, —C(O)OR¹⁵ and —C(O)NR¹⁵R^{15′};
R¹⁵ and R^{15′} are each independently H, or C₁-C₆ alkyl;
m is 1, 2, 3 or 4; and
n is 0 or 1;
wherein * represents a covalent bond to the rest of the compound.
Embodiment 18: The compound of any one of Embodiments 1 to 17, or a pharmaceutically acceptable salt thereof, wherein m is 1.
Embodiment 19: The compound of any one of Embodiments 1 to 18, or a pharmaceutically acceptable salt thereof, wherein X¹ is —NR¹¹—.
Embodiment 20: The compound of any one of Embodiments 1 to 19, or a pharmaceutically acceptable salt thereof, wherein X² is ═N—.
Embodiment 21: The compound of any one of Embodiments 1 to 20, or a pharmaceutically acceptable salt thereof, wherein Y¹ is ═O.
Embodiment 22: The compound of any one of Embodiments 1 to 21 or a pharmaceutically acceptable salt thereof, wherein X¹ is —NR¹¹—, and R¹¹ is H.
Embodiment 23: The compound of any one of Embodiments 1 to 22, or a pharmaceutically acceptable salt thereof, wherein X³ is —C(R^{11′})═.
Embodiment 24: The compound of Embodiment 23, or a pharmaceutically acceptable salt thereof, wherein R^{11′} is H.
Embodiment 25: The compound of any one of Embodiments 1 to 22, or a pharmaceutically acceptable salt thereof, wherein X⁴ is —C═.
Embodiment 26: The compound of any one of Embodiments 1 to 23, or a pharmaceutically acceptable salt thereof, wherein Y² is H.
Embodiment 27: The compound of any one of Embodiments 1 to 20, or a pharmaceutically acceptable salt thereof, wherein X³ is —N═.
Embodiment 28: The compound of any one of Embodiments 1 to 22 or 27, or a pharmaceutically acceptable salt thereof, wherein X⁴ is —N═.
Embodiment 29: The compound of any one of Embodiments 1 to 28, or a pharmaceutically acceptable salt thereof, wherein X⁵ is —NR¹²
Embodiment 30: The compound of any one of Embodiments 1 to 29, or a pharmaceutically acceptable salt thereof, wherein R¹² is H.
Embodiment 31: The compound of any one of Embodiments 1 to 30, or a pharmaceutically acceptable salt thereof, wherein R^{1′} and R^{2′} are H.
Embodiment 32: The compound of any one of Embodiments 1 to 31, or a pharmaceutically acceptable salt thereof, wherein each R¹ and R² is H.
Embodiment 33: The compound of any one of Embodiments 1 to 32, or a pharmaceutically acceptable salt thereof, wherein R³, R⁴, R⁵ and R⁶ are H.
Embodiment 34: The compound of any one of Embodiments 1 to 33, or a pharmaceutically acceptable salt thereof, wherein n is 1.
Embodiment 35: The compound of any one of Embodiments 1 to 16, or a pharmaceutically acceptable salt thereof, wherein BL is of the formula

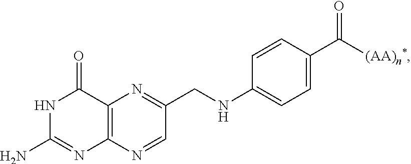

wherein n is 0 or 1, and AA is an amino acid residue.
Embodiment 36: The compound of any one of Embodiments 1 to 17, or pharmaceutically acceptable salt thereof, wherein BL is of formula

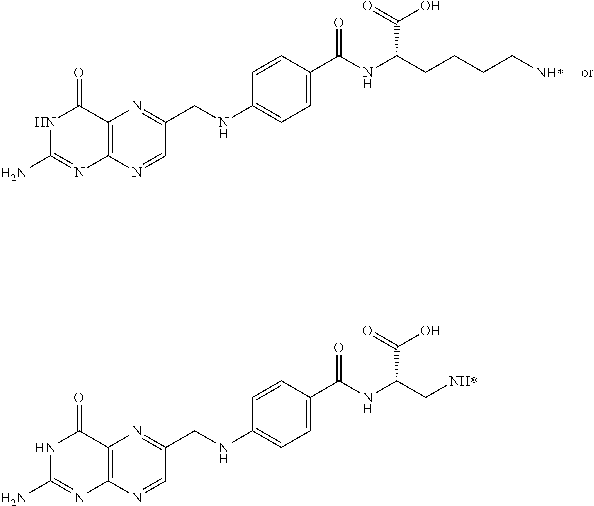

Embodiment 37: The compound of any one of Embodiments 1 to 36, or a pharmaceutically acceptable salt thereof, wherein Ch comprises a radioelement selected from the group consisting of ¹¹¹In, ^{99m}Tc, ^{94m}Tc, ⁶⁷Ga ⁶⁶Ga, ⁶⁸Ga ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.
Embodiment 38: The compound of any one of Embodiments 1 to 36, or a pharmaceutically acceptable salt thereof, wherein Ch comprises a radioelement selected from the group consisting of ⁶⁶Ga, ⁶⁷Ga, ⁶⁸Ga, ¹⁷⁷Lu, and ²²⁵Ac.
Embodiment 39: The compound of any one of the preceding Embodiments, or a salt thereof, wherein Ch is selected from the group consisting of

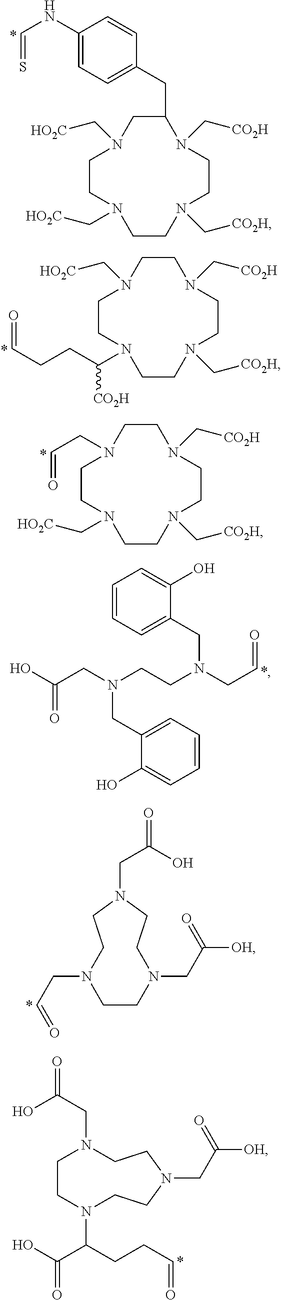

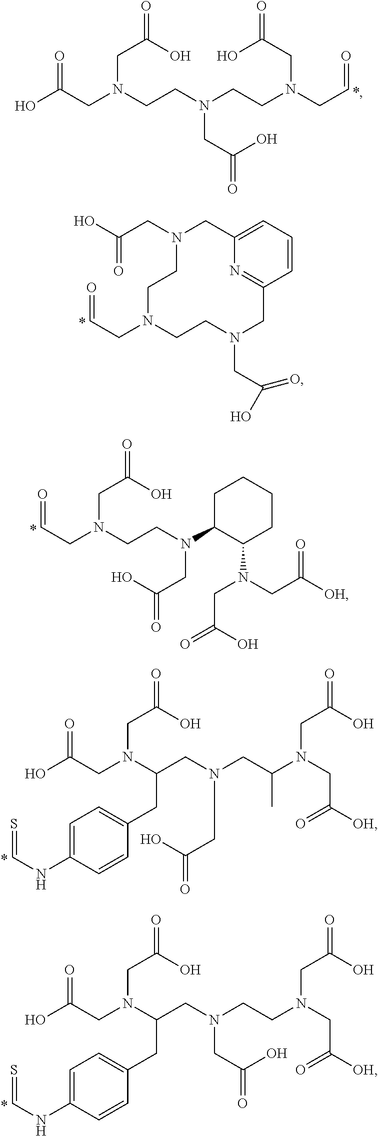

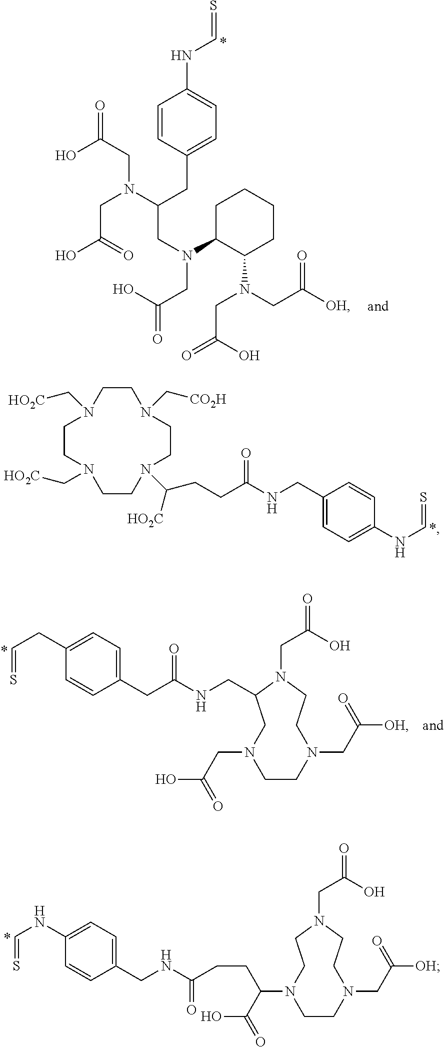

and Ch can comprise a radioelement, Si-¹⁸F, B-¹⁸F, or Al-¹⁸F.
Embodiment 40: The compound of any one of the preceding Embodiments, or a pharmaceutically acceptable salt thereof, wherein Ch is

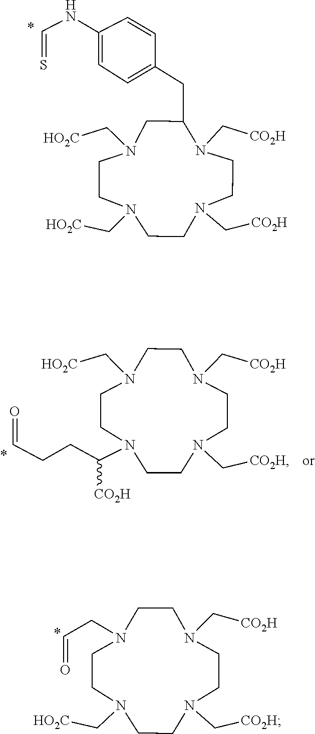 [SMILES: O=C(O)CN1CCN(CC(=O)O)CCN(CC(=O)O)C(Cc2ccc(NC=S)cc2)CN(CC(=O)O)CC1 ; O=CCCC(C(=O)O)N1CCN(CC(=O)O)CCN(CC(=O)O)CCN(CC(=O)O)CC1 ; O=CCN1CCN(CC(=O)O)CCN(CC(=O)O)CCN(CC(=O)O)CC1]

and Ch can comprise a radioelement, Si-¹⁸F, B-¹⁸F, or Al-¹⁸F.
Embodiment 41: The compound of any one of Embodiments 1 to 40, wherein BL comprises a pteryl group or a derivative thereof, and the pteryl group or derivative thereof is covalently bonded to a group selected from

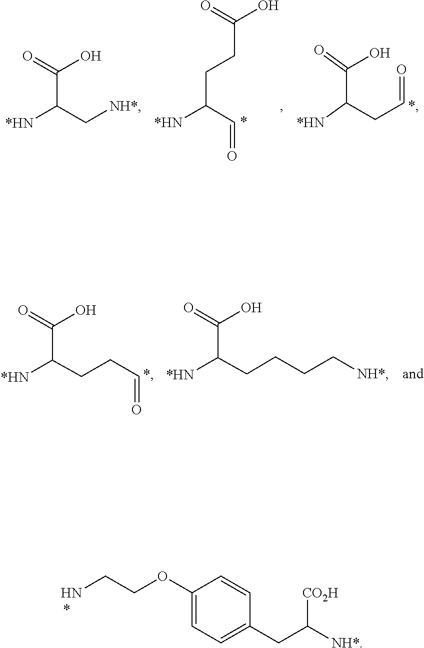

Embodiment 42: The compound of any one of Embodiments 1 to 41, wherein one, two or three L^{x} independently are L¹ in which independently w is 1 or 2, and R¹⁸ is C₆-C₁₀ aryl wherein each hydrogen is optionally substituted by halogen or C₁-C₆ alkyl.
Embodiment 43: The compound of Embodiment 42, wherein one, two or three L^{x} independently are of formula

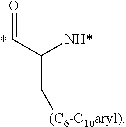

Embodiment 44: The compound of Embodiment 1, wherein the compound is of any one of formula (C1) to (C32):

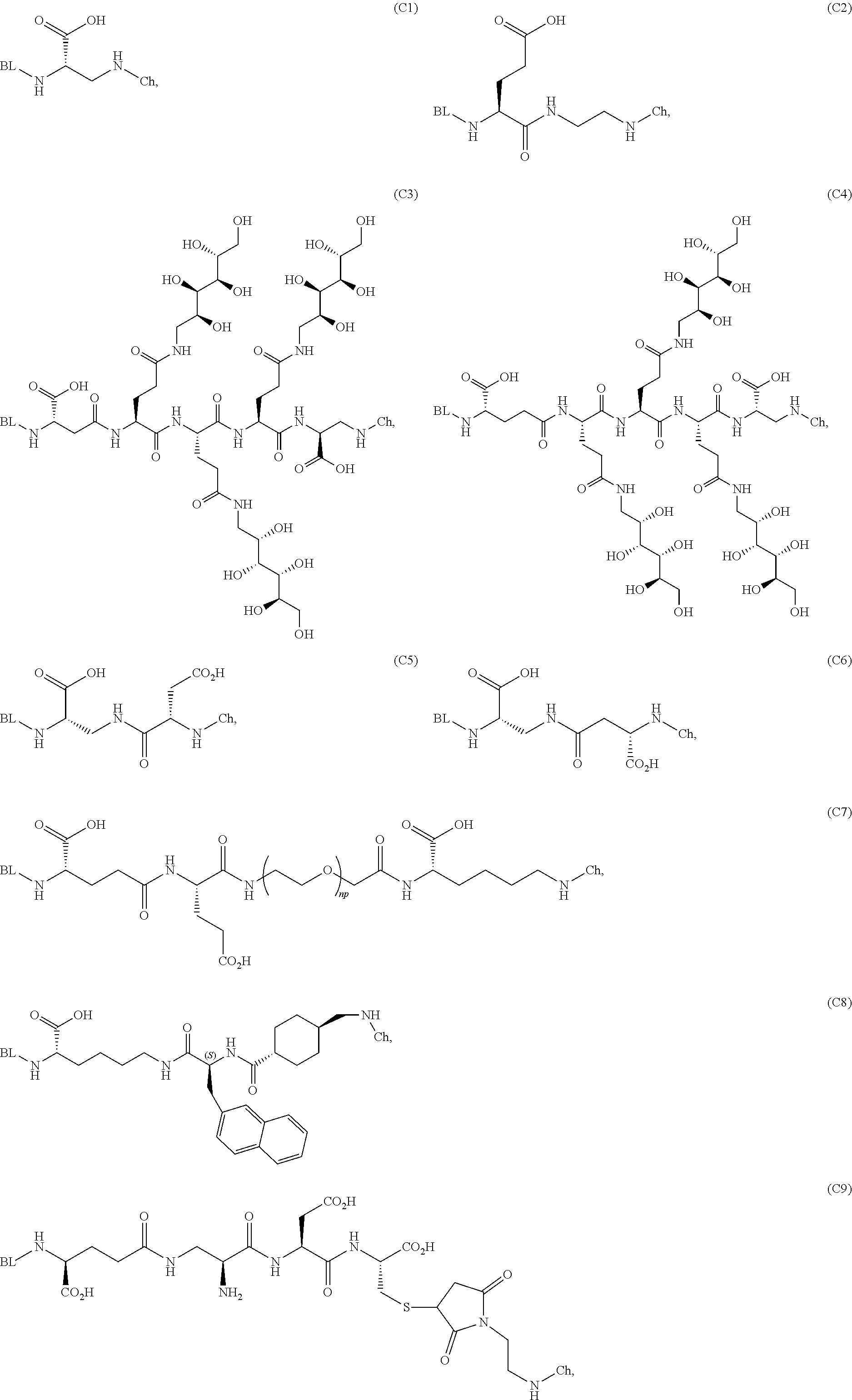

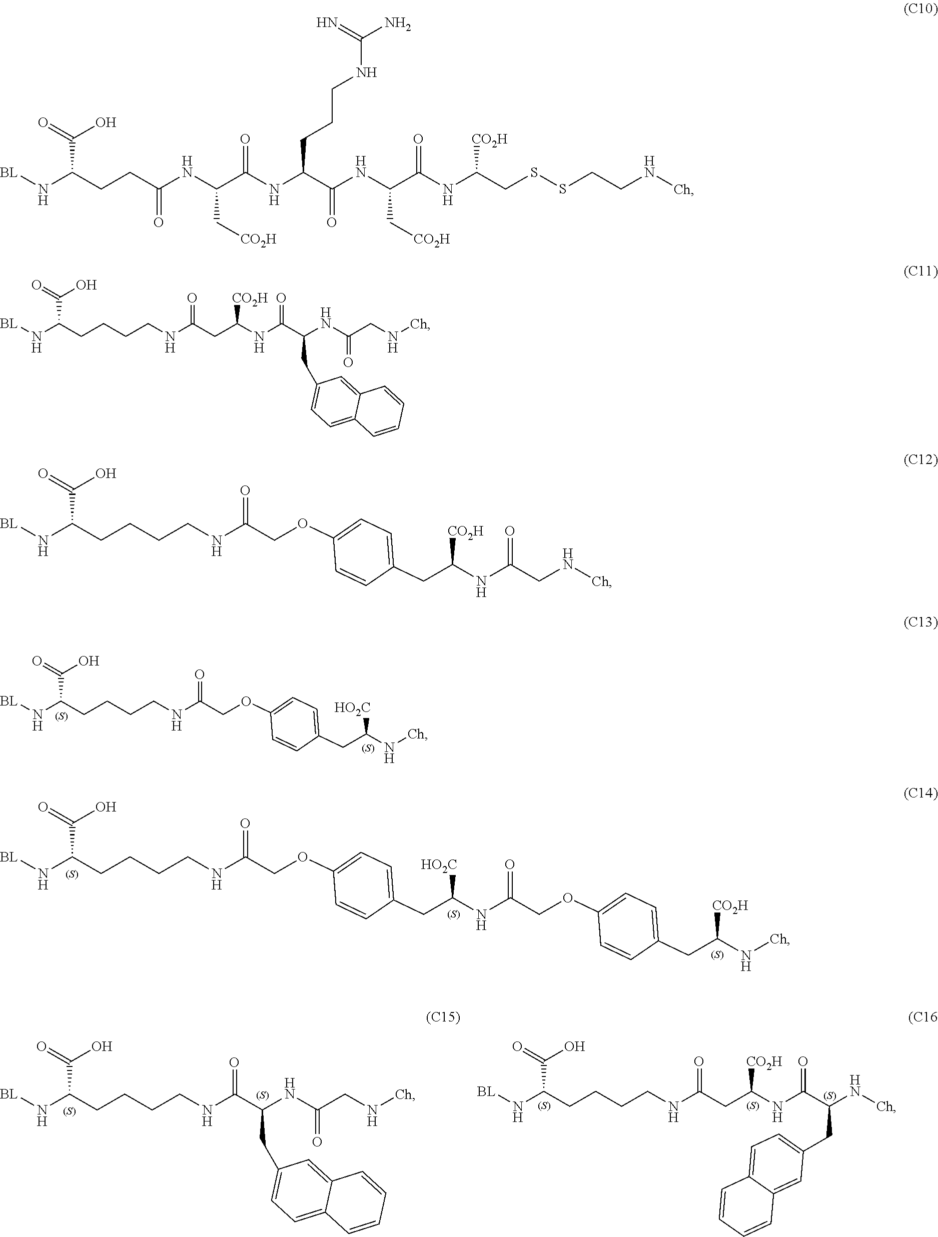

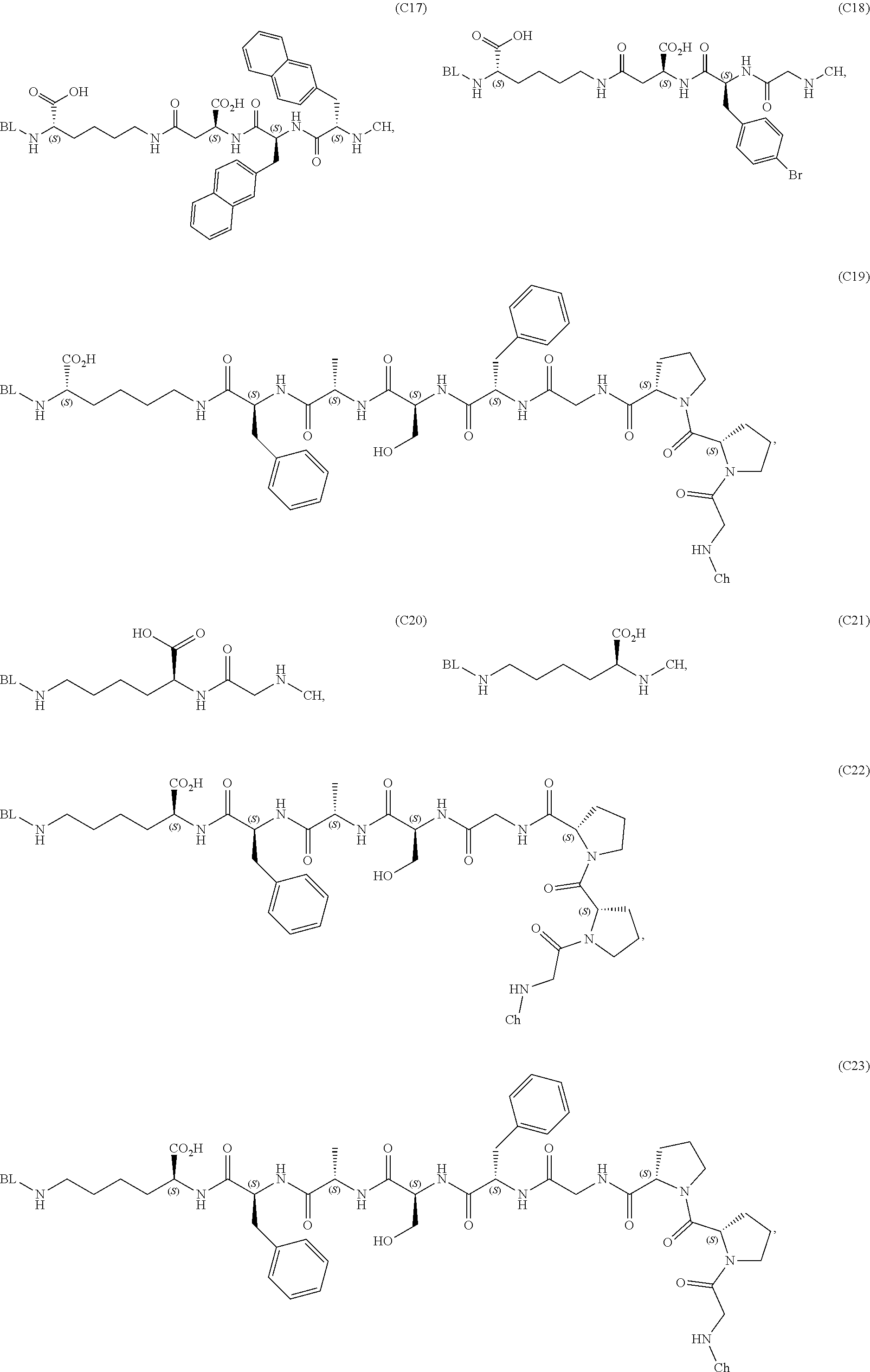

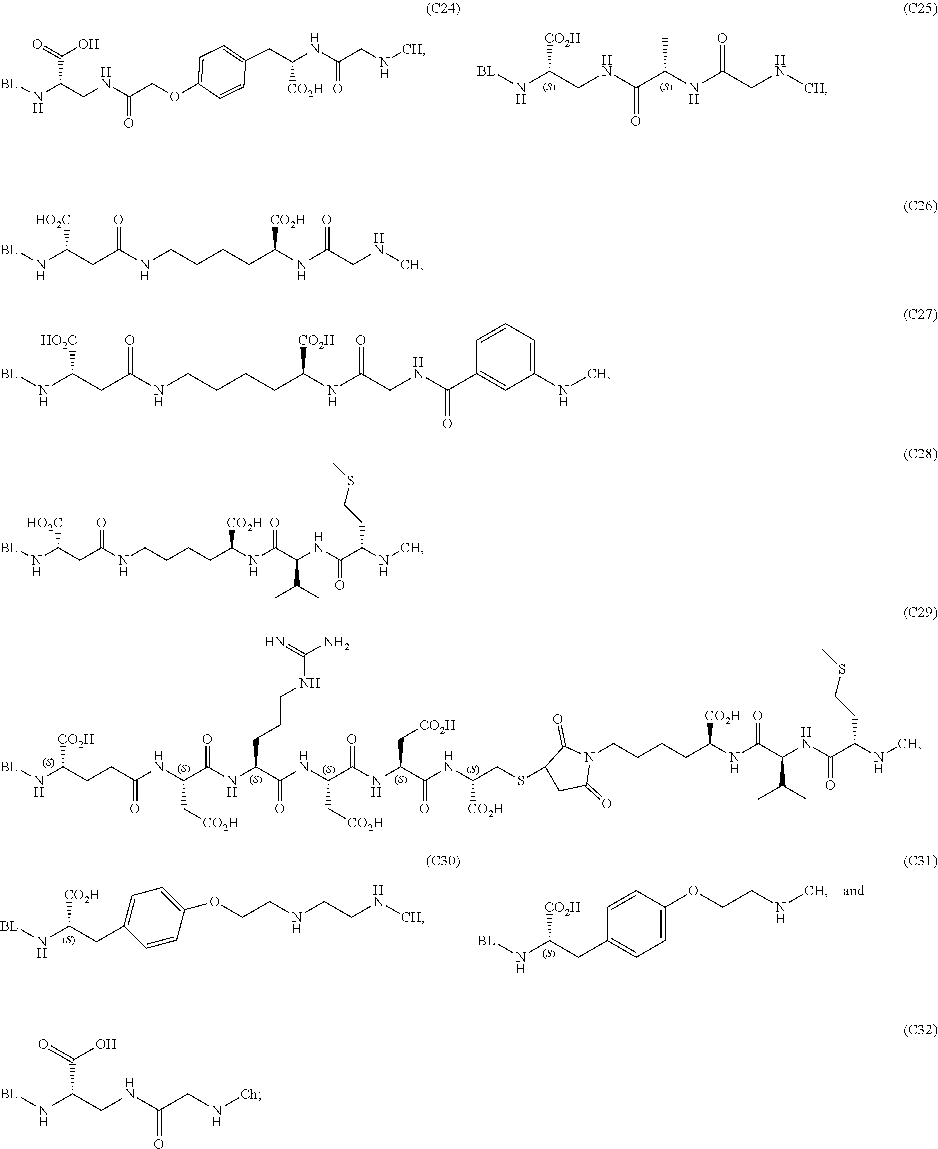

or a pharmaceutically acceptable salt thereof,
Embodiment 45: The compound of Embodiment 1, wherein the compound is a compound of any one of formula (C1) to (C32),

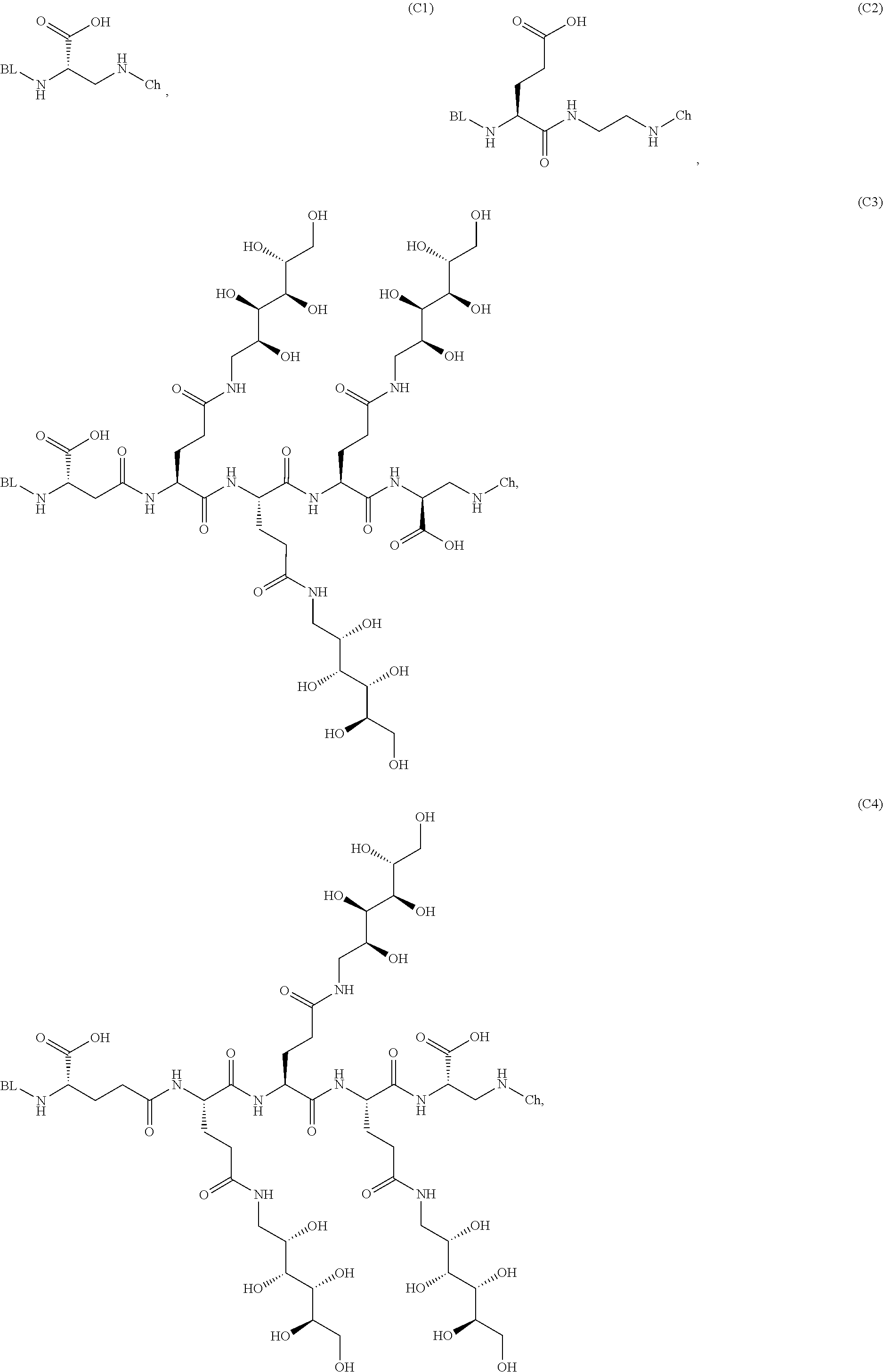

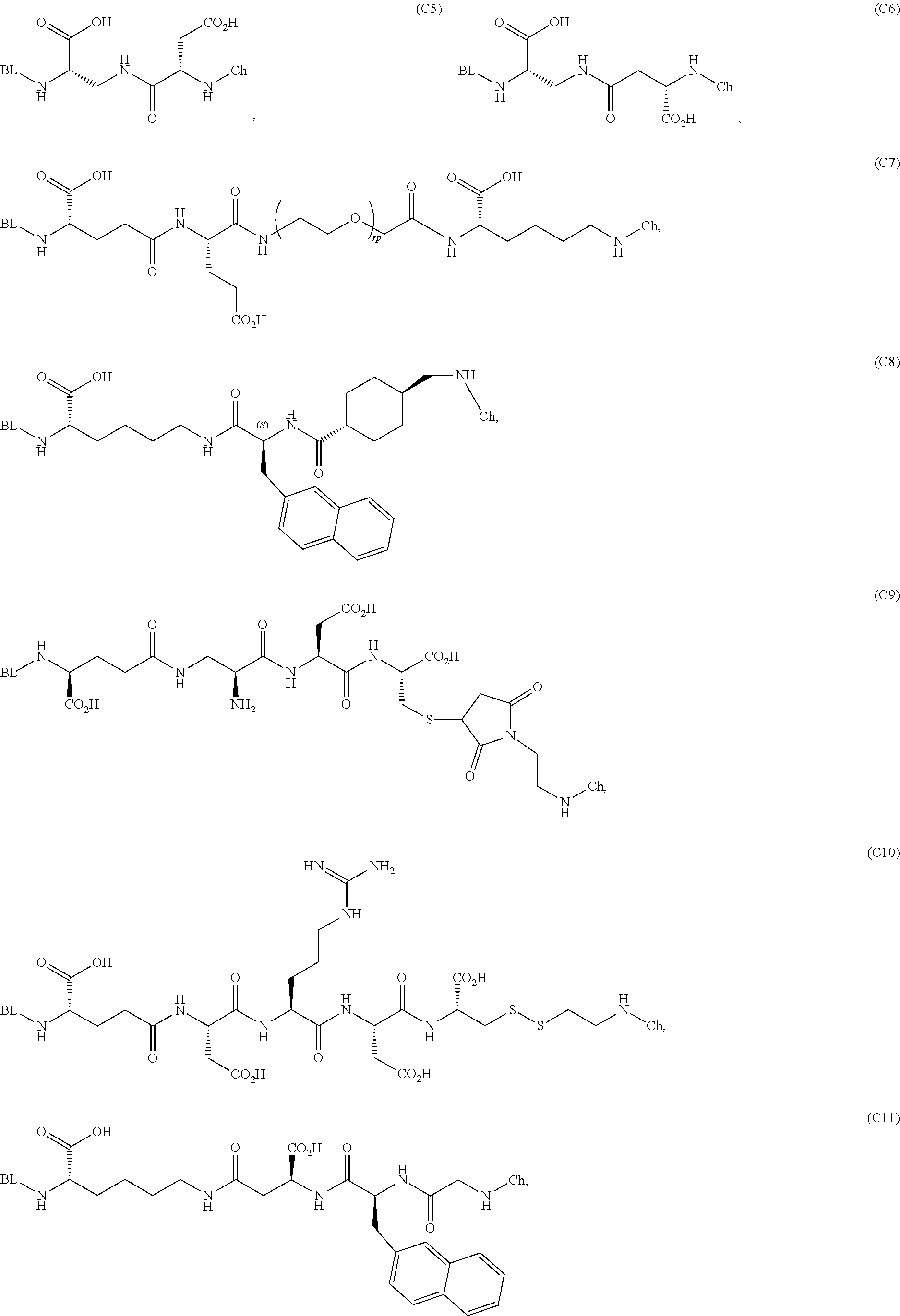

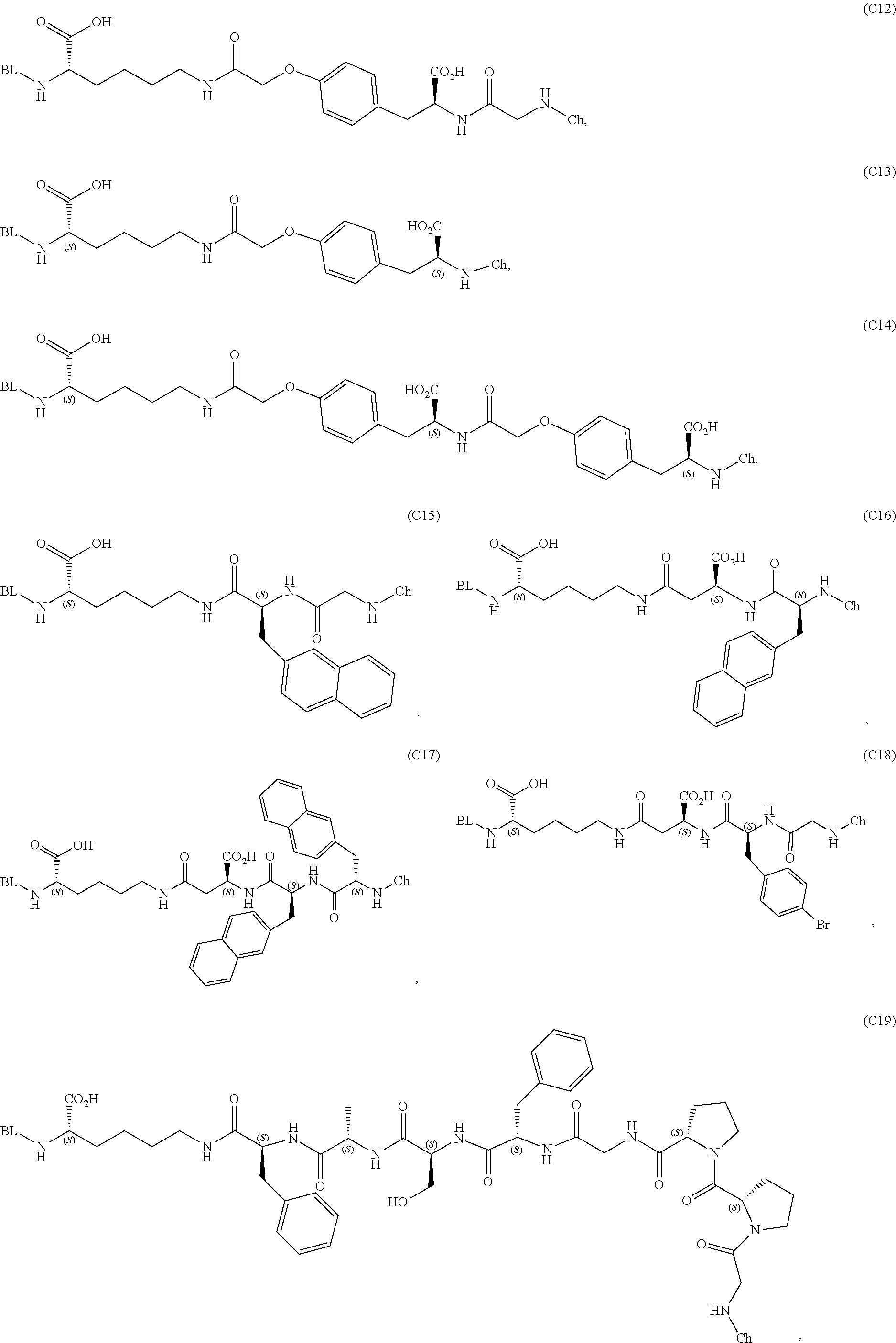

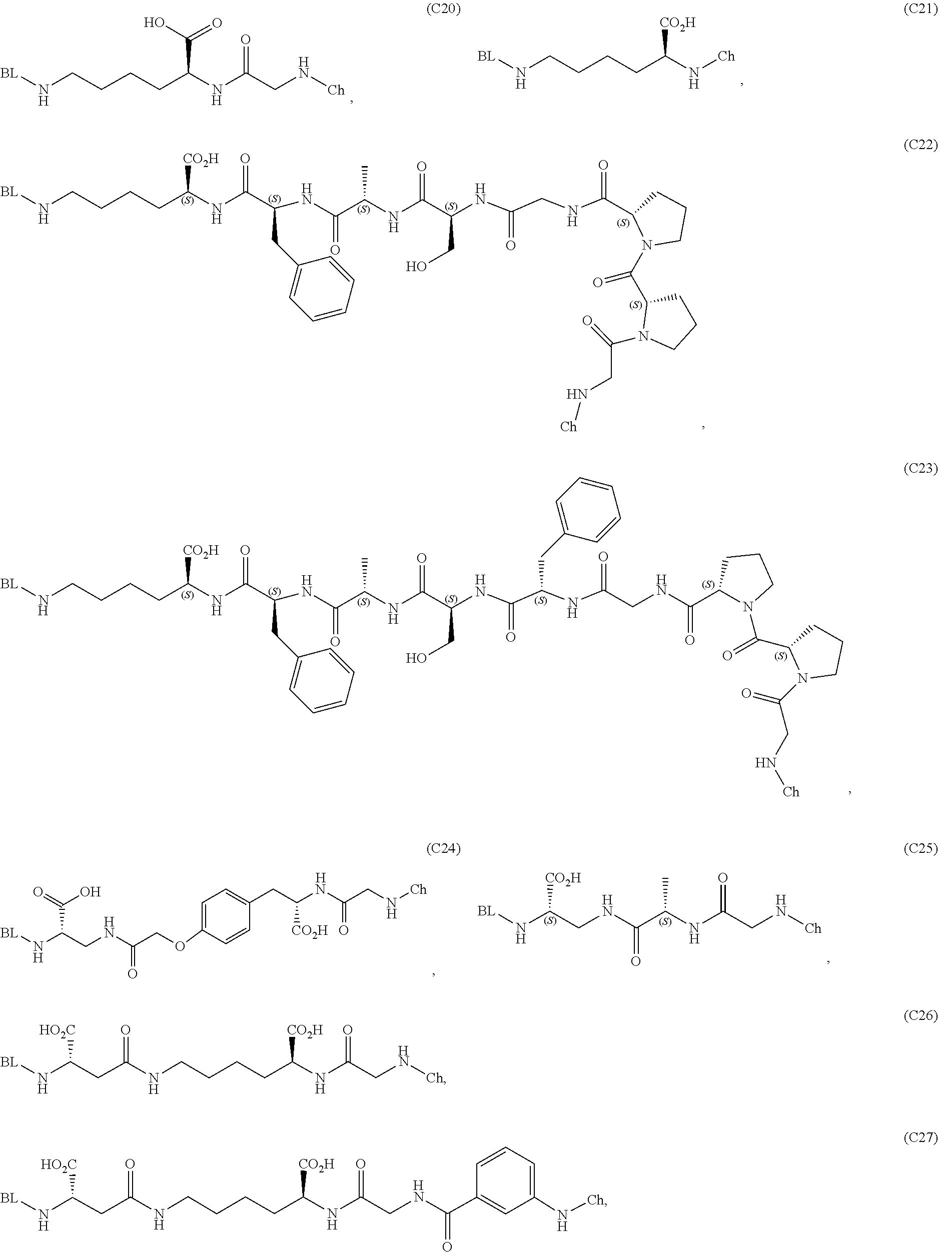

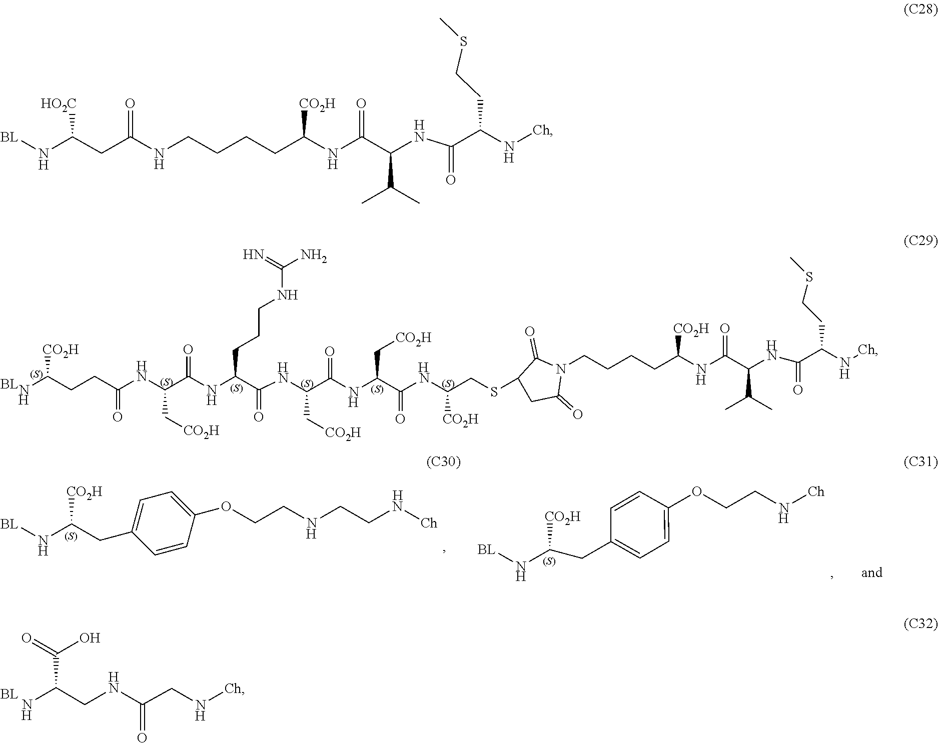

except that one group, corresponding to L^{x}, within said any one of formula (C1) to (C32) is replaced by a different L^{x}.
Embodiment 46: The compound of Embodiment 45, or a pharmaceutically acceptable salt thereof, wherein the one group, which is replaced by a different L^{x}, is an AA group, the different L^{x} is a different AA group, and the different AA group is a conservative amino acid substitution of the AA group.
Embodiment 47: The compound of any one of Embodiments 1 to 43, or a pharmaceutically acceptable salt thereof, wherein -(L^{x})ₖ- comprises a group of formula (III)

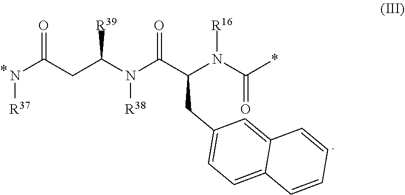

Embodiment 48: The compound of Embodiment 47, wherein R¹⁶, R³⁷ and R³⁸ are H.
Embodiment 49: The compound of Embodiment 47 or 48, wherein R³⁹ is —COOH.
Embodiment 50: The compound of Embodiment 1, wherein the compound is selected from

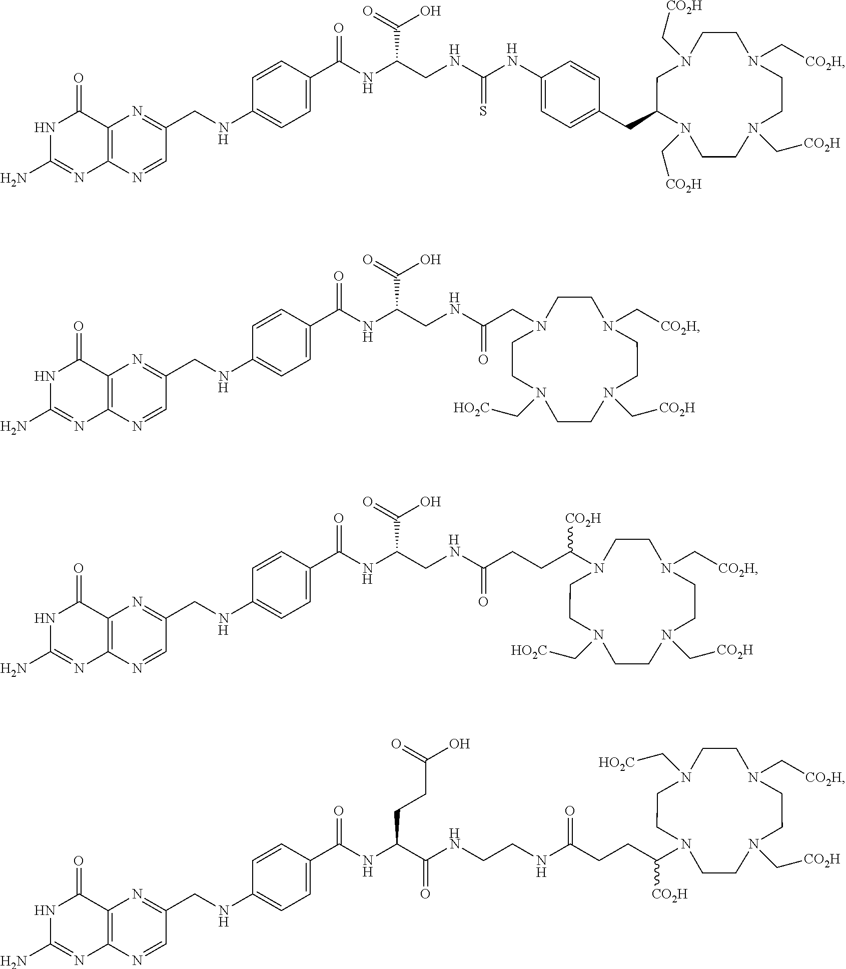

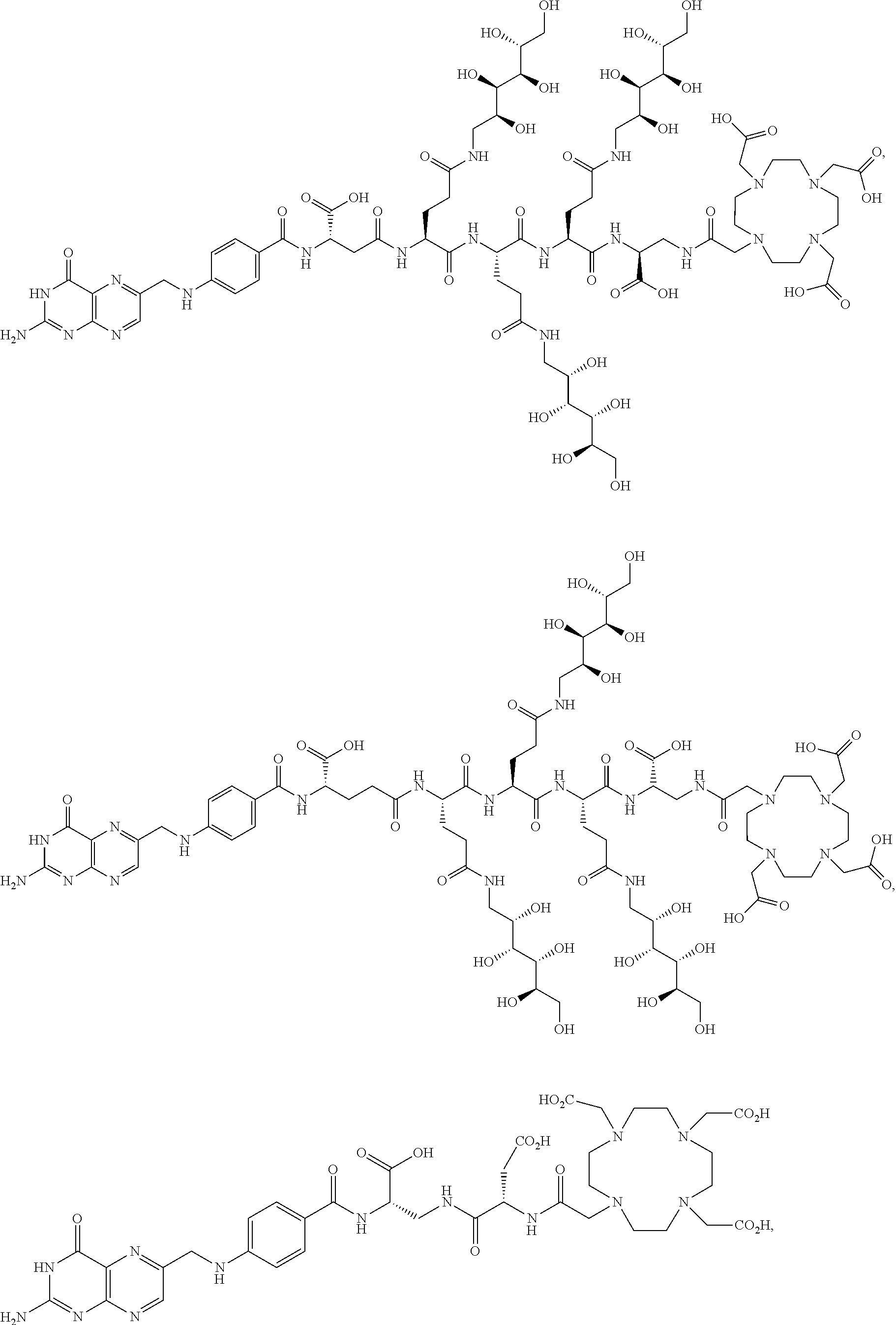

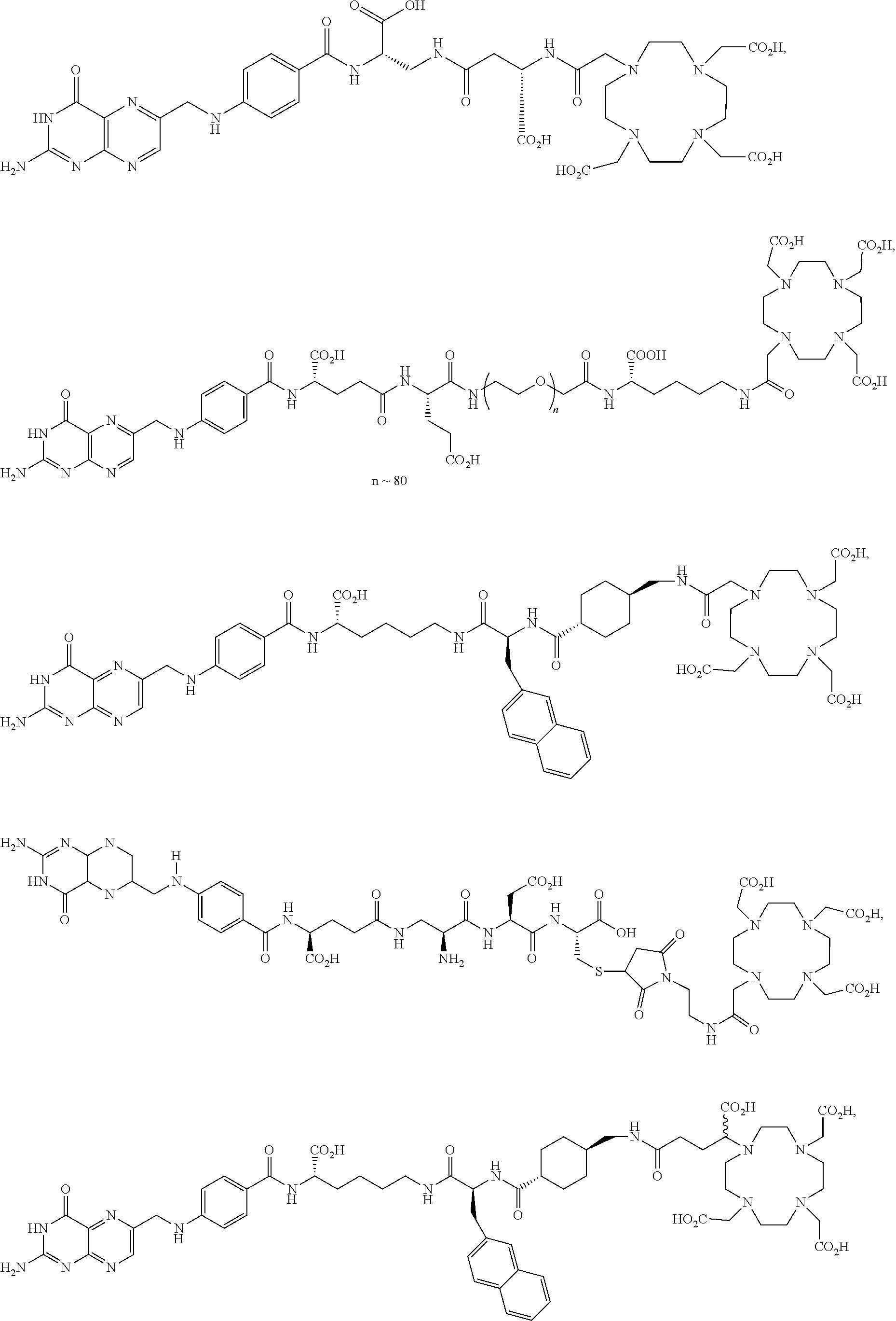

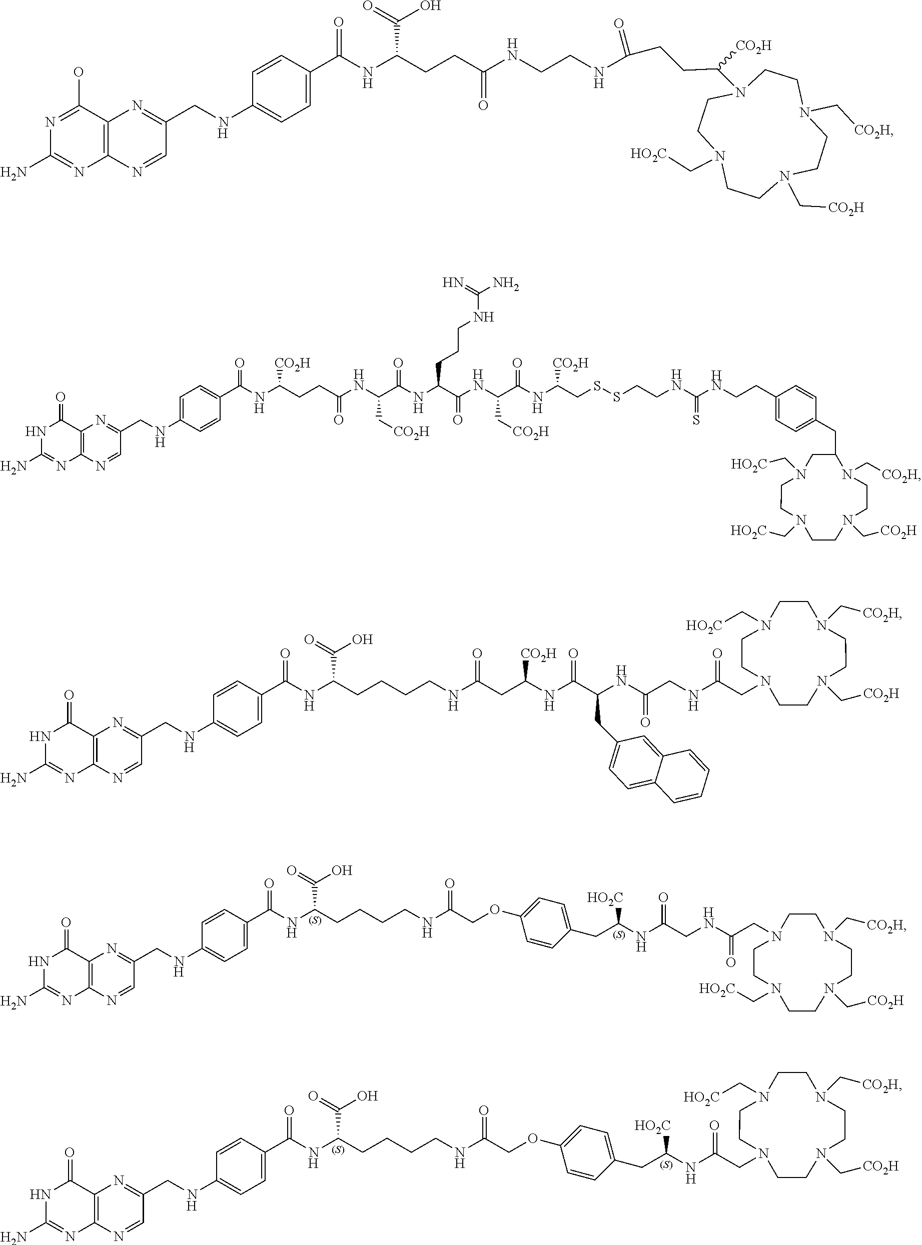

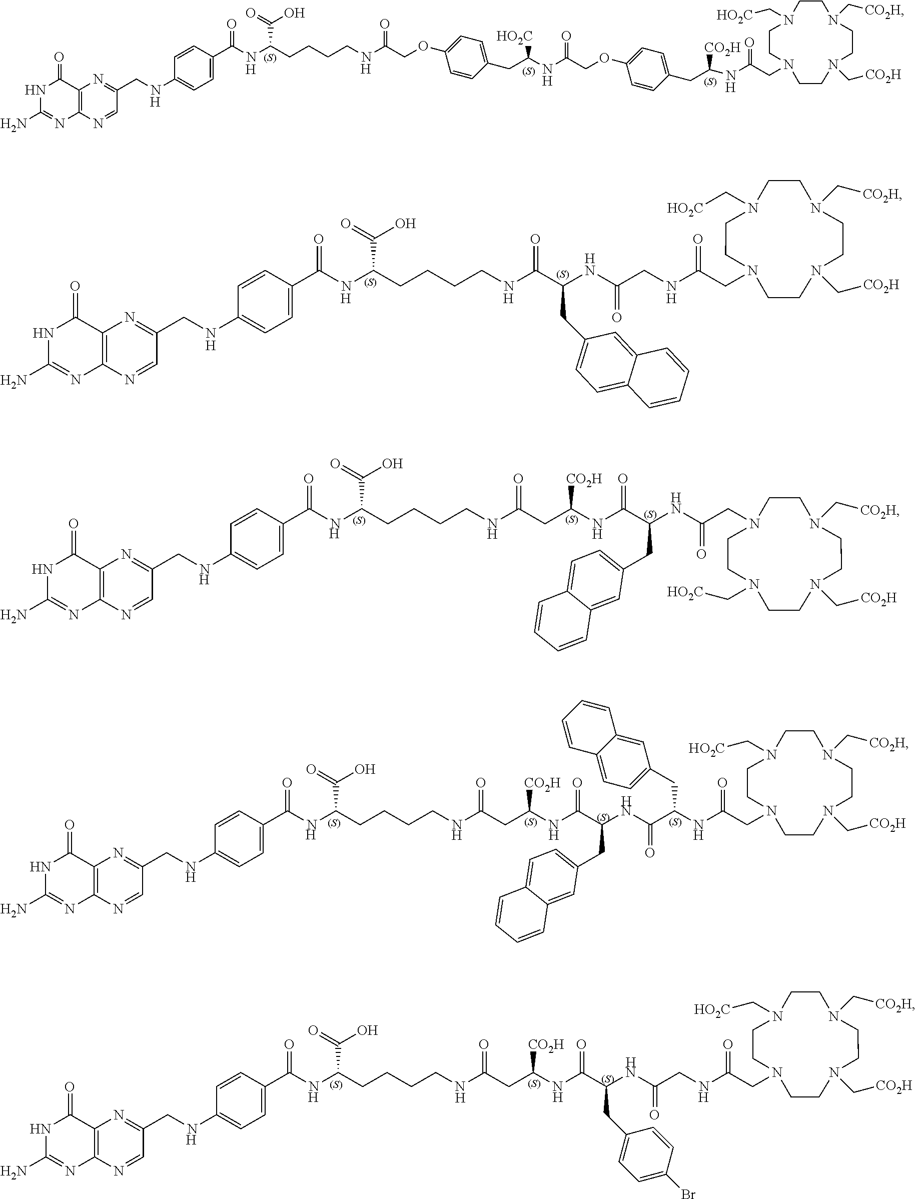

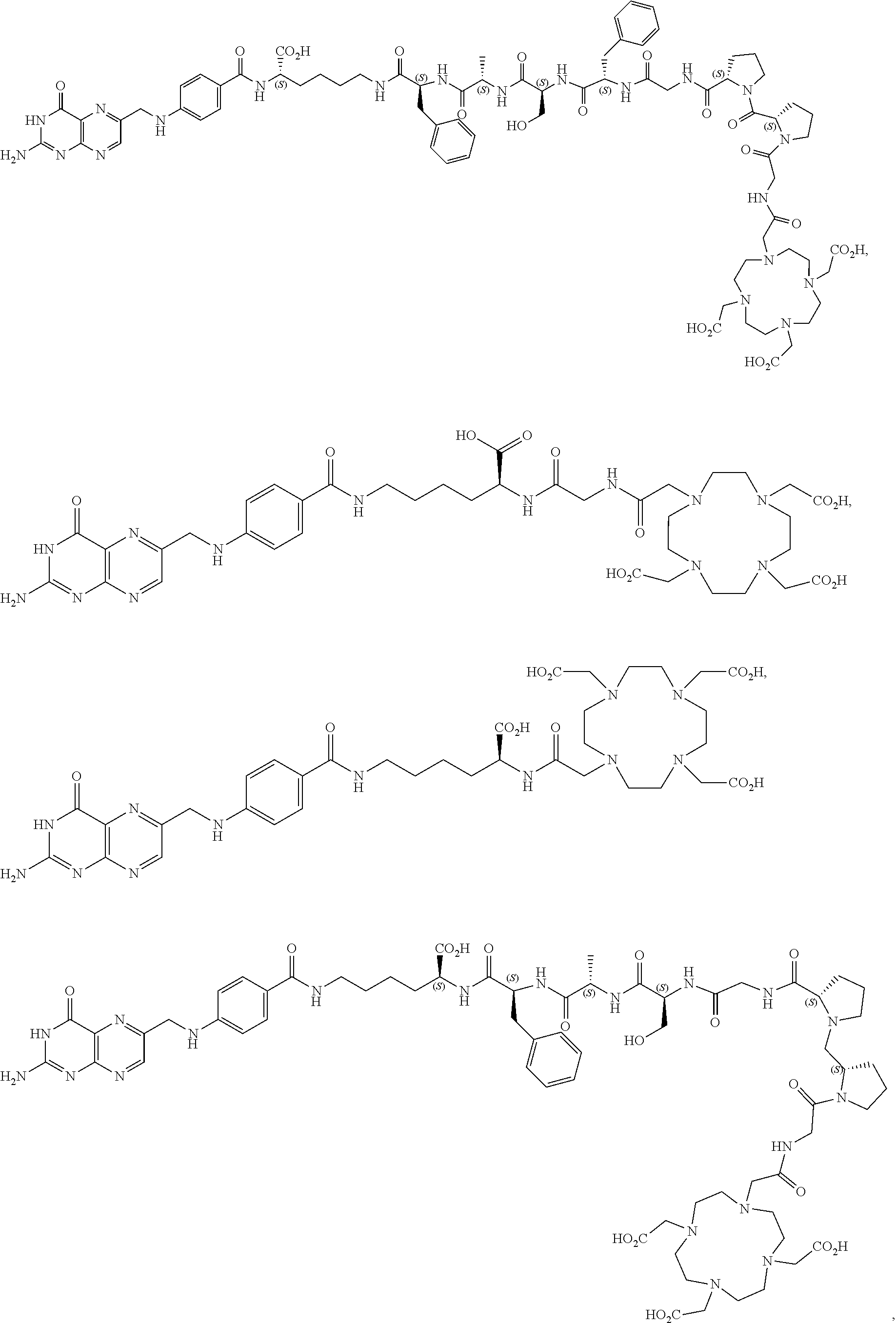

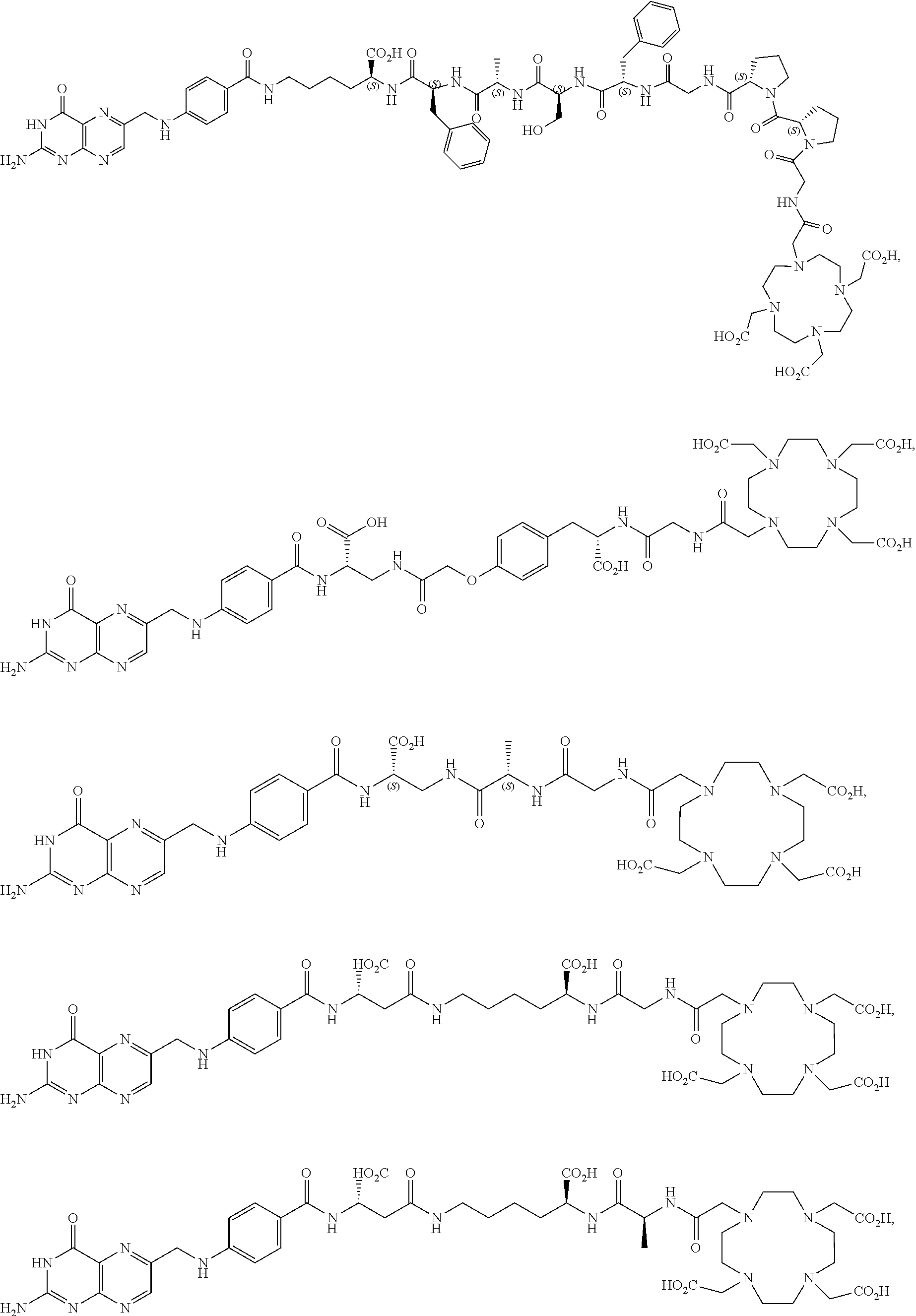

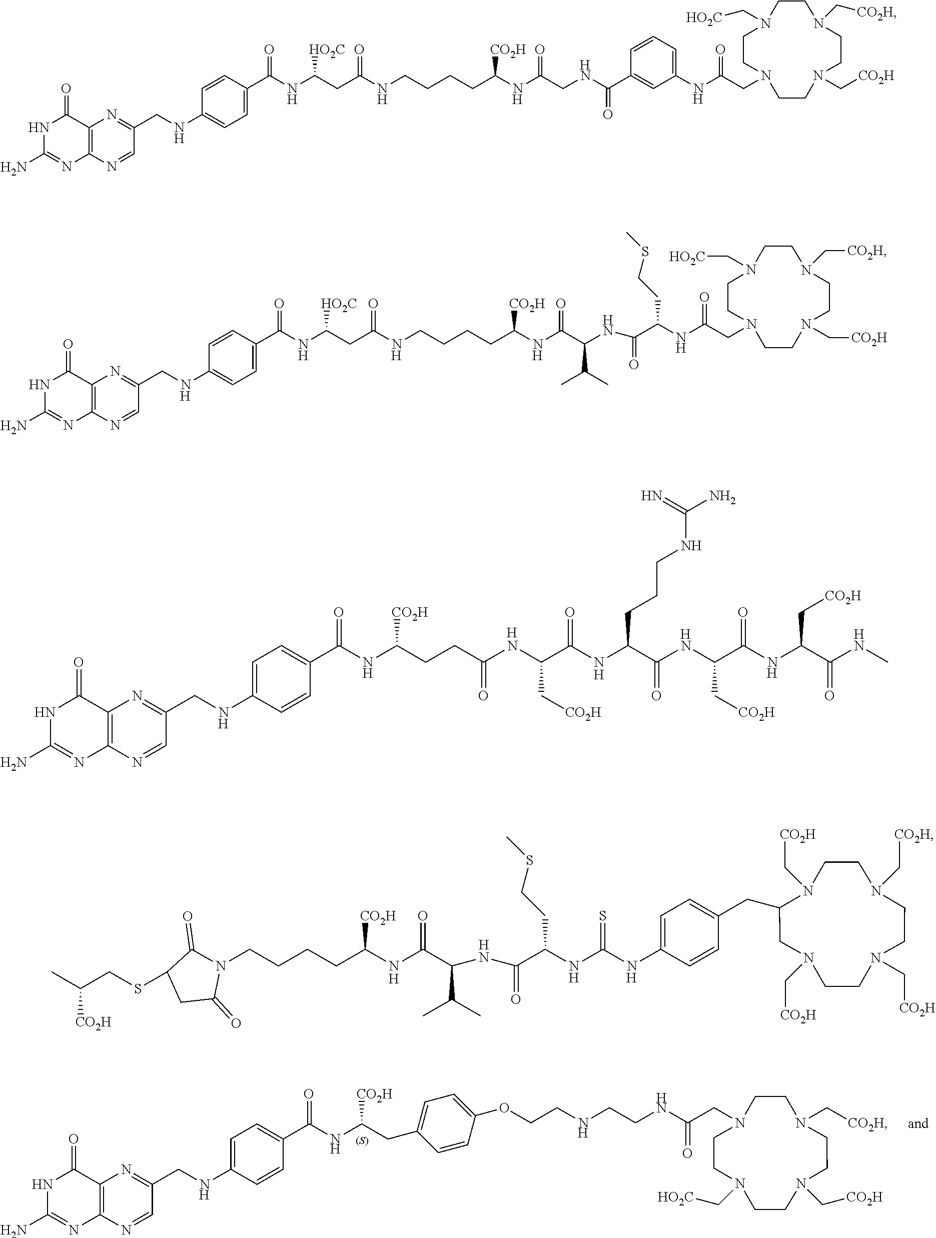

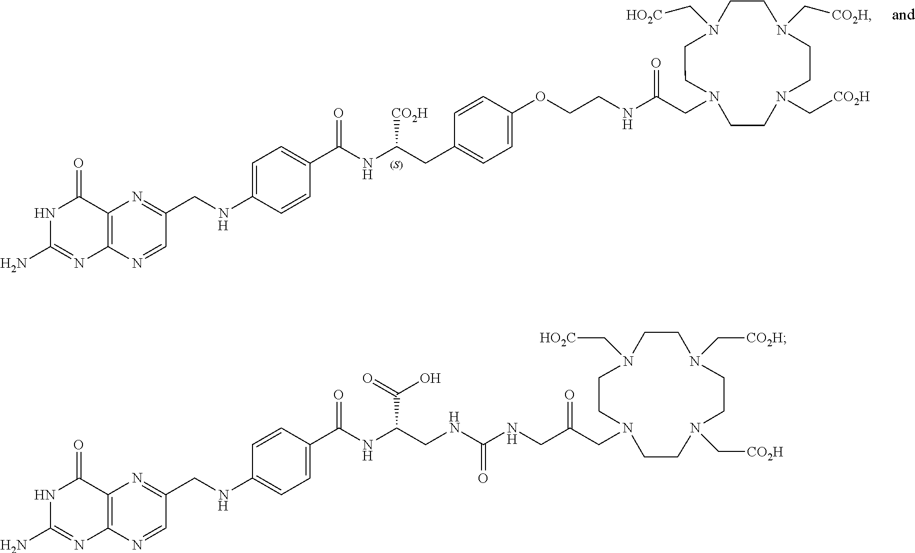

or a pharmaceutically acceptable salt thereof; wherein the chelating group exhibited in the above structural formulas can comprise a radioelement, Si-¹⁸F, B-¹⁸F, or Al-¹⁸F.
Embodiment 51: The compound of Embodiment 1, wherein the compound is a cold compound of formula

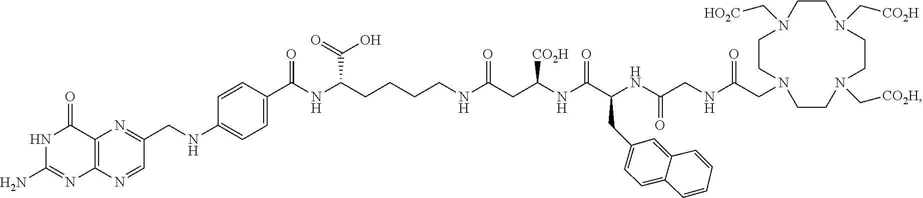 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof.
Embodiment 52: The compound of Embodiment 1, wherein the compound is a hot compound of formula

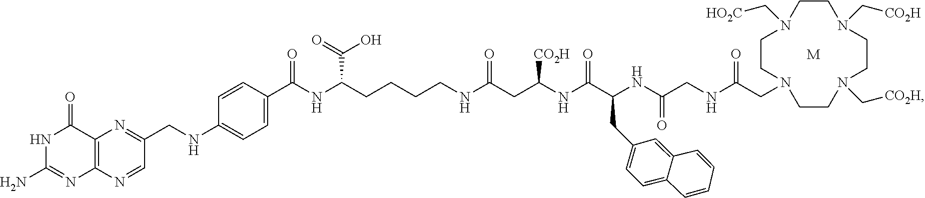

wherein M is ¹⁷⁷Lu or ²²⁵Ac; or a pharmaceutically acceptable salt thereof.
Embodiment 53: The compound of Embodiment 1, wherein the compound is a cold compound of formula

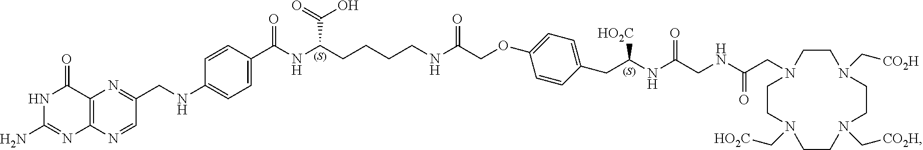

or a pharmaceutically acceptable salt thereof.
Embodiment 54: The compound of Embodiment 1, wherein the compound is hot compound of formula,

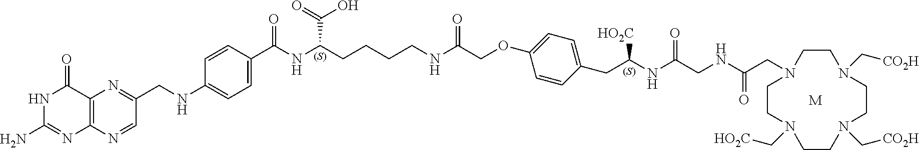

wherein M is ¹⁷⁷Lu or ²²⁵Ac; or a pharmaceutically acceptable salt thereof
Embodiment 55: The compound of any one of the preceding Embodiments, wherein the compound comprises a group of formula

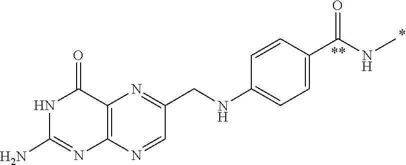 [SMILES: CNC(=O)c1ccc(NCc2cnc3nc(N)[nH]c(=O)c3n2)cc1]

and a carboxyl group in β, γδ, ε, or ζ position relative to the carbonyl indicated with “**” in above formula; or a pharmaceutically acceptable salt thereof.
Embodiment 56: The compound of any one of Embodiments 1, 2, 5, 6, 7, 11, 12, 16-36, 41-43, 47-49, and 50, or a pharmaceutically acceptable salt thereof, wherein PG is labeled with a radiohalogen selected from the group consisting of ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I and ²¹¹At.
Embodiment 57: A pharmaceutical composition comprising a compound according to any one of the preceding Embodiments, or a pharmaceutically acceptable salt thereof, and a pharmaceutically acceptable carrier.
Embodiment 58: A method of treating an FR expressing tumor or cell, the method comprising contacting the one or more FR expressing tumor or cell with an effective amount of a compound, or pharmaceutically acceptable salt thereof, according to any one of Embodiments 1 to 55 or with an effective amount of the pharmaceutical composition of Embodiment 57, wherein the compound comprises a chelating group which chelates a radioelement.
Embodiment 59: The method of Embodiment 58, wherein the FR expressing tumor or cell is in vitro, in-vivo, or ex vivo.
Embodiment 60: A method of treating a proliferative disease in a subject in need thereof, comprising administering to the subject a therapeutically effective amount of a compound, or a pharmaceutically acceptable salt thereof, according to any one of Embodiments 1 to 55, or a therapeutically effective amount of a pharmaceutical composition of Embodiment 57, wherein the compound comprises a chelating group which chelates a radioelement.
Embodiment 61: The method of Embodiment 60, wherein the proliferative disease is cancer.
Embodiment 62: The method of Embodiment 61, wherein the cancer is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.
Embodiment 63: The method of any one of Embodiments 60 to 62, further comprising administering to the subject an effective amount of folic acid.
Embodiment 64: The method of any one of Embodiments 60 to 63, further comprising administering to the subject an effective amount of an antifolate.
Embodiment 65: The method of any one of Embodiments 60 to 64, further comprising administering to the subject and effective amount of a radio-sensitizer.
Embodiment 66: The method of any one of Embodiments 60 to 65, wherein the subject is a human.
Embodiment 67: Use of a compound according to any one of Embodiments 1 to 55, or a pharmaceutically acceptable salt thereof, in the preparation of a medicament for the treatment of cancer.
Embodiment 68: A compound according to any one of Embodiments 1 to 55, or a pharmaceutically acceptable salt thereof, for use in a method of treating cancer in a subject.
Embodiment 69: A method for imaging FR expressing cells in a subject, comprising administering to the subject an effective amount of a compound, or a pharmaceutically acceptable salt thereof, according to any one of Embodiments 1 to 56, or an effective amount of a pharmaceutical composition of Embodiment 57, wherein the compound comprises a metal, a radioelement or radiohalogen.
Embodiment 70: A compound according to any one of Embodiments 1 to 55, wherein BL is of the formula

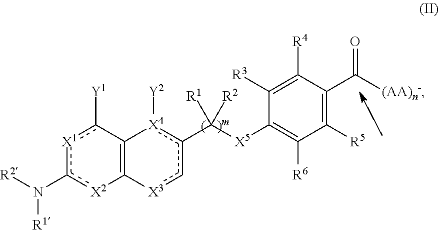

and the length in terms of number of atoms along the shortest path, counted from the atom belonging to (AA)ₙ or, if n is 0, belonging to (L^{x})ₖ, and covalently bonded to the carbonyl group shown adjacent to (AA)ₙ in Formula (II) (see arrow for the carbonyl group), to the atom covalently bonded to A, is between 6 and 50. In an aspect of Embodiment 70, the length is between 11 and 40 atoms. In a further aspect of Embodiment 70, the length is between 13 and 30 atoms. In a further aspect of Embodiment 70, the length is between 13 and 25 atoms. In a further aspect of Embodiment 70, the length is between 13 and 22 atoms. In a further aspect of Embodiment 70, the length is between 13 and 20 atoms. In a further aspect of Embodiment 70, the length is between 15 and 25 atoms. In a further aspect of Embodiment 70, the length is between 15 and 22 atoms.
Embodiment 71: A compound according to any one of Embodiments 1 to 55, wherein BL is of the formula

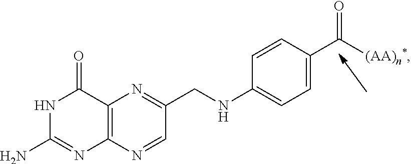

and the length in terms of number of atoms along the shortest path, counted from the atom belonging to (AA)ₙ or, if n is 0, belonging to (L^{x})ₖ, and covalently bonded to the carbonyl group shown adjacent to (AA)ₙ in above formula (see arrow for the carbonyl group), to the atom covalently bonded to A, is between 6 and 50. In an aspect of Embodiment 71, the length is between 11 and 40 atoms. In a further aspect of Embodiment 71, the length is between 13 and 30 atoms. In a further aspect of Embodiment 71, the length is between 13 and 25 atoms. In a further aspect of Embodiment 71, the length is between 13 and 22 atoms. In a further aspect of Embodiment 71, the length is between 13 and 20 atoms. In a further aspect of Embodiment 71, the length is between 15 and 25 atoms. In a further aspect of Embodiment 70, the length is between 15 and 22 atoms.
Embodiment 72: A compound according to any one of Embodiments 1 to 55, wherein the compound, when not radiolabeled, has a molecular weight of between 800 Da and 2500 Da.
Embodiment 73: A compound according to any one of Embodiments 1 to 55, wherein the compound, when not radiolabeled, has a molecular weight of between 1000 Da and 1500 Da.
Embodiment 74: A compound according to any one of Embodiments 1 to 55, wherein the compound, when not radiolabeled, has a molecular weight of between 1000 Da and 1300 Da. Embodiment 75: A compound according to any one of Embodiments 1 to 55, wherein the compound, when not radiolabeled, has a molecular weight of between 1000 Da and 1200 Da.
Embodiment 76: A compound according to any one of Embodiments 1 to 55, wherein the compound, when not radiolabeled, has a molecular weight of between 1000 Da and 1150 Da.
Embodiment 77: A compound according to any one of Embodiments 1 to 55, wherein the compound, when not radiolabeled, has a molecular weight of between 1100 Da and 1200 Da.
Embodiment 78: A compound of structural formula

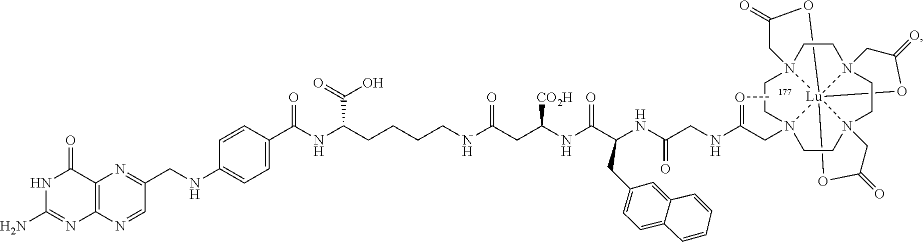

or a pharmaceutically acceptable salt thereof
Embodiment 80: A method of treating cancer in a subject in need thereof, comprising administering to the subject a therapeutically effective amount of a compound of the following structural formula,

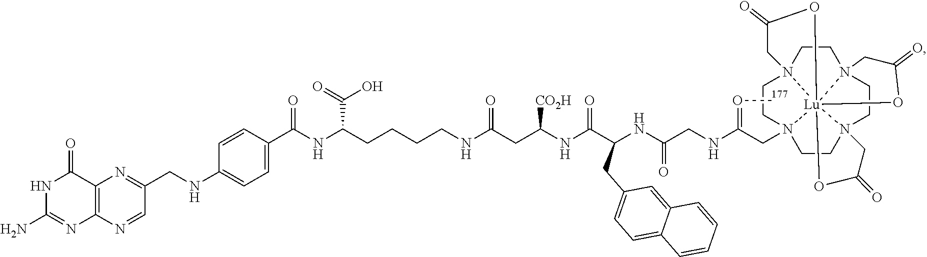

or a pharmaceutically acceptable salt thereof. In an aspect of this embodiment, the cancer is ovarian cancer. In a further aspect of this embodiment the cancer is non-small cell lung cancer.
Embodiment 81: A compound of structural formula

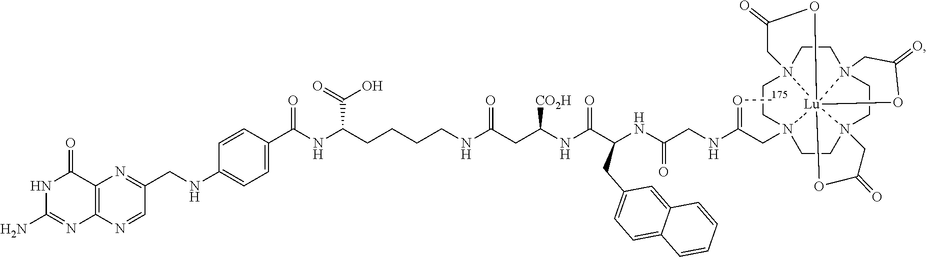

or a pharmaceutically acceptable salt thereof.
Embodiment 82: A compound of structural formula

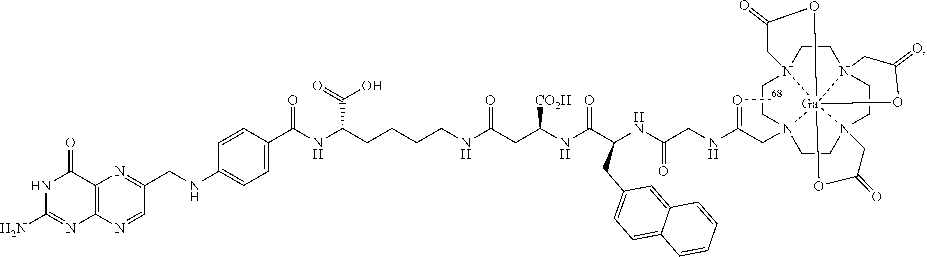

or a pharmaceutically acceptable salt thereof.
Embodiment 83: A method for diagnosing cancer in a subject, comprising administering to the subject an effective amount of a compound of the following structural formula,

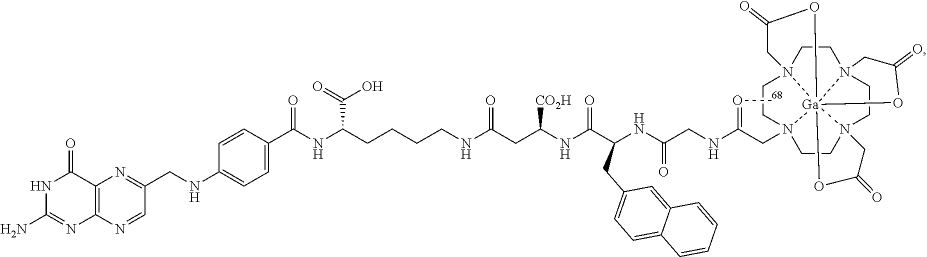

or a pharmaceutically acceptable salt thereof.
Embodiment 84: A compound of structural formula

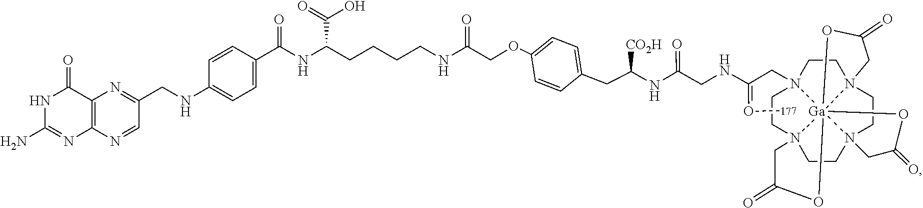

or a pharmaceutically acceptable salt thereof.
Embodiment 85: A method of treating cancer in a subject in need thereof, comprising administering to the subject a therapeutically effective amount of a compound of the following structural formula,

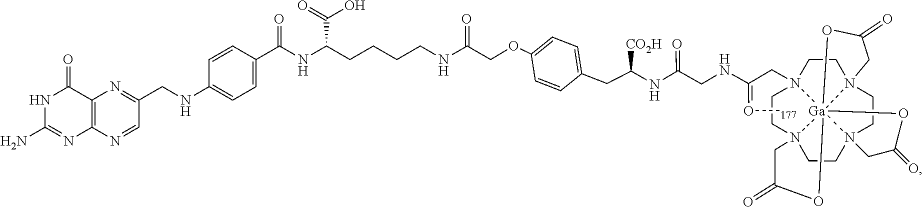

or a pharmaceutically acceptable salt thereof. In an aspect of this embodiment, the cancer is ovarian cancer. In a further aspect of this embodiment the cancer is non-small cell lung cancer.
Embodiment 86: A compound of structural formula

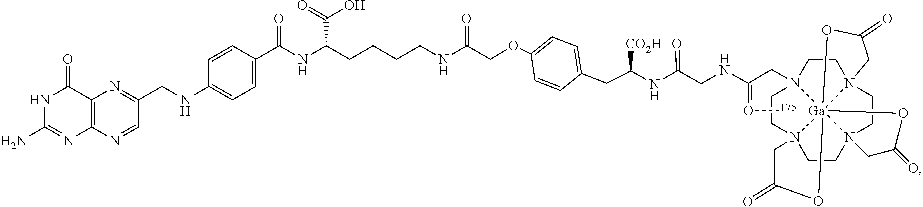

or a pharmaceutically acceptable salt thereof.
Embodiment 87: A compound of structural formula

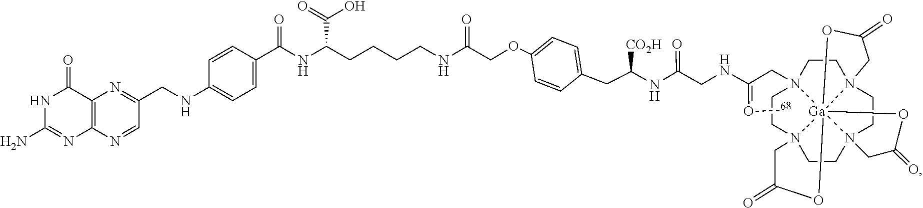

or a pharmaceutically acceptable salt thereof.
Embodiment 88: A method for diagnosing cancer in a subject, comprising administering to the subject an effective amount of a compound of the following structural formula,

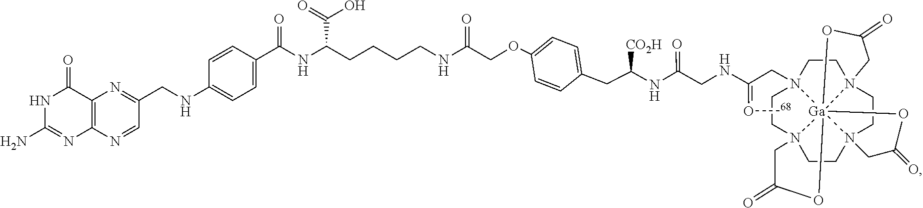

or a pharmaceutically acceptable salt thereof.

In some embodiments, the compound of the present disclosure, when not radiolabeled, has a molecular weight of between 800 Da and 4000 Da, between 800 Da and 3500 Da, between 800 Da and 3000 Da, between 800 Da and 2500 Da, between 800 Da and 2000 Da, between 800 Da and 1800 Da, between 800 Da and 1700 Da, between 800 Da and 1600 Da, between 800 Da and 1500 Da, between 800 Da and 1400 Da, between 800 Da and 1300 Da, between 1000 Da and 2000 Da, between 1000 Da and 1800 Da, between 1000 Da and 1700 Da, between 1000 Da and 1600 Da, between 1000 Da and 1500 Da, between 1000 Da and 1400 Da, between 1000 Da and 1300 Da, between 1000 Da and 1200 Da, or between 1000 Da and 1150 Da, or between 1100 Da and 1200 Da.

In some embodiments, the compound of the present disclosure, or a pharmaceutically acceptable salt thereof, does not comprise an L² group.

In some embodiments, the compound of the present disclosure, or a pharmaceutically acceptable salt thereof, has an (L^{x})ₖ group which only comprises AA, L¹ and L³ groups.

In another embodiment, a compound of the present disclosure, or pharmaceutically acceptable salt thereof, is of formula BL-L³-Ch, BL-L¹-L³-Ch, BL-L³-L³-L¹-L¹-L¹-L³-Ch, BL-L³-L¹-Ch, BL-L³-L³-L³-Ch, BL-L³-L³-L¹-L³-L³-Ch, BL-L³-L¹-L³-Ch, BL-L³-L³-AA-L¹-L²-L³-Ch, BL-L³-L³-L¹-L¹-L¹-L²-Ch, BL-L³-L³-L³-L¹-AA-Ch, BL-L³-L³-AA-Ch, BL-L³-L³-Ch, BL-L³-L¹-AA-Ch, BL-L³-L³-L³-L¹-Ch, BL-L³-L³-L³-L¹-L¹-Ch, BL-L³-L¹-L¹-L¹-L¹-AA-AA-AA-AA-Ch, BL-L³-AA-Ch, BL-L³-L¹-L¹-L¹-AA-AA-AA-AA-Ch, or BL-L³-L³-L³-AA-Ch, BL-L³-L³-L³-L³-Ch, wherein each AA independently is an amino acid residue (i.e., if more than one AA is indicated in a formula above, the AA groups can all be different, all be the same, or some are different and some are the same), and each of L¹, L², L³, BL, and Ch are independently as described herein (i.e., if more than one L¹ is indicated in a formula above, the L¹ groups can all be different, all be the same, or some are different and some are the same; if more than one L³ is indicated in a formula above, the L³ groups can all be different, all be the same, or some are different and some are the same).

In another embodiment, a compound of the present disclosure, or pharmaceutically acceptable salt thereof, is of formula BL-L³-Ch, BL-L¹-L¹-L¹-L³-Ch, BL-L¹-Ch, BL-L³-L³-Ch, BL-L¹-L³-L³-Ch, BL-L¹-L³-Ch, BL-L³-L³-AA-L¹-L²-L³-Ch, BL-L¹-L¹-L¹-L²-Ch, BL-L³-L³-L¹-AA-Ch, BL-L³-AA-Ch, BL-L¹-AA-Ch, BL-L³-L³-L¹-Ch, BL-L³-L¹-Ch, BL-L³-L³-L¹-L¹-Ch, BL-L³-L¹-L¹-Ch, BL-L¹-L¹-L¹-L¹-AA-AA-AA-AA-Ch, BL-AA-Ch, BL-L¹-L¹-L¹-AA-AA-AA-AA-Ch, or BL-L¹-L¹-L¹-L¹-L²-L³-L¹-L¹-Ch, and BL comprises one amino acid residue covalently attached to a pteryl group or derivative thereof; wherein each AA independently is an amino acid residue (i.e., if more than one AA is indicated in a formula above, the AA groups can all be different, all be the same, or some are different and some are the same), and each of L¹, L², L³, BL, and Ch are independently as described herein (i.e., if more than one L¹ is indicated in a formula above, the L¹ groups can all be different, all be the same, or some are different and some are the same; if more than one L³ is indicated in a formula above, the L³ groups can all be different, all be the same, or some are different and some are the same).

In another embodiment, a compound of the present disclosure, or a pharmaceutically acceptable salt thereof, is of formula BL-L^{x}-L^{a}-L^{x}-Ch, BL-L^{x}-L^{x}-L^{a}-L^{x}-Ch, BL-L^{x}-L^{x}-L^{a}-Ch, or BL-L^{x}-L^{x}-L^{a}-L^{a}-Ch, wherein L^{a} is

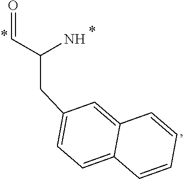 [SMILES: NC(C=O)Cc1ccc2ccccc2c1]

each L^{x} independently is AA, L¹, or L³, and AA, L¹, L³, BL and Ch are as described herein, for example, for Embodiment 1 or 2. More specifically, L^{a} can be

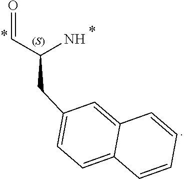

In another embodiment, a compound of the present disclosure, or a pharmaceutically acceptable salt thereof, is of formula BL-L^{a}-L^{x}-Ch, BL-L^{x}-L^{a}-L^{x}-Ch, BL-L^{x}-L^{a}-Ch, or BL-L^{x}-L^{a}-La-Ch, wherein L^{a} is

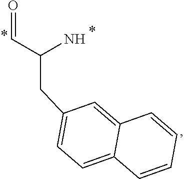 [SMILES: NC(C=O)Cc1ccc2ccccc2c1]

and each L^{x} independently is AA, L¹, or L³, and AA, L¹, L³, BL and Ch are as described herein, for example, for Embodiment 1 or 2. More specifically, L^{a} can be

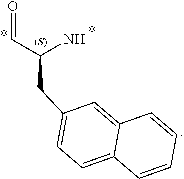

In another embodiment, a compound of the present disclosure, or a pharmaceutically acceptable salt thereof, is of formula BL-L^{x}-L^{b}-L^{x}-Ch, BL-L^{x}-L^{b}-Ch, or BL-L^{x}-L^{b}-L^{b}-Ch, wherein L^{b} is

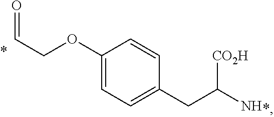 [SMILES: NC(Cc1ccc(OCC=O)cc1)C(=O)O]

and each L^{x} independently is AA, L¹, or L³, and AA, L¹, L³, BL and Ch are as described herein, for example, for Embodiment 1 or 2. More specifically, L^{b} can be

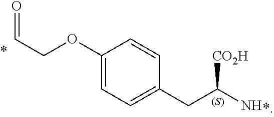

In another embodiment, a compound of the present disclosure, or a pharmaceutically acceptable salt thereof, is of formula BL-L^{b}-L^{x}-Ch, BL-L^{b}-Ch, or BL-L^{b}-L^{b}-Ch, wherein L^{b} is

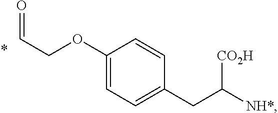

and each L^{x} independently is AA, L¹, or L³, and AA, L¹, L³, BL and Ch are as described herein, for example, for Embodiment 1 or 2. More specifically, L^{b} can be

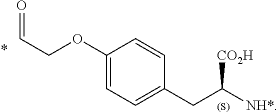

In another embodiment, a compound of the present disclosure is of any one of formula (C1) to (C32):

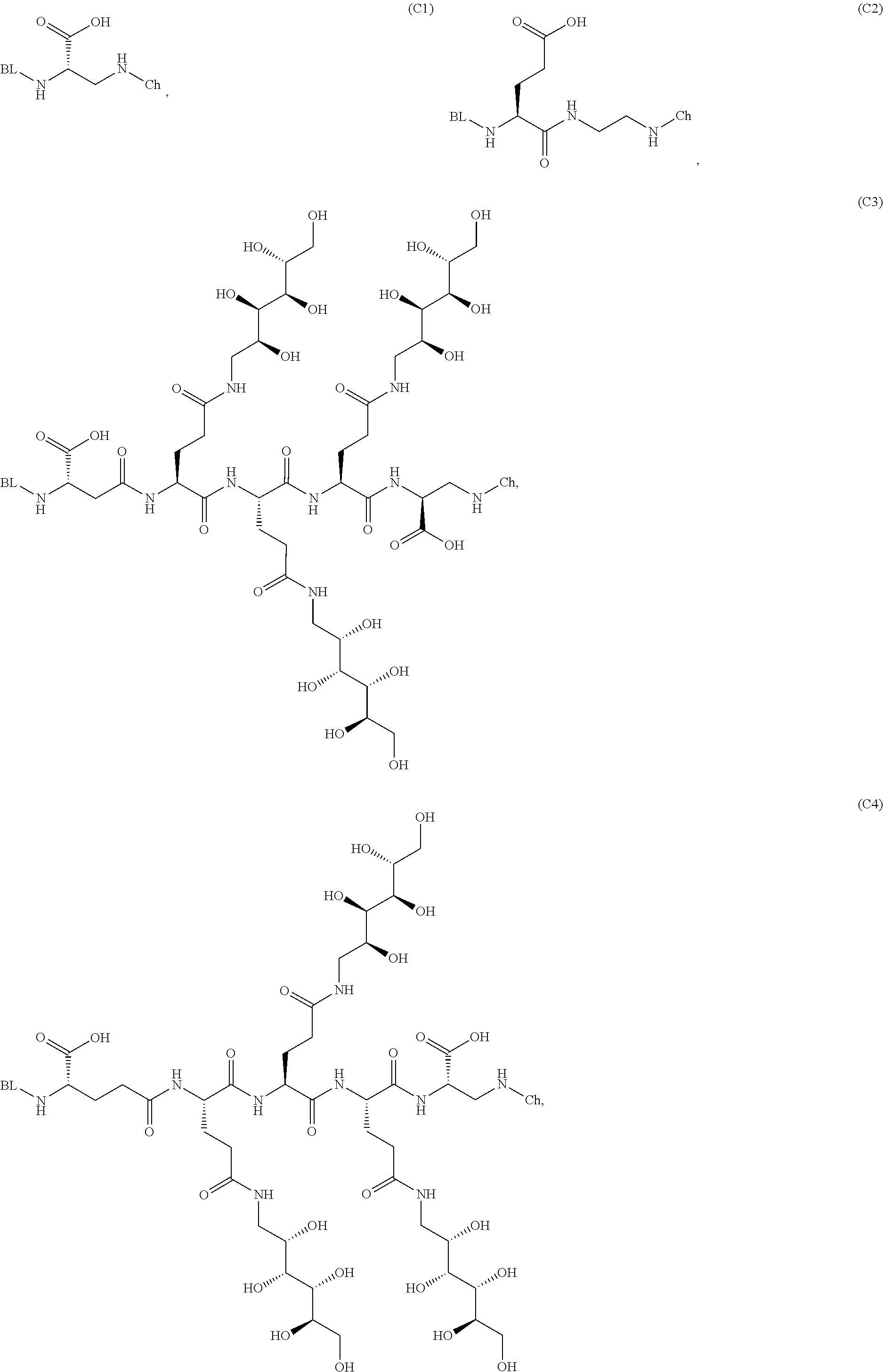

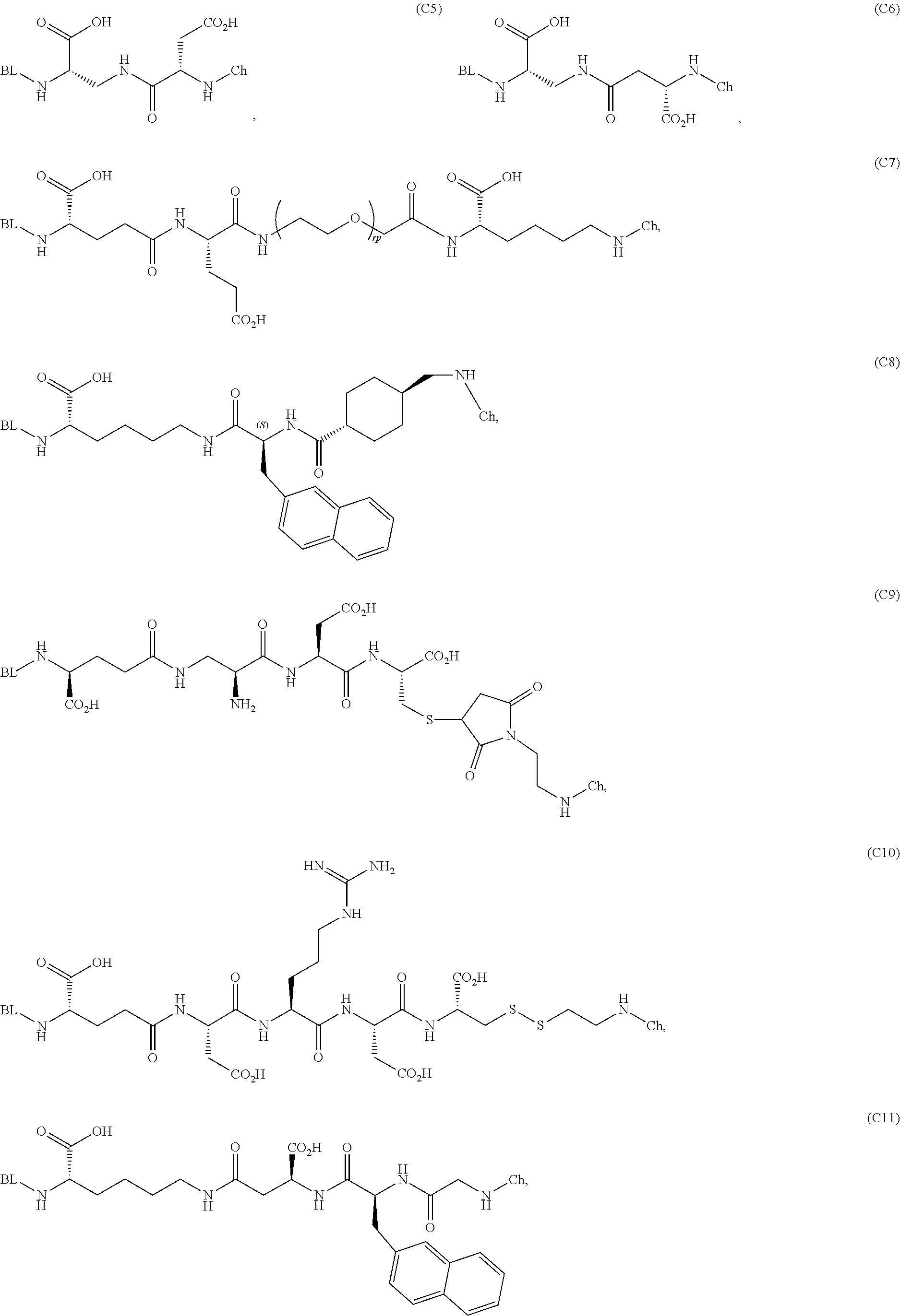

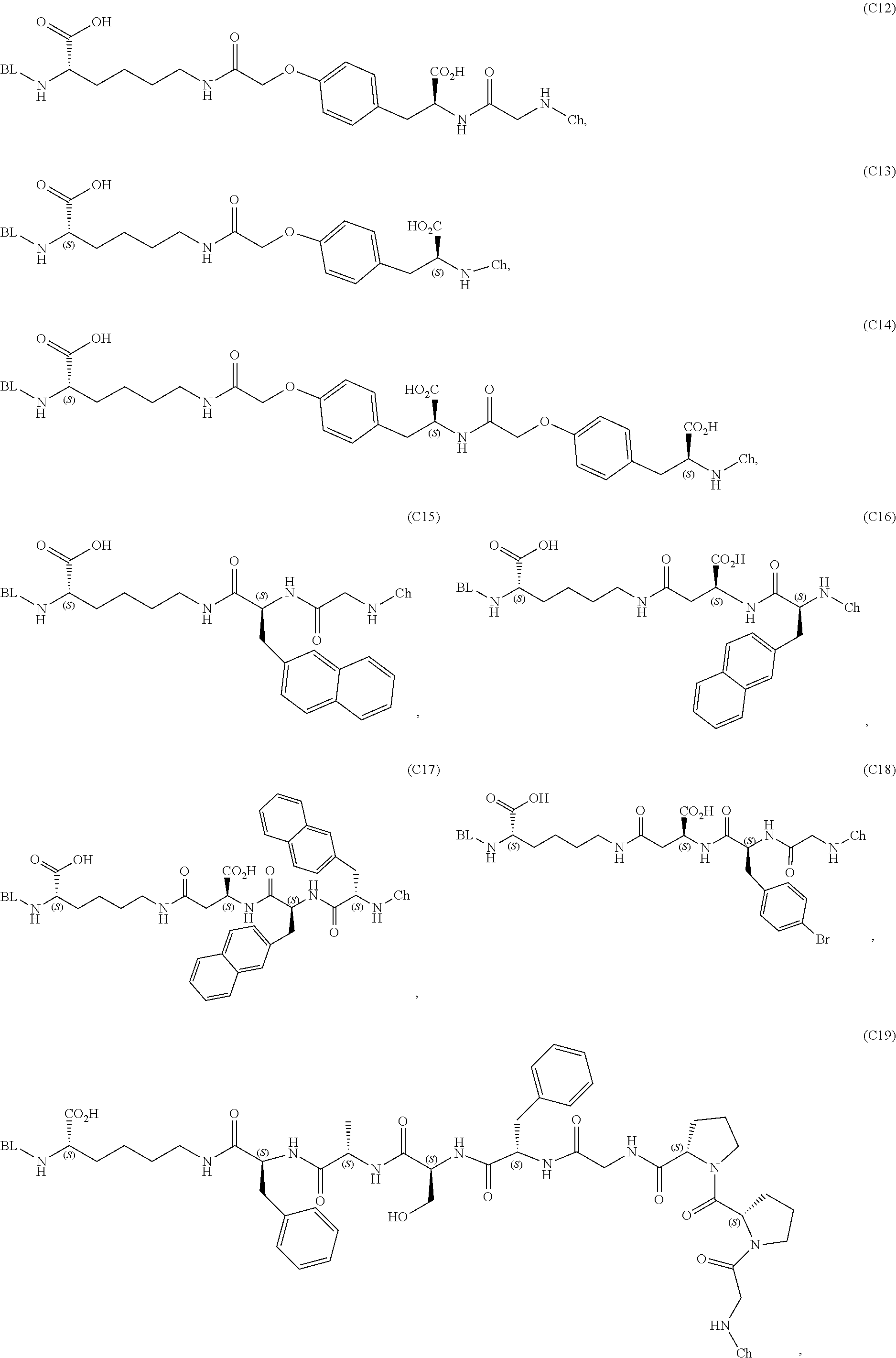

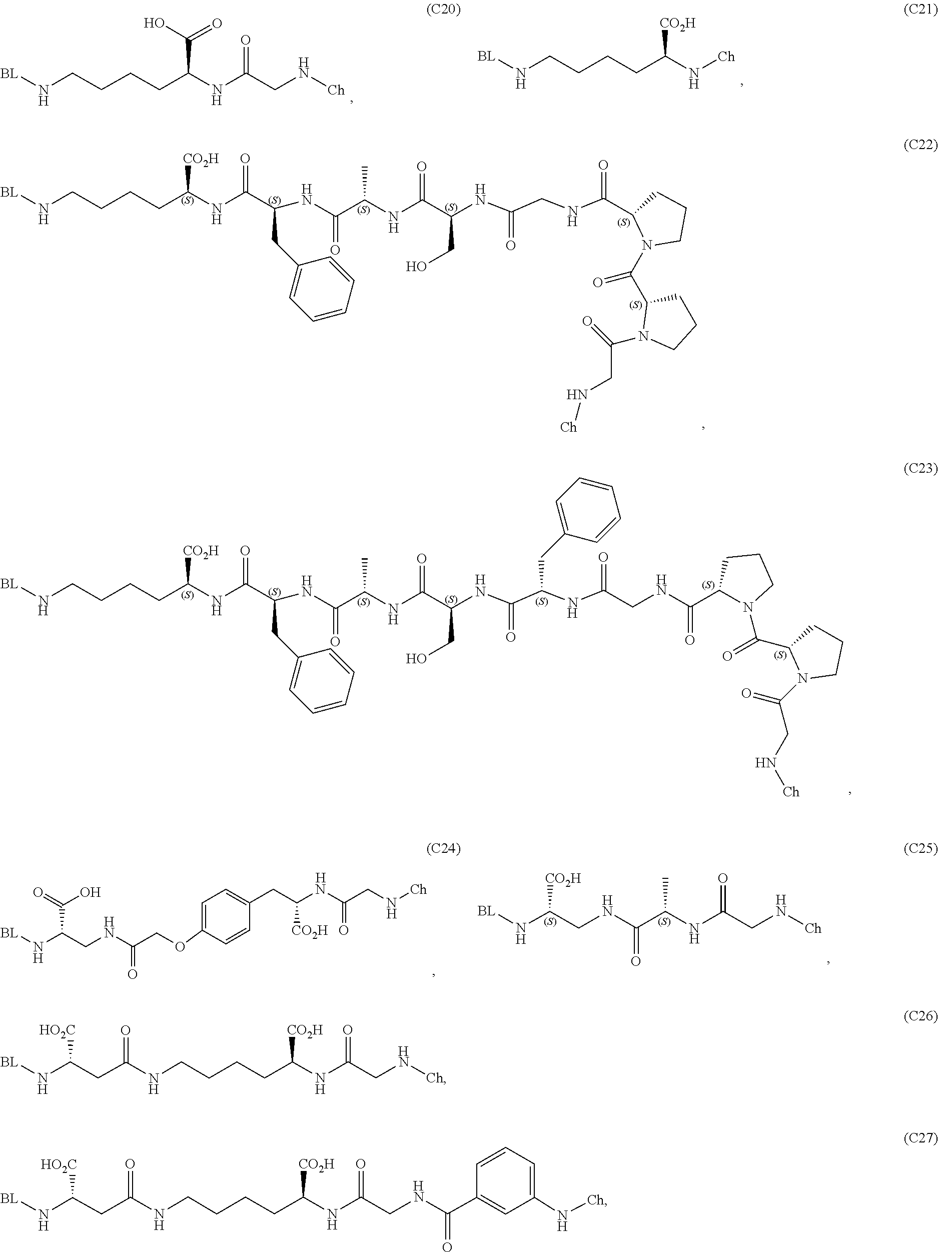

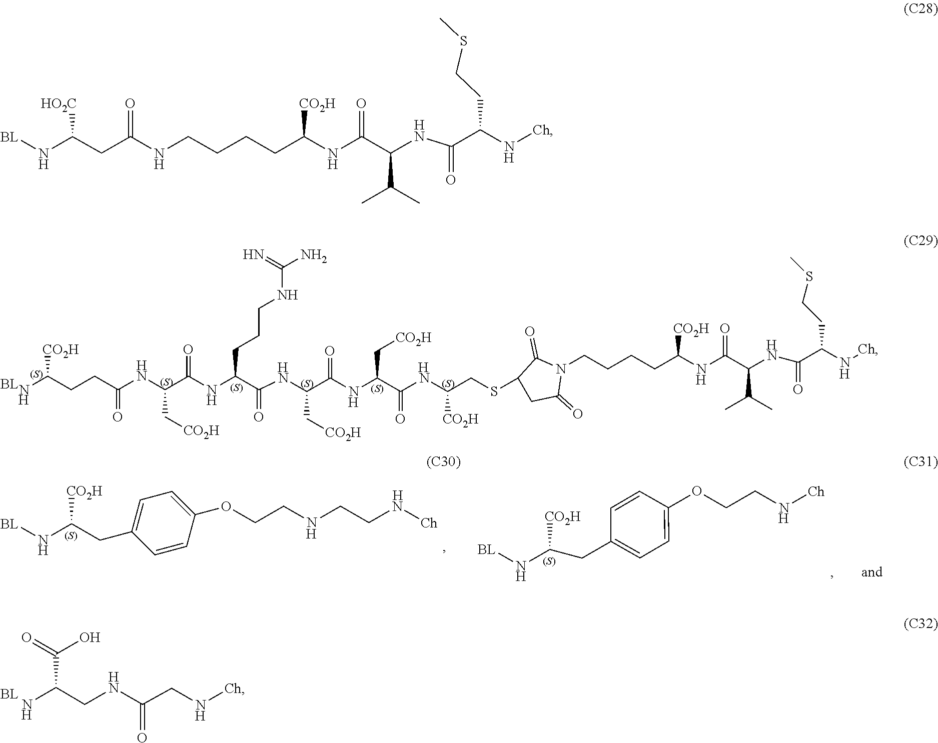

wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40.

In another embodiment, a compound of the present disclosure is of formula (C11) (see above), wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40.

In another embodiment, a compound of the present disclosure is of formula (Cl₂) (see above), wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40.

In another embodiment, a compound of the present disclosure is of any one of formula (C1) to (C32):

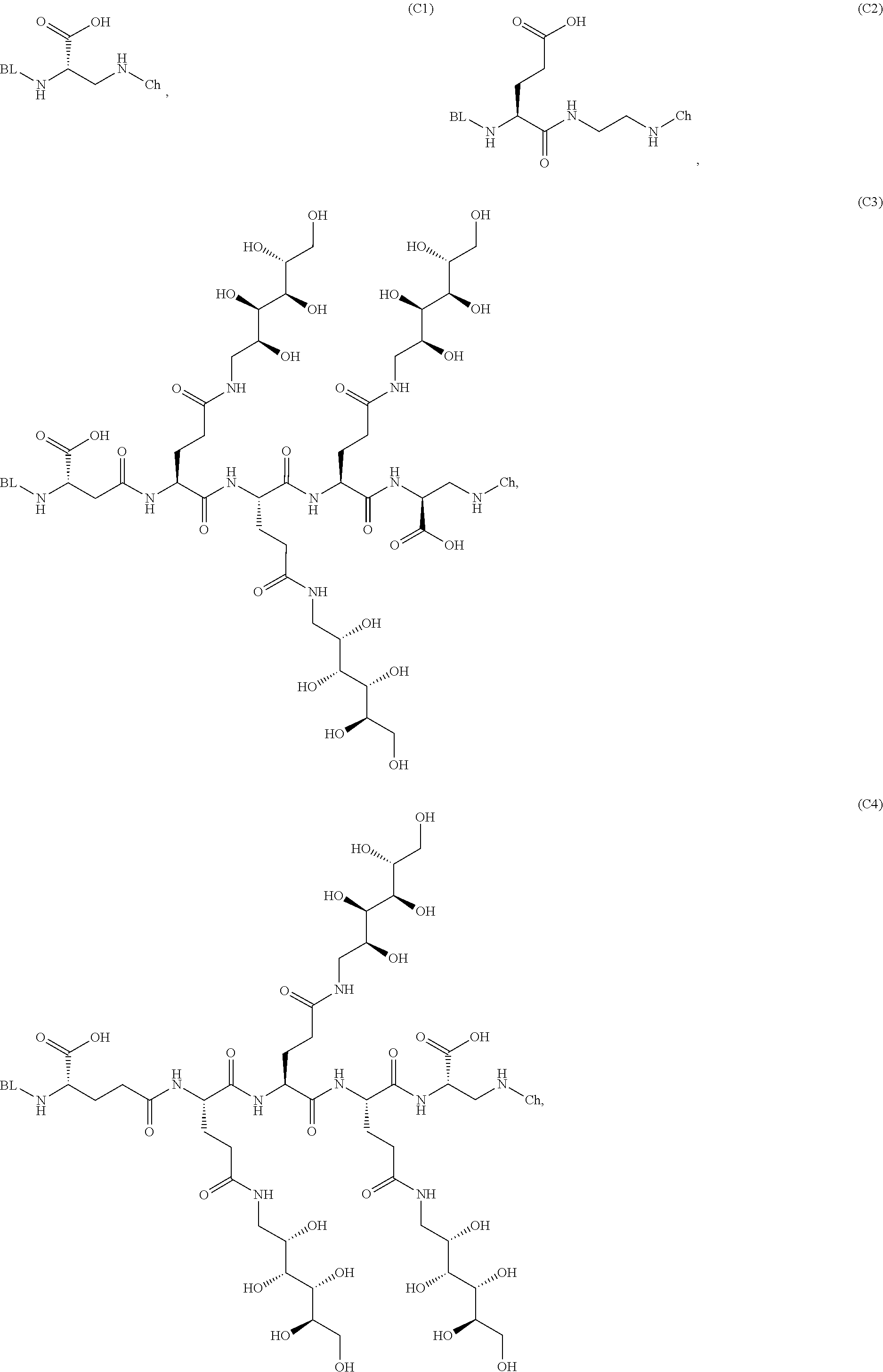

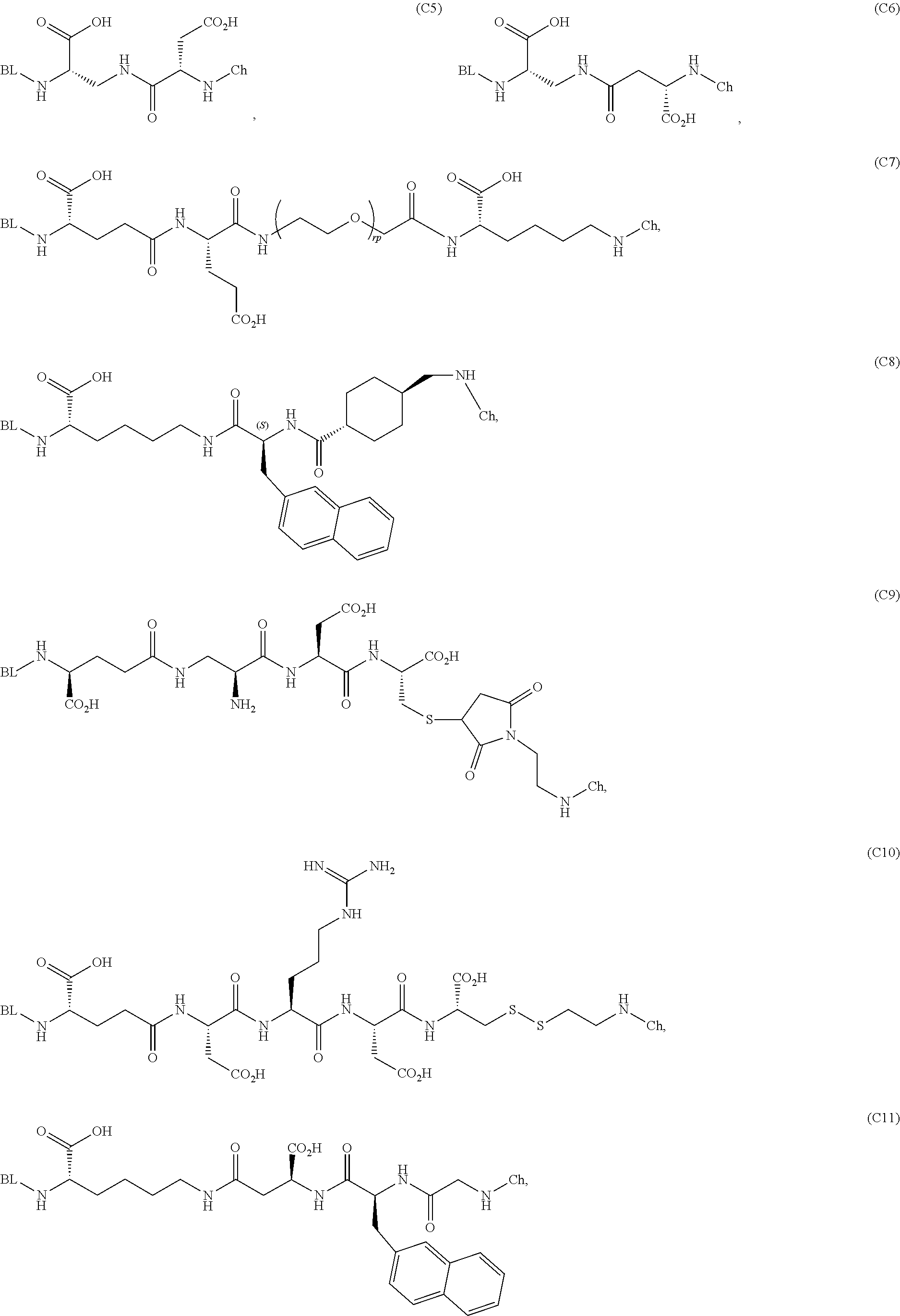

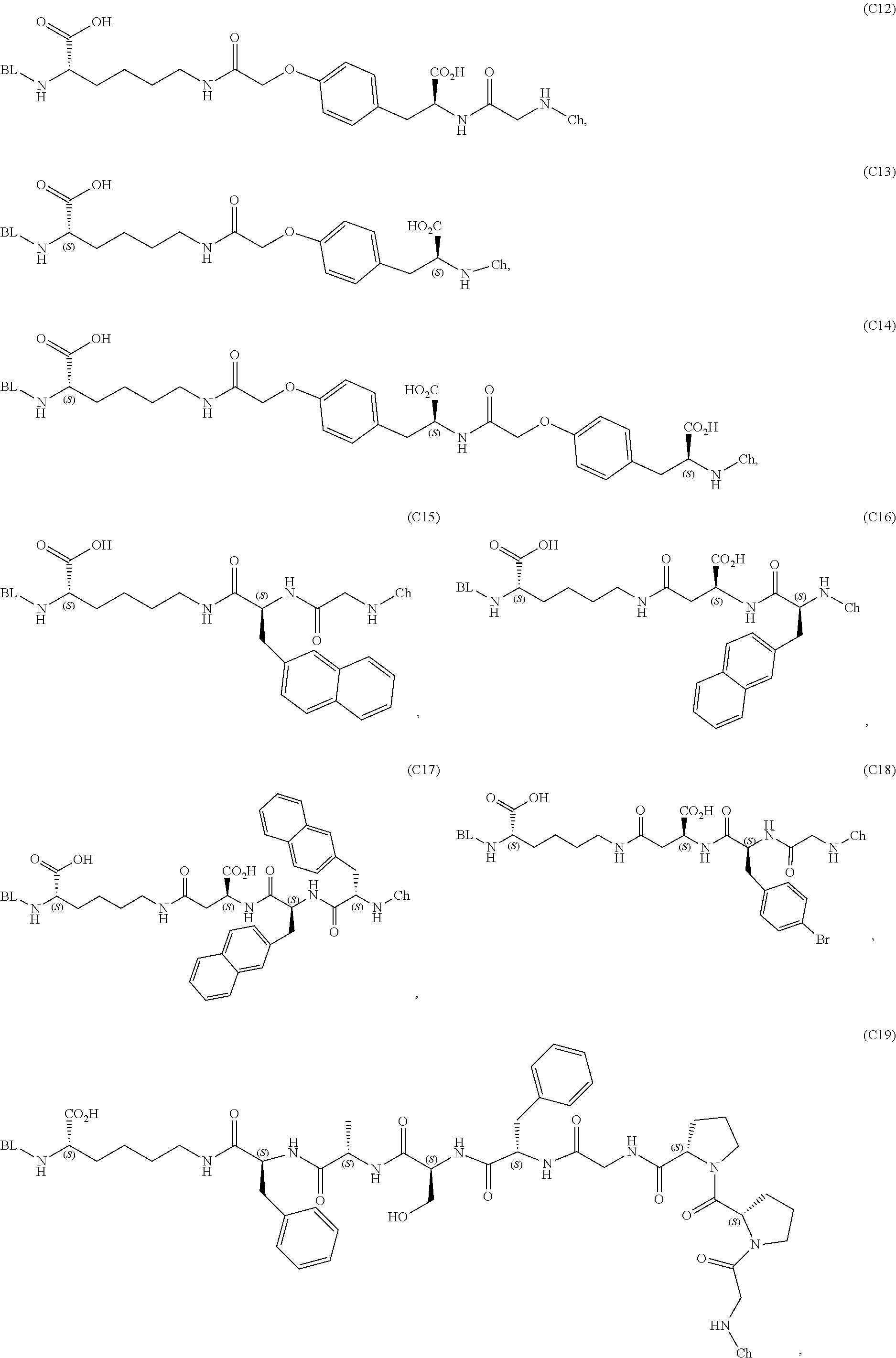

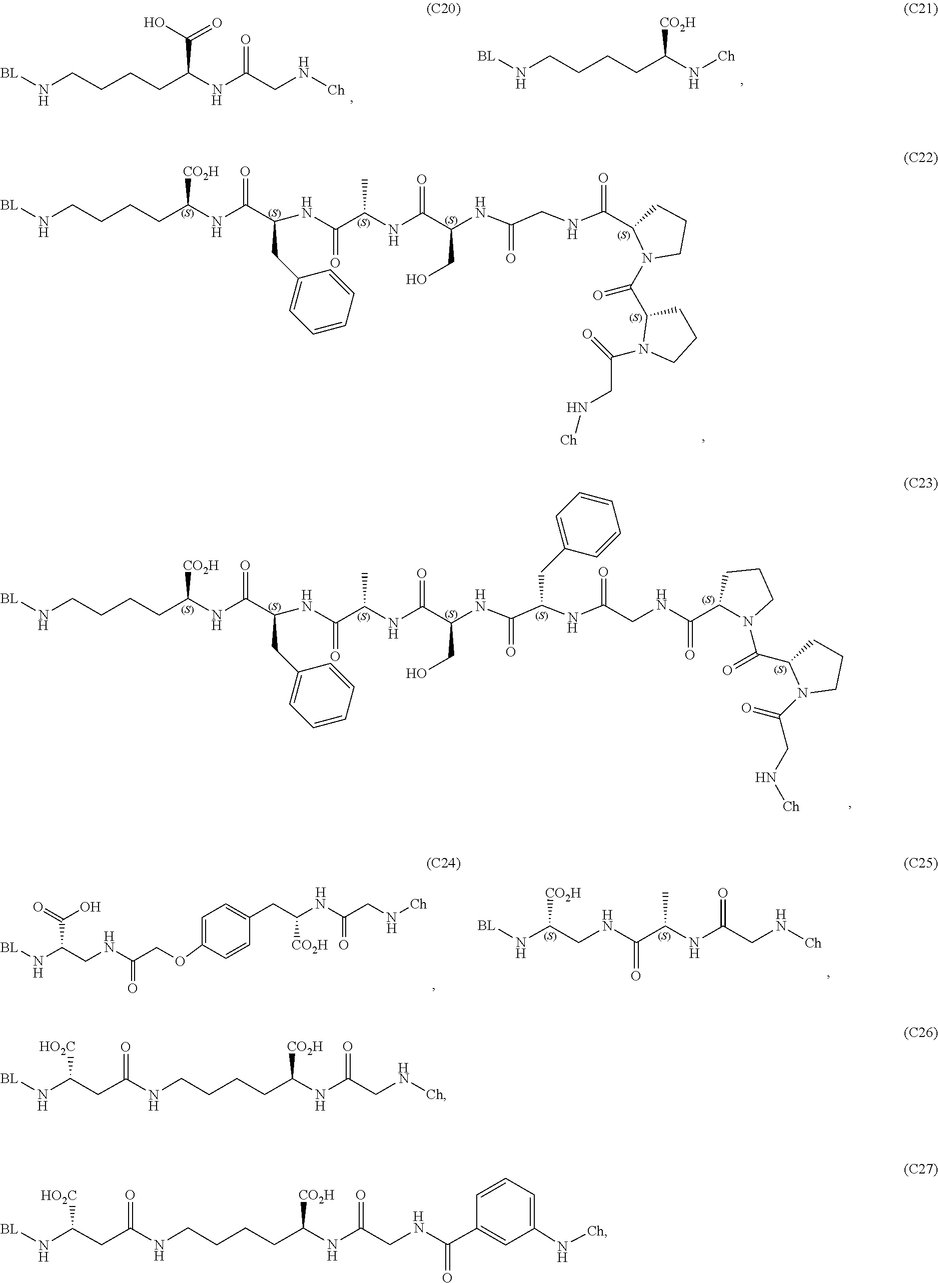

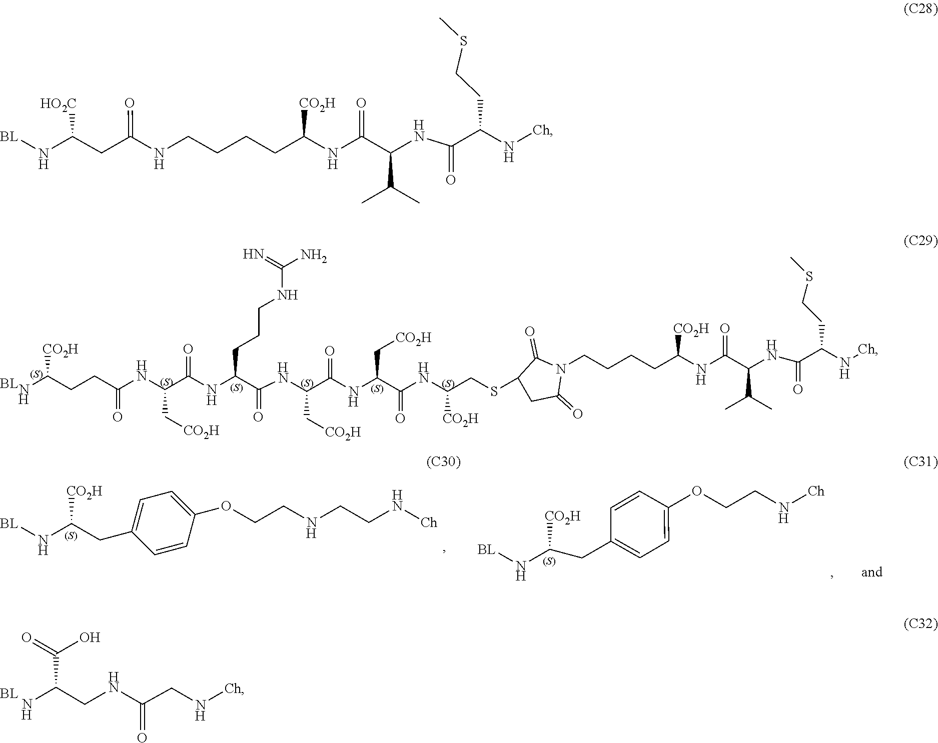

except that one group, corresponding to L^{x} (i.e., AA, L, L², or L³) as defined in Embodiment 1, within said any one of formula (C1) to (C32) is replaced by a different L^{x} as defined in Embodiment 1; wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40. In a further specific embodiment, the one group, which is replaced by a different L^{x}, is an AA group, the different L^{x} is a different AA group, and the different AA group is a conservative amino acid substitution of the AA group (e.g., this means that this embodiment encompasses compounds in which, for example, one aspartic acid residue (e.g., in formula (C11)) is replaced by a different AA and this replacement is a conservative amino acid substitution).

In another embodiment, a compound of the present disclosure is of formula (C11) (see above), except that one group, corresponding to L^{x} (i.e., AA, L, L, or L³) as defined in Embodiment 1, within said any one of formula (C1) to (C32) is replaced by a different L^{x} as defined in Embodiment 1; wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40. In a further specific embodiment, the one group, which is replaced by a different L^{x}, is an AA group, the different L^{x} is a different AA group, and the different AA group is a conservative amino acid substitution of the AA group (e.g., this means that this embodiment encompasses compounds in which, for example, one aspartic acid residue (e.g., in formula (C11)) is replaced by a different AA and this replacement is a conservative amino acid substitution).

In another embodiment, a compound of the present disclosure is of formula (Cl₂) (see above), except that one group, corresponding to L^{x} (i.e., AA, L, L, or L³) as defined in Embodiment 1, within said any one of formula (C1) to (C32) is replaced by a different L^{x} as defined in Embodiment 1; wherein BL and Ch are as described herein, for example, BL as described in any one of Embodiments 1 and 16-36 and Ch as described in Embodiments 1 or 40; or a pharmaceutically acceptable salt thereof. In a specific embodiment, BL is as described in any one of Embodiments 1 and 16-36, and Ch as described in Embodiment 1. In a further specific embodiment, BL is as described in any one of Embodiments 1 and 16-36, and Ch as described in Embodiment 40. In a further specific embodiment, BL is as described in Embodiment 16, and Ch as described in Embodiment 1 or 40. In a further specific embodiment, BL is as described in Embodiment 35, and Ch as described in Embodiment 1, 39 or 40. In a further specific embodiment, BL is as described in Embodiment 35, and Ch as described in Embodiment 39. In a further specific embodiment, BL is as described in Embodiment 35, and Ch as described in Embodiment 40. In a further specific embodiment, the one group, which is replaced by a different L^{x}, is an AA group, the different L^{x} is a different AA group, and the different AA group is a conservative amino acid substitution of the AA group (e.g., this means that this embodiment encompasses compounds in which, for example, one aspartic acid residue (e.g., in formula (C11)) is replaced by a different AA and this replacement is a conservative amino acid substitution).

In a further embodiment, the compound selected from

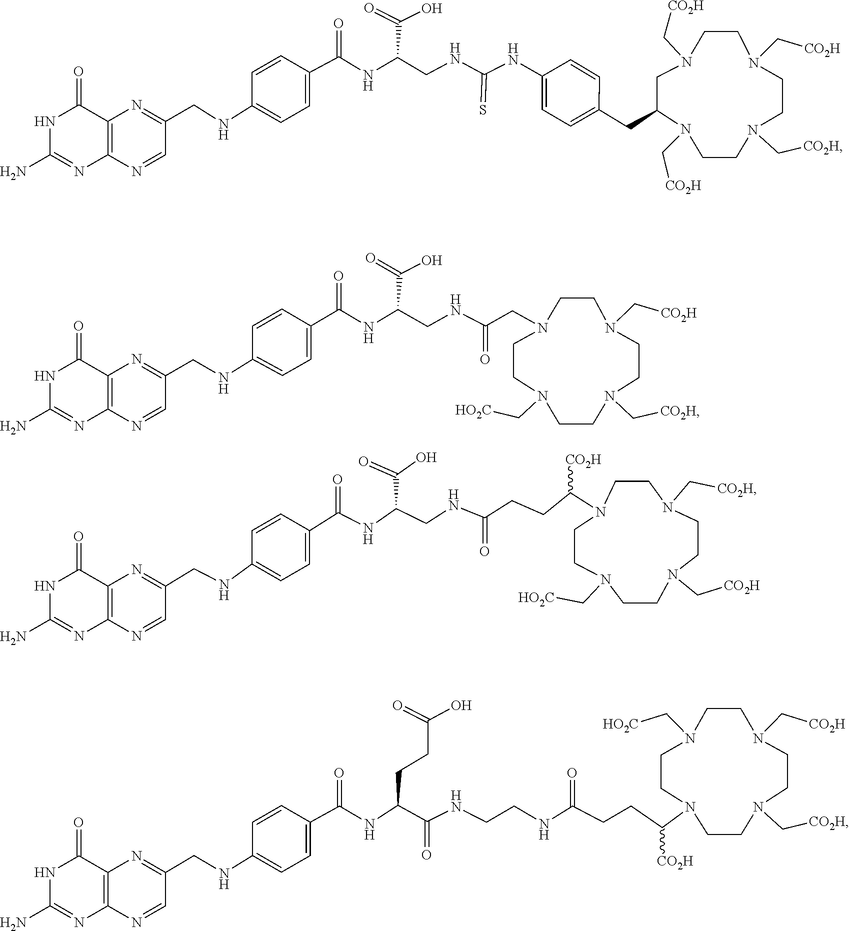

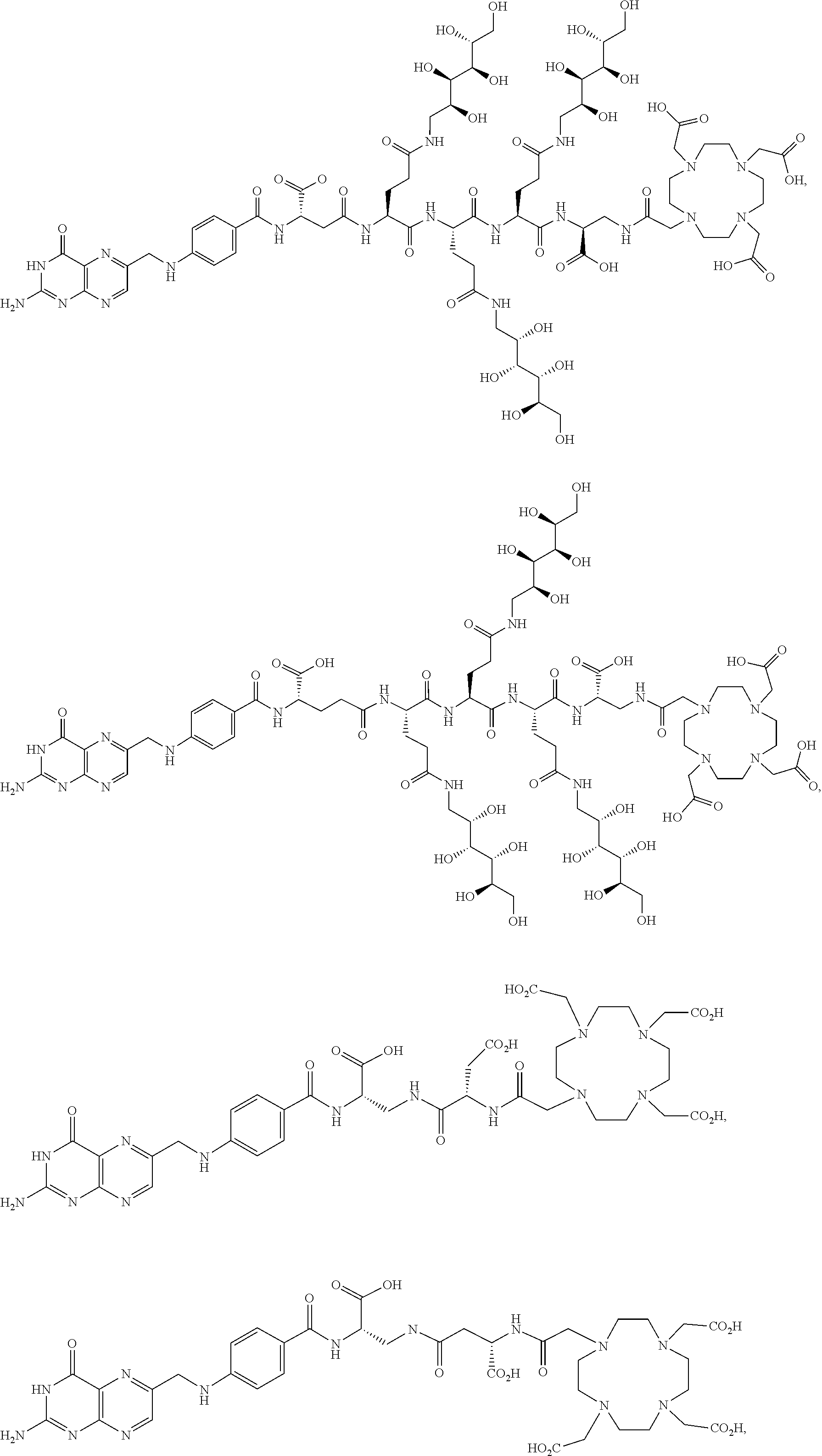

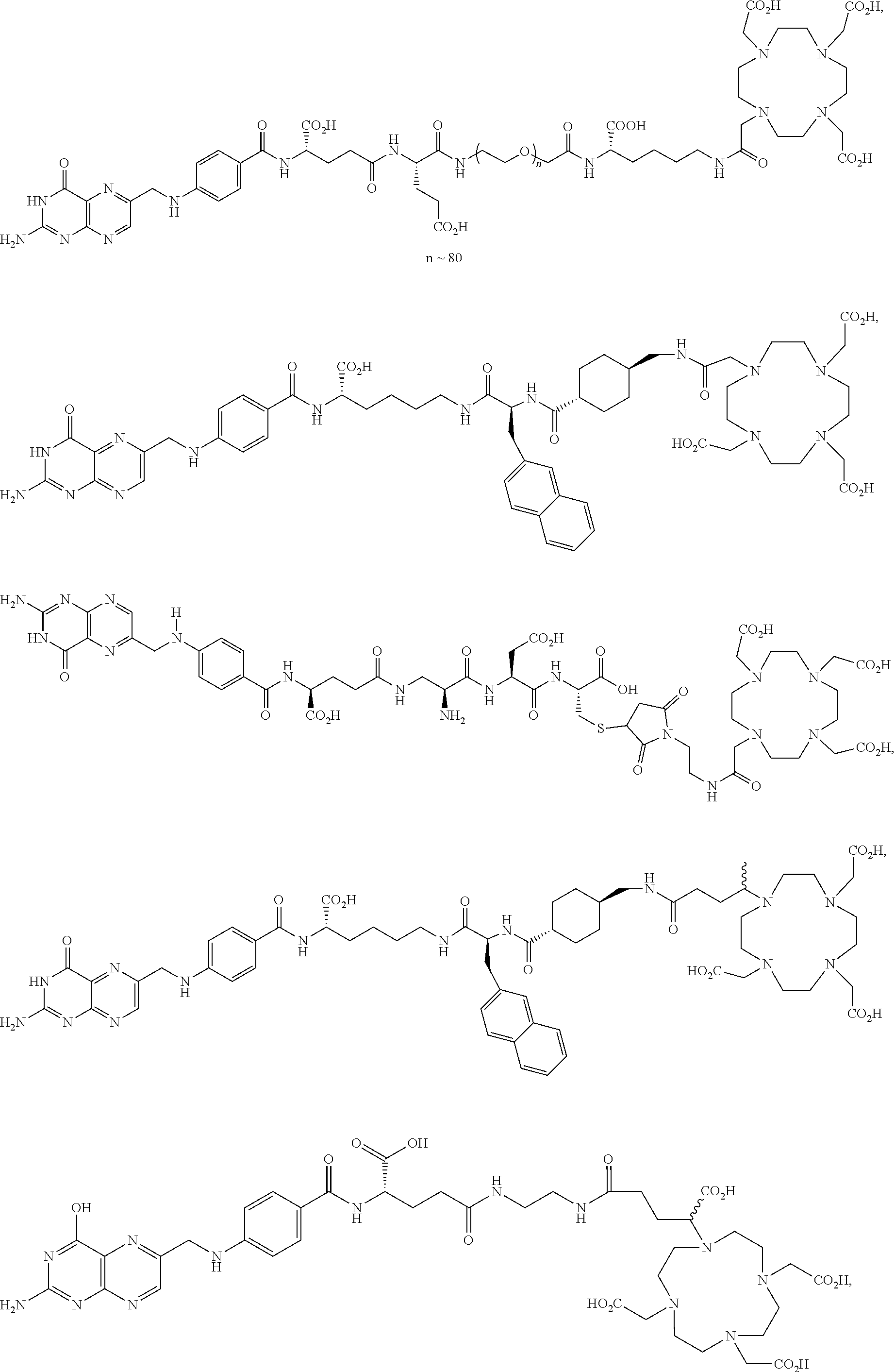

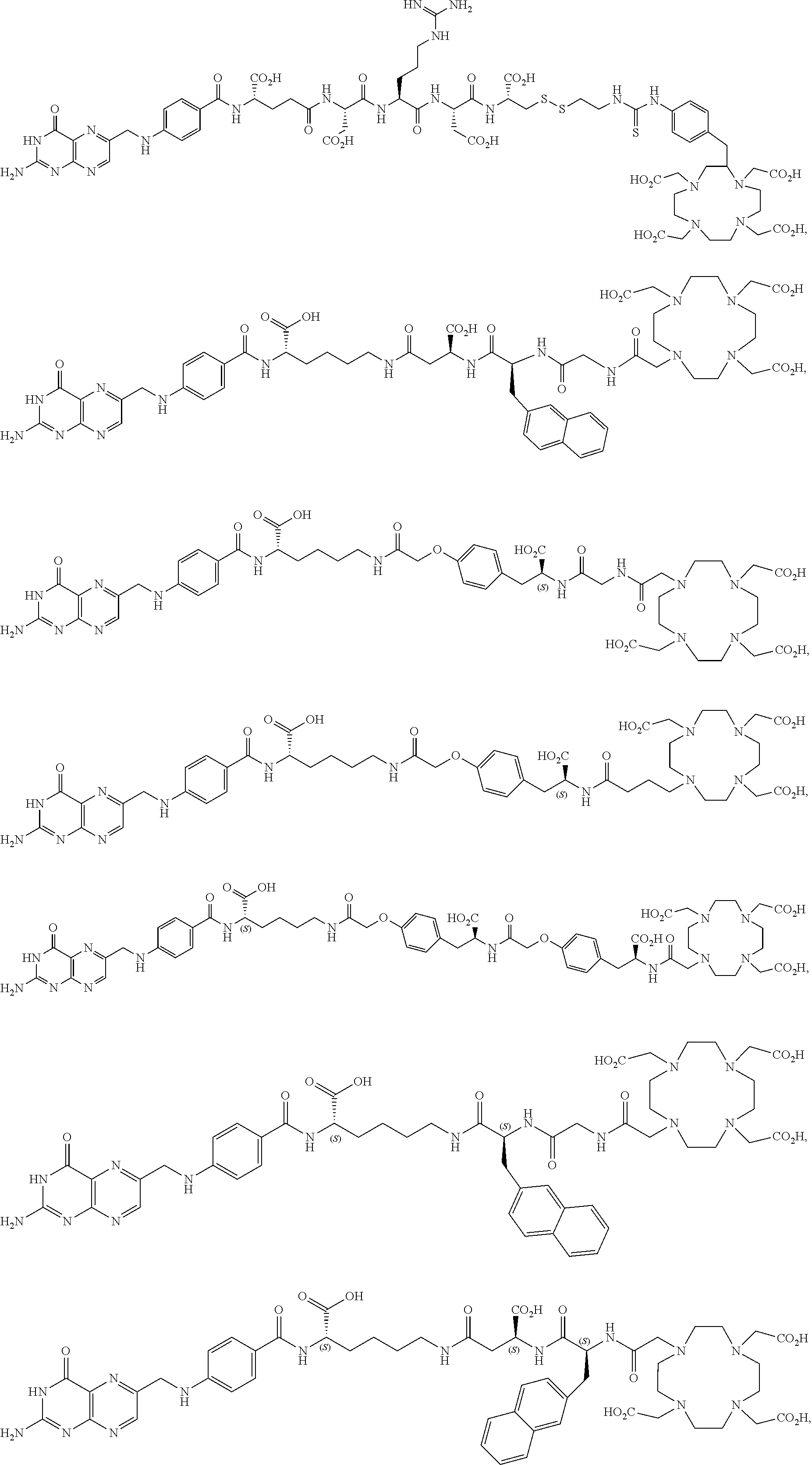

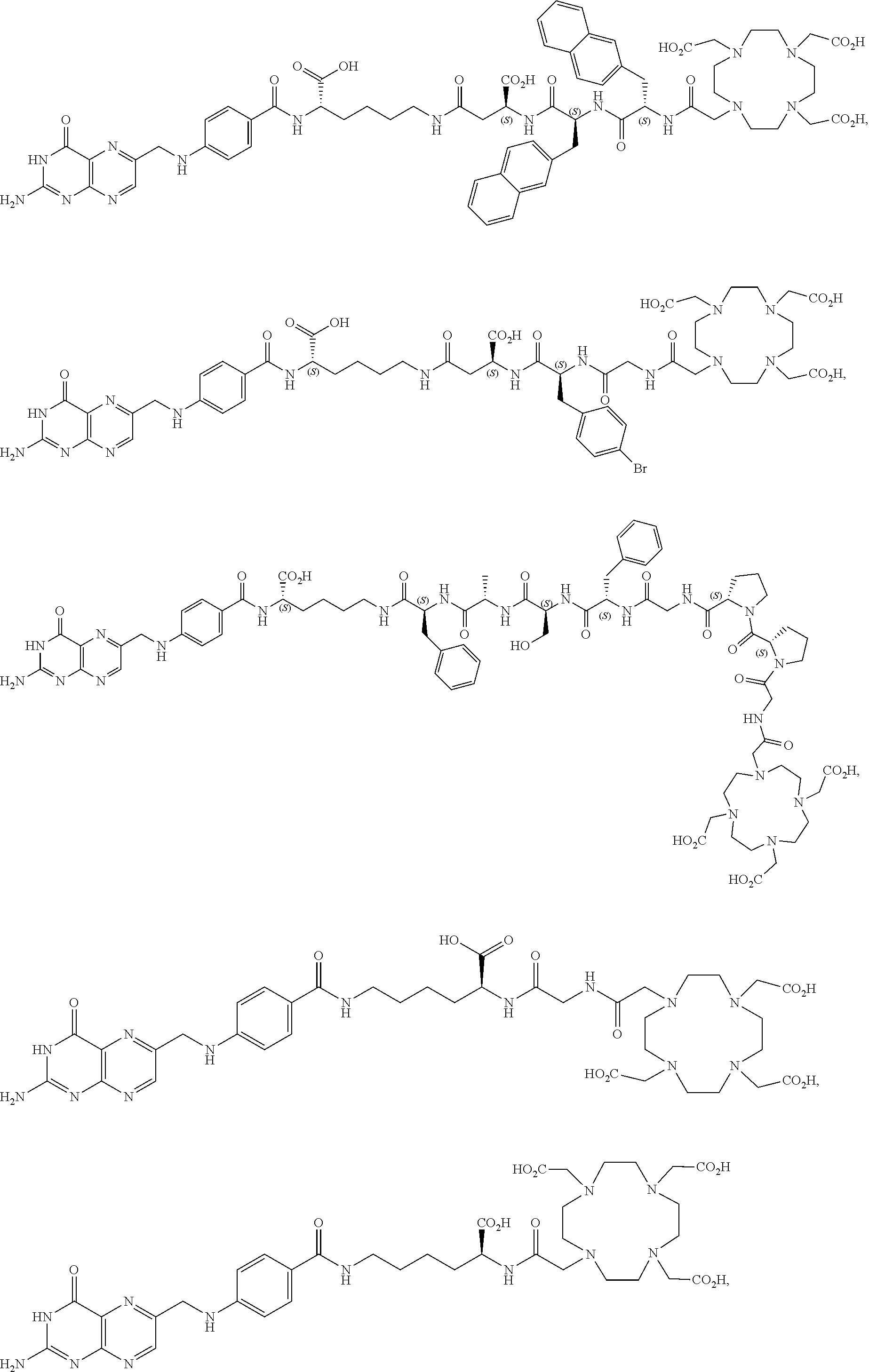

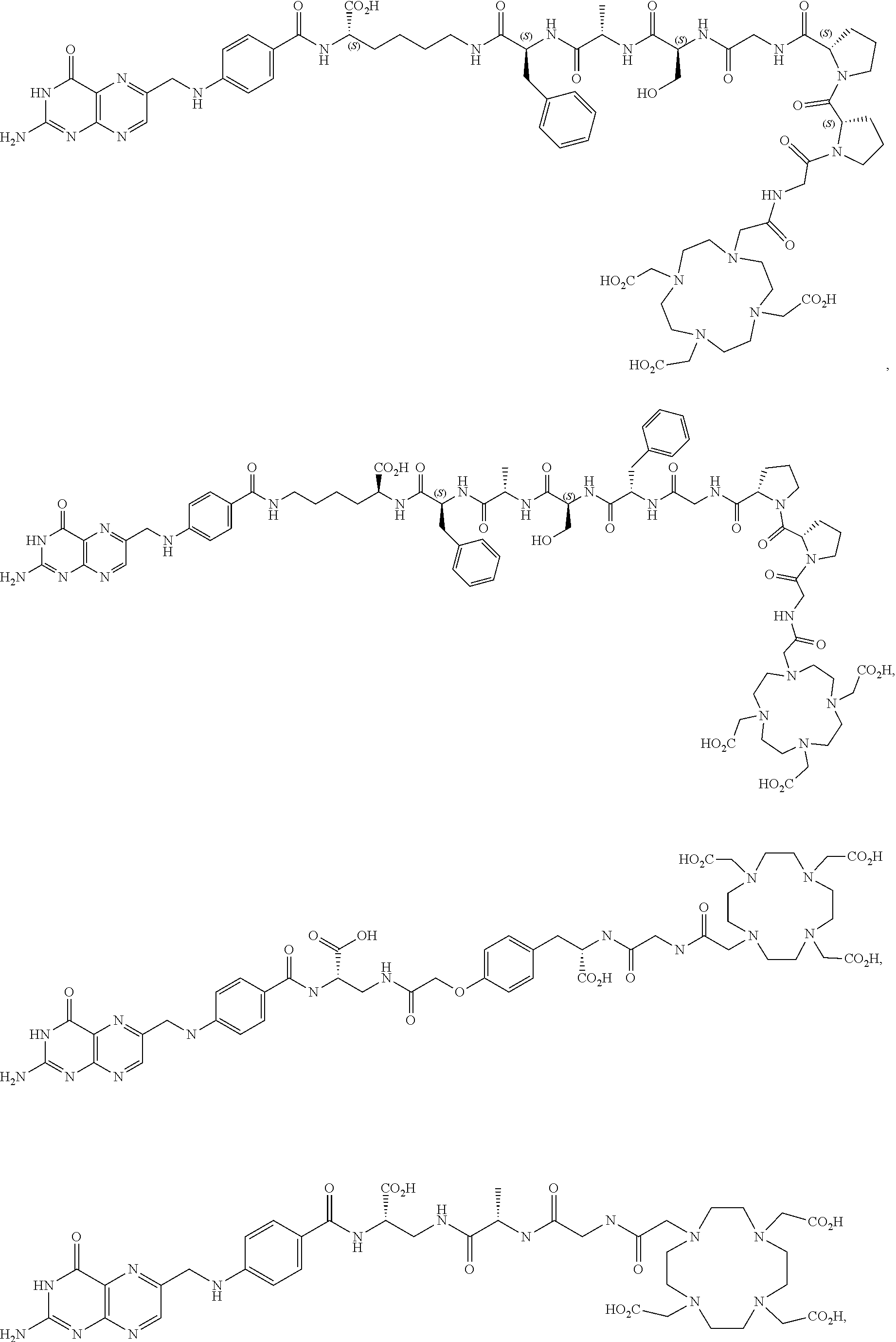

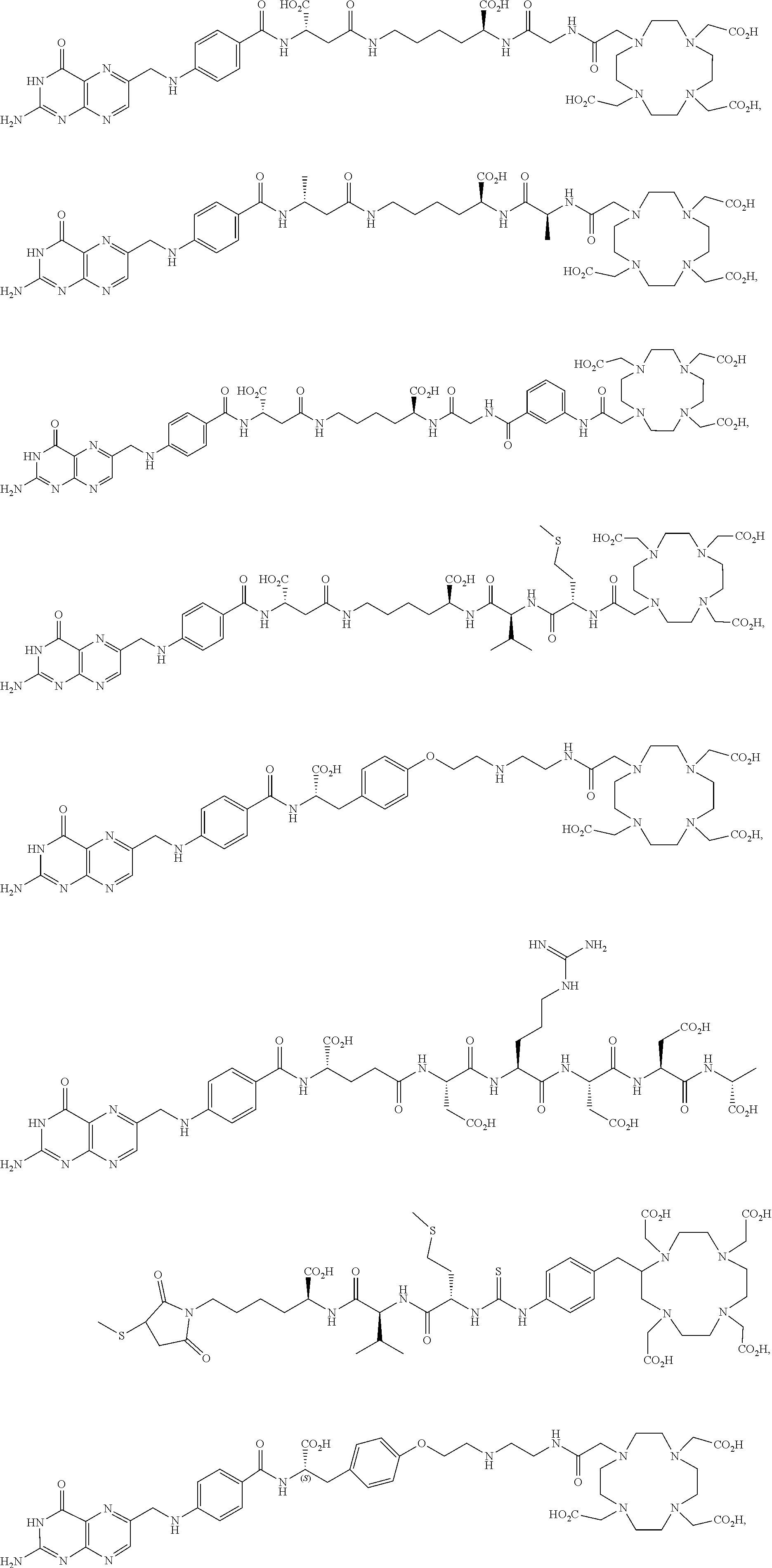

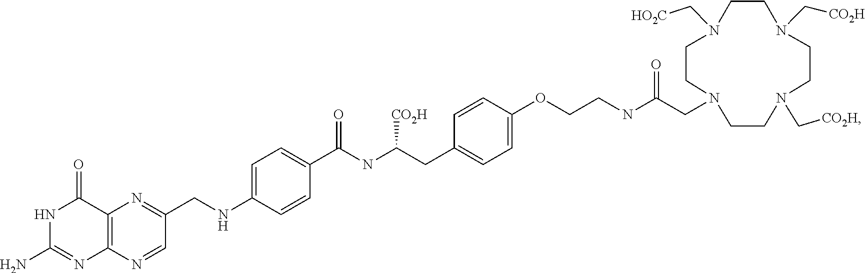

or a pharmaceutically acceptable salt thereof, wherein the chelating group exhibited in the above structural formulas can comprise a radioelement, Si-¹⁸F, B-¹⁸F, or Al-¹⁸F. In a specific embodiment the chelating group exhibited by the above structural formulas does not comprise a radioelement (i.e., the compounds are cold compounds). In an alternative specific embodiment, the chelating group exhibited by the above structural formulas comprises a radioelement, Si-¹⁸F, B-¹⁸F, or Al-¹⁸F (i.e., the compounds are hot compounds).

Another embodiment is a compound of formula (IV), or a pharmaceutically acceptable salt thereof,

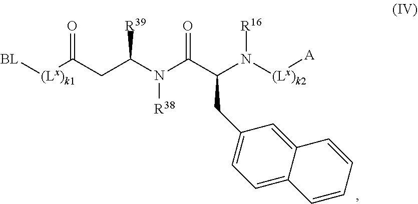

wherein each L^{x} is independently L¹, L³ or AA, and BL, A, L¹, L³, AA, R¹⁶, R³⁸, and R³⁹ are as described herein, for example, as defined in Embodiment 1 or 2; k1 is 1, 2, 3, 4, 5, 6, or 7; k2 is 1, 2, 3, 4, 5, 6, or 7; and k1+k2 is not greater than 8. In a specific embodiment, R¹⁶, R³⁷ and R³⁸ are H; and R³⁹ is —COOH. In a further more specific embodiment of the aforementioned embodiment or specific embodiment, each L¹ (when present) is independently of the formula

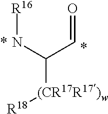

wherein
R¹⁶ is H,
each R¹⁷ and R^{17′} is independently H, C₁-C₆ alkyl, or —C(O)OH,
each R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen;
each L³ (when present) is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′})ₜ—O—(C₆-C₁₀ aryl)-(CR³⁶R^{36′})ᵣ—NH—; and AA (when present) is an amino acid residue; wherein
each R³⁶, R^{36′}, R^{36″}, R^{36′″}, R³⁹ and R^{39′} is independently H or —COOH;
each r independently is 1, 2, 3, 4, 5; and
each t independently is 1, 2, 3, 4, 5.

Another embodiment is a compound of formula (V), or a pharmaceutically acceptable salt thereof,

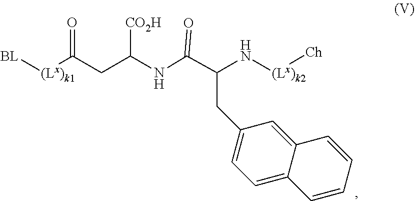

wherein each L^{x} is independently L¹, L³ or AA; and BL, Ch, L¹, L³, and AA, are as described herein, for example, as defined in Embodiment 1 or 2; k1 is 1, 2, 3, 4, 5, 6, or 7; k2 is 1, 2, 3, 4, 5, 6, or 7; and k1+k2 is not greater than 8. In a specific embodiment, R¹⁶, R³⁷ and R³⁸ are H; and R³⁹ is —COOH. In a further more specific embodiment of the aforementioned embodiment or specific embodiment, each L¹ (when present) is independently of the formula

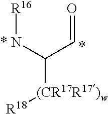

wherein
R¹⁶ is H,
each R¹⁷ and R^{17′} is independently H, C₁-C₆ alkyl, or —C(O)OH,
each R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen;
each L³ (when present) is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—; and AA (when present) is an amino acid residue; wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

Another embodiment is a compound of formula (VI), or a pharmaceutically acceptable salt thereof,

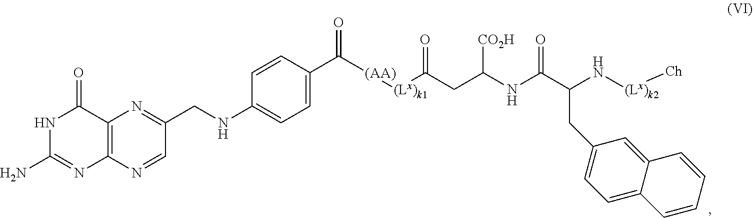

wherein each L^{x} is independently L¹, L³ or AA; and BL, Ch, L¹, L³, and AA, are independently as described herein, for example, as defined in Embodiment 1 or 2; k1 is 1, 2, 3, 4, 5, or t; k2 is 1, 2, 3, 4, 5, or 6; and k1+k2 is not greater than 8. In a specific embodiment of the aforementioned embodiment, each L¹ (when present) is independently of the formula

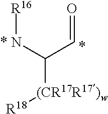

wherein
R¹⁶ is H,
each R¹⁷ and R^{17′} is independently H, C₁-C₆ alkyl, or —C(O)OH,
each R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen;
each L³ (when present) is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—; and AA (when present) is an amino acid residue; wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

Another embodiment is a compound of formula (VI), or a pharmaceutically acceptable salt thereof,

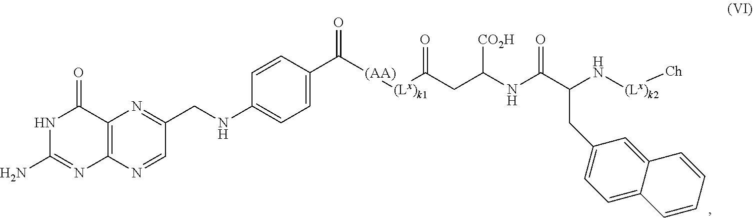

wherein each L^{x} is independently AA; BL is a folate receptor binding ligand, and Ch is a chelating group which can comprise a metal, a radioelement, Si-¹⁸F, B-¹⁸F, or Al-¹⁸F; k1 is 1, 2, 3, 4, 5, or t; k2 is 1, 2, 3, 4, 5, or 6; and k1+k2 is not greater than 8. In a specific embodiment, Ch is

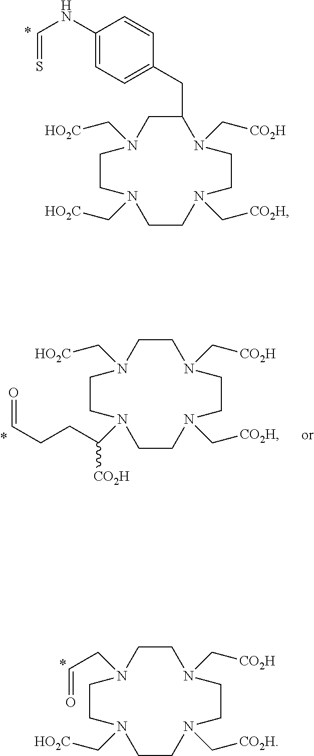 [SMILES: O=C(O)CN1CCN(CC(=O)O)CCN(CC(=O)O)C(Cc2ccc(NC=S)cc2)CN(CC(=O)O)CC1 ; O=CCCC(C(=O)O)N1CCN(CC(=O)O)CCN(CC(=O)O)CCN(CC(=O)O)CC1 ; O=CCN1CCN(CC(=O)O)CCN(CC(=O)O)CCN(CC(=O)O)CC1]

In some embodiments, the compound is not a compound of formulas (E1)-(E2) (as described herein), a tautomer of (E1)-(E5), a compound of (E1)-(E5) in which a metal or radioelement is chelated, or a pharmaceutical salt of (E1)-(E5).

### Folate Receptor Binding Ligand (BL)

The compounds of the present disclosure (also referred to as FR targeted/targeting compounds), for example, a compound of formula (I), or a pharmaceutically acceptable salt thereof, include a folate receptor binding ligand (BL). Typically, BL can bind to all functioning folate receptor isoforms, including, but not limited to, FR-α, FR-β, and FR-γ.

In some embodiments, BL binds to FR-α. FR-α is expressed or overexpressed in many cancers.

In some embodiments, BL binds to FR-β.

In some embodiments, BL binds to FR-γ.

In some embodiments, BL binds to FR-α and FR-β.

In some embodiments, BL binds to FR-α, FR-β, and FR-γ.

In some embodiments, the BL is a folate, or derivative thereof, a fragment thereof, or a radical thereof.

In some embodiments, BL is a pteryl group or derivative thereof (i.e., a pteroic acid, or derivative thereof, in which the carboxyl group has been reacted, typically, with an amino group of an amino acid).

In some embodiments, BL is of formula (IIa),

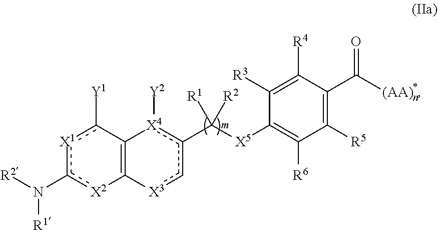

wherein R¹, R², R³, R⁴, R⁵, R⁶, R1′, R^{2′}, X¹, X², X³, X⁴, X⁵, Y¹, Y², m, n, AA and * are as defined in any one of Embodiments 17-34.

In some embodiments, BL is of the formula (IIb),

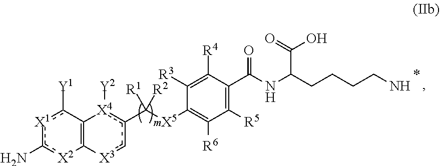

wherein R¹, R², R³, R⁴, R⁵, R⁶, X¹, X², X³, X⁴, X⁵, Y¹, Y², m, and * are as defined in any one of Embodiments 17-34.

In some embodiments, BL is of the formula (IIc),

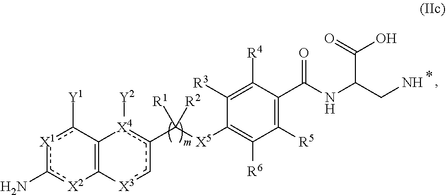

wherein R¹, R², R³, R⁴, R⁵, R⁶, X¹, X², X³, X⁴, X⁵, Y¹, Y², m, and * are as defined in any one of Embodiments 17-34.

In some embodiments, BL is of the formula

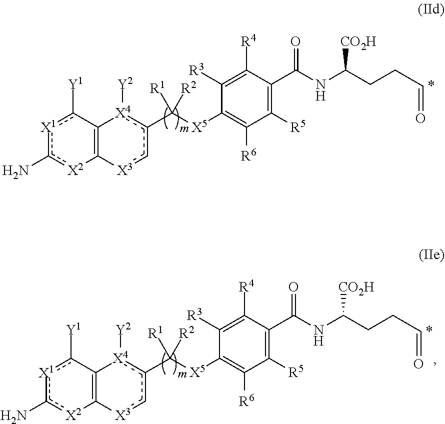

wherein R¹, R², R³, R⁴, R⁵, R⁶, Y¹, Y², X¹, X², X³, X⁴, X⁵, m and * are as defined in any one of Embodiments 17-34.

In some embodiments, BL is of the formula

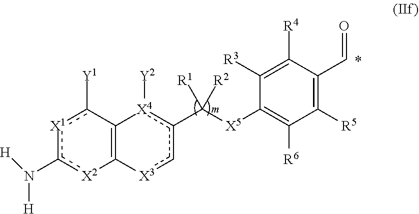

wherein R¹, R², R³, R⁴, R⁵, R⁶, Y¹, Y², X¹, X², X³, X⁴, X⁵, m and * are as defined in any one of Embodiments 17-34.

In some embodiments described herein, R¹ and R² are H. In some embodiments described herein, m is 1. In some embodiments described herein, R³ is H. In some embodiments described herein, R⁴ is H. In some embodiments described herein, R⁵ is H. In some embodiments described herein, R⁶ is H. In some embodiments described herein, R³, R⁴, R⁵ and R⁶ are H. In some embodiments described herein, X¹ is —NR¹¹, and R¹¹ is H. In some embodiments described herein, X² is ═N—. In some embodiments described herein, X³ is —N═. In some embodiments described herein, X⁴ is —N═. In some embodiments described herein, X¹ is —NR¹¹, and R¹¹ is H; X² is ═N—; X³ is —N═; and X⁴ is —N═. In some embodiments described herein, X⁵ is NR¹², and R¹² is H. In some embodiments, Y¹ is ═O. In some embodiments, Y² is absent.

In some embodiments, BL is of the formula

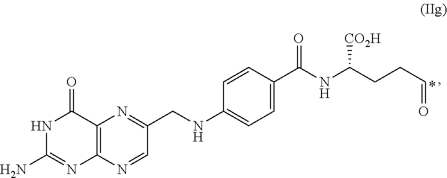 [SMILES: Nc1nc2ncc(CNc3ccc(C(=O)N[C@@H](CCC=O)C(=O)O)cc3)nc2c(=O)[nH]1]

wherein * is a covalent bond to the rest of the compound.

In some embodiments, BL is of the formula

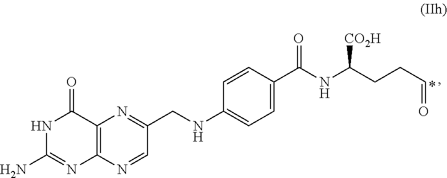 [SMILES: Nc1nc2ncc(CNc3ccc(C(=O)N[C@H](CCC=O)C(=O)O)cc3)nc2c(=O)[nH]1]

wherein * is a covalent bond to the rest of the compound.

In some embodiments, BL is of the formula

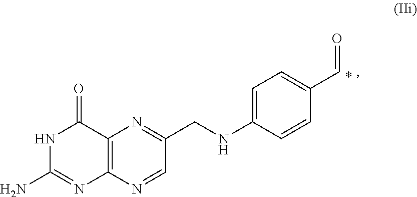 [SMILES: Nc1nc2ncc(CNc3ccc(C=O)cc3)nc2c(=O)[nH]1]

wherein * is a covalent bond to the rest of the compound.

In some embodiments, BL is of the formula

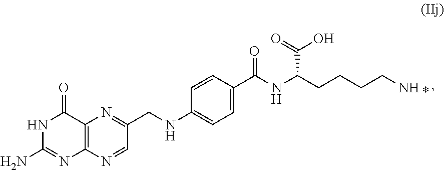 [SMILES: NCCCC[C@H](NC(=O)c1ccc(NCc2cnc3nc(N)[nH]c(=O)c3n2)cc1)C(=O)O]

wherein * is a covalent bond to the rest of the compound.

In some embodiments, BL is of the formula

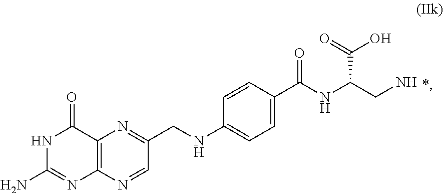 [SMILES: NC[C@H](NC(=O)c1ccc(NCc2cnc3nc(N)[nH]c(=O)c3n2)cc1)C(=O)O]

wherein * is a covalent bond to the rest of the compound.
In some embodiments, BL comprises a pteryl group or a derivative thereof, and the pteryl group or derivative thereof is covalently bonded to a group selected from

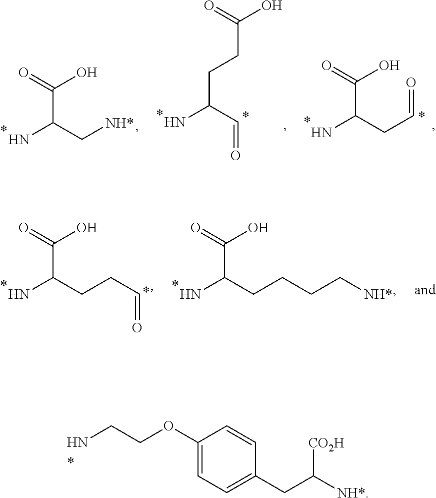

The Linker (L^{x})ₖ

The linker (L^{x})ₖ connects BL to A in the compounds described herein. It has k groups L^{x} which are covalently connected. This covalent connection can be the result, for example, of a condensation reaction between a carboxyl group of one L^{x} precursor and an amino group of another L^{x} precursor. Each L^{x} of (L^{x})ₖ can be independently selected from AA, L¹, ^{L} and L³ as defined herein.

In some embodiments, the compound of formula (I) comprises a linker (L^{x})ₖ in which each L^{x} of (L^{x})ₖ is independently selected from AA, L¹, L² and L³ as defined herein, and k is 1, 2, 3, 4, 5, 6, 7, 8, 9, 10, 11, 12, 13, 14, 15, 16, 17, 18, 19, or 20.

In some embodiments, k is 1, 2, 3, 4, 5, 6, 7, 8, 9, or 10. In some embodiments, k is 1, 2, 3, 4, 5, 6, 7, 8, or 9. In some embodiments, k is 1, 2, 3, 4, 5, 6, 7, or 8. In some embodiments, k is 1, 2, 3, 4, 5, 6, or 7. In some embodiments, k is 1, 2, 3, 4, 5, or 6. In some embodiments, k is 1, 2, 3, 4, or 5. In some embodiments, k is 1, 2, 3, or 4.

AA is an amino acid residue as defined herein. In certain embodiments, AA is a naturally occurring amino acid residue. In certain embodiments, AA is in the L-form. In certain embodiments, AA is in the D-form. It will be appreciated that in certain embodiments, the compounds described herein will comprise more than one amino acid as portions of the linker, and the amino acid residues can be the same or different, and can be selected from a group of amino acids residues It will be appreciated that in certain embodiments, the compounds described herein will comprise more than one amino acid residue as portions of the linker, and the amino acid residues can be the same or different, and can be selected from a group of amino acid residues in D- or L-form. In some embodiments, an AA can be covalently attached to BL, another linker portion, or A through an alpha-amino group of the amino acid corresponding to AA. In some embodiments, an AA can be covalently attached to BL, another linker portion, or A through a carboxyl group of an amino acid corresponding to AA. In some embodiments, an AA can be covalently attached to BL, another linker portion, or A through a side chain group of an amino acid corresponding to AA. In some embodiments, an AA can be covalently attached to BL, another linker portion, or A through a combination of an alpha-amino group of the amino acid corresponding to AA, a carboxyl group of the amino acid corresponding to AA, or a side chain of the amino acid corresponding to AA.

In some embodiments, each AA is independently selected from the group consisting of L-lysine, L-glycine, L-aspartic acid, L-glutamic acid, L-glutamine, L-cysteine, L-alanine, L-valine, L-leucine, L-isoleucine, L-3-amino-alanine, L-arginine, D-lysine, D-glycine, D-aspartic acid, D-glutamic acid, D-glutamine, D-cysteine, D-alanine, D-valine, D-leucine, D-isoleucine, D-3-amino-alanine, and D-arginine. In some embodiments, each AA is independently selected from the group consisting of L-3-amino-alanine, Lys, Asp, Arg, Glu and Cys.

One or more L¹ can be present or L¹ can be absent in the compounds described herein, for example, the compounds of formula (I), or a pharmaceutically acceptable salt thereof. In some embodiments, each L¹ each L¹ is independently of the formula

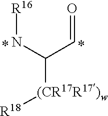

wherein
R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′} —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl, or a sugar;
w is 1, 2, 3, 4 or 5;
p is 1, 2, 3, 4 or 5;
q is 1, 2, 3, 4 or 5;
   wherein each * represents a covalent bond.

It will be appreciated that when L¹ is described according to the formula above, that both the R- and S-configurations are contemplated.

In some embodiments, each L¹ is independently selected from the group consisting of

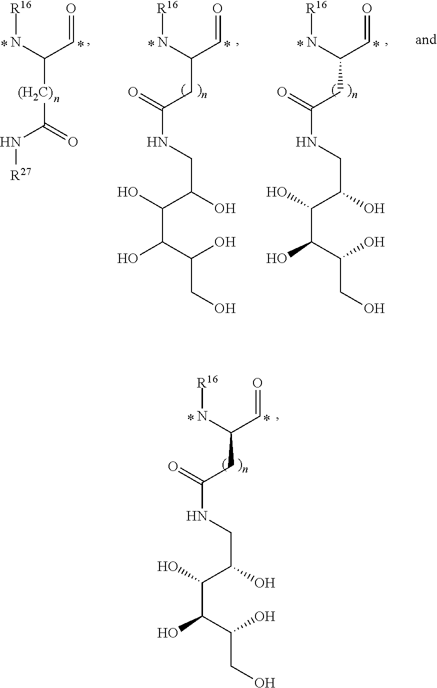

wherein R¹⁶ is defined as described herein, and each * represent a covalent bond to the rest of the compound.

In some embodiments, R¹⁶ is H. In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
w is 1, 2, 3, 4 or 5;
p is 1, 2, 3, 4 or 5;
q is 1, 2, 3, 4 or 5; and
each * represent a covalent bond to the rest of the compound.

In some embodiments, R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen.

In some embodiments, the compounds described herein comprise a L¹, wherein R¹⁷ and R^{17′} are H, and R¹⁸ is 5- to 7-membered heteroaryl. In some embodiments, the compounds described herein comprise a L¹, wherein R¹⁷ and R^{17′} are H, and R¹⁸ is 2-naphthyl.

In some embodiments of the conjugates described herein, L¹ is present. In some embodiments of the conjugates described herein, L¹ is absent. In some embodiments, z2 is 0. In some embodiments, z2 is 1. In some embodiments, z2 is 2. In some embodiments, z2 is 3.

One or more L² can be present, or L² can be absent in the compounds described herein.

In some embodiments, each L² can be of the formula

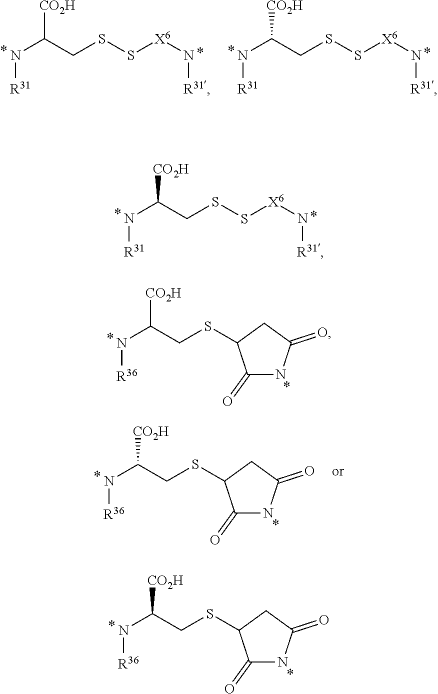

wherein
each or R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
X⁶ is C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} are independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
R³⁶ is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and
   each * is a covalent bond to the rest of the compound.

In some embodiments, R³¹ is H. In some embodiments, R³⁶ is H. In some embodiments, X⁶ is C₁-C₆ alkyl. In some embodiments, X⁶ is C₁-C₆ alkyl or C₆-C₁₀ aryl-(C₁-C₆ alkyl).

In some embodiments, each L² is independently of the formula

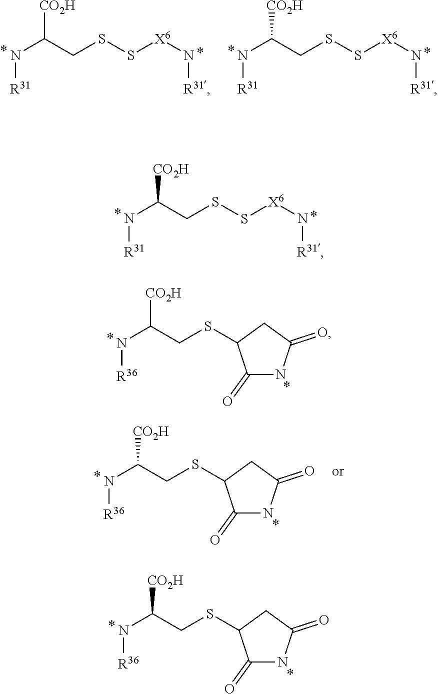

wherein
R³¹ and R^{31′} are H,
R³⁶ is H; and
   each * is a covalent bond to the rest of the compound.

One or more L³ can be present, or L³ can be absent in the compounds described herein.

In some embodiments, each L³ is independently C₁-C₆ alkylene, —OC₁-C₆ alkylene, —SC₁-C₆ alkylene, C₃-C₆ cycloalkylene, —C(O)C₃-C₆ cycloalkylene-, —C(O)C₃-C₆ cycloalkylene-(CR³⁹R^{39′})ᵣ—, —C(O)C₃-C₆ cycloalkylene-(CR³⁹R^{39′})ᵣNR³⁷—, 3- to 7-membered heterocycloalkylene, C₆-C₁₀ aryl, 5- to 7-membered heteroaryl, —NR³⁶(CR^{36′}R^{36″})ᵣ—S-(succinimid-1-yl)-, —(CR^{36′}R^{36″})ᵣC(O)NR³⁷—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ—, —C(O)O(CR³⁹R^{39′})ᵣ—, —NR³⁷C(O)(CR³⁹R^{39′})ᵣ—, —(CR³⁹R^{39′})ᵣC(O)NR³⁷—, —NR³⁷C(O)(CR^{39′}R^{39″})ᵣS—, —NR³⁷(CR³⁹R^{39′})ᵣ—, —(CR³⁹R^{39′})ᵣNR³⁸—, —NR³⁷(CR³⁹R^{39′})ᵣNR³⁸—, —NR³⁷(CR³⁹R^{39′})ᵣS—, —NR³⁷(CR³⁹R^{39′}CR³⁹R^{39′}O)ᵣ—, —NR³⁷(CR³⁹R^{39′}CR³⁹R^{39′}O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NR³⁷—, —(CR³⁹R^{39′}CR³⁹R^{39′}O)ᵣ—(CR³⁶R^{36′})ₜC(O)—, —C(O)(CR³⁶R^{36′})ₜ(OCR³⁹R^{39′}CR³⁹R^{39′}CR³⁹R^{39′})ᵣ—, —C(O)(CR³⁶R^{36′})ₜ(OCR³⁹R^{39′}CR³⁹R^{39′}CR³⁹R^{39′})ᵣNR³⁷—, —C(O)(CR³⁶R^{36′})ᵣ—O—(C₆-C₁₀ aryl)- (CR^{36″}R^{36′″})ₜNR³⁷—, —NR³⁷(CR³⁶R^{36′})ᵣ—(C₆-C₁₀ aryl)-O—(CR^{36″}R^{36′″})ₜC(O)—, —C(O)—(CR³⁶R^{36′})ᵣ-NR³⁷—C(O)—(C₆-C₁₀ aryl)-NR^{37′}—, —NR³⁷—(C₆-C₁₀ aryl)-C(O)— NR^{37′}—(CR³⁶R^{36′})ᵣ—C(O)—, —NR³⁷(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NR³⁷—, —NR³⁷(CR³⁶R^{36′})ᵣ—(C₆-C₁₀ aryl)-O—(CR^{36″}R^{36′″})ₜ—NR^{37′}—, or —NR^{37′}—(CR^{36″}R^{36′″})ₜ—O—(C₆-C₁₀ aryl)-(CR³⁶R^{36′})ᵣ—NR³⁷—, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
wherein
each R³⁶, R^{36′}, R^{36″} and R^{36′″} is independently selected from the group consisting of H, C₁-C₆ alkyl, C₂-C₆ alkenyl, C₂-C₆ alkynyl, C₃-C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰R^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR⁴⁰ and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each r independently is 1, 2, 3, 4, 5, 6, 7, 8, 9, or 10;
each rp independently is an integer from 1 to 80;
each t independently is 1, 2, 3, 4, 5, 6, 7, 8, 9, or 10; and
each * represents a covalent bond.

In some embodiments, each L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—;
wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

In some embodiments, each R^{39′}, when present, is H. In some embodiments, one R³⁹, when present, is not H. In some embodiments, one R³⁹, when present, is —OC(O)R⁴⁰. In some embodiments, R⁴⁰ is H. In some embodiments, R³⁸, when present, is H. In some embodiments, R³⁷, when present, is H. In some embodiments, R^{36′}, when present, is H. In some embodiments, R³⁶, when present, is H.

In some embodiments, L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—;
wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

In some embodiments, each L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36′})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—;
wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

In some embodiments, L³ is present. In some embodiments, L³ is absent.

In some embodiments, each L¹ is independently of the formula

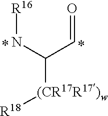

wherein
R¹⁶ is H,
each R¹⁷ and R^{17′} is independently H, C₁-C₆ alkyl, or —C(O)OH,
R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen;
   each L² is independently of the formula

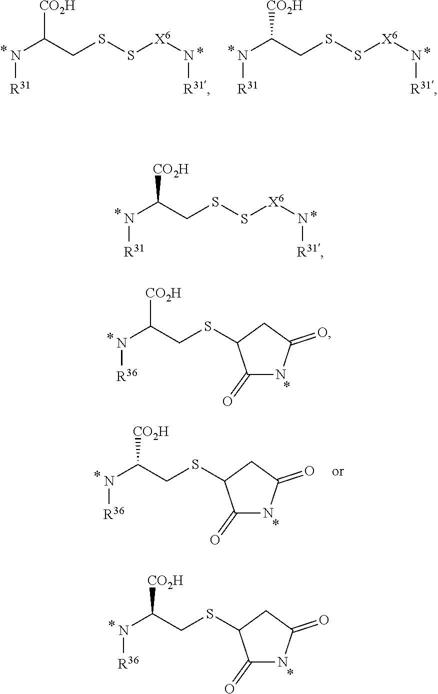

wherein
R³¹ and R^{31′} are H,
R³⁶ is H; and
   each L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ᵣ—(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36″}R^{36″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36′″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR^{36″}R^{36′″})ₜ—NH—, or —NH—(CR^{36″}R^{36′″})ₜ—O—(C₆-C₁₀ aryl)-(CR³⁶R^{36′})ᵣ—NH—;
wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.
   In some embodiments, when k is larger than 3, at least 2 of the L^{x} in formula (I) are independently selected from the following groups (also referred to herein as “particular L^{x}

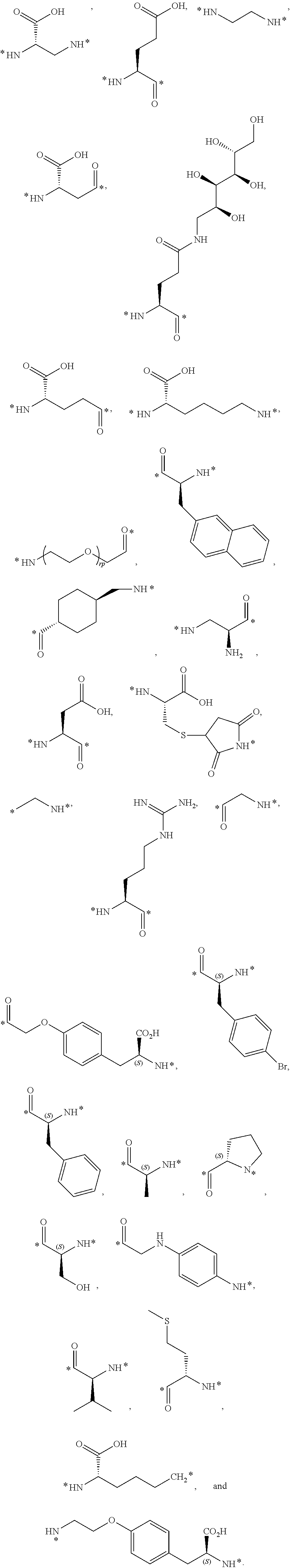

In some embodiments, when k is larger than 3, at least 3 of the L^{x} in formula (I) are independently selected from the particular L^{x} groups.

In some embodiments, when k is larger than 4, at least 3 of the L^{x} in formula (I) are independently selected from the particular L^{x} groups.

In some embodiments, when k is larger than 4, at least 3 of the L^{x} in formula (I) are independently selected from the particular L^{x} groups.

In some embodiments, when k is larger than 3, at least k−2 of the L^{x} in formula (I) are independently selected from the particular L^{x} groups.

In some embodiments, when k is larger than 3, at least k−1 of the L^{x} in formula (I) are independently selected from the particular L^{x} groups.

In some embodiments, when k is larger than 4, at least k−2 of the L^{x} in formula (I) are independently selected from the particular L^{x} groups.

In some embodiments, when k is larger than 4, at least k−1 of the L^{x} in formula (I) are independently selected from the particular L^{x} groups.

In some embodiments, when k is larger than 3, at least 2 of the L^{x} in formula (I) are independently selected from the following groups (also referred to herein as “further particular L^{x} groups”):

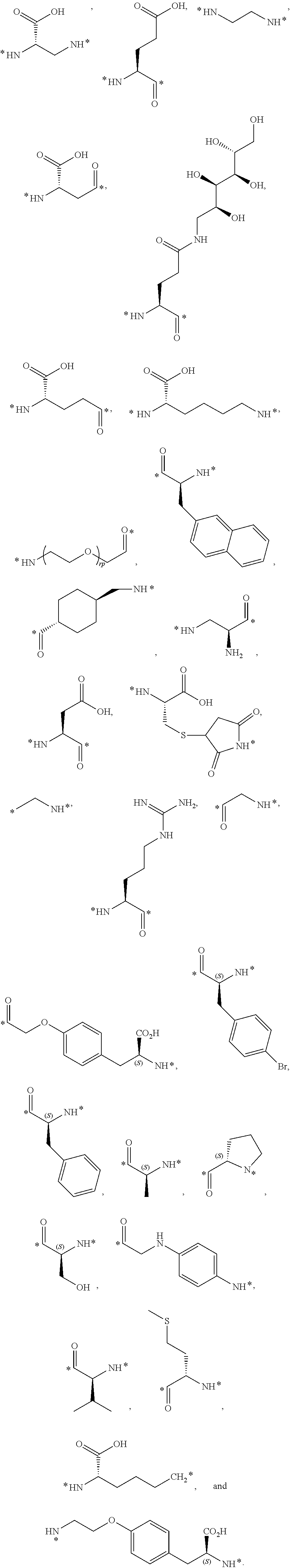

In some embodiments, when k is larger than 3, at least 3 of the L^{x} in formula (I) are independently selected from the further particular L^{x} groups.

In some embodiments, when k is larger than 4, at least 3 of the L^{x} in formula (I) are independently selected from the further particular L^{x} groups.

In some embodiments, when k is larger than 4, at least 3 of the L^{x} in formula (I) are independently selected from the further particular L^{x} groups.

In some embodiments, when k is larger than 3, at least k−2 of the L^{x} in formula (I) are independently selected from the further particular L^{x} groups.

In some embodiments, when k is larger than 3, at least k−1 of the L^{x} in formula (I) are independently selected from the further particular L^{x} groups.

In some embodiments, when k is larger than 4, at least k−2 of the L^{x} in formula (I) are independently selected from the further particular L^{x} groups.

In some embodiments, when k is larger than 4, at least k−1 of the L^{x} in formula (I) are independently selected from the further particular L^{x} groups.

In some embodiments, at least one L^{x} is

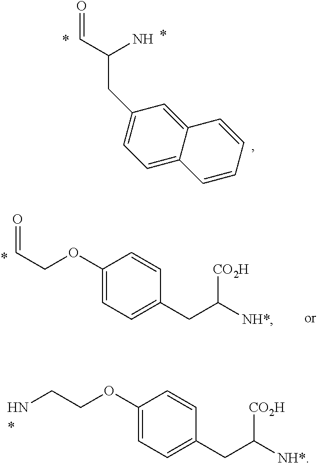

In some embodiments, at least one L^{x} is

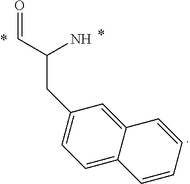 [SMILES: NC(C=O)Cc1ccc2ccccc2c1]

In some embodiments, at least one L^{x}

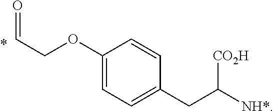

In some embodiments, at least one L^{x} is

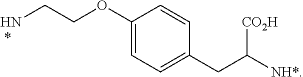

In some embodiments, at least one L^{x} is

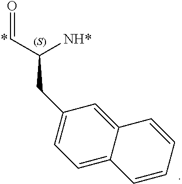

In some embodiments, one, two or three L^{x} are independently of formula

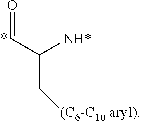

In some embodiments, -(L^{x})^{k}-comprises a group of formula (III)

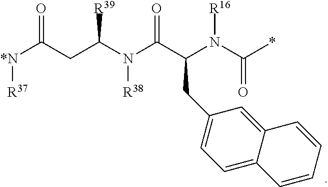

In some embodiments, -(L^{x})ₖ- comprises a group of formula (III) and R¹⁶, R³⁷ and R³⁸ in formula (III) are H.
In some embodiments, -(L^{x})ₖ- comprises a group of formula (III) and R³⁹ in formula (III) is —COOH.

### The A Group

The compounds described herein comprise a group A, which is a group which can comprise a radioelement. The linker (L^{x})ₖ connects BL with A. A can be a chelating group Ch which can comprise a metal, a radioelement, Si-¹⁸F, B-¹⁸F, or Al-¹⁸F, or A can be a radiolabeled prosthetic group PG. A compound as described herein having a chelating group with no radioelement coordinated thereto is sometimes referred to as “cold.” A compound as described herein having a chelating group with a radioelement coordinated thereto (chelated, complexed or bound within the Ch) is sometimes referred to as “hot”. Such a “hot” compound is also referred to as a radiolabeled compound.

It will be appreciated that the structure of the chelating group is not particularly restricted. Any chelating group known in the art that is capable of coordinating to a radioelement or Si-¹⁸F, B-¹⁸F, or Al-¹⁸F, known for diagnostic, imaging or therapeutic use is suitable. Preferably, the chelating group binds the radioelement or Si-¹⁸F, B-¹⁸F, or Al-¹⁸F stably such that no substantial loss of chelated radioactive particles occurs in vivo which would harm non-targeted cells.

In some embodiments, the Ch is selected from the group consisting of

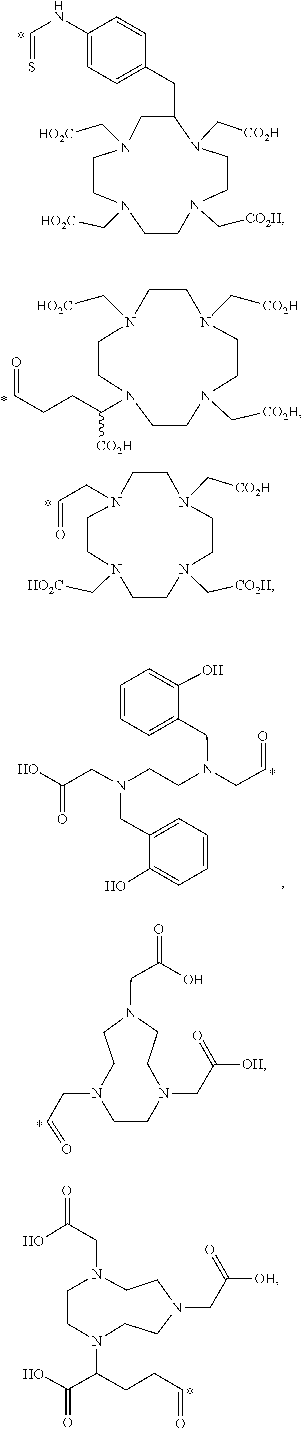

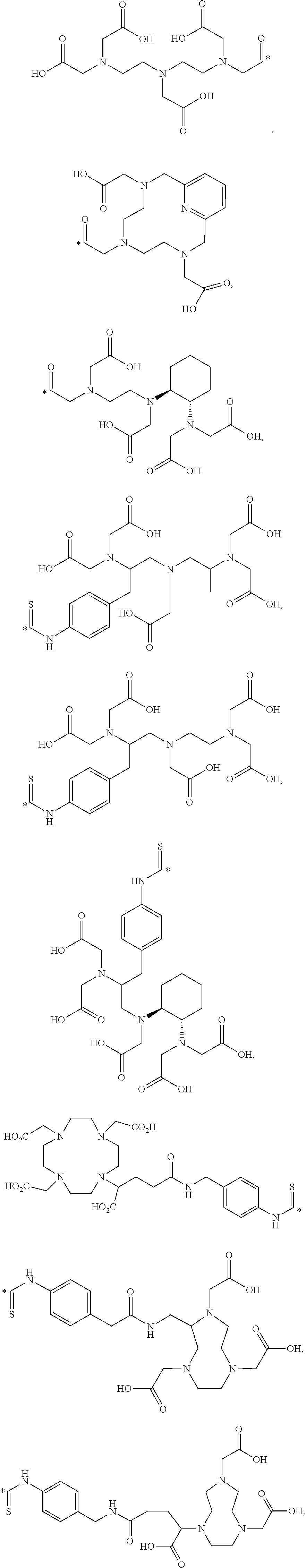

and Ch can comprise a radioelement, Si-¹⁸F, B-¹⁸F, or Al-¹⁸F; wherein * represents a covalent bond to the rest of compound.

In some embodiments, the Ch is selected from the group consisting of

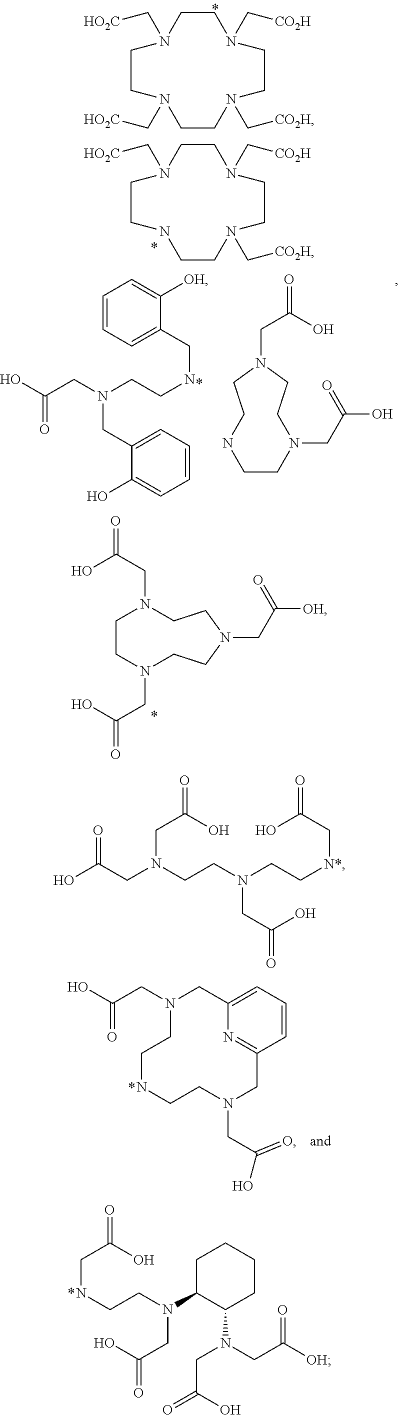

and Ch can comprise a radioelement, Si-¹⁸F, B-¹⁸F, or Al-¹⁸F; wherein * represents a covalent bond to the rest of compound.
In some embodiments, Ch is

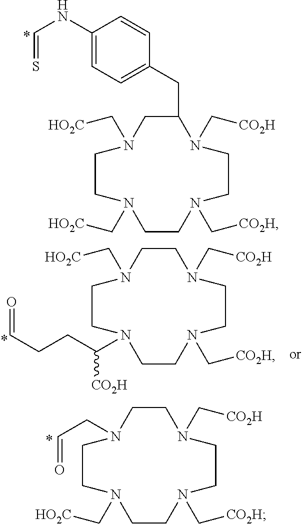

and Ch can comprise a radioelement, Si-¹⁸F, B-¹⁸F, or Al-¹⁸F; wherein * represents a covalent bond to the rest of compound.

Unless otherwise indicated herein, whenever a structural formula of a chelating group Ch is shown herein, Ch can comprise a radioelement, Si-¹⁸F, B-¹⁸F, or Al-¹⁸F even though such a radioelement Si-¹⁸F, B-¹⁸F, or Al-¹⁸F is not shown in the structural formula, that is, a compound of the present disclosure including such a Ch group can be either a cold or hot compound. For example, this means that a formula such as

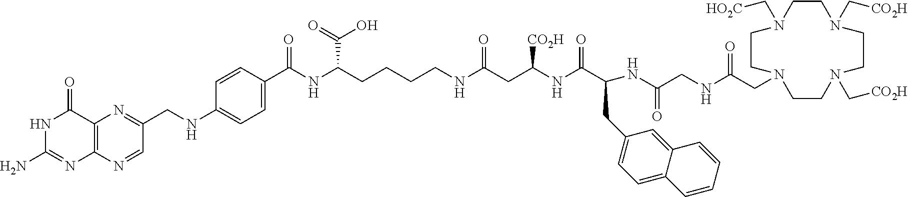 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

includes a hot compound such as

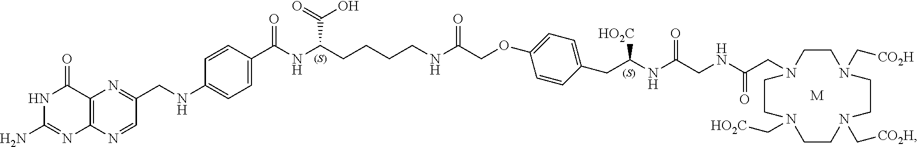

wherein M can be a radioelement, Si-¹⁸F, B-¹⁸F, or Al-¹⁸F, unless it is indicated otherwise (e.g., by referring to the compound as “cold”, “not radiolabeled” etc., or otherwise implied by the description, for example, where the synthesis of cold compounds is described).

The compounds of the present disclosure can include a chelating group Ch (i.e., A in compounds of formula (I) is Ch) which can comprise a metal, a radioelement, Si-¹⁸F, B-¹⁸F, or Al-¹⁸F, or the compounds can include a radiolabeled prosthetic group PG.

Compounds of the present disclosure in which A is a chelating group Ch comprising Si-¹⁸F, B-¹⁸F, or Al-¹⁸F stably bound within the chelating group, or A is a radiolabled prosthetic group, are particularly suitable for diagnosis and imaging of FR expressing cells in a subject, such as FR expressing cancer cells and tumors.

Radiolabeled prosthetic groups PG and methods for covalently attaching such prosthetic groups to amino acids and peptides are known in the art. See e.g., Fani et al., Theranostics 2012; 2(5):481-501; and Richter and Wuest, Molecules 2014, 19: 20536-20556. Such methods can be used for conjugation to form PG covalently attached to (L^{x})ₖ in formula (I) of compounds of the present disclosure.

PG can be radiolabeled with a radiohalogen selected from the group consisting of ¹⁸F, ⁷⁵Br, ⁷⁶Br, ⁷⁷Br, ⁸⁰Br, ^{80m} Br, ⁸²Br, ¹²³I, ¹²⁴I, ¹²⁵I, ¹³¹I and ²¹¹At.

Examples of radiolabeled prosthetic groups PG include, but are not limited to

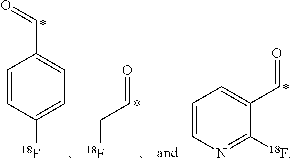

In some embodiments, A is a chelating group Ch which can comprise a metal, a radioelement, Si-¹⁸F, B-¹⁸F, or Al-¹⁸F.

In some embodiments, A is a chelating group Ch which can comprise a metal or a radioelement, but not a Si-¹⁸F, B-¹⁸F, or Al-¹⁸F group.

In some embodiments, A is a chelating group Ch which can comprise a radioelement, but not a Si-¹⁸F, B-¹⁸F, or Al-¹⁸F group.

In some embodiments, A is a chelating group Ch comprising a metal, a radioelement, Si-¹⁸F, B-¹⁸F, or Al-¹⁸F.

In some embodiments, A is a chelating group Ch comprising a metal.

In some embodiments, A is a chelating group Ch comprising a radioelement.

In some embodiments, A is a chelating group Ch comprising a radioelement selected from the group consisting of ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹b, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

In some embodiments, A is a chelating group Ch comprising a radioelement selected from the group consisting of ¹⁶⁹Er, ⁶⁴Cu, ⁶⁷Cu, ¹⁸⁶Re, ¹⁸⁸Re, ⁹⁰Y, ¹⁷⁷Lu, ¹⁶¹Tb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ¹⁴²Pr, ¹⁴³Pr, ¹⁹⁸Au, ¹⁹⁹Au, ¹⁴⁹Tb, ⁴⁷Sc, ²²⁵Ac, ²¹²Pb, ²¹¹At, ²²³Ra, ²²⁷Th, ¹³¹I, ⁷⁶As, ¹¹¹Ag, ¹⁶⁵Er, and ²²⁷Ac.

In some embodiments, A is a chelating group Ch comprising a radioelement selected from the group consisting of ¹¹¹In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁷⁷Lu, ¹⁶⁹Yb, ¹⁵¹Pm, ¹⁷²Tm, ^{117m}Sn, ¹²³I, ¹²⁴I, ¹²⁵I, ¹⁸F, ¹⁵²Tb, ¹⁵⁵Tb, ⁴⁴Sc, ⁴³Sc, ⁸²Rb, ⁸⁹Zr, and ⁶¹Cu.

In some embodiments, A is a chelating group Ch comprising a radioelement selected from the group consisting of ⁶⁶Ga, ⁶⁷Ga, ⁶⁸Ga, ¹⁷⁷Lu, and ²²⁵Ac.

In some embodiments, A is a chelating group Ch comprising a radioelement which is ¹⁷⁷Lu or ²²⁵Ac.

In some embodiments, A is a chelating group Ch comprising ¹⁷⁷Lu.

In some embodiments, A is a chelating group Ch comprising ²²⁵Ac.

In some embodiments, A is a chelating group Ch comprising a Si-¹⁸F, B-¹⁸F, or Al-¹⁸F.

In some embodiments, A is a prosthetic group PG.

### Compound Syntheses and Intermediates

The present disclosure further provides intermediate compounds (also referred to as intermediates) which are used to make the compounds described herein.

One embodiment is an intermediate compound described (explicitly or implicitly) in any one of Examples 1 to 52.

A further embodiment is an intermediate compound of formula

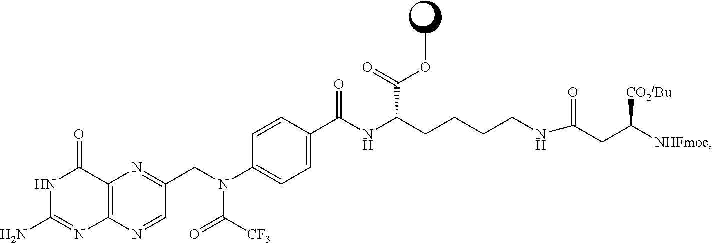

wherein [symbol] represents a resin suitable for solid phase peptide synthesis (e.g., a Wang resin) or H (e.g., if the intermediate is removed from the resin).

A further embodiment is an intermediate compound of formula

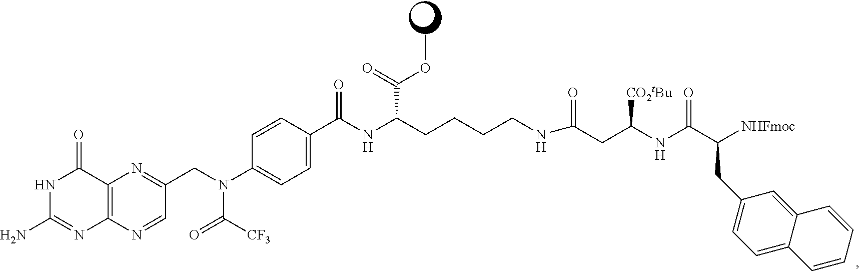

wherein [symbol] represents a resin suitable for solid phase peptide synthesis (e.g., a Wang resin) or H (e.g., if the intermediate is removed from the resin).

A further embodiment is an intermediate compound of formula

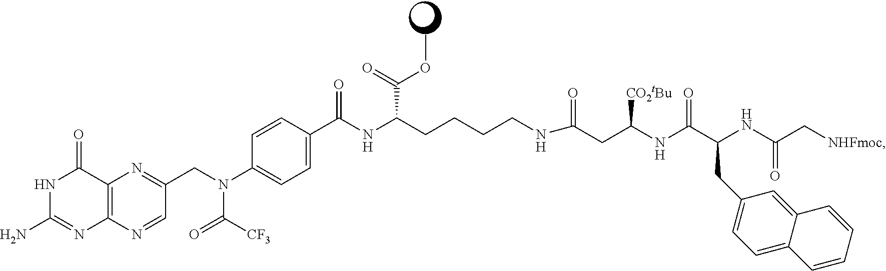

[symbol]

represents a resin suitable for solid phase peptide synthesis (e.g., a Wang resin) or H (e.g., if the intermediate is removed from the resin).

A further embodiment is an intermediate compound of formula

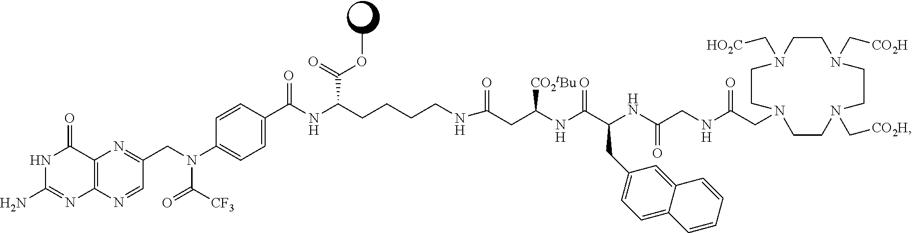

wherein [symbol] represents a resin suitable for solid phase peptide synthesis (e.g., a Wang resin) or H (e.g., if the intermediate is removed from the resin).

A further embodiment is an intermediate compound of formula

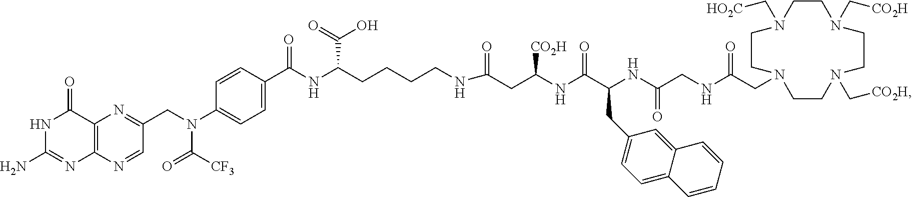

wherein [symbol] represents a resin suitable for solid phase peptide synthesis (e.g. a Wang resin) or H (e.g., if the intermediate is removed from the resin).

A further embodiment is an intermediate compound of formula

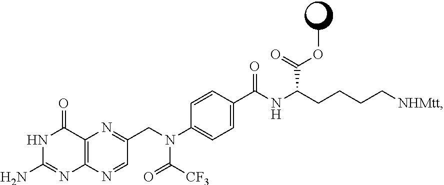

wherein [symbol] represents a resin suitable for solid phase peptide synthesis (e.g., a Wang resin) or H (e.g., if the intermediate is removed from the resin).

A further embodiment is an intermediate compound of formula

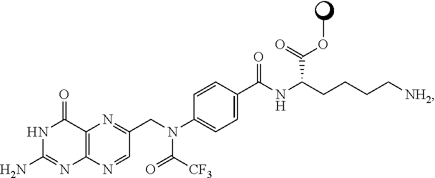

wherein [symbol] represents a resin suitable for solid phase peptide synthesis (e.g., a Wang resin) or H (e.g., if the intermediate is removed from the resin).

A further embodiment is an intermediate compound of formula

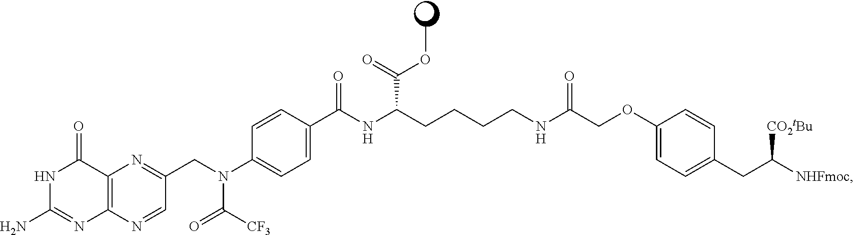

wherein [symbol] represents a resin suitable for solid phase peptide synthesis (e.g., a Wang resin) or H (e.g., if the intermediate is removed from the resin).

A further embodiment is an intermediate compound of formula

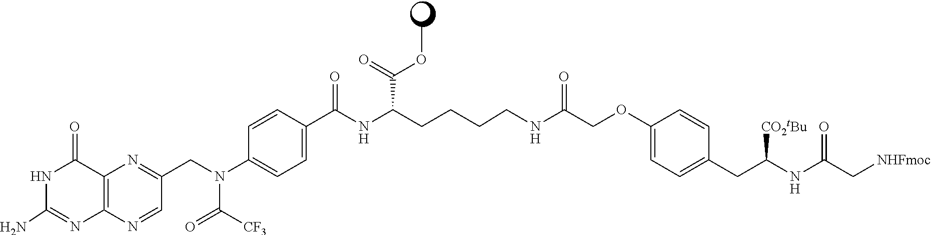

wherein [symbol] represents a resin suitable for solid phase peptide synthesis (e.g., a Wang resin) or H (e.g., if the intermediate is removed from the resin).

A further embodiment is an intermediate compound of formula

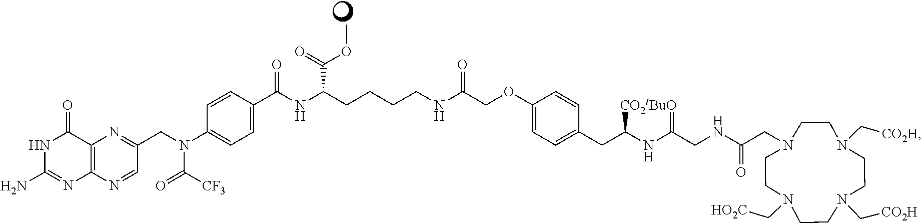

wherein [symbol] represents a resin suitable for solid phase peptide synthesis (e.g., a Wang resin) or H (e.g., if the intermediate is removed from the resin).

A further embodiment is an intermediate compound of formula

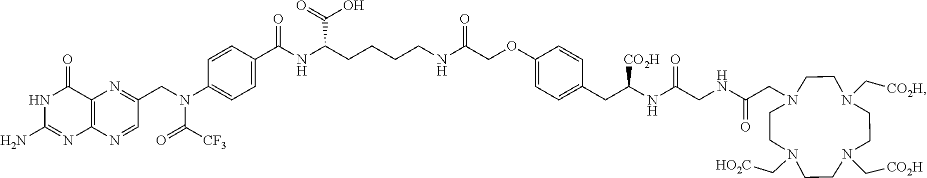

wherein [symbol] represents a resin suitable for solid phase peptide synthesis (e.g., a Wang resin) or H (e.g., if the intermediate is removed from the resin).

A further embodiment is an intermediate compound of formula

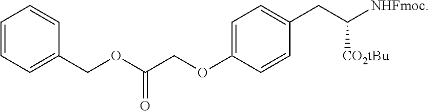

The present disclosure further provides methods of synthesis for the compounds and intermediate compounds of the present disclosure.

One embodiment is a method of synthesis as described (explicitly or implicitly) in any one of Examples 1 to 52.

One embodiment is a method comprising.
(a) providing an intermediate compound attached to a resin (e.g., a Wang resin) of formula (I-1)

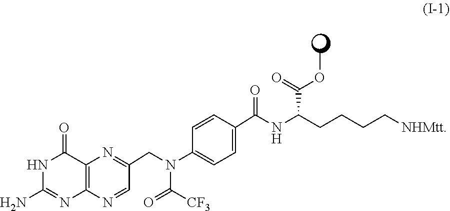

(b) coupling compound (I-1) with Fmoc-Asp-O^{t}Bu to form an intermediate compound of formula (I-2)

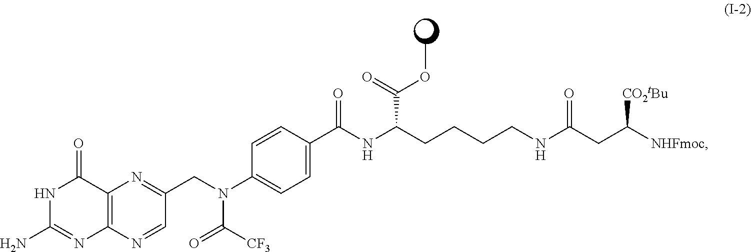

(c) coupling compound (I-2) with Fmoc-2-Nal-OH to form an intermediate compound of formula (I-3)

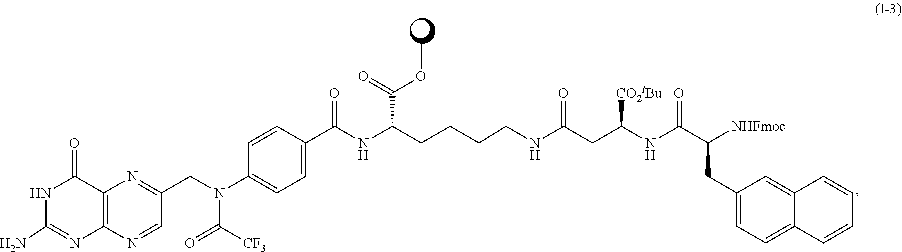

(d) coupling compound (I-3) with Fmoc-Gly-OH to form an intermediate compound of formula (I-4)

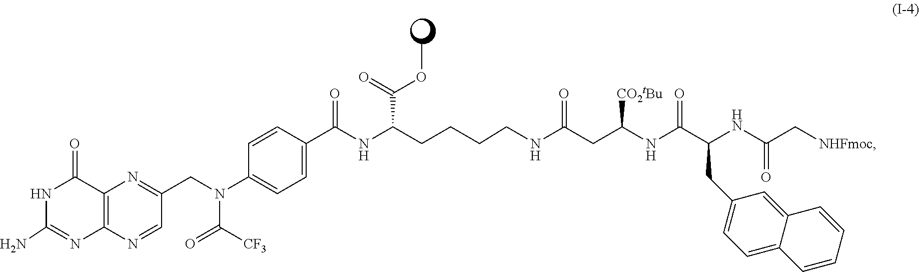

(e) coupling compound (I1-4) with DOTA-ONHS to form an intermediate compound of formula (I-5)

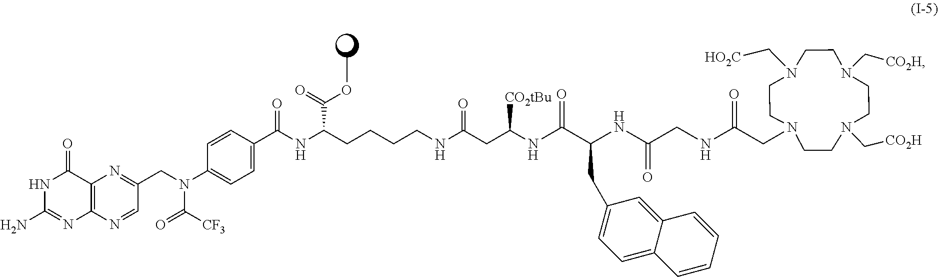 [SMILES: COC(=O)[C@H](CCCCNC(=O)C[C@H](NC(=O)[C@H](Cc1ccc2ccccc2c1)NC(=O)CNC(=O)CN1CCN(CC(=O)O)CCN(CC(=O)O)CCN(CC(=O)O)CC1)C(=O)OC(C)(C)C)NC(=O)c1ccc(N(Cc2cnc3nc(N)[nH]c(=O)c3n2)C(=O)C(F)(F)F)cc1]

(f) cleaving compound (I-5) from the resin, and
(g) deprotecting the N¹⁰-TFA group of the pteryl to form a compound of formula

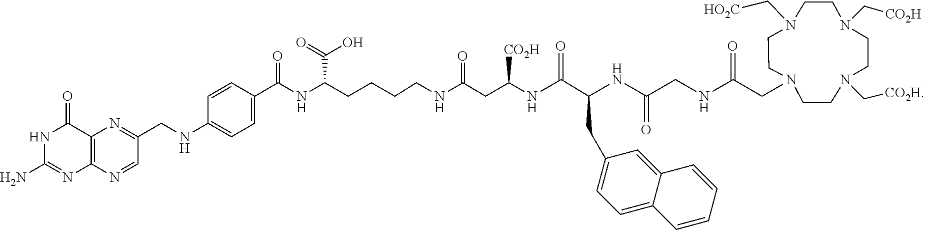 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

A further embodiment is a method comprising.
(a) providing an intermediate compound attached to a resin (e.g., a Wang resin) of formula (I′-1)

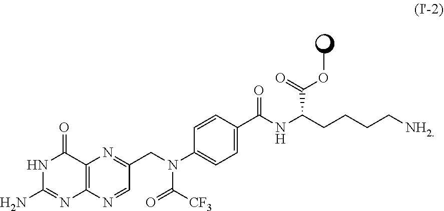 [SMILES: COC(=O)[C@H](CCCCN)NC(=O)c1ccc(N(Cc2cnc3nc(N)[nH]c(=O)c3n2)C(=O)C(F)(F)F)cc1]

(b) coupling compound (I′-1) with

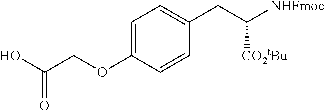

to form an intermediate compound of formula (I′-2)

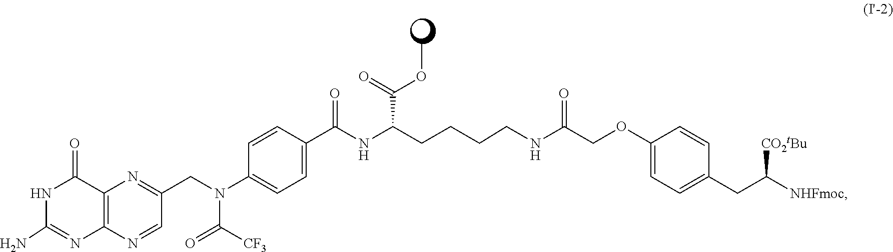

(c) coupling compound (I′-2) with Fmoc-Gly-OH to form an intermediate compound of formula (I′-3)

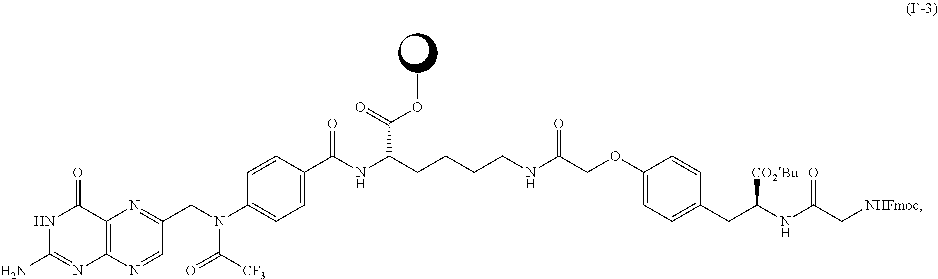

(d) coupling compound (I′-3) with DOTA-ONHS to form an intermediate compound of formula (I′-4)

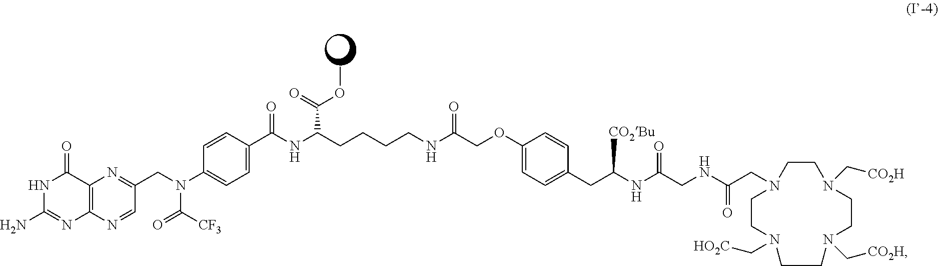

(e) cleaving compound (I′-4) from the resin, and
(f) deprotecting the N¹⁰-TFA group of the pteryl to form a compound of formula

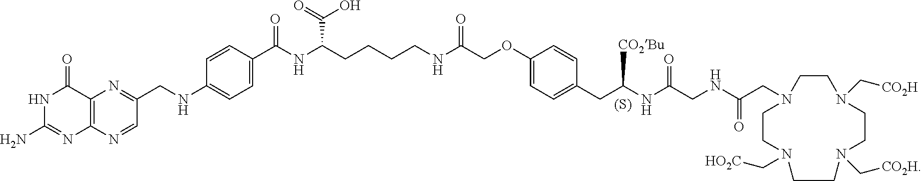

A further embodiment is a method comprising

reacting

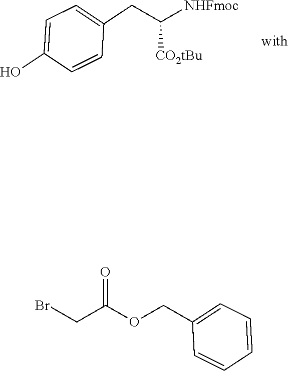

to form

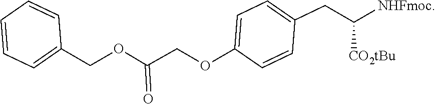

In a specific embodiment, the reacting is under strong basic conditions. In a further specific embodiment, the reacting is under strong basic conditions by use of K₂CO₃. In a further specific embodiment, the reacting is under strong basic conditions by use of K₂CO₃, at a temperature between about 15° C. and about 35° C., and the reacting is performed for about 2 to 6 hours. In a further specific embodiment, the reacting is under strong basic conditions by use of K₂CO₃, at a temperature between about 18° C. and about 28° C., and the reacting is performed for about 2.5 to 3.5 hours. In a further specific embodiment, the reacting is under strong basic conditions by use of K₂CO₃, at a temperature between about 20° C. and about 25° C., and the reacting is performed for about 3 hours. In a more specific embodiment of the above embodiment or any specific embodiment thereof, between about 2 and about 4 equivalents K₂CO₃ are used, preferably, about 3 equivalents K₂CO₃ (for example, for 3.0 mM Fmoc-Tyr-O^{t}Bu this would mean about 9 mM K₂CO₃). In a more specific embodiment of the above embodiment or any specfic embodiment thereof, between about 2 and about 4 equivalents K₂CO₃ are used, preferably, about 3 equivalents K₂CO₃ (for example, for 3.0 mM Fmoc-Tyr-O^{t}Bu this would mean about 9 mM K₂CO₃) and the reacting occurs in a mixture containing acetone (typically, dry acetone).

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or ““hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″— group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″— group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″— group, where R″ is any R group as described in the various embodiments provided herein.

As used herein, “nitro” refers to a —NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present disclosure is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms (for example, of a pteryl group) are also intended to be included.

For compounds that are specifically recited as excluded from the invention, all possible stereoisomers, mixtures of stereoisomers, tautomers, and salt forms of these compounds are also meant to be excluded.

As used herein, the terms “salt” or “salts” refers to an acid addition or base addition salt of a compound of the present disclosure. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present disclosure are capable of forming acid and/or base salts by virtue of the presence of amino and/or carboxyl groups or groups similar thereto. When both a basic group and an acid group are present in the same molecule, the compounds of the present disclosure may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present disclosure provides compounds of the present disclosure in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

As used herein, “amino acid” means any molecule, whether natural or synthetic (including non-protogeneic), that includes an alpha-carbon atom covalently bonded to an amino group and an acid group. The acid group can be a carboxyl group. Other suitable acid functionalities are those which are capable of being included in a polymer of naturally-occurring amino acids. The term “amino acid” includes molecules having one of the formulas:

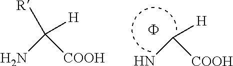

wherein R′ is a side group such as a linear or branched C₁-C₁₂ alkyl group in which one or more —H are optionally substituted by —NH₂, —CO₂H, —OH, —C(O)NH₂, —SH, —SCH₃, —NHC(═NH₂)NH₂, an aryl group such as a phenyl group or a hydroxyphenyl group, a heteroaryl group such as an imidazolyl group or indolyl group, a cycloalkyl group, or a heterocycloalkyl group such as a pyrrolidinyl group, and ring Φ includes at least 3 carbon atoms.

Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

Illustrative amino acid groups include, but are not limited to, the twenty endogenous human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, citrulline (CIT), 1,3-methyl-histidine (ME-HIS), alpha-amino-adipic acid (AAA), alpha-amino-butyric acid (BABA), L-allo-cystathionine (cystathionine-A; CYSTA-A), L-cystathionine (cystathionine-B; CYSTA-B), cystine, allo-isoleucine (ALLO-ILE), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), and the like.

As used herein the term “amino acid residue” refers to the part of an amino acid which remains after the amino acid has been covalently bonded to two portions of the compound containing the amino acid residue through (1) an alpha-acid group (typically, alpha-carboxyl) and an alpha-amino group of the amino acid (e.g., α-Asp) or (2) through a side-chain (R′) acid group (typically, carboxyl) or side chain (R′) amino group, and an alpha-acid group (typically, alpha-carboxyl) and an alpha-amino group of the amino acid (e.g., β-Asp).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

It will be understood that amino acids, when used in connection with the compounds and conjugates described herein, may exist as zwitterions in a conjugate in which they are incorporated.

As used herein, “sugar” refers to carbohydrates, such as monosaccharides, disaccharides, or oligosaccharides. In connection with the present disclosure, monosaccharides are preferred. Non-limiting examples of sugars include erythrose, threose, ribose, arabinose, xylose, lyxose, allose, altrose, glucose, mannose, galactose, ribulose, fructose, sorbose, tagatose, and the like. It will be understood that as used in connection with the present disclosure, sugar includes cyclic isomers of amino sugars, deoxy sugars, acidic sugars, and combinations thereof. Non-limiting examples of such sugars include, galactosamine, glucosamine, deoxyribose, fucose, rhamnose, glucuronic acid, ascorbic acid, and the like. In some embodiments, sugars for use in connection with the present disclosure include

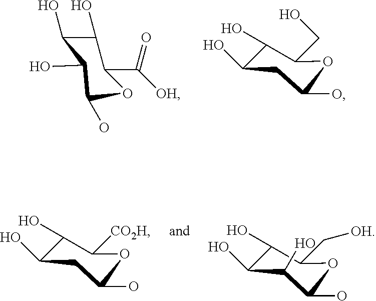

As used herein, the term “pharmaceutical composition” refers to a compound of the invention, or a pharmaceutically acceptable salt thereof, together with at least one pharmaceutically acceptable carrier, in a form suitable for oral or parenteral administration.

The pharmaceutical compositions of the invention may include a “therapeutically effective amount” or a “prophylactically effective amount” of a compound of the present invention.

A “therapeutically effective amount” refers to an amount effective, at dosages and for periods of time necessary, to achieve the desired therapeutic result. A therapeutically effective amount according to the invention may vary according to factors such as the disease state, age, sex, and weight of the individual, and the ability of the FR therapeutic agent, such as a radiolabeled (e.g., with ¹⁷⁷Lu) compound of formula (I), in optional combination with an additional therapeutic agent, such as the Immuno-Oncology therapeutic agent, to elicit a desired response in the individual. A therapeutically effective amount is also one in which any toxic or detrimental effects of the FR therapeutic agent, such as a radiolabeled (e.g., with ¹⁷⁷Lu) compound of formula (I), in optional combination with an additional therapeutic agent is outweighed by the therapeutically beneficial effects.

A “therapeutically effective dosage” can inhibit a measurable parameter, e.g., tumor growth rate by at least about 20%, by at least about 40%, by at least about 60%, or by at least about 80% relative to untreated subjects. The ability of the combination according to the invention to inhibit a measurable parameter, e.g., cancer, can be evaluated in an animal model system predictive of efficacy in human tumors. Alternatively, this property of a composition can be evaluated by examining the ability of the combination according to the invention to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “treat”, “treating” or “treatment” of any disease or disorder refer to the reduction or amelioration of the progression, severity and/or duration of a disorder, e.g., a proliferative disorder, such as cancer, or the amelioration of one or more symptoms (e.g., one or more discernible symptoms) of the disorder resulting from the administration of one or more therapies or therapeutic agents; or alleviating or ameliorating at least one physical parameter or biomarker associated with the disease or disorder, including those which may not be discernible to the patient. In specific embodiments, the terms “treat,” “treatment” and “treating” refer to the amelioration of at least one measurable physical parameter of a proliferative disorder, such as a cancer, for example, growth of a tumor, not necessarily discernible by the patient. In other embodiments the terms “treat”, “treatment” and “treating” refer to the inhibition of the progression of a proliferative disorder, such as a cancer, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

As used herein, the term “prevent”, “preventing” or “prevention” of any disease or disorder refers to the prophylactic treatment of the disease or disorder; or delaying the onset or progression of the disease or disorder.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the therapeutic agents described herein (e.g., peptides, polynucleotides, cells, small molecules, and antibodies to prevent the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells, but can include benign cancers. In various embodiments, cancer cells can spread locally or through the bloodstream and lymphatic system to other parts of the body. In some aspects, the cancer is a FR expressing cancer. In some embodiments, the cancer is a FR-α expressing cancer. Examples of various cancers are described herein. For example, cancers can include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and the like. Other exemplary cancers include, but are not limited to, small cell lung cancer, bone cancer, cancer of the head or neck, hepatocellular carcinoma, cutaneous or intraocular melanoma, uterine cancer, stomach cancer, colon cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the parathyroid gland, cancer of the adrenal gland, cancer of the urethra, cancer of the penis, cancer of the ureter, neoplasms of the central nervous system (CNS), primary CNS lymphoma, spinal axis tumors, brain stem glioma, pituitary adenoma, inflammatory myofibroblastic tumors, and combinations thereof.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors and benign cancers. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

As used herein, a subject is “in need of” a treatment if such subject would benefit biologically, medically or in quality of life from such treatment.

As used herein, the term “a,” “an,” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present disclosure can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present disclosure can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present disclosure or racemic intermediates can also be resolved by chiral chromatography, e.g., high pressure liquid chromatography (HPLC) using a chiral adsorbent.

### Radioelements

The chelating groups of the FR targeting compounds described herein, can comprise a radioelement.

In some embodiments, the radioelement is ²²⁵Ac or ¹⁷⁷Lu.

¹⁷⁷Lu has a half-life of 6.7 days. It emits 0.5 MeV energy consisting of negatively charged β particles (electrons) that travel chaotically through tissues for approximately 20-80 cells or 0.5-2 mm and cause predominantly base damage and single strand breaks (i.e., lesions). At high dose these lesions can interact to convert sublethal damage (SLD) or potentially lethal damage (PLD) to irreparable, lethal damage. ¹⁷⁷Lu also emits 113Kv and 208 kV radiation which can be used for imaging.

²²⁵Ac has a half-life of 9.9 days, and in contrast emits 8.38 MV energy alpha particles. Only 0.5% of the energy is emitted as 142Kv photon emissions. The majority of radiation particles are therefore positively charged, and about 8,000 times larger than β particles. Furthermore, the energy from these particles is deposited over relatively short distances (2-3 cells). As a result, there is dense and severe tissue damage in the form of double strand breaks with multiply damaged sites that represent irreparable lethal damage. This is called High Linear Energy Transfer (LET) or densely ionizing ionization and it delivers 3-7× more absorbed dose than β particles. The type of cellular damage inflicted by either isotope (¹⁷⁷Lu or ²²⁵Ac) is expected to be different due to the difference of the characteristics of each warhead. ¹⁷⁷Lu is believed to provide a longer path length of radiation and therefore can be effective in delivering radiation to adjacent cells. The preponderance of single strand breaks, especially in the presence of oxygen, provides the opportunity to repair sub lethal damage (SLD) and or potentially lethal damage (PLD) providing the optimal conditions for normal tissue repair. On the contrary, ²²⁵Ac delivers extremely powerful, high LET radiation, and the potential for repair of normal tissue is much more limited. The radiological biological effectiveness of alpha radiation is at least 5 times that of beta irradiation and for administered doses the relative biological effectiveness (RBE) has to be taken into account. With ²²⁵Ac therapy, the type of DNA damage inflicted does not require the presence of oxygen so it will also be more effective in hypoxic tumor regions.

Suitable radioelements include ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

Radioelements suitable for therapeutic uses of the FR targeting compounds disclosed herein, include, but are not limited to, ¹⁶⁹Er, ⁶⁴Cu, ⁶⁷Cu, ¹⁸⁶Re, ¹⁸⁸Re, ⁹⁰Y, ¹⁷⁷Lu, ¹⁶¹Tb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ¹⁴²Pr, ¹⁴³Pr, ¹⁹⁸Au, ¹⁹⁹Au, ¹⁴⁹Tb, ⁴⁷Sc, ²²⁵Ac, ²¹²Pb, ²¹¹At, ²²³Ra, ²²⁷Th, ¹³¹I, ⁷⁶As, ¹¹¹Ag, ¹⁶⁵Er, and ²²⁷Ac.

Radioelements suitable for diagnostic uses of the FR targeting compounds disclosed herein, include, but are not limited to, ¹¹¹In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹C, ^{52m}Mn, ¹⁷⁷Lu, ¹⁶⁹Yb, ¹⁵¹Pm, ¹⁷²Tm, ^{117m}Sn, ¹²³I, ¹²⁴I, ¹²⁵I, ¹⁸F, ¹⁵²Tb, ¹⁵⁵Tb, ⁴⁴Sc, ⁴³Sc, ⁸²Rb, ⁸⁹Zr, and ⁶¹Cu.

### Metals

The chelating groups of the FR targeting compounds described herein, can comprise a metal suitable for imaging.

Metals suitable for nuclear magnetic resonance diagnostic uses or the like of the FR targeting compounds disclosed herein, include, a metal ion exhibiting paramagnetism (e.g., a paramagnetic ion of a metal selected from the group consisting of Co, Mn, Cu, Cr, Ni, V, Au, Fe, Eu, Gd, Dy, Tb, Ho, and Er)

Metals suitable for x-ray diagnostic uses or the like of the FR targeting compounds disclosed herein, include a metal ion absorbing x-rays (e.g., an ion of a metal selected from the group consisting of Re, Sm, Ho, Lu, Pm, Y, Bi, Pb, Os, Pd, Gd, L^{a}, Au, Yb, Dy, Cu, Rh, Ag, and Ir).

### Albumin Binding Moieties

The FR targeting compounds of the present disclosure, for example, of any one of Embodiments 1-56, can be optionally substituted with an albumin-binding moiety (such as Evans blue and derivatives thereof, and 4-(p-iodophenyl)butyric acid). This substitution can be made at the group L^{x} or A (a chelating group Ch or prosthetic group PG). Albumin-binding moieties and associated connection chemistry is known in the art. See, for example, the review article by Lau et al., Bioconjugate Chem. 2019, 30, 487-502, and references cited therein.

### Combinations

The combinations of the present disclosure include a FR targeting compound of the present disclosure (e.g., a compound of formula (I) which can include a radioelement complexed by the compound's chelating group) and one or more additional therapeutic agents as described below, which can be administered to a patient to treat a proliferative disease such as cancer, particularly FR expressing cancer. The additional therapeutic agent(s) can be any of the therapeutic agents described herein.

In one embodiment of the combinations for use or the methods described herein, wherein the FR targeting compound is radiolabeled (i.e., includes complexed radioelement), the compound includes a radioelement selected from ¹⁷⁷Lu and ²²⁵Ac. In one specific embodiment, the compound radiolabeled with ¹⁷⁷Lu is administered. In another embodiment, compound radiolabeled with ²²⁵Ac is administered. In yet another embodiment, compound radiolabeled with ¹⁷⁷Lu, and compound radiolabeled with ²²⁵Ac, are both administered. The FR targeting compound can be administered in a parenteral dosage form. In some embodiments, the parenteral dosage form is selected from the group consisting of intradermal, subcutaneous, intramuscular, intraperitoneal, intravenous, and intrathecal.

In various embodiments, where the FR targeting compound (e.g., of formula (I)) is radiolabeled with ¹⁷⁷Lu, the amount administered is from about 0.1 GBq to about 15 GBq. In some embodiments, the total dose of the FR targeting compound radiolabeled with ¹⁷⁷Lu ranges from about 1 GBq to about 200 GBq.

In various embodiments, where the FR targeting compound (e.g., of formula (I)) is radiolabeled with ²²⁵Ac, the amount administered is from about 1 MBq to about 20 MBq

In other aspects, the combinations and methods described herein further comprise imaging FR expression by the cancer. In some embodiments, the step of imaging occurs before the step of administering the FR targeting compound, such as radiolabeled compound of formula (I). In other embodiments, the step of imaging occurs after the step of administering the FR targeting compound, such as radiolabeled compound of formula (I).

In various embodiments, the imaging method is selected from the group consisting of single-photon emission computed tomography (SPECT) imaging, positron-emission tomography imaging, immunohistochemistry (IHC), and fluorescence in-situ hybridization (FISH). In some embodiments, the imaging is performed by SPECT imaging.

In some embodiments, the combinations described herein include an FR targeting compound described herein, which is not radiolabeled.

In some embodiments, the combinations described herein include an FR targeting compound described herein, which comprises a radioelement, or Si-¹⁸F, B-¹⁸F, or Al-¹⁸F.

### Additional Therapeutic Agents

The combination according to the invention comprises a FR targeting compound as described above, such as radiolabeled Compound I and one or more additional therapeutic agent, such as immuno-oncology (I-0) therapeutic agents, as described below.

### Immuno-Oncology Therapeutic Agents

In various preferred embodiments I-O agents can be used as additional therapeutic agent with the FR targeting compound, such as a radiolabeled compound of formula (I), described herein. Any of the I-O agents described in this section titled “Immuno-Oncology Therapeutic Agents” can be used with a FR targeting compound, such as a radiolabeled compound of formula (I) described herein, to treat cancer.

For example, PD-1 inhibitors can be used. The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

In certain embodiments, a combination or method as described herein comprises a PD-1 inhibitor as I-O agent. In some embodiments, the PD-1 inhibitor is chosen from PDR001 (Novartis), Pembrolizumab (Merck & Co), Pidilizumab (CureTech), Durvalomab, Atezolizumab, Avelumab, Nivolumab (Bristol-Myers Squibb Company), MK-3475, MPDL3280A, MEDI4736, ipilimumab (Bristol-Myers Squibb Company), tremelimumab, MEDI0680 (Medimmune), REGN2810 (Regeneron), TSR-042 (Tesaro), PF-06801591 (Pfizer), BGB-A317 (Beigene), BGB-108 (Beigene), INCSHRI210 (Incyte), or AMP-224 (Amplimmune). In some embodiments, the PD-1 inhibitor is PDR001. PDR001 is also known as Spartalizumab. In other embodiments, the PD-1 inhibitor is not Pembrolizumab.

In an embodiment, the combination or combination therapy comprises, in addition to an FR targeting compound of the present disclosure, one or more other therapeutic agents selected from an mTOR inhibitor, a LAG-3 inhibitor, a TIM-3 inhibitor, a GITR agonist (e.g., anti-GITR antibody molecule), a TGF-β Inhibitor, and an IL-15/IL-15Ra complex.

In certain instances, the combination or combination therapy comprises, in addition to an FR targeting compound of the present disclosure, one or more other therapeutic agents, such as other anti-cancer agents, anti-allergic agents, anti-nausea agents (or anti-emetics), chemotherapeutic agents, pain relievers, cytoprotective agents, and combinations thereof.

In other embodiments, the combination or combination therapy comprises, in addition to an FR targeting compound of the present disclosure, one or more other therapeutic agents selected from the group consisting of: a tyrosine kinase inhibitor; a vascular endothelial growth factor (VEGF) receptor inhibitor; a platelet-derived growth factor (PDGF) receptor inhibitor; a fibroblast growth factor receptor (FGFR) inhibitor; am aurora kinase inhibitor; a cyclin-dependent kinase (CDK) inhibitor; a checkpoint kinase (CHK) inhibitor; a 3-phosphoinositide-dependent kinase-1 (PDK1 or PDPK1) inhibitor; a pyruvate dehydrogenase kinase (PDK) inhibitor; a protein kinase B (PKB) or AKT inhibitor; a protein kinase C (PKC) activator; a B-RAF inhibitor; a C-RAF inhibitor; a KRAS inhibitor; a human granulocyte colony-stimulating factor (G-CSF) modulator; a RET inhibitor; an FMS-like tyrosine kinase 3 (FLT3) inhibitor or CD135; c-KIT inhibitor; a Bcr/Abl kinase inhibitors; an IGF-1R inhibitor; an IGF-1R antibody; a PIM kinase inhibitor; a MET inhibitor; a human epidermal growth factor receptor 2 (HER2 receptor) (also known as Neu, ErbB-2, CD340, or p185 inhibitor); an epidermal growth factor receptor (EGFR) inhibitor; an EGFR antibody; a hedgehog antagonists; an mTOR inhibitor; a phosphoinositide 3-kinase (PI3K) inhibitor; a BCL-2 inhibitor; a mitogen-activated protein kinase (MEK) inhibitor; a P38 MAPK inhibitor; a JAK inhibitor; an alkylating agent; an aromatase inhibitor; a topoisomerase I inhibitor; a topoisomerase II inhibitor; a DNA synthesis inhibitor; a folate antagonists or antifolates; an immunomodulators; a proapoptotic receptor agonists (PARAs) including DR4 (TRAILR1) and DR5 (TRAILR2); a phospholipase A2 (PLA₂) inhibitor; a SRC inhibitor; an osteoclastic bone resorption inhibitor; a G-protein-coupled somatostain receptors inhibitor; an interleukin-11 and synthetic interleukin-11 (IL-11); a cell growth stimulator; a receptor activator for nuclear factor κB (RANK) inhibitor; a thrombopoietin mimetic peptibody; a histone deacetylase (HDAC) inhibitor; an anti-tumor antibiotic; an anti-microtubule or anti-mitotic agent; a plant alkaloid; a taxane anti-neoplastic agent; a cathepsin K inhibitor; an epothilone B analog; a heat shock protein (HSP) inhibitor; a farnesyl transferase inhibitors (FTI); a thrombopoietin (TpoR) agonist; a proteosome inhibitor; a kinesis spindle protein (KSP) inhibitor (also known as Eg5 inhibitor); a polo-like kinase (Plk) inhibitor; an adrenal steroid inhibitor; an anti-androgen; an anabolic steroid; a gonadotropin-releasing hormone (GnRH) receptor agonist; an HPV vaccine; an iron chelating agent; a bisphosphonate; a demethylating agent; a retinoid; a cytokine; an estrogen receptor downregulator; an anti-estrogen; a selective estrogen receptor modulator (SERMs); a selective estrogen receptor degrader (SERD); a leutinizing hormone releasing hormone (LHRH) agonist; aprogesterone; a 17α-hydroxylase/C17,20 lyase (CYP17A1) inhibitor; a C—C chemokine receptor 4 (CCR4) antibody; a CD20 antibody; a CD20 antibody drug conjugates; a CD22 antibody drug conjugate; a CD30 mAb-cytotoxin conjugate; a CD33 antibody drug conjugate; a CD40 antibody; a CD52 antibody; an anti-CS1 antibody; a CTLA-4 antibody; a TPH inhibitor; a PARP (poly ADP ribose polymerase) inhibitor; and a radio-sensitizer.

Examples of PARP (poly ADP ribose polymerase) inhibitors include, but are not limited to, olaparib (Lynparza), rucaparib (Rubraca), Niraparib (Zeluja), Talazoparib, and Veliparib.

Examples of radio-sensitizers include, but are not limited to, Idronoxil (Veyonda, also known as NOX-66), Sodium glycididazole, Nimorazole, NBTXR3 (also known as PEP503), [89Zr]AGuIX, Lucanthone, Telomelysin (OBP-301), lonidamine, nimorazole, panobinostat, celecoxib, cilengitide, entinostat, etanidazole, and ganetespib (STA-9090).

Examples of folate Antagonists or antifolates include, but are not limited to, Trimetrexate glucuronate (Neutrexin®); Piritrexim isethionate (BW201U); Pemetrexed (LY231514); Raltitrexed (Tomudex®); and Methotrexate (Rheumatrex®, Trexal®).

In other embodiments, the combination or combination therapy comprises, in addition to an FR targeting compound of the present disclosure, a DNA repair inhibitor. DNA repair inhibitors include single strand repair inhibitors (e.g. PARP inhibitors) and inhibitor of double strand (e.g., DNA-PK) repair mechanisms.

Some patients may experience allergic reactions to the compounds of the present disclosure and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present disclosure and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a FR targeting compound of the present disclosure, can be prepared and administered as described in the art, such as in the documents cited above.

In one embodiment, the present disclosure provides pharmaceutical compositions comprising the combination according to the invention or a pharmaceutically acceptable salt thereof together with a pharmaceutically acceptable carrier suitable for administration to a human or animal subject, either alone or together with other anti-cancer agents as previously described.

In one embodiment, the present disclosure provides methods of treating human or animal subjects suffering from a cellular proliferative disease, such as cancer, preferably FR expressing cancers

The present disclosure provides methods of treating a human or animal subject in need of such treatment, comprising administering to the subject a therapeutically effective amount of a combination according to the invention) or a pharmaceutically acceptable salt thereof, either alone or in combination with other anti-cancer agents.

In particular, combinations will either be formulated together as a combination therapeutic or administered separately.

In combination therapy, the compound of the present disclosure and other anti-cancer agent(s) may be administered either simultaneously, concurrently or sequentially with no specific time limits, wherein such administration provides therapeutically effective levels of the two compounds in the body of the patient.

In a preferred embodiment, the combination of the present disclosure and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The combination of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

The combination comprising a FR therapeutic, such as radiolabeled Compound I described herein may also be used to advantage in combination with known therapeutic processes, for example, the administration of hormones or especially radiation. A compound of the present disclosure may in particular be used as a radiosensitizer, especially for the treatment of tumors which exhibit poor sensitivity to radiotherapy.

### Uses of the FR-Targeting Compounds

In one aspect, the FR-targeting compounds of the present disclosure, for example, of formula (I), or a pharmaceutically acceptable salt thereof, can be used, for example, for treatment, diagnosis and imaging of a proliferative disease associated with FR expressing cells. Typically, the proliferative disease is cancer. Examples of compounds of formula (I) include, but are not limited, to the compounds of Embodiments 1-56, and embodiments thereof (including other specific and more specific embodiments thereof).

One embodiment is a method of treating FR expressing cancer in a subject in need thereof, comprising administering to the subject a therapeutically effective amount of a FR-targeting compound of the present disclosure, for example, a compound of formula (I), or a pharmaceutically acceptable salt thereof, or a therapeutically effective amount of a pharmaceutical composition of the present disclosure. Typically, at least some of the effective amount of the compound which is being administered to the subject comprises a radioelement bound within the chelating group of the compound. In more specific embodiments, such radioelement is ¹⁷⁷Lu or ²²⁵Ac.

A further embodiment is a method of treating FR expressing cancer in a subject in need thereof, comprising administering to the subject a therapeutically effective amount of a FR-targeting compound of the present disclosure, for example, a compound of any one of Embodiments 1 to 55, or a pharmaceutically acceptable salt thereof, or a therapeutically effective amount of a pharmaceutical composition thereof, wherein the compound has a chelating group Ch which comprises ¹⁷⁷Lu.

A further embodiment is a method of treating FR expressing cancer in a subject in need thereof, comprising administering to the subject a therapeutically effective amount of a FR-targeting compound of the present disclosure, for example, a compound of any one of Embodiments 1 to 55, or a pharmaceutically acceptable salt thereof, or a therapeutically effective amount of a pharmaceutical composition thereof, wherein the compound has a chelating group Ch which comprises ²²⁵Ac.

A further embodiment is a method of treating FR expressing cancer in a subject in need thereof, comprising administering to the subject a therapeutically effective amount of a FR-targeting compound of the following structural formula,

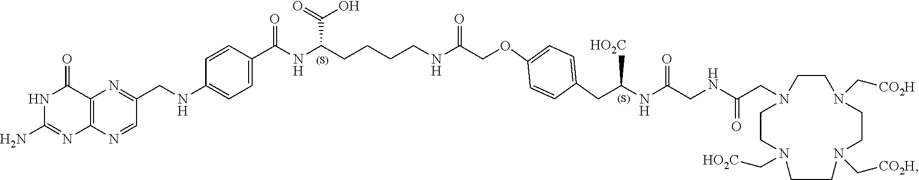

or a pharmaceutically acceptable salt thereof, wherein the FR-targeting compound has a chelating group which comprises ¹⁷⁷Lu.

A further embodiment is a method of treating FR expressing cancer in a subject in need thereof, comprising administering to the subject a therapeutically effective amount of a FR-targeting compound of the following structural formula,

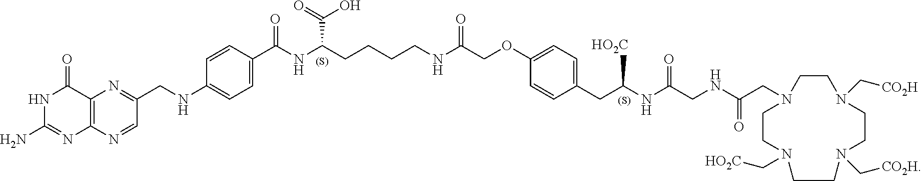

or a pharmaceutically acceptable salt thereof, wherein the FR-targeting compound has a chelating group which comprises ²²⁵Ac.

A further embodiment is a method of treating FR expressing cancer in a subject in need thereof, comprising administering to the subject a therapeutically effective amount of a FR-targeting compound of the following structural formula,

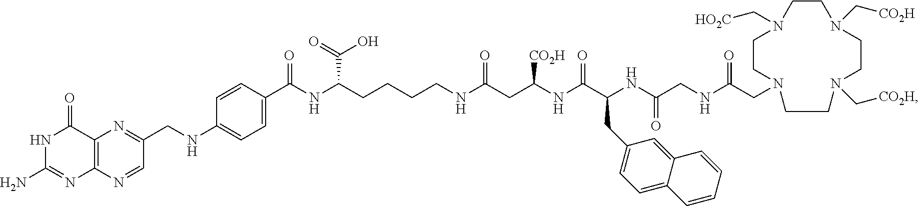 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises ¹⁷⁷Lu.

A further embodiment is a method of treating FR expressing cancer in a subject in need thereof, comprising administering to the subject a therapeutically effective amount of a FR-targeting compound of the following structural formula,

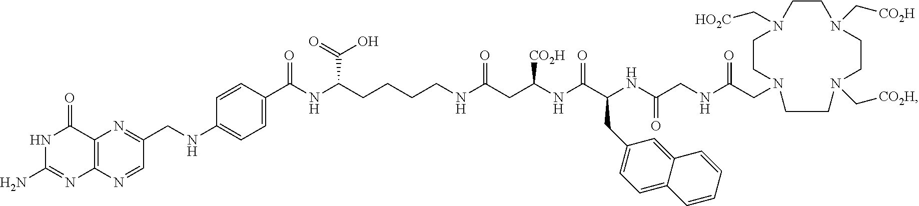 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises ²²⁵Ac.

In some embodiments, the cancer is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

In some embodiments, the FR expressing cancer is selected from the group consisting of ovarian cancer, endometrial cancer, brain cancer, lung cancer, renal cancer, head and neck cancer, breast cancer, stomach cancer, and cancer of the colon-rectum.

In some embodiments, the FR expressing cancer is selected from the group consisting of ovarian cancer, endometrial cancer, brain cancer, lung cancer, and renal cancer.

In some embodiments, the FR expressing cancer is selected from the group consisting of ovarian cancer and non-small cell lung cancer.

In some embodiments, the FR expressing cancer is ovarian cancer.

In some embodiments, the FR expressing cancer is non-small cell lung cancer.

A further embodiment is a method of treating an FR expressing tumor or cell, the method comprising contacting the one or more FR expressing tumor or cell with an effective amount of a FR-targeting compound of the present disclosure, for example, a compound of formula (I), or a pharmaceutically acceptable salt thereof, or a therapeutically effective amount of a pharmaceutical composition of the present disclosure. Typically, at least some of the effective amount of the compound which is being contacted with the FR expressing tumor or cell comprises a radioelement bound within the chelating group of the compound. In more specific embodiments, such radioelement is ¹⁷⁷Lu or ²²⁵Ac.

A further embodiment is a method of treating an FR expressing tumor or cell, the method comprising contacting the one or more FR expressing tumor or cell with an effective amount of a FR-targeting compound of any one of Embodiments 1 to 55, or a pharmaceutically acceptable salt thereof, or a therapeutically effective amount of a pharmaceutical composition thereof, wherein the compound has a chelating group Ch which comprises ¹⁷⁷Lu.

A further embodiment is a method of treating an FR expressing tumor or cell, the method comprising contacting the one or more FR expressing tumor or cell with an effective amount of a FR-targeting compound of any one of Embodiments 1 to 55, or a pharmaceutically acceptable salt thereof, or a therapeutically effective amount of a pharmaceutical composition thereof, wherein the compound has a chelating group Ch which comprises ²²⁵Ac.

A further embodiment is a method of treating an FR expressing tumor or cell, the method comprising contacting the one or more FR expressing tumor or cell with an effective amount of a FR-targeting compound of the following structural formula,

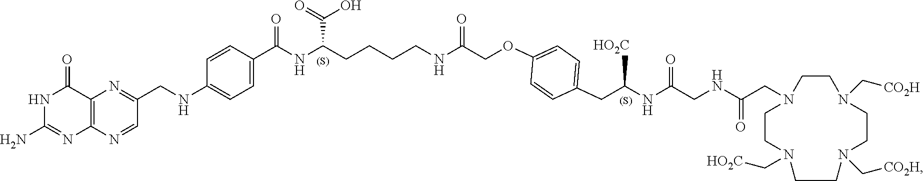

or a pharmaceutically acceptable salt thereof, wherein the FR-targeting compound has a chelating group which comprises ¹⁷⁷Lu.

A further embodiment is a method of treating an FR expressing tumor or cell, the method comprising contacting the one or more FR expressing tumor or cell with an effective amount of a FR-targeting compound of the following structural formula,

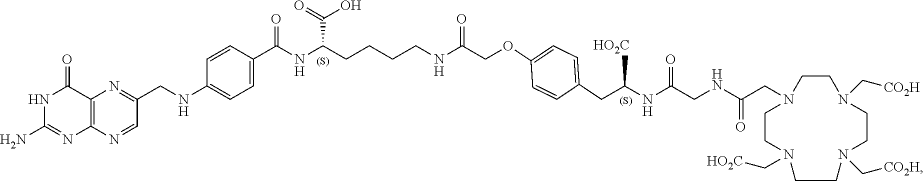

or a pharmaceutically acceptable salt thereof; wherein the FR-targeting compound has a chelating group which comprises ²²⁵Ac.

A further embodiment is a method of treating an FR expressing tumor or cell, the method comprising contacting the one or more FR expressing tumor or cell with an effective amount of a FR-targeting compound of the following structural formula,

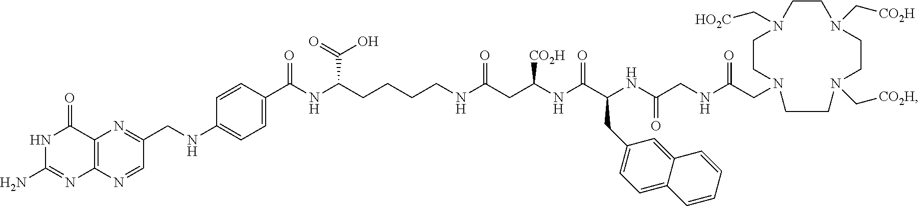 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises ¹⁷⁷Lu.

A further embodiment is a method of treating an FR expressing tumor or cell, the method comprising contacting the one or more FR expressing tumor or cell with an effective amount of a FR-targeting compound of the following structural formula,

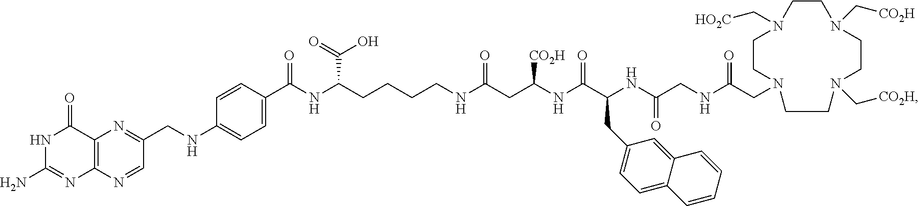 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR targeting compound has a chelating group which comprises ²²⁵Ac.

In some embodiments, the tumor or cell is associated with a cancer which is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

In some embodiments, the FR expressing tumor or cell is associated with a cancer which is selected from the group consisting of ovarian cancer, endometrial cancer, brain cancer, lung cancer, renal cancer, head and neck cancer, breast cancer, stomach cancer, and cancer of the colon-rectum.

In some embodiments, the FR expressing tumor or cell is associated with a cancer which is selected from the group consisting of ovarian cancer, endometrial cancer, brain cancer, lung cancer, and renal cancer.

In some embodiments, the cancer is selected from the group consisting of ovarian cancer and non-small cell lung cancer.

In some embodiments, the cancer is ovarian cancer.

In some embodiments, the cancer is non-small cell lung cancer.

A further embodiment is a method for imaging FR expressing cells in a subject, comprising administering to the subject an effective amount of an FR-targeting compound of the present disclosure, for example, a compound of formula (I), or a pharmaceutically acceptable salt thereof, or a therapeutically effective amount of a pharmaceutical composition of the present disclosure, wherein the compound comprises a metal, a radioelement or radiohalogen.

A further embodiment is a method method for imaging FR expressing cells in a subject, comprising administering to the subject an effective amount of an FR-targeting compound of any one of Embodiments 1 to 55, or a pharmaceutically acceptable salt thereof, or a therapeutically effective amount of a pharmaceutical composition thereof, wherein the compound has a chelating group Ch which comprises ¹⁷⁷Lu.

A further embodiment is a method method for imaging FR expressing cells in a subject, comprising administering to the subject an effective amount of an FR-targeting compound of any one of Embodiments 1 to 55, or a pharmaceutically acceptable salt thereof, or a therapeutically effective amount of a pharmaceutical composition thereof, wherein the compound has a chelating group Ch which comprises ²²⁵Ac.

A further embodiment is a method for imaging FR expressing cells in a subject, comprising administering to the subject an effective amount of an FR-targeting compound of the following structural formula,

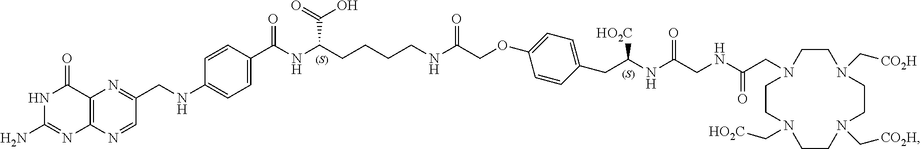

or a pharmaceutically acceptable salt thereof, wherein the FR-targeting compound has a chelating group which comprises ¹⁷⁷Lu.

A further embodiment is a method for imaging FR expressing cells in a subject, comprising administering to the subject an effective amount of an FR-targeting compound of the following structural formula,

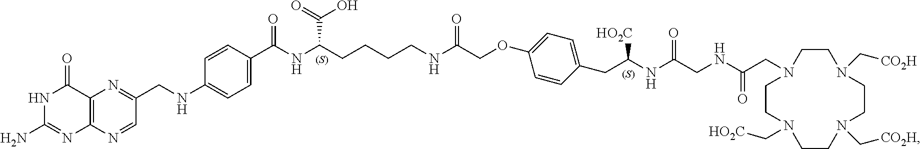

or a pharmaceutically acceptable salt thereof, wherein the FR-targeting compound has a chelating group which comprises ⁶⁸Ga.

A further embodiment is a method for imaging FR expressing cells in a subject, comprising administering to the subject an effective amount of an FR-targeting compound of the following structural formula,

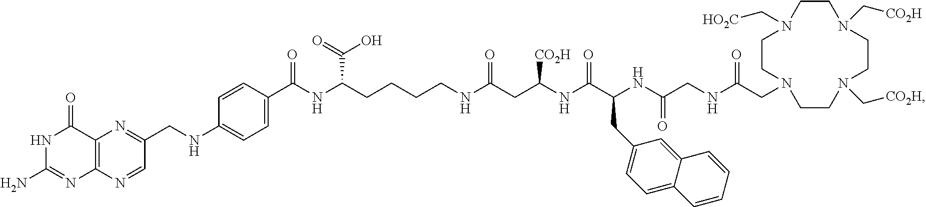 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises ¹⁷⁷Lu.

A further embodiment is a method method for imaging FR expressing cells in a subject, comprising administering to the subject an effective amount of an FR-targeting compound of the following structural formula,

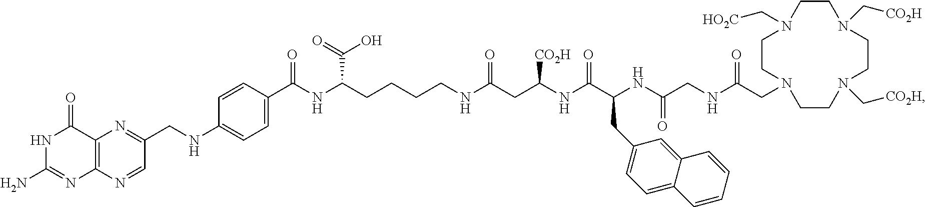 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises ⁶⁸Ga.

A further embodiment is a method method for imaging FR expressing cells in a subject, comprising administering to the subject an effective amount of an FR-targeting compound of any one of Embodiments 1 to 55, or a pharmaceutically acceptable salt thereof, or a therapeutically effective amount of a pharmaceutical composition thereof, wherein the compound has a chelating group Ch which comprises a radioelement or metal suitable for imaging.

A further embodiment is a method for imaging FR expressing cells in a subject, comprising administering to the subject an effective amount of an FR-targeting compound of the following structural formula,

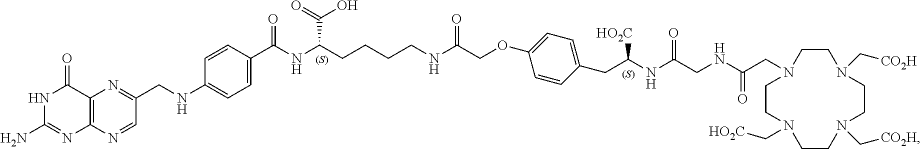

or a pharmaceutically acceptable salt thereof, wherein the FR-targeting compound has a chelating group which comprises a radioelement or metal suitable for imaging.

A further embodiment is a method for imaging FR expressing cells in a subject, comprising administering to the subject an effective amount of an FR-targeting compound of the following structural formula,

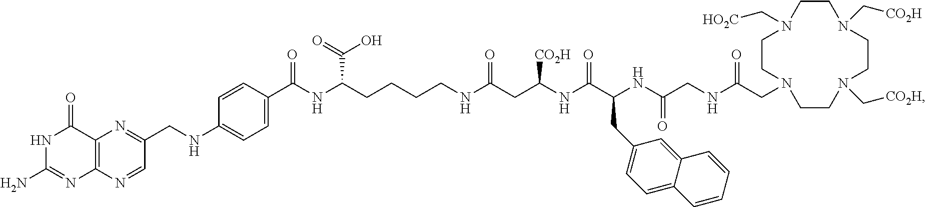 [SMILES: Nc1nc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]

or a pharmaceutically acceptable salt thereof, wherein the FR-targeting compound has a chelating group which comprises a radioelement or metal suitable for imaging.

In a further aspect, the disclosure relates to treatment of a subject in vivo using a combination comprising a FR-targeting compound of the present disclosure, such as a radiolabeled compound of formula (I) (e.g., of anyone of Embodiments 1 to 56), and additional therapeutic agents disclosed herein, or a composition or formulation comprising a combination disclosed herein, such that growth of cancerous tumors is inhibited or reduced.

In some embodiments, the FR-targeting compound of the present disclosure, such as a radiolabeled compound of formula (I), or pharmaceutically acceptable salt thereof, can be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest. In one embodiment, the combination disclosed herein can be administered in any order or simultaneously.

In one embodiment, the therapies described herein can include a composition of the present disclosure co-formulated with, and/or co-administered with, one or more additional therapeutic agents as previously described, e.g., one or more anti-cancer agents, cytotoxic or cytostatic agents, hormone treatment, vaccines, and/or other immunotherapies as previously described.

In a further embodiment, the FR-targeting compound of the present disclosure, such as a radiolabeled compound of formula (I), or pharmaceutically acceptable salt thereof, is further administered or used in combination with other therapeutic treatment modalities, including surgery, radiation, cryosurgery, and/or thermotherapy. In one aspect, such combination therapies can advantageously utilize lower dosages of the administered therapeutic agents, thus avoiding possible toxicities or complications associated with the various monotherapies.

### Pharmaceutical Compositions

In another aspect, the present disclosure provides compositions, e.g. pharmaceutically acceptable compositions, which include a radiolabeled compound of formula (I) (e.g., a compound of any one of Embodiments 1-56), or pharmaceutically acceptable salt thereof, and a radical scavenger such as gentisic acid and/or ascorbic acid.

In another aspect, the present disclosure provides a pharmaceutically acceptable composition comprising [¹⁷⁷Lu]-Compound 34, or pharmaceutically acceptable salt thereof. In a specific embodiment of this aspect, the composition further includes a radical scavenger. In further specific embodiment of this aspect, the composition further includes a gentisic acid/acetate buffer, DTPA (diethylenetriaminepentaacetic acid), and sodium ascorbate.

In another aspect, the present disclosure provides a composition comprising [¹⁷⁵Lu]-Compound 34, or pharmaceutically acceptable salt thereof.

In another aspect, the present disclosure provides a pharmaceutically acceptable composition comprising [²²⁵Ac]-Compound 37, or pharmaceutically acceptable salt thereof.

In another aspect, the present disclosure provides a pharmaceutically acceptable composition comprising [¹⁷⁷Lu]-Compound 37, or pharmaceutically acceptable salt thereof. In a specific embodiment of this aspect, the composition further includes a radical scavenger. In further specific embodiment of this aspect, the composition further includes a gentisic acid/acetate buffer, DTPA (diethylenetriaminepentaacetic acid), and sodium ascorbate.

In another aspect, the present disclosure provides a composition comprising [¹⁷⁵Lu]-Compound 37, or pharmaceutically acceptable salt thereof.

In another aspect, the present disclosure provides a pharmaceutically acceptable composition comprising [²²⁵Ac]-Compound 37, or pharmaceutically acceptable salt thereof.

In another aspect, the present disclosure provides compositions, e.g., pharmaceutically acceptable compositions, which include one or more of, e.g., two, three, four, five, six, seven, eight, or more of, a FR-targeting compound of the present disclosure, such as a radiolabeled compound of formula (I) (e.g., a compound of any one of Embodiments 1-56), or pharmaceutically acceptable salt thereof and a pharmaceutically acceptable carrier. In a specfic embodiment of this aspect, the composition includes a further therapeutic agent described herein.

In another aspect, the FR-targeting compound of the present disclosure, such as a compound of formula (I) (e.g., a compound of any one of Embodiments 1-56) for diagnosis or treatment, etc., of the present invention may be provided by (1) a method for providing a labeled preparation containing a radiolabeled FR-targeting and (2) a method for providing a kit preparation containing the FR-targeting compound, or a salt thereof. When the FR-targeting compound for diagnosis or treatment is provided as an already labeled preparation, the preparation can be used directly in administration. When a kit preparation is used, the FR-targeting compound is labeled with a desired radioactive metal in clinical settings and then used in administration. The kit preparation can be provided in the form of an aqueous solution or a freeze-dried preparation. Use of the kit preparation can eliminate the need of a special purification step, and a reaction solution can be prepared just before use as a dosing solution by merely performing reaction by the addition of a radioactive metal obtained from a generator stocked regularly in clinical settings or a radioactive metal provided by a drug manufacturer aside from or in set with the kit preparation.

In embodiments, the pharmaceutically acceptable carrier can be suitable for intravenous, intramuscular, subcutaneous, parenteral, rectal, spinal or epidermal administration (e.g. by injection or infusion).

The compositions described herein may be in a variety of forms.

In various embodiments, these include, for example, liquid, semi-solid and solid dosage forms, such as liquid solutions (e.g., injectable and infusible solutions), dispersions or suspensions, liposomes and suppositories. The form depends on the intended mode of administration and therapeutic application.

In one aspect, compositions are in the form of injectable or infusible solutions. In certain embodiments, the mode of administration is parenteral (e.g., intravenous, subcutaneous, intraperitoneal, or intramuscular).

In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In one aspect, therapeutic compositions should be sterile and stable under the conditions of manufacture and storage.

In various embodiments, the composition can be formulated as a solution, microemulsion, dispersion, liposome, or other ordered structure.

In one embodiment, the composition is suitable for high antibody concentration. Sterile injectable solutions can be prepared by incorporating the active Compound I and the additional therapeutic agent in the required amount in an appropriate solvent with one or a combination of ingredients enumerated above, as required, followed by filtered sterilization.

In one embodiment, dispersions are prepared by incorporating the FR-targeting compound of the present disclosure, for example a compound of formula (I), and any additional therapeutic agent, if desired, into a sterile vehicle that contains a basic dispersion medium and the required other ingredients from those enumerated above. In the case of sterile powders for the preparation of sterile injectable solutions, suitable methods of preparation are vacuum drying and freeze-drying that yields a powder of the active ingredient plus any additional desired ingredient from a previously sterile-filtered solution thereof.

In one aspect, the proper fluidity of a solution can be maintained, for example, by the use of a coating such as lecithin, by the maintenance of the required particle size in the case of dispersion and by the use of surfactants.

In another aspect, prolonged absorption of injectable compositions can be brought about by including in the composition an agent that delays absorption, for example, monostearate salts and gelatin.

In certain embodiments, the composition is a drug substance formulation. In other embodiments, the formulation is a lyophilized formulation, e.g., lyophilized or dried from a drug substance formulation. In other embodiments, the formulation is a reconstituted formulation, e.g., reconstituted from a lyophilized formulation. In other embodiments, the formulation is a liquid formulation.

Other exemplary buffering agents that can be used in the formulations described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulations described herein may also contain a tonicity agent, e.g., sodium chloride, and/or a stabilizing agent, e.g., an amino acid (e.g., glycine, arginine, methionine, or a combination thereof).

The combination according to the invention, inhibitors, antagonist or binding agents, can be administered by a variety of methods known in the art, although for many therapeutic applications, a suitable route/mode of administration is intravenous injection or infusion. For example, the FR therapeutic agent, such as radiolabeled compound of formula (I), or other therapeutic agents can be administered by intravenous infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. In various embodiments, biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems, J. R. Robinson, ed., Marcel Dekker, Inc., New York, 1978.

In certain embodiments, combination according to the invention can be orally administered, for example, with an inert diluent or an assimilable edible carrier. In another embodiment, any of the therapeutic agents described herein (and other ingredients, if desired) may also be enclosed in a hard or soft shell gelatin capsule, compressed into tablets, or incorporated directly into the subject's diet. For oral therapeutic administration, the therapeutic agents may be incorporated with excipients and used in the form of ingestible tablets, buccal tablets, troches, capsules, elixirs, suspensions, syrups, wafers, and the like. In one embodiment, to administer a therapeutic agent of the disclosure by other than parenteral administration, it may be necessary to coat the therapeutic agent with, or co-administer the therapeutic agent with, a material to prevent its inactivation. In another aspect, therapeutic compositions can also be administered with medical devices known in the art.

Dosage regimens can be adjusted to provide the optimum desired response (e.g., a therapeutic response). For example, a single bolus may be administered, several divided doses may be administered overtime or the dose may be proportionally reduced or increased as indicated by the exigencies of the therapeutic situation. In one embodiment, parenteral compositions can be formulated in dosage unit form for ease of administration and uniformity of dosage. Dosage unit form as used herein refers to physically discrete units suited as unitary dosages for the subjects to be treated; each unit may contain a predetermined quantity of active compound calculated to produce the desired therapeutic effect in association with the required pharmaceutical carrier. In various aspects, the specification for the dosage unit forms of the invention are dictated by and directly dependent on (a) the unique characteristics of the active compound and the particular therapeutic effect to be achieved, and (b) the limitations inherent in the art of compounding such an active compound for the treatment of the subject.

### EXAMPLES

Abbreviations: DOTA=1,4,7,10-Tetraazacyclododecane-1,4,7,10-tetraacetic acid
2-Nal=3-(2-Naphthyl)-alanine
HFIP=Hexafluoroisopropanol
TIPS=Triisopropylsilane
TFA=Trifluroacetic acid
IPA=Isopropyl alcohol
DMF=Dimethylformamide
DMSO=Dimethyl sulfoxide
DCM=Dichloromethane
TEA=Triethylamime
DIPEA=N,N-Diisopropylethylamine
FDRPMI or RPMI=Folate deficient Roswell Park Memorial Institute
FCS=Fetal calf serum

### CHEMICAL EXAMPLES

Compounds of the present disclosure can be prepared as described in the below Examples. TFA-labile Wang resins are standard supports for batch synthesis of peptide acids following the Fmoc-/tBu-protection scheme. The Fmoc-amino acids can be coupled to the 4-hydroxymethylphenoxyacetic acid linkers in such a way that epimerization and dipeptide formation are minimized. Preloaded Wang resins (e.g., preloaded with N-α-Fmoc-protected amino acids) are commercially available (e.g. from Sigma Aldrich) high-quality supports, which allow to start directly with automated protocols. Typically, the polymer matrix for the preloaded Wang resins is polystyrene cross-linked with 1% DVB.

### Example 1: Pte(N¹⁰-TFA)-Dap-OH (1)

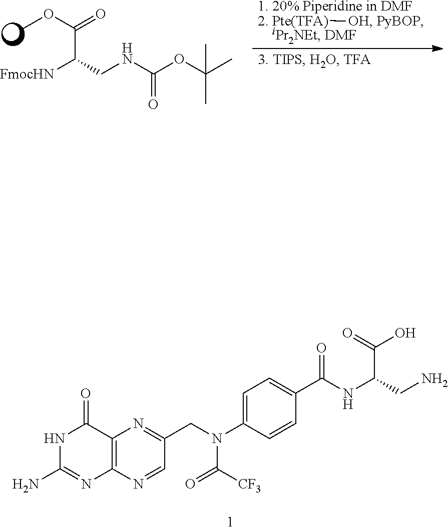

1 was synthesized by solid phase peptide synthesis (SPPS) on Wang resin. In a peptide synthesis vessel was added 1.10 g of N/3-Boc-Na-Fmoc-L-2,3-diaminopropanoic acid resin (Fmoc-Dap(Boc)-Resin, commercially obtained) (0.500 mmol, 1 equiv). A solution of 20% piperidine in dimethylformamide (DMF) (˜20 mL) for Fmoc deprotection was added. Argon was bubbled through the solution for 15 min and then drained. 20% piperidine in DMF (˜20 mL) was added and bubbling continued for 5 min before draining (repeated 2×). The resin was washed with DMF (˜20 mL×3) followed by isopropyl alcohol (IPA, ˜20 mL×3) and again with DMF (˜20 mL×3). 20 mL of DMF was then added to the peptide synthesis vessel. Solid N¹⁰-(trifluoroacetyl)pteroic acid (Pte(TFA)-OH) was added to the reaction vessel followed closely by 0.125 mL (2.00 mmol, 4 equiv) of diisopropylethylamine (ⁱPr₂NEt), and then 520 mg (1.00 mmol, 2 equiv) of benzotriazol-1-yloxy)tripyrrolidinophosphonium hexafluorophosphate (PyBOP). Argon was bubbled through the solution for 1 h and then drained. The resin was washed with DMF (˜20 mL×3) followed by IPA (˜20 mL×3) and with DMF (˜20 mL×3) again. 25 mL of cleavage reagent (95% trifluoroacetic acid (TFA), 2.5% H₂0, 2.5% triisopropylsilane (TIPS)) was added to the peptide synthesis vessel and argon was bubbled for 1 h, the vessel drained, and the sequence repeated with the cleavage reagent (10 mL for 15 min). The filtrate was concentrated under reduced pressure until ˜10 mL remained. The product was triturated in 40 mL of diethyl ether and centrifuged. The solution was decanted from the resulting pellet. The previous step was repeated twice by resuspending the pellet in 50 mL of diethyl ether and centrifuging. The pellet was dried over a stream of argon and then high vacuum. The 152 mg (62.5%) of desired product was isolated as a yellow powder and used without any further purification: LC/MS (ESI-QMS): m/z=495.2 (M+H)⁺.

### Example 2

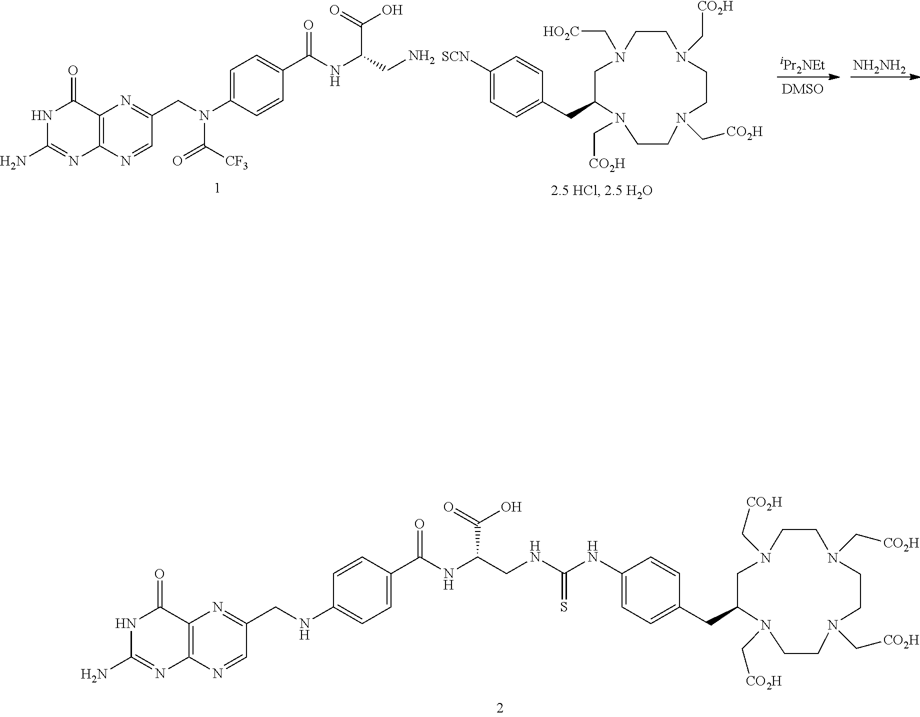

25 mg (0.0506 mmol, 1.0 equiv) of 1 was added to a solution of 0.0360 mL (0.202 mmol, 4 equiv) of ⁱPr₂NEt in 0.500 mL of dimethylsulfoxide (DMSO). 35 mg (0.0506 mmol, 1 equiv) of S-2-(4-isothiocyanatobenzyl)-1,4,7,10-tetraazacyclododecane tetraacetic acid (p-SCN-Bn-DOTA(H₄), commercially obtained) was added to the stirring reaction mixture. The reaction was monitored via LS/MS and after complete consumption of starting material 1, 0.016 mL (0.506 mmol, 10 equiv) of hydrazine (NH₂NH₂) was added to the reaction mixture. The reaction progress was again monitored via LS/MS and after complete deprotection of the TFA group, the reaction mixture was loaded onto a C18 silica column and purified by reverse phase chromatography (0-35% acetonitrile (ACN) in aqueous NH₄HCO₃ buffer (pH=7)). The 12 mg (25%) of desired product was collected as a yellow powder after lyophilization: LC/MS (ESI-QMS): m/z=950.57 (M+H)⁺.

### Example 3

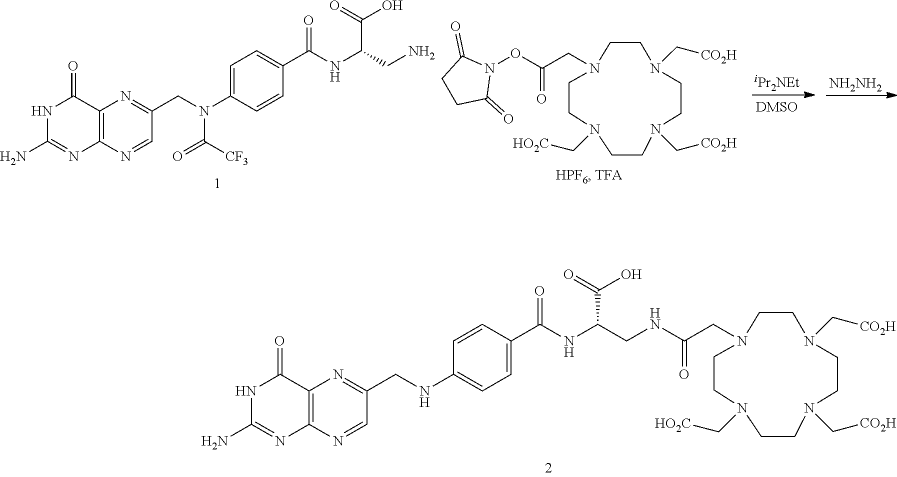

25 mg (0.0506 mmol, 1.0 equiv) of 1 was added to a solution of 0.0360 mL (0.202 mmol, 4 equiv) of ⁱPr₂NEt in 0.500 mL of DMSO. 39 mg (0.0506 mmol, 1 equiv) of 1,4,7,10-tetraazacyclododecane-1,4,7,10-tetraacetic acid mono-N-hydroxysuccinimide ester HPF₆TFA salt (DOTA(H₃)—NHS, commercially obtained) was added to the stirring reaction mixture. The reaction was monitored via LS/MS and after complete consumption of starting material 1, 0.016 mL (0.506 mmol, 10 equiv) of hydrazine (NH₂NH₂) was added to the reaction mixture. The reaction progress was again monitored via LS/MS and after complete deprotection of the TFA group, the reaction mixture was loaded onto a C18 silica column and purified by reverse phase chromatography (0-35% acetonitrile (ACN) in aqueous NH₄HCO₃ buffer (pH=7 buffer)). The 8 mg (20%) of desired product was collected as a yellow powder after lyophilization: LC/MS (ESI-QMS): m/z=785.20 (M+H)⁺.

### Example 4

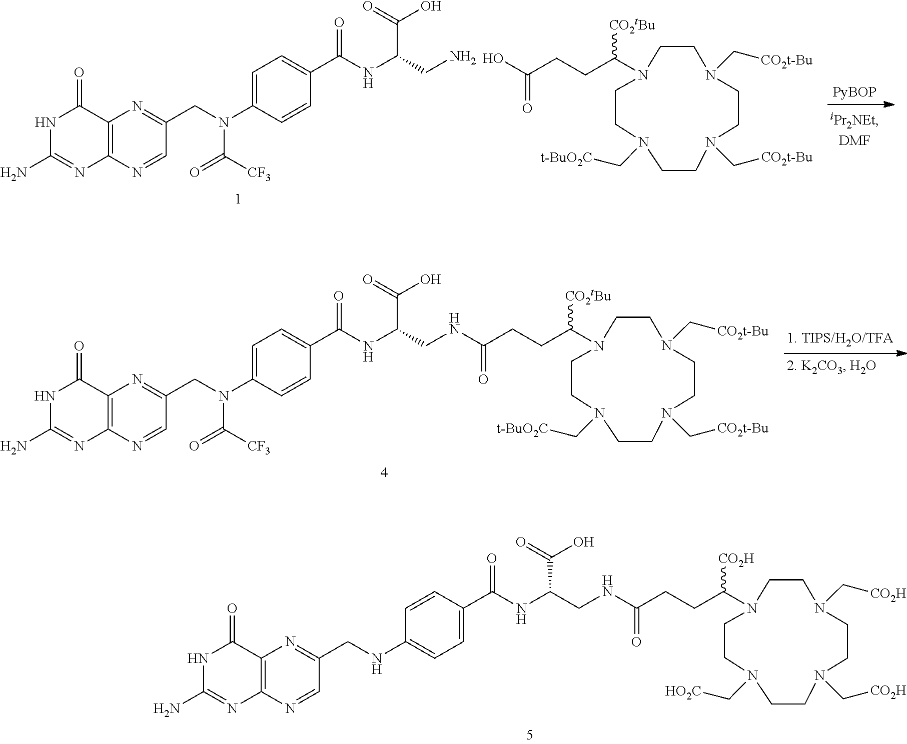

100 mg (0.202 mmol, 1.0 equiv) of 1 was added to a solution of 0.110 mL (0.809 mmol, 4 equiv) of Et₃N in 2.02 mL of DMF. 170 mg (0.242 mmol, 1.2 equiv) of 2-[1,4,7,10-tetraazacyclododecane-4,7,10-tris(t-butyl acetate)]-pentanedioic acid-It-butyl ester (DOTAGA(tBu₄)-NHS, commercially obtained) followed by 126 mg (0.242 mmol, 1.2 equiv) of PyBOP was added to the stirring reaction mixture. The reaction was monitored via LS/MS and after complete consumption of starting material 1 (3 hours), the reaction mixture was concentrated to dryness. 0.500 mL of CH₂Cl₂ was added to the crude residue and vigorously agitated. The solvent was removed under reduced pressure and the previous steps were repeated twice. The resulting residue was dissolved in a minimal amount of DMSO and loaded onto a C18 column. The product was purified via reverse phase chromatography (10-80% ACN/0.1% TFA) and lyophilized to yield 152 mg (64%) of the desired product as a pale-yellow powder: LC/MS (ESI-QMS): m/z=1178.8 (M+H)⁺.

0.200 mL of cleavage reagent (95% TFA, 2.5% H₂0, 2.5% TIPS) was added to 100 mg of 4 (0.0850 mmol, 1.0 equiv). The reaction mixture was stirred overnight (˜19 hours) at room temperature. The product was triturated in 10 mL of diethyl ether and centrifuged. The solution was decanted from the resulting pellet. The previous step was repeated twice by resuspending the pellet in 10 mL of diethyl ether and centrifuging. The pellet was dried over a stream of argon and then high vacuum. The resulting powder was dissolved in water and potassium carbonate was added until the pH of the solution reached 10. The reaction mixture was stirred for one hour under argon and analyzed via LC/MS for complete deprotection of the pteroate. The crude reaction mixture was loaded onto a C18 column. The product was purified via reverse phase chromatography (0-30% ACN/0.10% TFA). The fraction containing the desired product (analyzed via LC/MS) was concentrated until the solution became turbid. A small amount of DMSO was added until the solution became homogenous. The solution was loaded onto a C18 column and purified via HPLC (0-30% ACN/NH₄HCO₃ (pH=7) buffer) and lyophilized to yield 22 mg (30% over two steps) of the desired product as a yellow powder: LC/MS (ESI-QMS): m/z=857.4 (M+H)⁺.

### Example 5

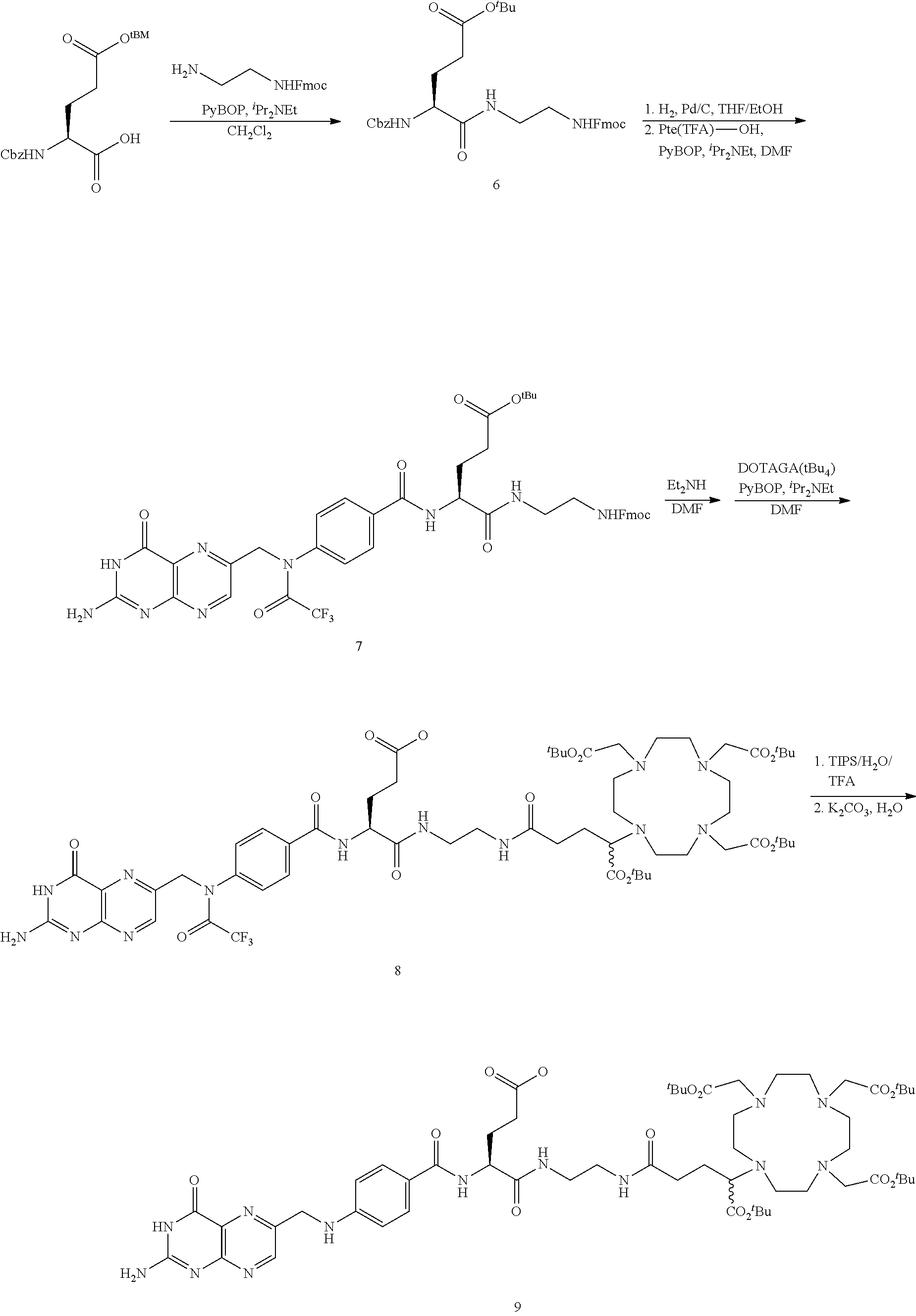

To a mixture of 1.00 g of N-benzyloxycarbonyl-L-glutamic acid 5-tert-butyl ester (Cbz-Glu(OtBu)-OH (commercially obtained), 2.964 mmol, 1 equiv), 1.040 g. of mono-Fmoc ethylene diamine hydrochloride (Fmoc-EDA (commercially obtained), 3.261 mmol, 1.1 equiv), and 1.697 g. of PyBOP (3.261 mmol, 1.1 equiv) in 29.6 mL of DMF was added 1.163 mL of ⁱPr₂NEt (6.528 mmol, 2.2 equiv) dropwise. The reaction progress was monitored via LC/MS and after one hour the starting material Cbz-Glu(OtBu)-OH was consumed. The reaction mixture was then concentrated under high vacuum. The residue was brought into 50 mL of ethyl acetate (EtOAc) and 50 mL of brine. The solution was shaken vigorously and an emulsion formed. After allowing the layers to separate, the organic layer was isolated, and the extraction was repeated twice. The combined organic layers were dried over sodium sulfate (Na₂SO₄) and filtered. Celite was added to the filtrate and the heterogenous solution was concentrated to dryness. The resulting impregnated celite was loaded onto a silica column, and the product was purified via silica chromatography (5-85% EtOAc in petroleum ether). 1.28 g (72%) of desired product was isolated as a white solid: LC/MS (ESI-QMS): m/z=602.3 (M+H)⁺.

84 mg of 10% palladium on carbon (Pd/C, 10% w/w, 0.0831 mmol, 0.1 equiv) was added to a solution of 500 mg of 6 (0.831 mmol, 1.0 equiv) in 20% ethyl alcohol in tetrahydrofuran (EtOH/THF) (8.3 mL) under argon. The headspace was evacuated and backfilled with hydrogen gas twice. The reaction mixture was stirred under hydrogen for two hours. The reaction mixture was filtered through a pad of celite and washed with ethanol. The filtrate was concentrated under reduced pressure and dried under high vacuum to yield the desired product as a light brown solid. The crude residue from the previous reaction was dissolved in 8.3 mL of DMF. To the reaction mixture was added 0.326 mL of ⁱPr₂NEt (1.828 mmol, 2.2 equiv) and 407 mg of Pte(TFA)-OH (0.997 mmol, 1.2 equiv) followed by 518 mg of PyBOP (0.997 mmol, 1.2 equiv). After the protected pteroic acid slowly went into solution, the reaction was monitored via LC/MS. The reaction mixture was stirred for four hours, before the reaction mixture was concentrated to dryness. The residue was dissolved in a minimal amount of DMSO and loaded onto a C18 column. The product was purified via reverse phase chromatography (10-85% ACN/H₂O) and lyophilized to yield 364 mg (51% over two steps) of the desired product as a yellow powder: LC/MS (ESI-QMS): m/z=858.3 (M+H)⁺.

0.230 mL of diethylamine (Et₂NH) was added to a solution of 200 mg of 7 (0.233 mmol, 1.0 equiv) in 2.10 mL of DMF. The reaction mixture was stirred at room temperature under argon for 1.5 h and concentrated under reduced pressure. The residue is co-evaporated with CH₂Cl₂ (1 mL×3), dried under high vacuum for 30 min, and dissolved in anhydrous DMF (2.3 mL). To the reaction mixture was added 0.0910 mL of ⁱPr₂NEt (0.513 mmol, 2.2 equiv) and 196 mg of 2-[1,4,7,10-tetraazacyclododecane-4,7,10-tris(t-butyl acetate)]-pentanedioic acid-It-butyl ester (DOTAGA(tBu₄) (commercially obtained), 0.280 mmol, 1.2 equiv) followed by 145 mg of PyBOP (0.280 mmol, 1.2 equiv). The reaction mixture was stirred for four hours, before the reaction mixture was concentrated to dryness. The residue was dissolved in a minimal amount of DMSO and loaded onto a C18 column. The product was purified via reverse phase chromatography (5-65% ACN/0.1% TFA) and lyophilized to yield 165 mg (54%) of the desired product as a pale-yellow powder: LC/MS (ESI-QMS): m/z=1318.9 (M+H)⁺.

0.200 mL of cleavage reagent (95% TFA, 2.5% H₂0, 2.5% TIPS) was added to the 100 mg of 8 (0.0758 mmol, 1.0 equiv). The reaction mixture was stirred overnight (˜19 h) at room temperature. The product was triturated in 10 mL of diethyl ether and centrifuged. The solution was decanted from the resulting pellet. The previous step was repeated twice by resuspending the pellet in 10 mL of diethyl ether and centrifuging. The pellet was dried over a stream of argon and then high vacuum. The resulting powder was dissolved in water and potassium carbonate was added until the pH of the solution reached 10. The reaction mixture was stirred for one hour and analyzed via LC/MS for complete deprotection of the pteroate. The crude reaction mixture was loaded onto a C18 column. The product was purified via reverse phase chromatography (0-30% ACN/0.1% TFA). The fraction containing the desired product (analyzed via LC/MS) was concentrated until the solution became turbid. A small amount of DMSO was added until the solution became homogenous. The solution was loaded onto a C18 column and purified via HPLC (0-30% ACN/NH₄HCO₃ buffer (pH=7)) and lyophilized to yield 12 mg (13% over two steps) of the desired product as a yellow powder: LC/MS (ESI-QMS): m/z=942.6 (M+H)⁺.

### Example 6

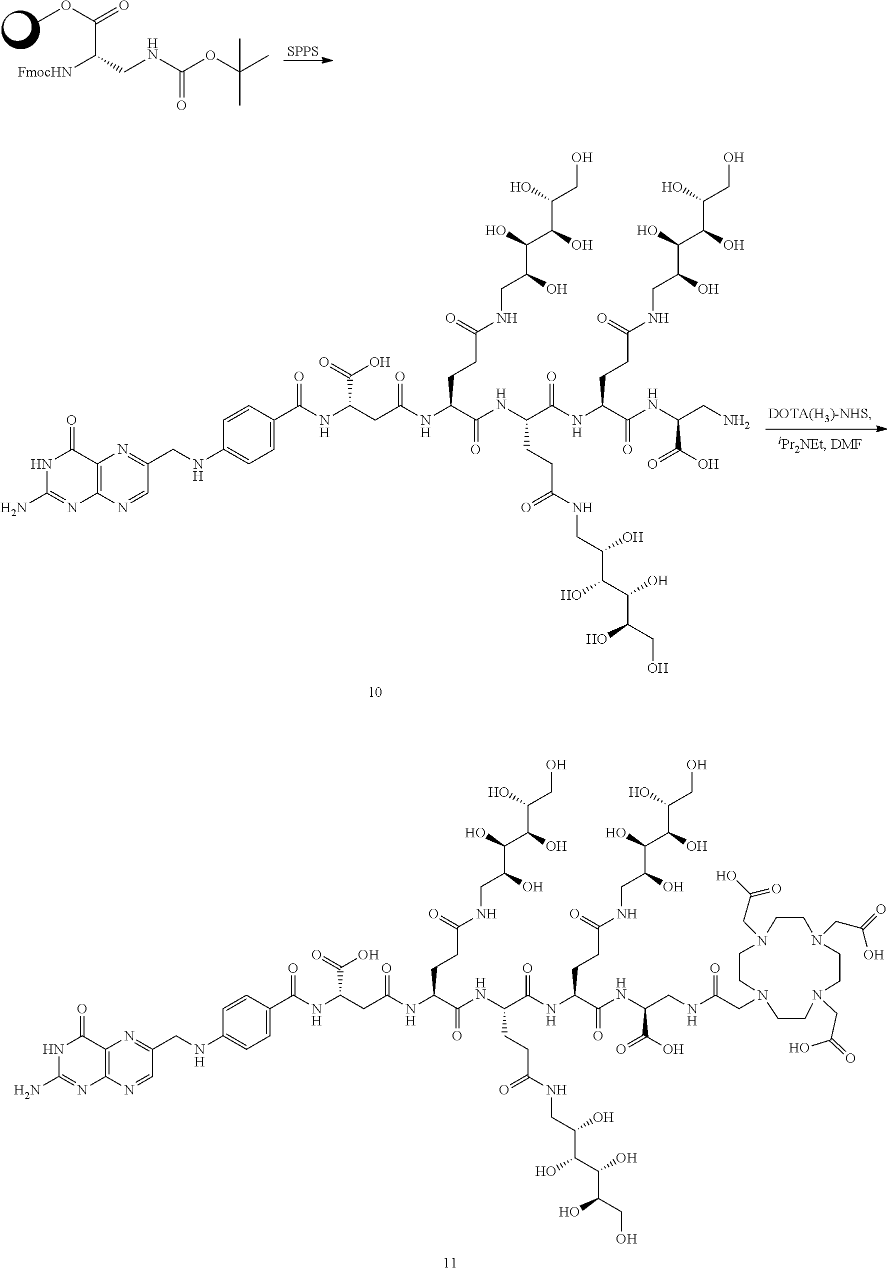

10 was synthesized by solid phase in ten steps starting from Fmoc-Dap(Boc)-Wang-Resin (Table 1). In a peptide synthesis vessel was added 1.10 g of Fmoc-Dap(Boc)-R (0.500 mmol, 1 equiv).

TABLE 1
Solid phase reaction steps for 10

| | | | MW | | |
|---|---|---|---|---|---|
| Compound | mmol. | equiv. | (g/mol) | amount | |
| Fmoc-Dap(Boc)-Wang-Resin | 0.5 | 1 | — | 1.10 | g |
| (loading 0.45 mol/g), | | | | | |
| (commercially obtained) | | | | | |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| (commercially obtained) | | | | | |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| (commercially obtained) | | | | | |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc-aspartic acid alpha-t-butyl ester
(Fmoc-Asp-OtBu) Coupling:

| | | | | | |
|---|---|---|---|---|---|
| Fmoc-Asp-OtBu | 1 | 2 | 411 | 411 | mg |
| (commercially obtained) | | | | | |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: N¹⁰-(Trifluoroacetyl)pteroic acid
(Pte(TFA)—OH) Coupling:

| | | | | | |
|---|---|---|---|---|---|
| Pte(TFA)—OH | 0.625 | 1.25 | 408 | 255 | mg |
| ⁱPr₂NEt | 2 | 4 | 129 | 0.356 | mL |
| PyBOP | 1 | 2 | 520 | 520 | mg |

Procedure C: Cleavage - 2.5% TIPS, 2.5% H₂O, TFA
Procedure D: N10 TFA Deprotection -
K₂CO₃, H₂O

### Procedure A: Fmoc Deprotection

The deprotection step was performed before each amino acid coupling steps. A solution of 20% piperidine in DMF (˜20 mL) for Fmoc deprotection was added. Argon was bubbled through the solution for 15 min and then drained. 20% piperidine in DMF (˜20 mL) was added and bubbling continued for 5 min before draining (2×). The resin was washed with DMF (˜20 mL×3) followed by IPA (˜20 mL×3) and with DMF again (˜20 mL×3).

### Procedure B: Amino Coupling

An amino acid solution in DMF (˜20 mL), ⁱPr₂NEt, and PyBOP were added to a peptide synthesis vessel. Argon was bubbled through the solution for 1 h and then drained. The resin was washed with DMF (˜20 mL×3) followed by IPA (˜20 mL×3) and with DMF (˜20 mL×3) again.

### Procedure C: Resin Cleavage

The resin was washed with CH₂Cl₂ (˜20 mL×3). 25 mL of cleavage reagent (95% TFA, 2.5% H₂0, 2.5% TIPS) was added to the peptide synthesis vessel and Argon was bubbled for 1 h, drain, and repeated twice with cleavage reagent (10 mL for 15 min). The reaction mixture was concentrated under reduced pressure until 10 mL remained. The product was triturated in 40 mL of diethyl ether and centrifuged. The solution was decanted from the resulting pellet. The previous step was repeated twice by re-suspending the pellet in 50 mL of diethyl ether and centrifuging. The pellet was dried over a stream of argon and then high vacuum.

### Procedure D: N¹⁰ TFA Deprotection

The resulting powder was dissolved in water and potassium carbonate was added until the pH of the solution reached 10. The reaction mixture was stirred for one hour under Argon and analyzed via LC/MS for complete deprotection of the pteroate.

### Purification

The crude reaction mixture was loaded onto a C18 column. The product was purified via reverse phase chromatography (0-35% ACN/0.1% TFA). The fraction containing the desired product (analyzed via LC/MS) was concentrated, frozen using a dry ice/acetone bath, and lyophilized to yield 355 mg (51% over twelve steps) of the desired product as a yellow powder: LC/MS (ESI-QMS): m/z=1390.8 (M+H)⁺.

50 mg (0.0360 mmol, 1.0 equiv) of 10 was added to a solution of 0.026 mL (0.144 mmol, 4 equiv) of ⁱPr₂NEt in 0.400 mL of DMF. 27 mg (0.0360 mmol, 1 equiv) of DOTA(H₃)—NHS was added to the stirring reaction mixture. The reaction was monitored via LS/MS and after complete consumption of starting material 10, the reaction mixture was concentrated. 0.500 mL of CH₂C₁₂ was added to the crude residue and vigorously agitated. The solvent was removed under reduced pressure and the previous steps were repeated twice. The resulting solid was dissolved in a minimal amount of DMSO and loaded onto a C18 silica column. The product was purified via reverse phase chromatography (0-30% ACN/pH2 buffer). The fraction containing the desired product (analyzed via LC/MS) was concentrated until the solution became turbid. A small amount of DMSO was added until the solution became homogenous. The solution was loaded onto a C18 column and purified via HPLC (0-30% ACN/NH₄HCO₃ buffer pH=7)) and lyophilized to yield 10 mg (15%) of the desired product as a yellow powder: LC/MS (ESI-QMS): m/z=1777.7 (M+H)⁺.

### Example 7

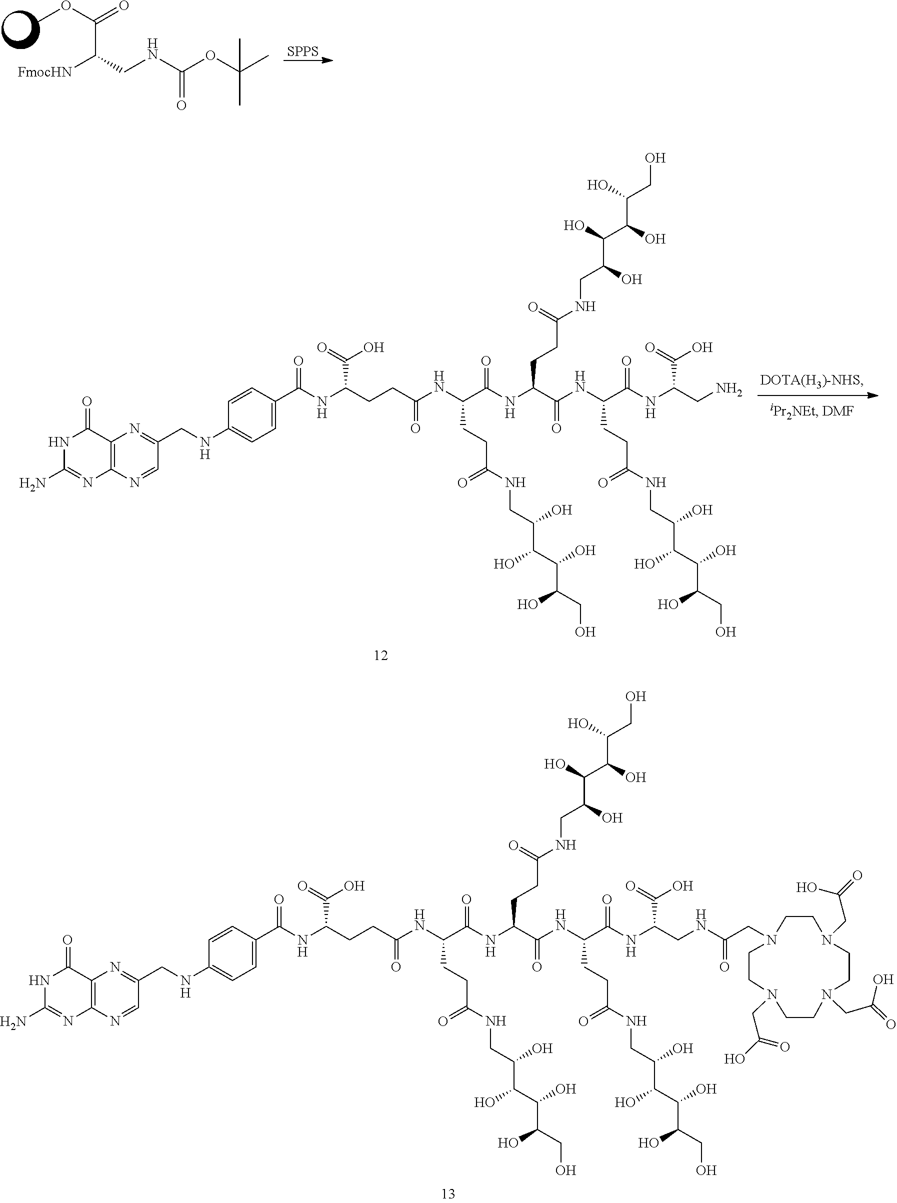

12 was synthesized by solid phase in ten steps starting from Fmoc-Dap(Boc)-Wang-Resin (Table 2). The procedure followed the same sequence as that of 10 except Fmoc-Glu-OtBu was used in lieu of Fmoc-Asp-OtBu. 310 mg (44% over twelve steps) of the desired product was isolated as a yellow powder: LC/MS (ESI-QMS): m/z=1404.7 (M+H)⁺.

TABLE 2
Solid phase reaction steps for 12

| | | | MW | | |
|---|---|---|---|---|---|
| | mmol. | equiv. | (g/mol) | amount | |
| Fmoc-Dap(Boc)-Wang-Resin | 0.5 | 1 | — | 1.10 | g |
| (loading 0.45 mol/g.) | | | | | |
| (commercially obtained) | | | | | |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc—N-(D-glucaminyl bisactonide)-aspargine
(Fmoc-Asn(D-Glucamine)-OH) Coupling:

| | | | | | |
|---|---|---|---|---|---|
| Fmoc-Asn(D-Glucamine)-OH | 1 | 2 | 613 | 613 | mg |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: L-Fmoc-glutamic acid alpha-t-butyl ester
(Fmoc-Glu-OtBu) Coupling:

| | | | | | |
|---|---|---|---|---|---|
| Fmoc-Glu-OtBu | 1 | 2 | 411 | 411 | mg |
| (commercially obtained) | | | | | |
| ⁱPr₂NEt | 2 | 4 | 129 | 0.356 | mL |
| PyBOP | 1 | 2 | 520 | 520 | mg |

Procedure A: Fmoc Deprotection - 20% Piperidine in DMF (3X)
Procedure B: N¹⁰-(Trifluoroacetyl)pteroic acid
(Pte(TFA)—OH) Coupling:

| | | | | | |
|---|---|---|---|---|---|
| Pte(TFA)—OH | 0.625 | 1.25 | 408 | 255 | mg |
| ⁱPr₂NEt | 2 | 4 | 129 | 0.356 | mL |
| PyBOP | 1 | 2 | 520 | 520 | mg |

Procedure C: Cleavage - 2.5% TIPS, 2.5% H₂O, TFA
Procedure D: N10 TFA Deprotection -
K₂CO₃, H₂O

50 mg (0.0356 mmol, 1.0 equiv) of 12 was added to a solution of 0.025 mL (0.142 mmol, 4 equiv) of ⁱPr₂NEt in 0.400 mL of DMF. 27 mg (0.0356 mmol, 1 equiv) of DOTA(H₃)—NHS was added to the stirring reaction mixture. The reaction was monitored via LS/MS and after complete consumption of starting material 12, the reaction mixture was concentrated. 0.500 mL of CH₂Cl₂ was added to the crude residue and vigorously agitated. The solvent was removed under reduced pressure and the previous steps were repeated twice. The resulting solid was dissolved in a minimal amount of DMSO and loaded onto a C18 silica column. The product was purified via reverse phase chromatography (0-30% ACN/0.1% TFA). The fraction containing the desired product (analyzed via LC/MS) was concentrated until the solution became turbid. A small amount of DMSO was added until the solution became homogenous. The solution was loaded onto a C18 column and purified via HPLC (0-30% ACN/NH₄HCO₃ buffer (pH=7)) and lyophilized to yield 12 mg (19% o) of the desired product as a yellow powder: LC/MS (ESI-QMS): m/z=1791.5 (M+H)⁺.

### Example 8

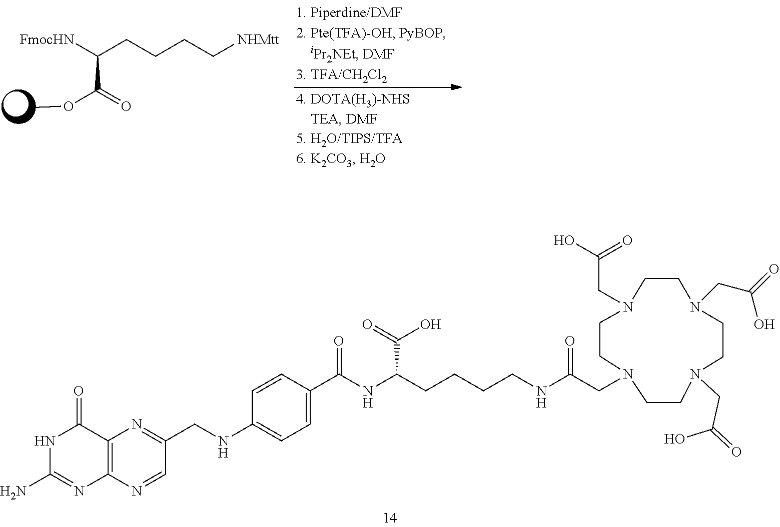

14 was synthesized by solid phase in six steps starting from Fmoc-Lys(N-4-methoxytrityl)-2-chlorotrityl-Resin (Table 3). In a peptide synthesis vessel was added 1.47 g of Fmoc-Lys(N-4-methoxytrityl)-2-chlorotrityl-Resin (0.500 mmol, 1 equiv).

TABLE 3
Solid phase reaction steps for 14

| | | | MW | | |
|---|---|---|---|---|---|
| | mmol. | equiv. | (g/mol) | amount | |
| Fmoc-Lys(N-4-methoxytrityl)- | 0.5 | — | — | 1.47 | g |
| 2-chlorotrityl-Resin | | | | | |
| (loading 0.34 mmol/g.) | | | | | |
| (commercially obtained) | | | | | |

Procedure A: Fmoc Deprotection - 20% Piperidine in DMF (3X)
Procedure B: N¹⁰-(trifluoroacetyl)pteroic acid
(Pte(TFA)—OH) Coupling:

| | | | | | |
|---|---|---|---|---|---|
| Pte(TFA)—OH | 0.625 | 1.25 | 408 | 255 | mg |
| ⁱPr₂NEt | 2 | 4 | 129 | 0.356 | mL |
| PyBOP | 1 | 2 | 520 | 520 | mg |

Procedure C: Mtt Deprotection - 2% TFA in
CH₂Cl₂ (6X)
Procedure D: 1,4,7,10-tetraazacyclododecane-
1,4,7,10-tetraacetic acid mono-N-
hydroxysuccinimide ester HPF₆/TFA salt
(DOTA(H₃)—NHS) Coupling:

| | | | | | |
|---|---|---|---|---|---|
| DOTA(H₃)—NHS | 0.750 | 1.5 | 762 | 572 | mg |
| (commercially obtained) | | | | | |
| ⁱPr₂NEt | 1.50 | 3 | 129 | 0.267 | mL |

Procedure E: Cleavage - 2.5% TIPS, 2.5% H₂O, TFA
Procedure F: N10 TFA Deprotection -
K₂CO₃, H₂O

### Procedure A: Fmoc Deprotection

A solution of 20% piperidine in DMF (˜20 mL) for Fmoc deprotection was added. Argon was bubbled through the solution for 15 min and then drained. 20% piperidine in DMF (˜20 mL) was added and bubbling continued for 5 min before draining (2×). The resin was washed with DMF (˜20 mL×3) followed by IPA (˜20 mL×3) and with DMF again (˜20 mL×3).

### Procedure B: Amino Coupling

An amino acid solution in DMF (˜20 mL), ⁱPr₂NEt, and PyBOP were added to a peptide synthesis vessel. Argon was bubbled through the solution for 1 h and then drained. The resin was washed with DMF (˜20 mL×3) followed by IPA (˜20 mL×3) and finally with CH₂Cl₂ (˜20 mL×3).

### Procedure C: Mtt Cleavage

2% TFA in CH₂Cl₂ (˜20 mL) was added and argon was bubbled through the solution for 5 min. A small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. A 2% solution of TFA in CH₂Cl₂ was added once more. If the solution remained clear, the reaction mixture was drained, and the next coupling step was performed. If the solution turned yellow the resin was washed with fresh CH₂Cl₂ until clear and the process was repeated until a clear reaction solution was achieved. The resin was then washed with DMF (˜20 mL×3).

### Procedure D: Chelator Coupling

ⁱPr₂NEt was added to a solution of DOTA(H₃)—NHS in DMF (˜20 mL) in a peptide synthesis vessel. Argon was bubbled through the solution for 1 h and then drained. The resin was washed with DMF (˜20 mL×3) followed by IPA (˜20 mL×3) and finally with CH₂Cl₂ (˜20 mL×3).

### Procedure E: Resin Cleavage

25 mL of cleavage reagent (95% TFA, 2.5% H₂0, 2.5% TIPS) was added to the peptide synthesis vessel and argon was bubbled for 1 h, drain, and repeated twice with cleavage reagent (10 mL for 15 min). The reaction mixture was concentrated under reduced pressure until 10 mL remained. The product was triturated in 40 mL of diethyl ether and centrifuged. The solution was decanted from the resulting pellet. The previous step was repeated twice by re-suspending the pellet in 50 mL of diethyl ether and centrifuging. The pellet was dried over a stream of argon and then high vacuum.

### Procedure F: N¹⁰ TFA Deprotection

The resulting powder was dissolved in water and potassium carbonate was added until the pH of the solution reached 10. The reaction mixture was stirred for one hour under Argon and analyzed via LC/MS for complete deprotection of the pteroate.

### Purification

The crude reaction mixture was loaded onto a C18 column. The product was purified via reverse phase chromatography (0-30% ACN/0.1% TFA). The fraction containing the desired product (analyzed via LC/MS) was concentrated until the solution became turbid, frozen (with a dry ice/acetone bath), and lyophilized to yield 255 mg (62%) of the desired product as a yellow powder: LC/MS (ESI-QMS): m/z=827.6 (M+H)⁺.

### Example 9

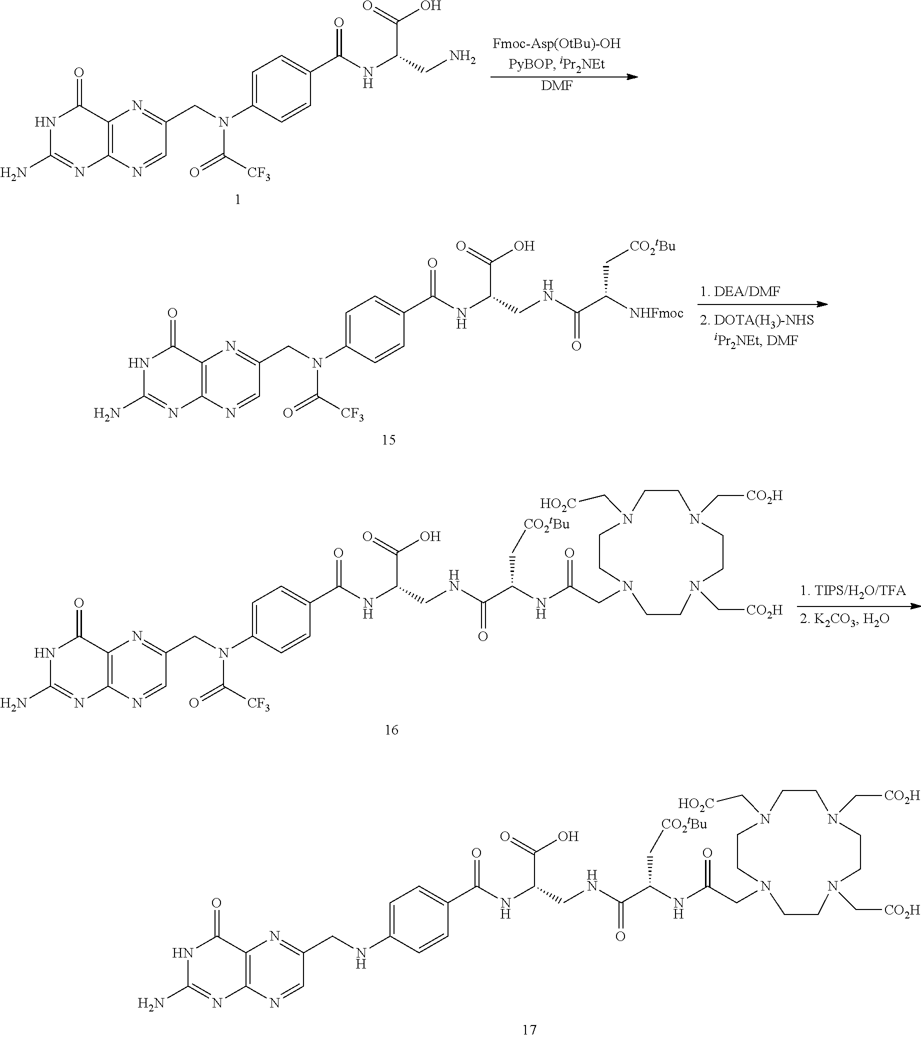

200 mg (0.404 mmol, 1.0 equiv) of 1 was added to a solution of 0.173 mL (0.970 mmol, 2.4 equiv) of ⁱPr₂NEt in 4.04 mL of DMF. 199 mg (0.485 mmol, 1.2 equiv) of Fmoc-Asp(OtBu)-OH (commercially obtained) followed by 252 mg (0.485 mmol, 1.2 equiv) of PyBOP was added to the stirring reaction mixture. The reaction was monitored via LS/MS, and after complete consumption of starting material 1 (one hour), the product was triturated in 12 mL of 1M aqueous hydrochloric acid at 0° C. and centrifuged. The solution was decanted from the resulting pellet. The previous step was repeated twice by re-suspending the pellet in 12 mL of aqueous HCl and centrifuging. The pellet was frozen in a dry ice/acetone bath and lyophilized overnight. 275 mg (77%) of resulting pale-yellow powder isolated and used without further purification: LC/MS (ESI-QMS): m/z=888.8 (M+H)⁺.

0.282 mL of diethylamine (Et₂NH) was added to a solution of 250 mg of 15 (0.282 mmol, 1.0 equiv) in 2.54 mL of DMF. The reaction mixture was stirred at room temperature under argon for one hour and then concentrated under reduced pressure. The residue is co-evaporated with CH₂Cl₂ (1 mL×3), dried under high vacuum for 30 min, and dissolved in anhydrous DMF (2.3 mL). 0.121 mL (0.677 mmol, 2.4 equiv) of ⁱPr₂NEt was added followed by 237 mg (0.338 mmol, 1.2 equiv) of DOTAGA(tBu₃) (commercially obtained) and 176 mg (0.338 mmol, 1.2 equiv) of PyBOP. The reaction was monitored via LS/MS, and after complete consumption of starting material 15, the reaction mixture was concentrated. 1.0 mL of CH₂Cl₂ was added to the crude residue and vigorously agitated. The solvent was removed under reduced pressure and the previous steps were repeated twice. The resulting solid was dissolved in a minimal amount of DMSO and loaded onto a C18 silica column. The product was purified via reverse phase chromatography (0-35% ACN/0.1% TFA). The fraction containing the desired product (analyzed via LC/MS) was concentrated until the solution became turbid, frozen, and lyophilized to yield 120 mg (40%) of the desired product as a pale-yellow powder: LC/MS (ESI-QMS): m/z=1052.3 (M+H)⁺.

0.200 mL of cleavage reagent (95% TFA, 2.5% H₂0, 2.5% TIPS) was added to the 100 mg of 16 (0.0951 mmol, 1.0 equiv). The reaction mixture was stirred for 5.5 h at room temperature. The product was triturated in 10 mL of diethyl ether and centrifuged. The solution was decanted from the resulting pellet. The previous step was repeated twice by resuspending the pellet in 10 mL of diethyl ether and centrifuging. The pellet was dried over a stream of argon and then high vacuum. The resulting powder was dissolved in water and potassium carbonate was added until the pH of the solution reached 10. The reaction mixture was stirred for one hour and analyzed via LC/MS for complete deprotection of the pteroate. The crude reaction mixture was loaded onto a C18 column. The product was purified via reverse phase chromatography (0-35% ACN/0.1% TFA). The fraction containing the desired product (analyzed via LC/MS) was concentrated until the solution became turbid. A small amount of DMSO was added until the solution became homogenous. The solution was loaded onto a C18 column and purified via reverse phase chromatography (0-20% ACN/NH₄HCO₃ buffer (pH=7)), lyophilized to yield 9 mg (11% over two steps) of the desired product as a yellow powder: LC/MS (ESI-QMS): m/z=900.7 (M+H)⁺.

### Example 10

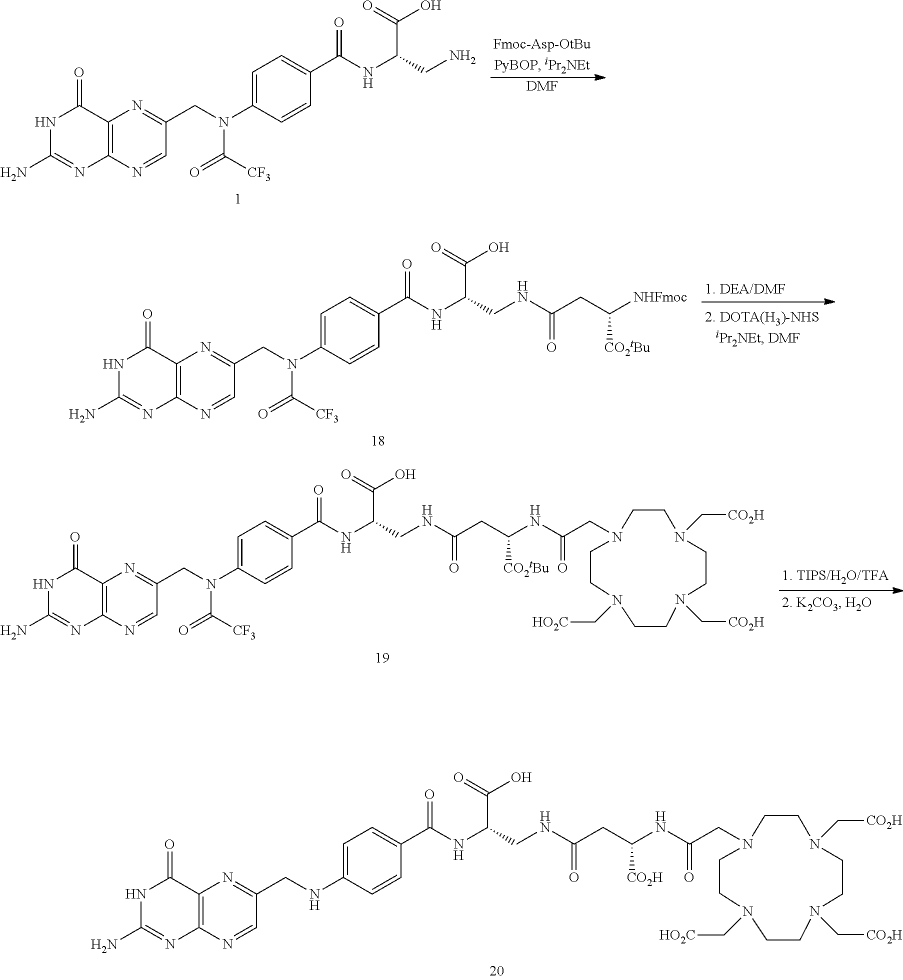

200 mg (0.404 mmol, 1.0 equiv) of 1 was added to a solution of 0.173 mL (0.970 mmol, 2.4 equiv) of ⁱPr₂NEt in 4.04 mL of DMF. 199 mg (0.485 mmol, 1.2 equiv) of Fmoc-Asp-OtBu (commercially obtained) followed by 252 mg (0.485 mmol, 1.2 equiv) of PyBOP was added to the stirring reaction mixture. The reaction was monitored via LS/MS and after complete consumption of starting material 1 (one hour), the product was triturated in 12 mL of 1M aqueous hydrochloric acid at 0° C. and centrifuged. The solution was decanted from the resulting pellet. The previous step was repeated twice by resuspending the pellet in 12 mL of aqueous HCl and centrifuging. The pellet was frozen in a dry ice/acetone bath and lyophilized overnight. 255 mg (71%) of resulting pale-yellow powder isolated and used without further purification: LC/MS (ESI-QMS): m/z=888.6 (M+H)⁺.

0.282 mL of diethylamine (Et₂NH) was added to a solution of 250 mg of 18 (0.282 mmol, 1.0 equiv) in 2.54 mL of DMF. The reaction mixture was stirred at room temperature under argon for one hour and then concentrated under reduced pressure. The residue is co-evaporated with CH₂Cl₂ (1 mL×3), dried under high vacuum for 30 min, and dissolved in anhydrous DMF (2.3 mL). 0.121 mL (0.677 mmol, 2.4 equiv) of ⁱPr₂NEt was added followed by 237 mg (0.338 mmol, 1.2 equiv) of DOTAGA(tBu₃) (commercially obtained) and 176 mg (0.338 mmol, 1.2 equiv) of PyBOP. The reaction was monitored via LS/MS and after complete consumption of starting material 18, the reaction mixture was concentrated. 1.0 mL of CH₂Cl₂ was added to the crude residue and vigorously agitated. The solvent was removed under reduced pressure and the previous steps were repeated twice. The resulting solid was dissolved in a minimal amount of DMSO and loaded onto a C18 silica column. The product was purified via reverse phase chromatography (0-35% ACN/0.1% TFA). The fraction containing the desired product (analyzed via LC/MS) was concentrated until the solution became turbid, frozen, and lyophilized to yield 112 mg (38%) of the desired product as a pale-yellow powder: LC/MS (ESI-QMS): m/z=1052.3 (M+H)⁺.

0.200 mL of cleavage reagent (95% TFA, 2.5% H₂0, 2.5% TIPS) was added to the 90 mg of 19 (0.0856 mmol, 1.0 equiv). The reaction mixture was stirred for 5.5 h at room temperature. The product was triturated in 10 mL of diethyl ether and centrifuged. The solution was decanted from the resulting pellet. The previous step was repeated twice by re-suspending the pellet in 10 mL of diethyl ether and centrifuging. The pellet was dried over a stream of argon and then high vacuum. The resulting powder was dissolved in water and potassium carbonate was added until the pH of the solution reached 10. The reaction mixture was stirred for one hour and analyzed via LC/MS for complete deprotection of the pteroate. The crude reaction mixture was loaded onto a C18 column. The product was purified via reverse phase chromatography (0-35% ACN/pH=2 buffer). The fraction containing the desired product (analyzed via LC/MS) was concentrated until the solution became turbid. A small amount of DMSO was added until the solution became homogenous. The solution was loaded onto a C18 column and purified via reverse phase chromatography (0-20% ACN/NH₄HCO₃ buffer (pH=7)), lyophilized to yield 8 mg (10% over two steps) of the desired product as a yellow powder: LC/MS (ESI-QMS): m/z=900.7 (M+H)⁺.

### Example 11

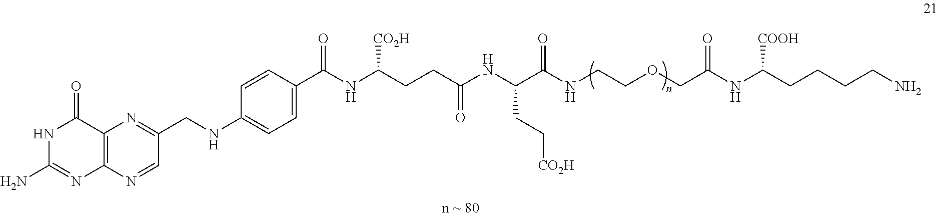

Folate spacer 21 was synthesized by standard Fmoc-solid phase peptide synthesis (SPPS) techniques following the general procedures outlined for 10 from Fmoc-L-Lys(Boc)-Wang resin using the following quantities of materials:

TABLE 4
Constituent compounds used for 21

| | Amount | mmol or | |
|---|---|---|---|
| Compound | (mg) | equivalents | |
| Fmoc-L-Lys(Boc)-Wang resin | 150 | 0.0500 | mmol |
| (commercially obtained) | | | |
| Fmoc—NH-Peg₃₅₀₀-CH₂CO—NHS ester | 262 | 1 | eq. |
| (commercially obtained) | | | |
| Fmoc-Glu(Ot-Bu)—OH | 42 | 2 | eq. |
| (commercially obtained) | | | |
| Fmoc-Glu-OtBu | 43 | 2 | eq. |
| (commercially obtained) | | | |
| N¹⁰-TFA-Pteroic acid | 41 | 2 | eq. |

As described in the general procedure, 2 equivalents of PyBOP and 4 equivalents of ⁱPr₂NEt were used to couple each of the carboxylic acids listed above to the growing peptide chain. Coupling of Peg was carried out overnight without added PyBOP. After cleavage, UPLC analysis showed a mixture of desired compound and spacer without Peg moiety. The crude material was purified by C18 silica reversed-phase column chromatography (0.1% TFA and ACN as eluents) to provide 20 mg of desired product after lyophilization.

¹H NMR (500 MHz, DMSO-d₆/D₂O): δ 8.64 (s, 1H), 7.62 (d, 2H, J=8.5 Hz), 6.62 (d, 2H, J=8.5 Hz), 4.47 (s, 2H), 4.25 (m, 3H), 4.18 (dd, 1H, J=5.5, 8 Hz), 3.91 (s, 3H), 3.65-3.3 (m), 3.16, (m, 3H), 2.74 (t, 2H, J=8 Hz), 2.3-2.15 (m, 4H), 2.02 (m, 1H), 1.9-1.6 (m, 6H), 1.5 (m, 2H), 1.3 (m, 2H).

### Example 12

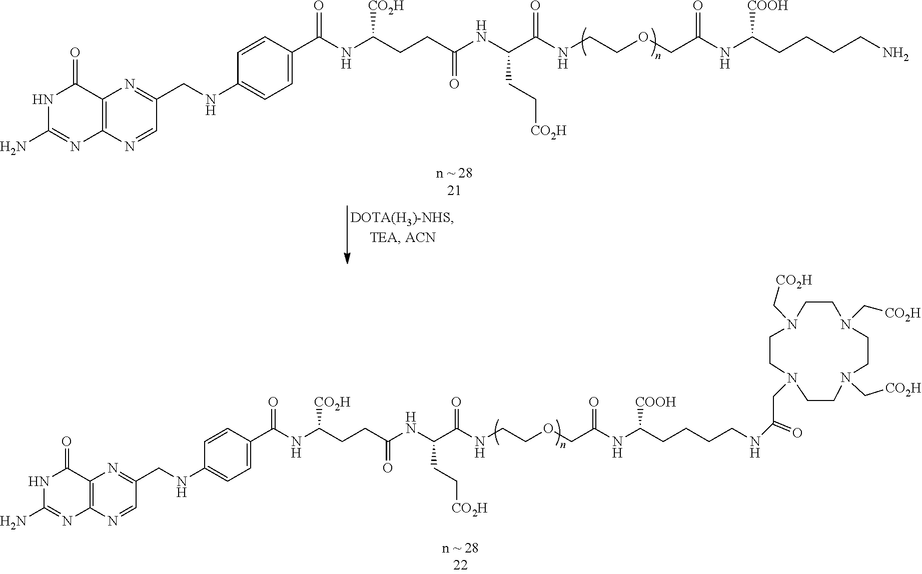

The folate spacer 21 (5 mg, assumed to be 0.0012 mmol) and 1,4,7,10-tetraazacyclododecane-1,4,7, 10-tetraacetic acid mono-N-hydroxysuccinimide ester HPF₆ TFA salt (DOTA(H₃)—NHS (commercially obtained), 4.7 mg, 4 eq.) were dissolved in ACN (125 μL). To this solution was added triethylamine (TEA, 5 mL, 29 eq.). The reaction was stirred for 2 hrs. The reaction was diluted with H₂O and loaded onto a C18 silica reversed-phase column (0.1% TFA and ACN eluents) to give 2.9 mg of conjugate after lyophilization.

¹H NMR (500 MHz, DMSO-d₆/D₂O): δ 8.63 (s, 1H), 7.63 (d, 2H, J=8.5 Hz), 6.63 (d, 2H, J=8.5 Hz), 4.48 (s, 2H), 4.3-4.1 (m, 4H), 3.92 (s, 3H), 3.9-3.3 (m), 3.2-2.8 (m, 22H), 2.3-2.2 (m, 4H), 2.05 (m, 1H), 1.95-1.6 (m, 6H), 1.4 (m, 2H), 1.3 (m, 2H).

### Example 13

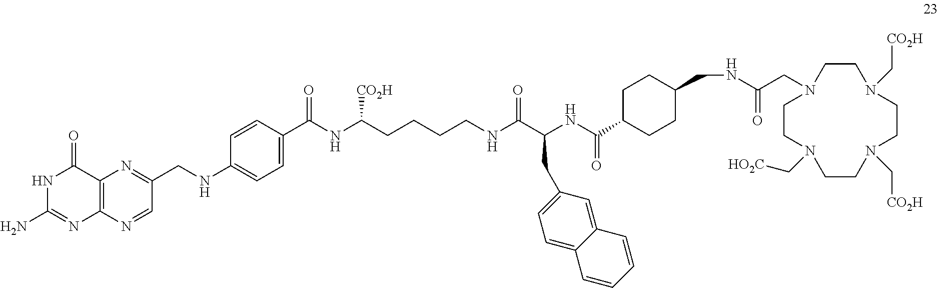 [SMILES: Nc1nc2ncc(CNc3ccc(C(=O)N[C@@H](CCCCNC(=O)[C@H](Cc4ccc5ccccc5c4)NC(=O)[C@H]4CC[C@H](CNC(=O)CN5CCN(CC(=O)O)CCN(CC(=O)O)CCN(CC(=O)O)CC5)CC4)C(=O)O)cc3)nc2c(=O)[nH]1]

23 was synthesized by standard Fmoc-SPPS techniques following the general procedures outlined for 14 from Fmoc-L-Lys(Mtt)-Wang resin using the following materials:

TABLE 5
Constituent compounds used for 23

| | Amount | mmol or |
|---|---|---|
| Compound | (mg) | eqivalents |
| Fmoc-Lys(Mtt)-Wang resin | 395 | 0.387 |
| (commercially obtained) | | |
| N¹⁰-TFA-pteroic acid | 93 | 1.5 eq. |
| Fmoc-Ala(2-naphthyl)-OH | 133 | 2 eq. |
| (commercially obtained) | | |
| Fmoc-tranexamic acid* | 173 | 3 eq. |
| (commercially obtained) | | |
| DOTA(H₃)-NHS** | 173 | 1.5 eq |
| (commercially obtained) | | |

*Modified resin was treated with Fmoc-transexamic acid two times: once with 115 mg (2 eq.) and a second time with 58 mg (1 eq.) to assure complete coupling.
**No PyBOP was used during this coupling. 158 μL of DIPEA (6 eq.) was used.

Unless otherwise noted, 2 equivalents of PyBOP and 4 equivalents of ⁱPrNEt were used for each coupling step. The crude material was purified by C18 silica reversed-phase column chromatography (0.1% TFA and ACN as eluents) to provide 15 mg of desired product after lyophilization. LC/MS (ESI-QMS): m/z=1163.6 (M+H)⁺, calculated m/z=1163.6 ¹H NMR (500 MHz, DMSO-d₆/D₂O): δ 8.68 (s, 1H), 7.81 (d, 1H, J=7 Hz), 7.74 (m, 2H), 7.62 (m, 3H), 7.4 (m, 2H), 7.32 (d, 1H, J=8.5 Hz), 6.58 (d, 2H, J=8.5 Hz), 4.43 (m, 3H), 4.16 (t, 1H, J=7.5 Hz), 3.5-2.5 (m, 27H), 1.97 (bt, 1H), 1.7-1.45 (m, 6H), 1.38 (d, 1H, J=9.5 Hz), 1.35-0.9 (m, 8H), 0.75 (t, 2H, J=12 Hz).

### Example 14

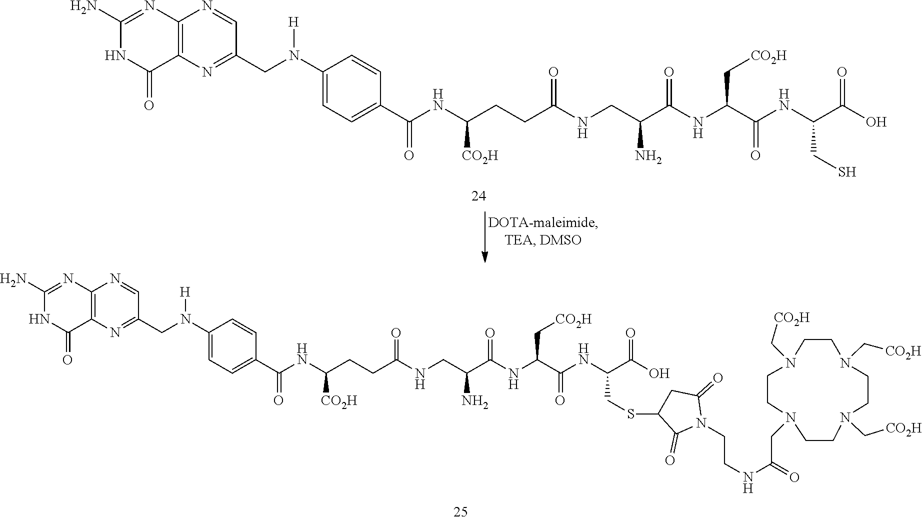 [SMILES: Nc1nc2ncc(CNc3ccc(C(=O)N[C@@H](CCC(=O)NC[C@H](N)C(=O)N[C@@H](CC(=O)O)C(=O)N[C@@H](CS)C(=O)O)C(=O)O)cc3)nc2c(=O)[nH]1 ; Nc1nc2ncc(CNc3ccc(C(=O)N[C@@H](CCC(=O)NC[C@H](N)C(=O)N[C@@H](CC(=O)O)C(=O)N[C@@H](CSC4CC(=O)N(CCNC(=O)CN5CCN(CC(=O)O)CCN(CC(=O)O)CCN(CC(=O)O)CC5)C4=O)C(=O)O)C(=O)O)cc3)nc2c(=O)[nH]1]

24 (5.0 mg, 0.0067 mmol) was dissolved in DMSO (700 μL) and TEA (9.3 μL, 10 eq.) followed by 1,4,7,10-tetraazacyclododecane-1,4,7-tris-acetic acid-10-maleimidoethylacetamide HPF₆ TFA salt (DOTA-maleimide (commercially obtained), 7.4 mg, 1.4 eq.) in DMSO (500 μL). The reaction was stirred for 1 hr. The reaction was loaded directly onto a C18 silica reversed-phase column (0.1% TFA and ACN eluents) to give 7.5 mg of product after lyophilization. LC/MS (ESI-QMS): m/z=1272.1 (M+H)⁺, calculated m/z=1272.5 ¹H NMR (500 MHz, DMSO-d₆/D₂O): δ 8.63 (s, 1H), 7.62 (d, 2H, J=8.5 Hz), 6.63 (d, 2H, J=8.5 Hz), 4.63 (dd, 1H, J=6.5, 14 Hz), 4.5-4.4 (m, 3H), 4.34 (dd, 1H, J=5, 10 Hz), 4.00 (m, 2H), 3.81 (t, 2H, J=7.5 Hz), 3.45-3.0 (m, 22H), 2.91 (m, 1H), 2.72 (m, 1H), 2.22 (bt, 2H, J=7.5 Hz), 2.10 (m, 1H), 1.88 (m, 1H).

### Example 15

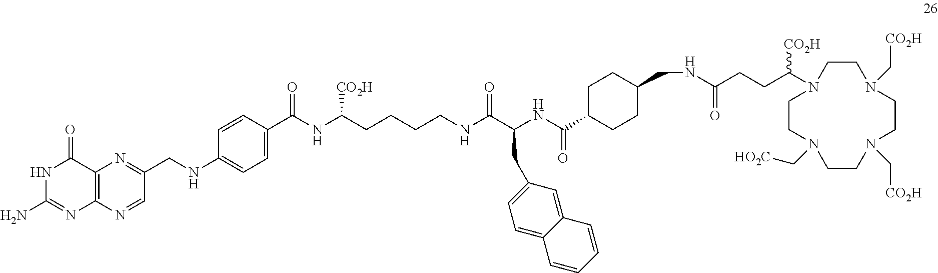 [SMILES: Nc1nc2ncc(CNc3ccc(C(=O)N[C@@H](CCCCNC(=O)[C@H](Cc4ccc5ccccc5c4)NC(=O)[C@H]4CC[C@H](CNC(=O)CCC(C(=O)O)N5CCN(CC(=O)O)CCN(CC(=O)O)CCN(CC(=O)O)CC5)CC4)C(=O)O)cc3)nc2c(=O)[nH]1]

26 was synthesized by standard Fmoc-SPPS techniques following the general procedures outlined for 14 from Fmoc-L-Lys(Mtt)-Wang resin using the following materials:

TABLE 6
Constituent compounds used for 26

| | Amount | mmol or | |
|---|---|---|---|
| Compound | (mg) | equivalents | |
| Fmoc-L-Lys(Mtt)-Wang resin | 372 | 0.2 | mmol |
| (commercially obtained) | | | |
| N¹⁰-TFA-pteroic acid | 163 | 2 | eq. |
| Fmoc-Ala(2-naphthyl)-OH | 175 | 2 | eq. |
| (commercially obtained) | | | |
| Fmoc-tranexamic acid | 152 | 2 | eq. |
| (commercially obtained) | | | |
| DOTAGA-tetra-tBu ester* | 245 | 1.75 | eq. |
| (commercially obtained) | | | |

*2-[1,4,7,10-tetraazacyclododecane-4,7,10-tris-(t-Butyl acetate)]-pentanedioic acid-1-tert-butyl ester

2 equivalents of PyBOP and 4 equivalents of ⁱPrNEt were used for each coupling step. After standard resin cleavage, deprotection of the DOTAGA associated tert-butyl esters was achieved by heating the cleavage solution to 35° C. for 2 hrs. The crude material was purified by C18 silica reversed-phase column chromatography (0.1% TFA and ACN as eluents) to provide 9 mg of clean product and 7 mg of mixed fractions. LC/MS (ESI-QMS): m/z=1235.5 (M+H)⁺, calculated m/z=1235.6 ¹H NMR (500 MHz, DMSO-d₆/D₂O): δ 8.58 (s, 1H), 7.78 (d, 1H, J=8 Hz), 7.75 (t, 2H, J=15 Hz), 7.61 (m, 3H), 7.39 (m, 2H), 7.31 (d, 1H, J=8 Hz).

### Example 16: Pte(N¹⁰-TFA)-γGlu-EDA-NH₂ (27)

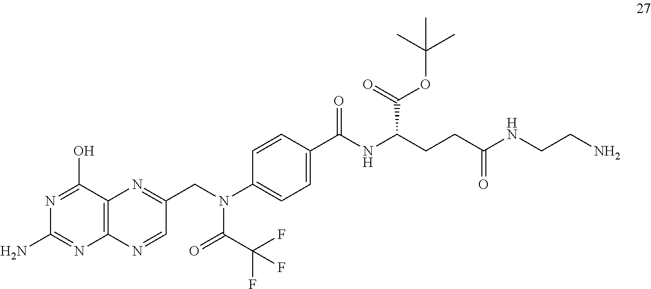 [SMILES: CC(C)(C)OC(=O)[C@H](CCC(=O)NCCN)NC(=O)c1ccc(N(Cc2cnc3nc(N)nc(O)c3n2)C(=O)C(F)(F)F)cc1]

27 was synthesized by standard Fmoc-SPPS techniques as described in the general synthetic procedures outlined for 10 from 1,2-diaminoethane trityl resin.

TABLE 7
Compounds using in the synthesis of 27.

| | | | Molecular | |
|---|---|---|---|---|
| | | Equiv- | Weight | Quantity |
| Compound | mmol | alent | (g/mol) | (g) |
| 1,2-diaminoethane trityl resin | 0.182 | 1 | | 0.285 |
| (commercially obtained) | | | | |
| (Loading ~0.64 mmol/g) | | | | |
| Fmoc-Glu-O^{t}Bu | 0.365 | 2 | 425.5 | 0.155 |
| (commercially obtained) | | | | |
| N¹⁰-TFA-Pteroic Acid | 0.365 | 2 | 408 | 0.149 |
| PyBOP | 0.365 | 2 | 520.31 | 0.190 |
| ⁱPr₂NEt | 0.547 | 3 | 129.24 (d = | 0.071 |
| | | | 0.742) | |

In a peptide synthesis vessel, 1,2-diaminoethane trityl resin (0.285 g, 0.182 mmol) was placed and washed with DMF (3×10 ml). Initial Fmoc deprotection was performed using 20% piperidine in DMF (3×10 ml) solution for 10 mins per cycle. Subsequent washes of DMF (3×10 ml) and IPA (3×10 ml), a Kaiser test was done to determine reaction completion. Following another DMF wash (3×10 ml); an amino acid solution (2.0 eq.) in DMF, PyBOP (2.0 eq.) and ⁱPr₂NEt (3.0 eq.) were added to the vessel and the solution bubbled with Argon for 1 hour. The coupling solution was filtered, the resin was washed with DMF (3×10 ml) and IPA (3×10 ml) and a Kaiser test was done to assess reaction completion. The above process was performed successively for the additional coupling. Resin cleavage was performed with 1,1,1,3,3,3 hexafluoro-2-propanol (10 ml) poured onto the resin and bubbled with argon for 30 mins, followed by filtration into a clean flask. Further cleavage was performed twice successively with fresh cleavage cocktail for 10 mins of bubbling. The combined filtrate was concentrated under reduced pressure and the crude residue was collected to yield the amine (0.105 g, 90%). LC/MS (ESI-QMS): m/z=636.4 (M+H)⁺, calculated m/z=635.6 ¹H NMR (500 MHz DMSO-d₆) Pivotal signals: δ 8.49 (s, 1H), 7.78 (d, 2H), 7.45 (d, 2H), 5.05 (s, 2H), 1.32 (s, 9H).

### Example 17

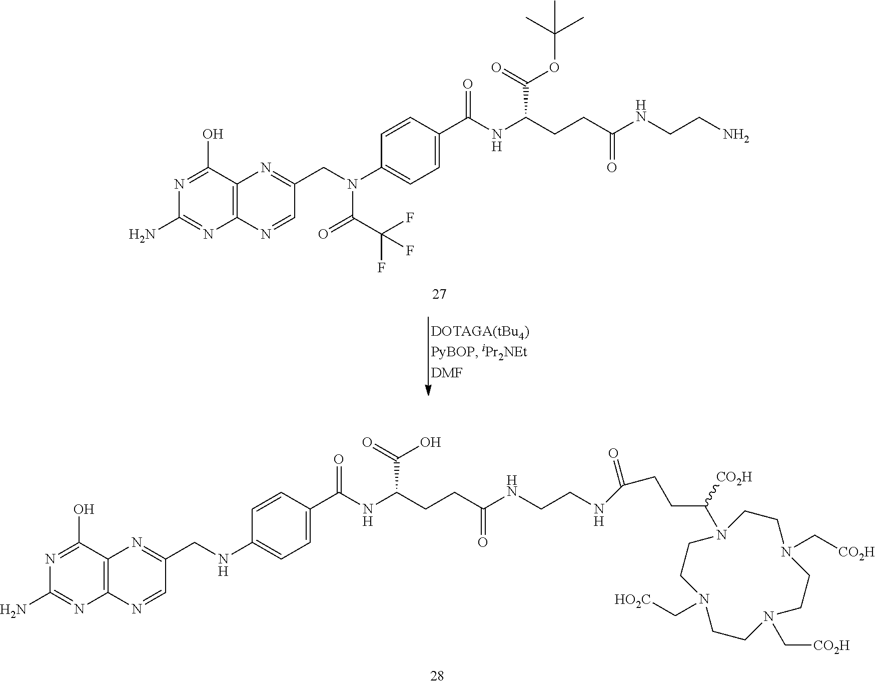 [SMILES: CC(C)(C)OC(=O)[C@H](CCC(=O)NCCN)NC(=O)c1ccc(N(Cc2cnc3nc(N)nc(O)c3n2)C(=O)C(F)(F)F)cc1 ; Nc1nc(O)c2nc(CNc3ccc(C(=O)N[C@@H](CCC(=O)NCCNC(=O)CCC(C(=O)O)N4CCN(CC(=O)O)CCN(CC(=O)O)CCN(CC(=O)O)CC4)C(=O)O)cc3)cnc2n1]

In a dry flask, 27 (102 mg, 0.161 mmol, 1.0 eq.), DOTAGA(tBu₄) (commercially obtained, 169 mg, 0.242 mmol, 1.5.0 eq.), and PyBOP (168 mg, 0.323 mmol, 2.0 eq.) were dissolved in DMF (5 ml) under argon. ⁱPr₂NEt (0.12 ml, 0.645 mmol, 4 eq.) was added to the solution, and stirred for an addition hour. The reaction was monitored until completion by LCMS and purified using C18 silica reversed phase column chromatography (NH₄HCO₃ (pH=7) and ACN eluents) to yield the N¹⁰-TFA protected conjugate. The N¹⁰-TFA protected conjugate was dissolved in a solution of Na₂CO₃ and monitored for the N¹⁰-TFA deprotection. Upon completion of the reaction, the deprotected amine was isolated using the C18 silica reversed phase column and lyophilized. Further deprotection of the t-butyl esters was performed by dissolving the conjugate in a solution of TFA and stirring for 1 hour. Upon full deprotection, the reaction was concentrated under reduced pressure, and purified using C18 reversed phase silica to yield conjugate 28 (12 mg, 8%). LC/MS (ESI-QMS): m/z=942.4 (M+H)⁺, calculated m/z=941.9

### Example 18

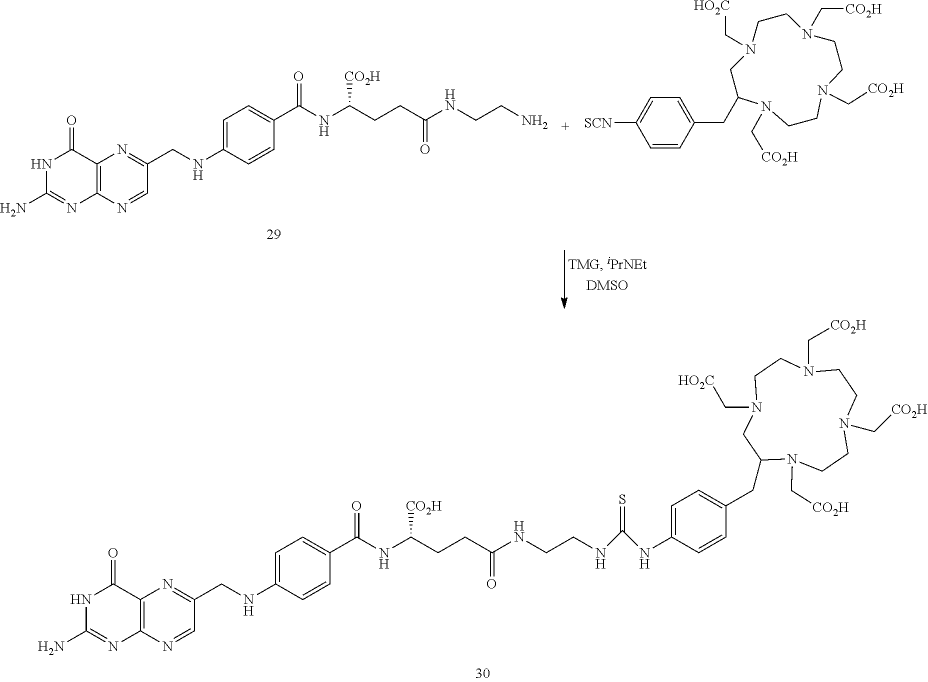

To a solution of 29 (25.5 mg, 0.053 mM) in DMSO (2.0 mL) and tetramethylguanidine (TMG, 0.007 mL, 0.053 mM) was added DOTA-benzyl isocyanate (commercially obtained, 34.98 mg, 0.063 mM) and ⁱPr₂NEt (0.046 mL, 0.264 mM). The resulting homogeneous solution was stirred at ambient temperature under argon for 1 h. LCMS analysis confirmed the product formation. Reaction mixture was loaded directly onto a preparatory HPLC (Mobile phase A=50 mM Ammonium bicarbonate, pH=7.0. B=ACN. Method: 0-30% B in 25 min.) for purification. Fractions containing the desired product were collected, combined, and freeze-dried to afford the conjugate Compound 30 (37.0 mg) as a yellow solid. LC/MS (ESI-QMS): m/z=1035.8 (M+H)⁺, calculated m/z=1035.4

### Example 19

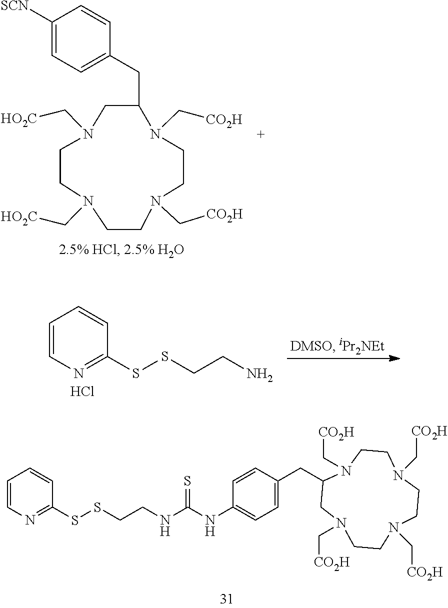

To a solution of pyridyl dithioethylamine hydrochloride (commercially obtained, 10.8 mg, 0.049 mM) and ⁱPr₂NEt (0.130 mL, 0.730 mM) was added p-SCN-Bn-DOTA(H₄) (commercially obtained, 50.00 mg, 0.073 mM) portion wise over 15 min. Reaction mixture was stirred for 1 h. LCMS analysis confirmed the product formation. Reaction mixture was loaded directly onto a preparatory HPLC (Mobile phase A=0.1% TFA in water, pH=2.0. B=ACN. Method: 1-50% B in 25 min.) for purification. Fractions containing the desired product were collected, combined, and freeze-dried to afford the compound 31 (16.0 mg). LC/MS (ESI-QMS): m/z=760.3 (M+Na)⁺, calculated m/z=760.2

### Example 20

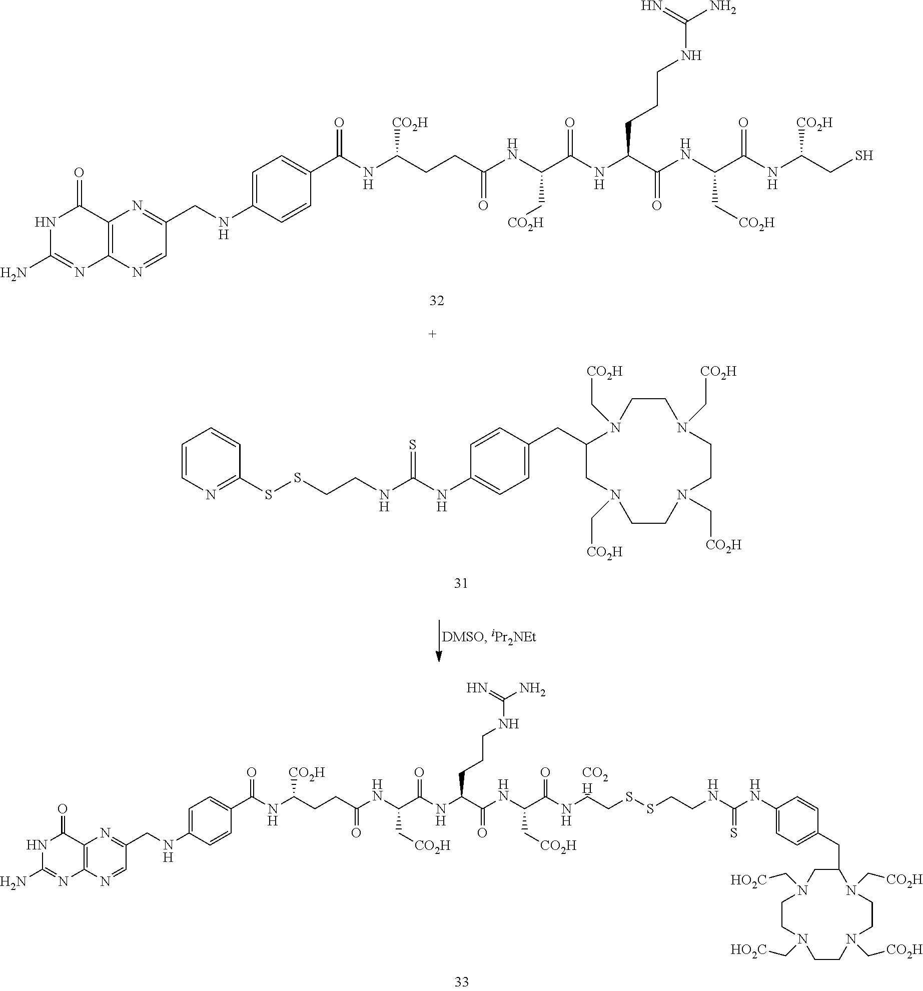 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CS)C(=O)O ; O=C(O)CN1CCN(CC(=O)O)CCN(CC(=O)O)C(Cc2ccc(NC(=S)NCCSSc3ccccn3)cc2)CN(CC(=O)O)CC1 ; N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CSSCCNC(=S)Nc1ccc(CC2CN(CC(=O)O)CCN(CC(=O)O)CCN(CC(=O)O)CCN2CC(=O)O)cc1)C(=O)O]

To a solution of 32 (20.8 mg, 0.022 mM) in DMSO (2.0 mL) were added DOTA derivative 31 (16 mg, 0.022 mM) and ⁱPr₂NEt (0.037 mL, 0.22 mM). The resulting homogeneous solution was stirred at ambient temperature under argon for 1 h. LCMS analysis confirmed the product formation. Reaction mixture was loaded directly onto a preparatory HPLC (Mobile phase A=0.10% TFA in water, pH=2.0. B=ACN. Method: 0-30% B in 25 min.) for purification. Fractions containing the desired product were collected, combined, and freeze-dried to afford the conjugate 33 (7.5 mg) as a yellow solid. LC/MS (ESI-QMS): m/z=779.9 (M+2H)²⁺, calculated m/z=779.8

### N-α-Pteroyl-Lysine Based DOTA Conjugates

### Example 21: Synthesis of Pte-Lys(β-Asp-2-Nal-Gly-DOTA)-OH (Compound 34): 2,2′,2″-(10-((3S,11S,14S)-1-(4-(((2-amino-4-oxo-3,4-dihydropteridin-6-yl)methyl)amino)phenyl)-3,11-dicarboxy-14-(naphthalen-2-ylmethyl)-1,9,13,16,19-pentaoxo-2,8,12,15,18-pentaazaicosan-20-yl)-1,4,7,10-tetraazacyclododecane-1,4,7-triyl)triacetic acid

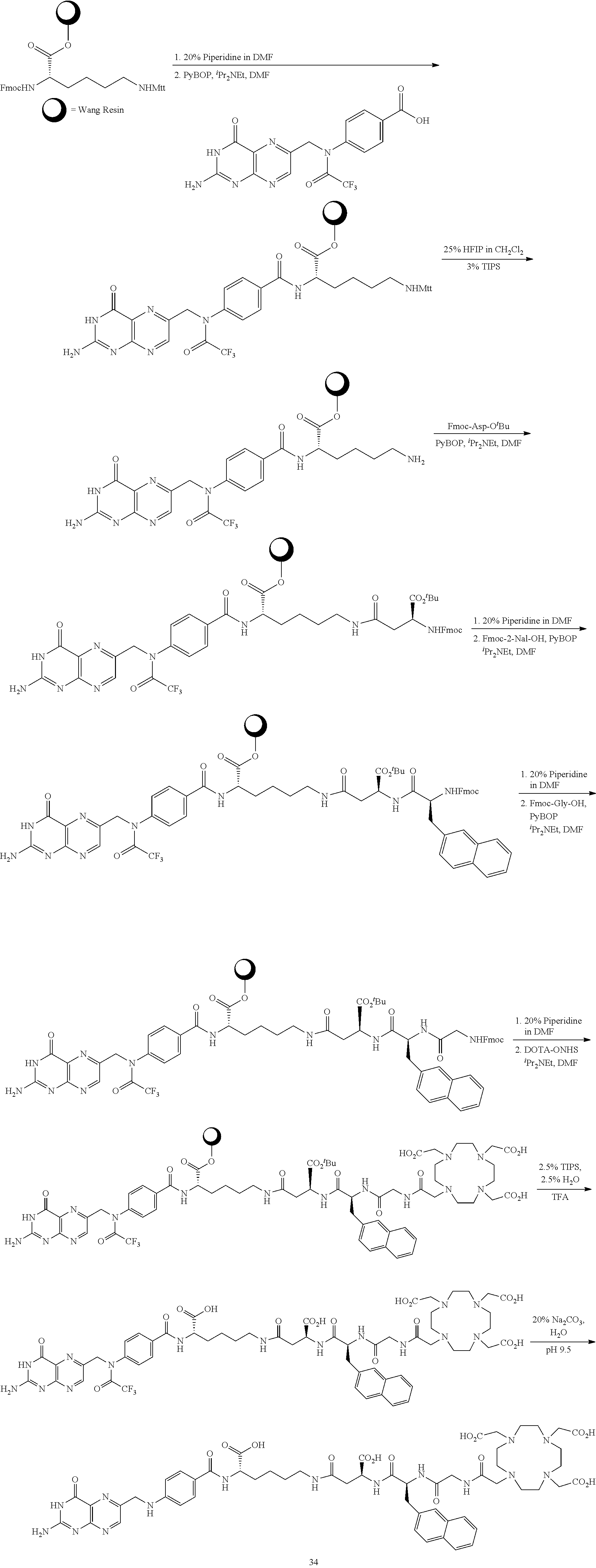

Compound 34 was synthesized by solid phase in seven steps starting from Fmoc-Lys(N-4-methoxytrityl)-Wang-Resin (Table 8).

TABLE 8
Solid phase reaction steps:

| | | | MW | | |
|---|---|---|---|---|---|
| | mmol. | equiv. | (g/mol) | amount | |
| Fmoc-Lys(N-4-methoxytrityl)- | 0.22 | — | — | 0.333 | g |
| Wang Resin | | | | | |
| (loading 0.66 mmol/g.) | | | | | |
| (commercially obtained) | | | | | |

Procedure A: Fmoc Deprotection - 20% Piperidine in DMF (3X)
Procedure C: N¹⁰-TFA-Pteroic Acid Coupling

| | | | | | |
|---|---|---|---|---|---|
| N¹⁰-TFA-Pteroic Acid | 0.44 | 2 | 408 | 180 | mg |
| ⁱPr₂NEt | 0.88 | 4 | 129 | 0.153 | mL |
| PyBOP | 0.44 | 2 | 520 | 229 | mg |

Procedure B: Mtt Deprotection - 25% HFIP in CH₂Cl₂ (6X)
Procedure C: Fmoc-L-Asp-O^{t}Bu Coupling

| | | | | | |
|---|---|---|---|---|---|
| Fmoc-Asp-O^{t}Bu | 0.44 | 2 | 412 | 181 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Fmoc-3-(2-naphthyl)-L-alanine
(Fmoc-2-Nal-OH) Coupling

| | | | | | |
|---|---|---|---|---|---|
| Fmoc-2-Nal-OH | 0.44 | 2 | 438 | 193 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.44 | 2 | 297 | 131 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: N-Hydroxysuccinamidyl 1,4,7,10-tetraazacyclododecane-
1,4,7,10-tetraacetate {DOTA-ONHS} Coupling

| | | | | | |
|---|---|---|---|---|---|
| DOTA-ONHS | 0.33 | 1.5 | 762 | 251 | mg |
| (commercially obtained) | | | | | |
| ⁱPr₂NEt | 0.88 | 4 | 129 | 0.153 | mL |

Procedure D: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

### Procedure E: Deprotection of N¹⁰-TFA Group in Pteroic Acid and Purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

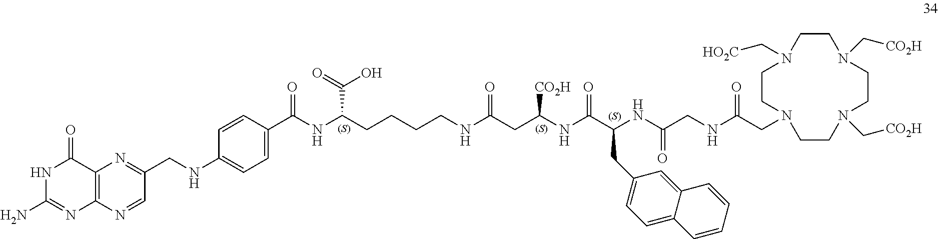

LCMS (ESI): [M+H]⁺=Calculated for C₅₅H₇₀N₁₅O₁₆, 1196.50; found 1196.7.

Selected data ¹H NMR (500 MHz, DMSO-d6, D₂O): δ 8.62 (s, 1H), 7.78 (m, 2H), 7.70 (d, J=9 Hz, 1H), 7.68 (s, 1H), 7.60 (d, J=9 Hz, 2H), 7.39 (m, 1H), 7.38 (m, 2H), 6.61 (d, J=8.5 Hz, 2H), 4.46 (s, 2H).

Utilizing the above SPPS procedures, the below DOTA conjugates were prepared:

### Example 22: Synthesis of Fmoc-Tyr(O-benzyloxycarbonylmethyl)-O^{t}Bu (Compound 35)

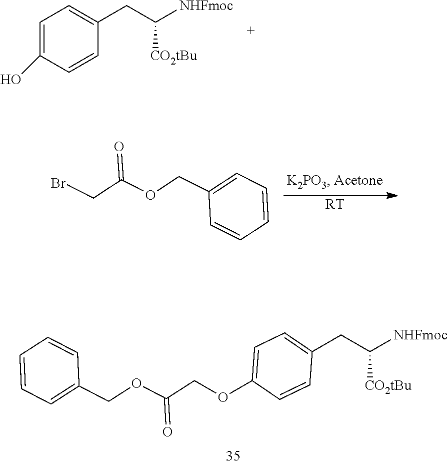

To a solution of Fmoc-Tyr-O^{t}Bu (commercially obtained, 1.38 g, 3.0 mM) in dry acetone (10 mL) was added potassium carbonate (1.24 g, 9.0 mM) and stirred for 5 min. Bromo-benzyl acetate (commercially obtained, 0.52 mL, 3.3 mM) was added. The reaction was allowed to stir at RT for 3 h, LCMS analysis (20 mM NH₄HCO₃, pH 7.4) indicated that the reaction was complete. The reaction mixture was filtered and concentrated. Residue was dissolved in EtOAc, washed with water (2×) and brine. Organic layer was dried over Na₂SO₄, concentrated and purified by combi-flash chromatography (0-100% EtOAc in petroleum ether) to yield Compound 35.

LCMS (ESI): [M+H]⁺=Calculated for C₃₇H₃₈NO₇, 608.26; found 608.25.

¹H NMR (500 MHz, CDCl₃): δ 7.70 (d, J=7.5 Hz, 2H), 7.58 (t, J₁=6.0 Hz, J₂=6.5 Hz, 2H), 7.36 (m, 9H), 7.06 (d, J=8.5 Hz, 2H), 6.82 (d, J=9.0 Hz, 2H), 5.26 (d, J=8.5 Hz, 1H), 5.24 (s, 2H), 4.63 (s, 2H), 4.51 (ABq, J₁=13.5 Hz, J₂=6.0 Hz, 5.5 Hz, 1H), 4.45 (dd, J₁=10.5 Hz, J₂=7.0 Hz, 7.5 Hz, 1H), 4.34 (dd, J₁=10.75 Hz, J₂=7.0 Hz, 7.5 Hz, 1H), 4.21 (t, J₁=6.5 Hz, J₂=7.0 Hz, 1H), 3.04 (d, J=6.0 Hz, 2H), 1.43 (s, 9H).

### Example 23: Synthesis of Fmoc-Tyr(O-carboxymethyl)-O^{t}Bu (Compound 36)

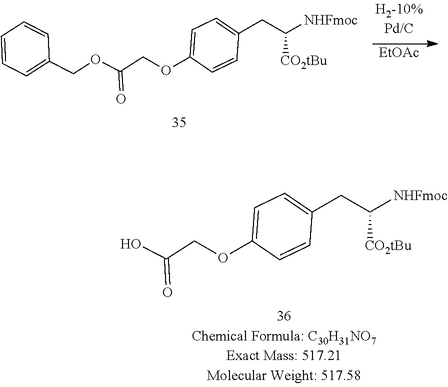

To a solution of Compound 35 (0.40 g, 0.66 mM) in ethyl acetate (30 mL) was added 10% Pd/C (0.15 g) and stirred for 15 min under H₂ atmosphere (balloon). LCMS analysis (20 mM NH₄HCO₃, pH 7.4) indicated that the reaction was complete. The reaction mixture was filtered, concentrated and dried to yield Compound 36. Material was directly used for next solid phase coupling reactions.

LCMS (ESI): [M+Na]⁺=Calculated for C₃₀H₃₁NO₇Na, 540.21; found 540.59.

¹H NMR (500 MHz, CDCl₃): δ 7.77 (d, J=7.0 Hz, 2H), 7.58 (t, J₁=7.0 Hz, J₂=7.5 Hz, 2H), 7.41 (t, J₁=7.5 Hz, J₂=7.5 Hz, 2H), 7.29-7.35 (m, 2H), 7.08 (d, J=8.0 Hz, 2H), 6.84 (d, J=8.0 Hz, 2H), 5.38 (d, J=8.0 Hz, 1H), 4.63 (s, 2H), 4.53 (ABq, J₁=14.0 Hz, J₂=6.0 Hz, 1H), 4.45 (dd, J₁=10.75 Hz, J₂=7.0 Hz, 7.5 Hz, 1H), 4.35 (dd, J₁=11.0 Hz, J₂=7.0 Hz, 1H), 4.21 (t, J₁=6.5 Hz, J₂=7.5 Hz, 1H), 2.75-3.12 (m, 2H), 1.43 (s, 6H), 1.39 (s, 3H).

### Example 24: Synthesis of Pte-Lys(DOTA-Gly-Tyr(O-carbonylmethyl)-OH)—OH (Compound 37): 2,2′,2″-(10-(2-((2-(((S)-2-(4-(2-(((S)-5-(4-(((2-amino-4-oxo-3,4-dihydropteridin-6-yl)methyl)amino)benzamido)-5-carboxypentyl)amino)-2-oxoethoxy)phenyl)-1-carboxyethyl)amino)-2-oxoethyl)amino)-2-oxoethyl)-1,4,7,10-tetraazacyclododecane-1,4,7-triyl)triacetic acid

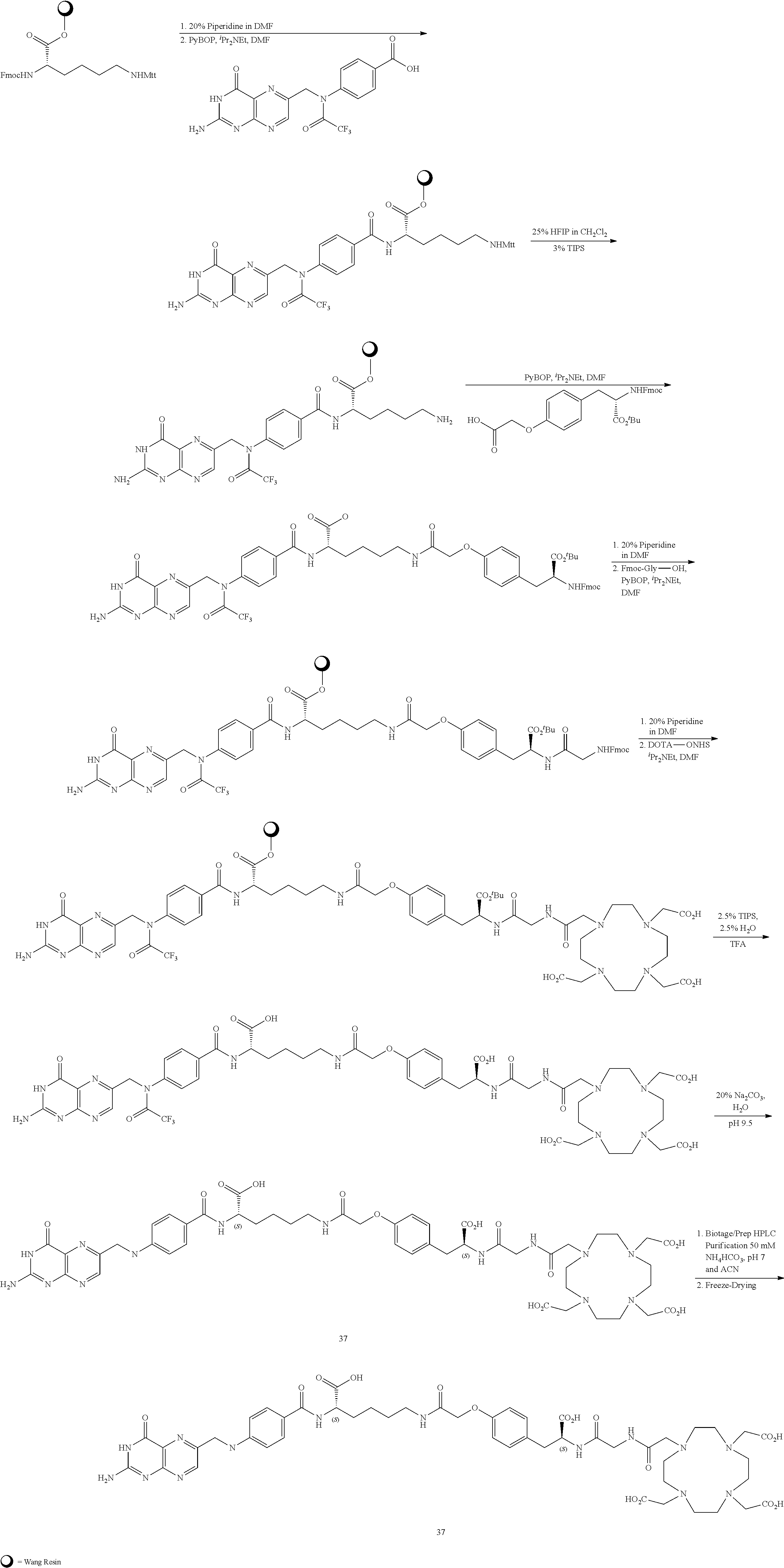

Compound 37 was synthesized by solid phase in six steps starting from Fmoc-Lys(N-4-methoxytrityl)-Wang-Resin

Solid phase reaction steps:

| | | | MW | | |
|---|---|---|---|---|---|
| | mmol. | equiv. | (g/mol) | amount | |
| Fmoc-Lys(N-4-methoxytrityl)- | 0.22 | — | — | 0.333 | g |
| Wang Resin | | | | | |
| (loading 0.66 mmol/g.) | | | | | |
| (commercially obtained) | | | | | |

Procedure A: Fmoc Deprotection - 20% Piperidine in DMF (3X)
Procedure C: N¹⁰-TFA-Pteroic Acid Coupling

| | | | | | |
|---|---|---|---|---|---|
| N¹⁰-TFA-Pteroic Acid | 0.44 | 2 | 408 | 180 | mg |
| ⁱPr₂NEt | 0.88 | 4 | 129 | 0.153 | mL |
| PyBOP | 0.44 | 2 | 520 | 229 | mg |

Procedure B: Mtt Deprotection - 25% HFIP in CH₂Cl₂ (6X)
Procedure C: Fmoc-L-Tyr(OCH₂CO₂H)-O^{t}Bu Coupling

| | | | | | |
|---|---|---|---|---|---|
| Fmoc-L-Tyr(OCH₂CO₂H)-O^{t}Bu | 0.44 | 2 | 517.6 | 227.7 | mg |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.44 | 2 | 297 | 131 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: N-Hydroxysuccinamidyl 1,4,7,10-tetraazacyclododecane-
1,4,7,10-tetraacetate {DOTA-ONHS} Coupling

| | | | | | |
|---|---|---|---|---|---|
| DOTA-ONHS | 0.33 | 1.5 | 762 | 251 | mg |
| (commercially obtained) | | | | | |
| ⁱPr₂NEt | 0.88 | 4 | 129 | 0.153 | mL |

Procedure D: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

### Procedure E: Deprotection of N¹⁰-TFA Group in Pteroic Acid and Purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

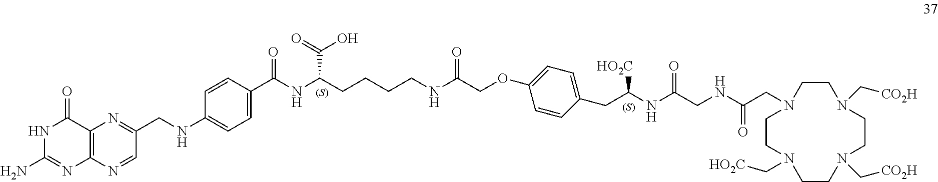

LCMS (ESI): [M+H]⁺=Calculated for C₄₉H₆₅N₁₄O₁₆, 1105.46; found 1105.3.

Selected data ¹H NMR (500 MHz, DMSO-d6, D₂O): δ 8.61 (s, 1H), 7.61 (d, J=9 Hz, 2H), 7.08 (d, J=8 Hz, 2H), 6.74 (d, J=8.5 Hz, 2H), 6.61 (d, J=8.5 Hz, 2H), 4.48 (s, 2H), 4.31 (s, 2H).

Examples 25-31 are synthesized using similar procedures described in the Examples, above, using appropriate starting materials:

### Example 25: Pte-Lys(DOTA-Tyr(O-carbonylmethyl)-OH)—OH (Compound 38)

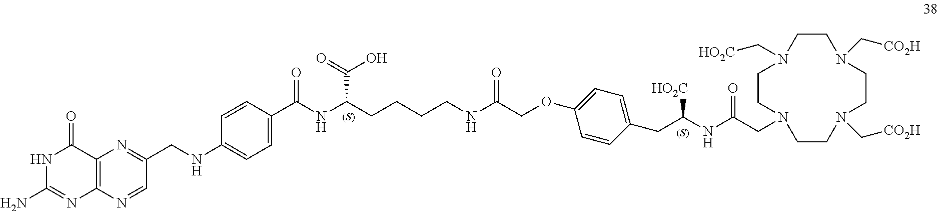

LCMS (ESI): [M+H]⁺=Calculated for C₄₇H₆₂N₁₃O₁₅, 1048.44; found 1048.5.

Selected data ¹H NMR (500 MHz, DMSO-d6, D₂O): δ 8.60 (s, 1H), 7.58 (d, J=8.5 Hz, 2H), 7.18 (d, J=8.5 Hz, 2H), 6.78 (d, J=9.0 Hz, 2H), 6.54 (d, J=9.0 Hz, 2H), 4.31 (s, 2H).

### Example 26: Pte-Lys(DOTA-(Tyr(O-carbonylmethyl)-OH)₂)—OH (Compound 39)

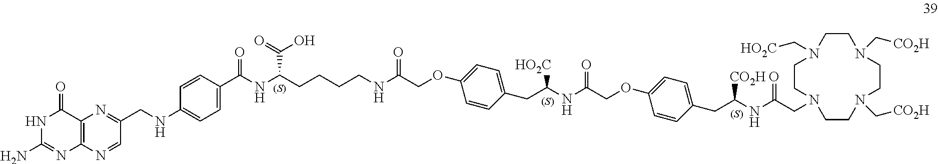

LCMS (ESI): [M+H]⁺=Calculated for C₅₈H₇₃N₁₄O₁₉, 1269.51; found 1269.5.

Selected data ¹H NMR (500 MHz, DMSO-d6, D₂O): δ 8.60 (s, 1H), 7.58 (d, J=8.0 Hz, 2H), 7.06 (d, J=8.5 Hz, 2H), 7.00 (d, J=8.0 Hz, 2H), 6.71 (d, J=8.5 Hz, 2H), 6.69 (d, J=9.5 Hz, 2H), 6.61 (d, J=8.0 Hz, 2H), 4.48 (s, 2H), 4.36 (s, 2H), 4.29 (s, 2H).

### Example 27: Pte-Lys(2-Nal-Gly-DOTA)-OH (Compound 40)

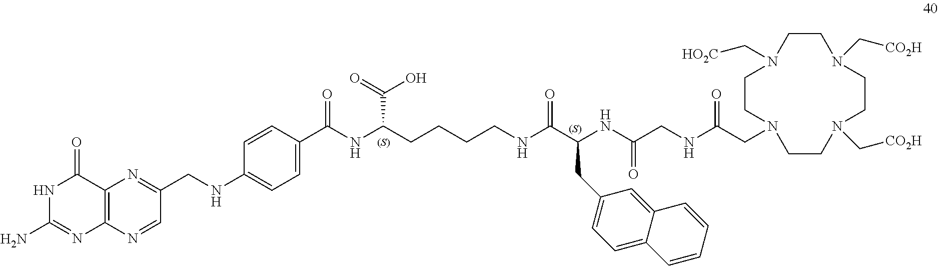

LCMS (ESI): [M+H]⁺=Calculated for C₅₁H₆₅N₁₄O₁₃, 1081.48; found 1081.6.

Selected data ¹H NMR (500 MHz, DMSO-d6, D₂O): δ 8.59 (s, 1H), 7.74 (m, 2H), 7.65 (d, J=9.0 Hz, 2H), 7.62 (d, J=8.5 Hz, 2H), 7.38 (m, 2H), 7.30 (d, J=8.5 Hz, 1H), 6.60 (d, J=9.0 Hz, 2H), 4.44 (s, 2H), 4.35 (m, 1H), 4.16 (m, 1H).

### Example 28: Pte-Lys(β-Asp-2-Nal-DOTA)-OH (Compound 41)

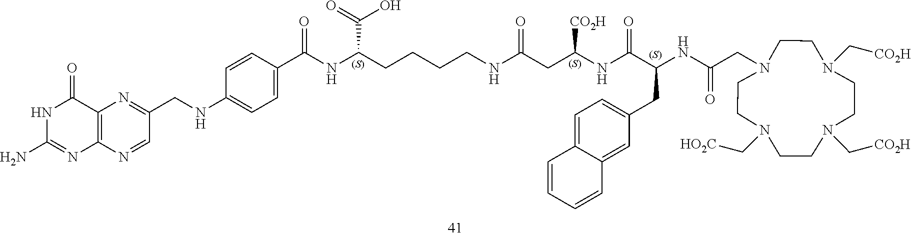

LCMS (ESI): [M+H]⁺=Calculated for C₅₃H₆₇N₁₄O₁₅, 1139.48; found 1139.70.

Selected data ¹H NMR (500 MHz, DMSO-d6, D₂O): δ 8.61 (s, 1H), 7.77 (dd, J₁=7.5 Hz, J₂=7.0 Hz, 2H), 7.73 (d, J=8.5 Hz, 1H), 7.60 (s, 1H), 7.59 (d, J=8.5 Hz, 2H), 7.39 (m, 2H), 7.31 (d, J=7.5 Hz, 1H), 6.61 (d, J=8.5 Hz, 2H), 4.67 (br s, 1H), 4.46 (s, 2H), 4.40 (br s, 1H), 4.16 (dd, J₁=7.0 Hz, J₂=6.5 Hz, 1H).

### Example 29: Pte-Lys(β-Asp-2-Nal-2-Nal-DOTA)-OH (Compound 42)

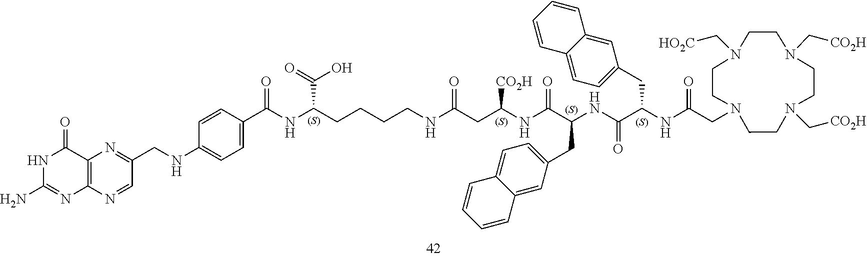

LCMS (ESI): [M+H]⁺=Calculated for C₆₆H₇₈N₁₅O₁₆, 1336.57; found 1336.70.

Selected data ¹H NMR (500 MHz, DMSO-d6, D₂O): δ 8.62 (s, 1H), 7.87 (br s, 1H), 7.80 (m, 4H), 7.75-7.50 (m, 2H), 7.64 (d, J=8.5 Hz, 2H), 7.50-7.35 (m, 6H), 7.17 (br s, 1H), 6.62 (d, J=8.5 Hz, 2H), 4.46 (s, 4H), 4.31 (br s, 1H), 4.23 (t, J=6.5 Hz, 1H).

### Example 30: Pte-Lys(β-Asp-4-Br-Phe-Gly-DOTA)-OH (Compound 43)

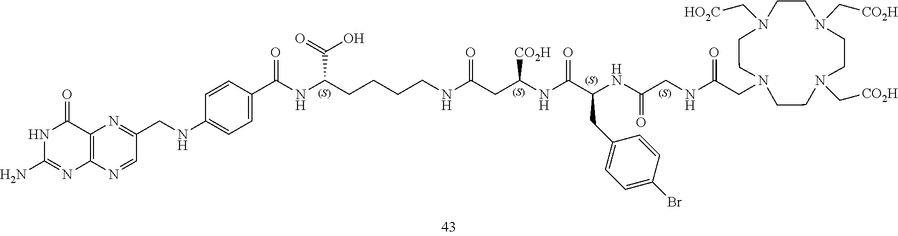

LCMS (ESI): [M+H]⁺=Calculated for C₅₁H₆₇BrN₁₅O₁₆, 1224.40; found 1224.40.

Selected data ¹H NMR (500 MHz, DMSO-d6, D₂O): δ 8.62 (s, 1H), 7.58 (d, J=8.5 Hz, 2H), 7.31 (d, J=8.0 Hz, 2H), 7.14 (d, J=8.5 Hz, 2H), 6.62 (d, J=8.5 Hz, 2H), 4.46 (s, 2H), 4.29 (dd, J₁=9.0 Hz, J₂=5.0 Hz, 1H), 4.19 (dd, J₁=10.5 Hz, J₂=4.0 Hz, 1H), 4.13 (dd, J₁=8.0 Hz, J₂=4.5 Hz, 5.5 Hz, 1H).

### Example 31: Pte-Lys(Phe-Ala-Ser-Phe-Gly-Pro-Pro-Gly-DOTA)-OH (Compound 44)

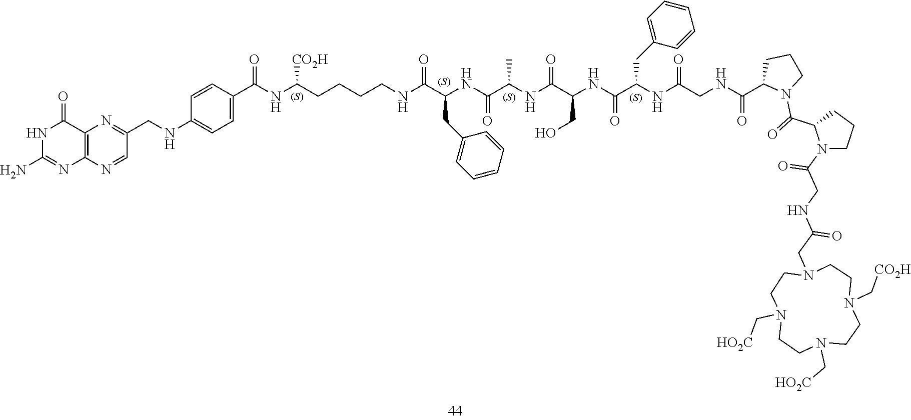

LCMS (ESI): [M+H]⁺=Calculated for C₇₄H₉₉N₂₀O₂₀, 1587.73; found 1587.70.

Selected data ¹H NMR (500 MHz, DMSO-d6, D₂O): δ 8.60 (s, 1H), 7.59 (d, J=9.0 Hz, 2H), 7.23-7.07 (m, 10H), 6.60 (d, J=9.0 Hz, 2H), 4.45 (s, 2H), 4.51-4.42 (m, 1H), 4.37-4.17 (m, 4H), 4.02-3.94 (m, 1H), 3.93-3.85 (m, 1H).

### N-ε-Pteroyl-Lysine Based DOTA Conjugates

### Example 32: Synthesis of DOTA-Gly-Lys(Pte)-OH (Compound 45)

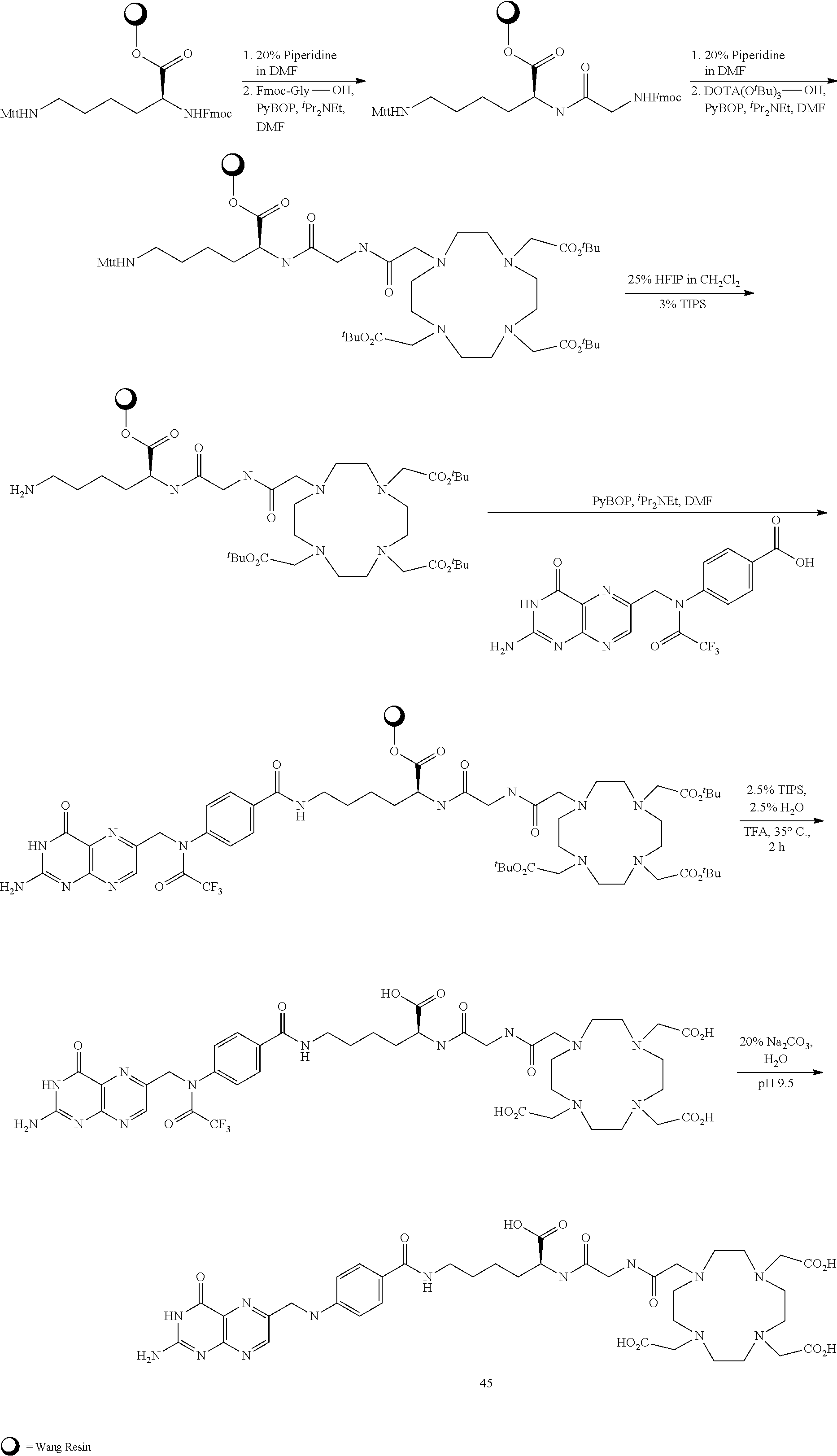

Compound 45 was synthesized by solid phase in five steps starting from Fmoc-Lys(N-4-methoxytrityl)-Wang-Resin (Table 9).

TABLE 9
Solid phase reaction steps:

| | | | MW | | |
|---|---|---|---|---|---|
| | mmol. | equiv. | (g/mol) | Amount | |
| Fmoc-Lys(N-4-methoxytrityl)- | 0.22 | — | — | 0.333 | g |
| Wang Resin | | | | | |
| (loading 0.66 mmol/g.) | | | | | |
| (commercially obtained) | | | | | |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.44 | 2 | 297 | 131 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Tri-tert-butyl 1,4,7,10-tetraazacyclododecane-
1,4,7,10-tetraacetate {DOTA(O^{t}Bu)₃-OH} Coupling

| | | | | | |
|---|---|---|---|---|---|
| DOTA(O^{t}Bu)₃-OH | 0.33 | 1.5 | 573 | 189 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure B: Mtt Deprotection - 25% HFIP in CH₂Cl₂ (6X)
Procedure C: N¹⁰-TFA-Pteroic Acid Coupling

| | | | | | |
|---|---|---|---|---|---|
| N¹⁰-TFA-Pteroic Acid | 0.44 | 2 | 408 | 180 | mg |
| ⁱPr₂NEt | 0.88 | 4 | 129 | 0.153 | mL |
| PyBOP | 0.44 | 2 | 520 | 229 | mg |

Procedure D₂: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min.

Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D₂: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The filtrate was stirred at 35° C. under argon for 2 h. The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

Procedure E: Deprotection of N¹⁰-TFA group in pteroic acid and purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

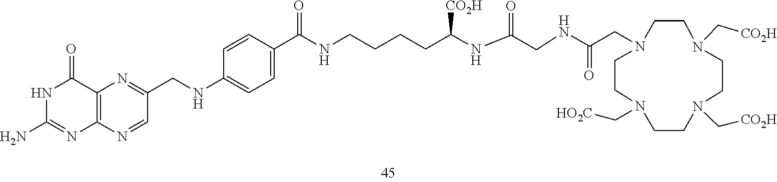 [SMILES: Nc1nc2ncc(CNc3ccc(C(=O)NCCCC[C@H](NC(=O)CNC(=O)CN4CCN(CC(=O)O)CCN(CC(=O)O)CCN(CC(=O)O)CC4)C(=O)O)cc3)nc2c(=O)[nH]1]

LCMS (ESI): [M+H]⁺=Calculated for C₃₈H₅₄N₁₃O₁₂, 884.39; found 884.406.

Selected data ¹H NMR (500 MHz, DMSO-d6, D₂O): δ 8.61 (s, 1H), 7.56 (d, J=9.0 Hz, 2H), 6.59 (d, J=9.0 Hz, 2H), 4.45 (s, 2H), 3.96 (dd, J₁=7.5 Hz, J₂=6.0 Hz, 1H).

Utilizing the above SPPS procedures, the following DOTA conjugates were prepared (see Examples 33-35):

### Example 33: DOTA-Lys(Pte)-OH (Compound 46)

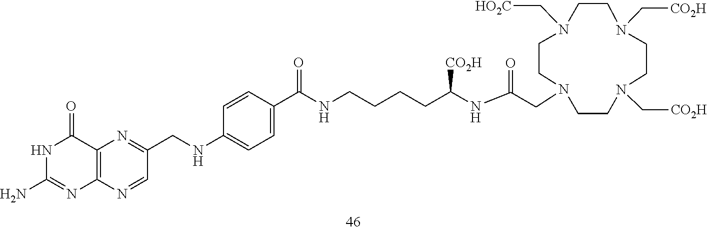 [SMILES: Nc1nc2ncc(CNc3ccc(C(=O)NCCCC[C@H](NC(=O)CN4CCN(CC(=O)O)CCN(CC(=O)O)CCN(CC(=O)O)CC4)C(=O)O)cc3)nc2c(=O)[nH]1]

LCMS (ESI): [M+H]⁺=Calculated for C₃₆H₅₁N₁₂O₁₁, 827.37; found 827.30.

Selected data ¹H NMR (500 MHz, DMSO-d6, D₂O): δ 8.60 (s, 1H), 7.52 (d, J=9.0 Hz, 2H), 6.62 (d, J=9.0 Hz, 2H), 4.47 (s, 2H), 3.99 (dd, J₁=9.0 Hz, J₂=4.0, 5.0 Hz, 1H).

### Example 34: DOTA-Gly-Pro-Pro-Gly-Ser-Ala-Phe-Lys(Pte)-OH (Compound 47)

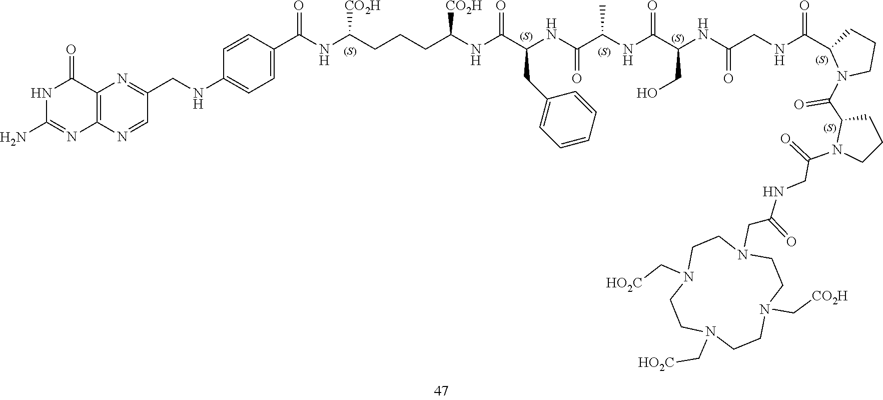

LCMS (ESI): [M+H]⁺=Calculated for C₆₅H₉₀N₁₉O₁₉, 1440.66; found 1440.73.

Selected data ¹H NMR (500 MHz, DMSO-d6, D₂O): δ 8.61 (s, 1H), 7.56 (d, J=8.5 Hz, 2H), 7.24-7.10 (m, 5H), 6.61 (d, J=8.0 Hz, 2H), 4.47 (s, 2H), 4.52-4.40 (m, 2H), 4.40-4.18 (m, 4H).

### Example 35: DOTA-Gly-Pro-Pro-Gly-Phe-Ser-Ala-Phe-Lys(Pte)-OH (Compound 48)

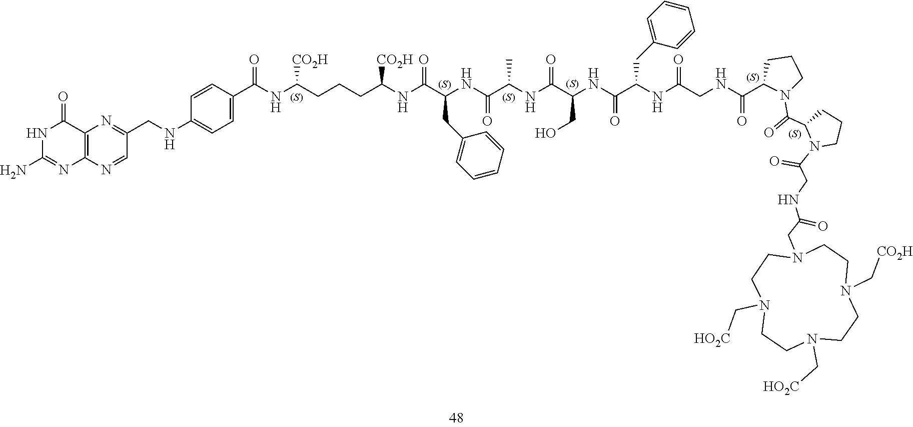

LCMS (ESI): [M+H]⁺=Calculated for C₇₄H₉₉N₂₀O₂₀, 1587.73; found 1587.64.

Selected data ¹H NMR (500 MHz, DMSO-d6, D₂O): δ 8.60 (s, 1H), 7.56 (d, J=9.0 Hz, 2H), 7.24-7.08 (m, 10H), 6.59 (d, J=9.0 Hz, 2H), 4.45 (s, 2H), 4.52-4.42 (m, 2H), 4.42-4.36 (m, 1H), 4.32-4.26 (m, 1H), 4.23-4.18 (m, 1H), 4.09-4.00 (m, 1H).

### N-α-Pteroyl-2,3-Diaminopropionic Acid Based DOTA Conjugates

### Example 36: Synthesis of Pte-Dap(DOTA-Gly-Tyr(O-carbonylmethyl)-OH)—OH (Compound 49)

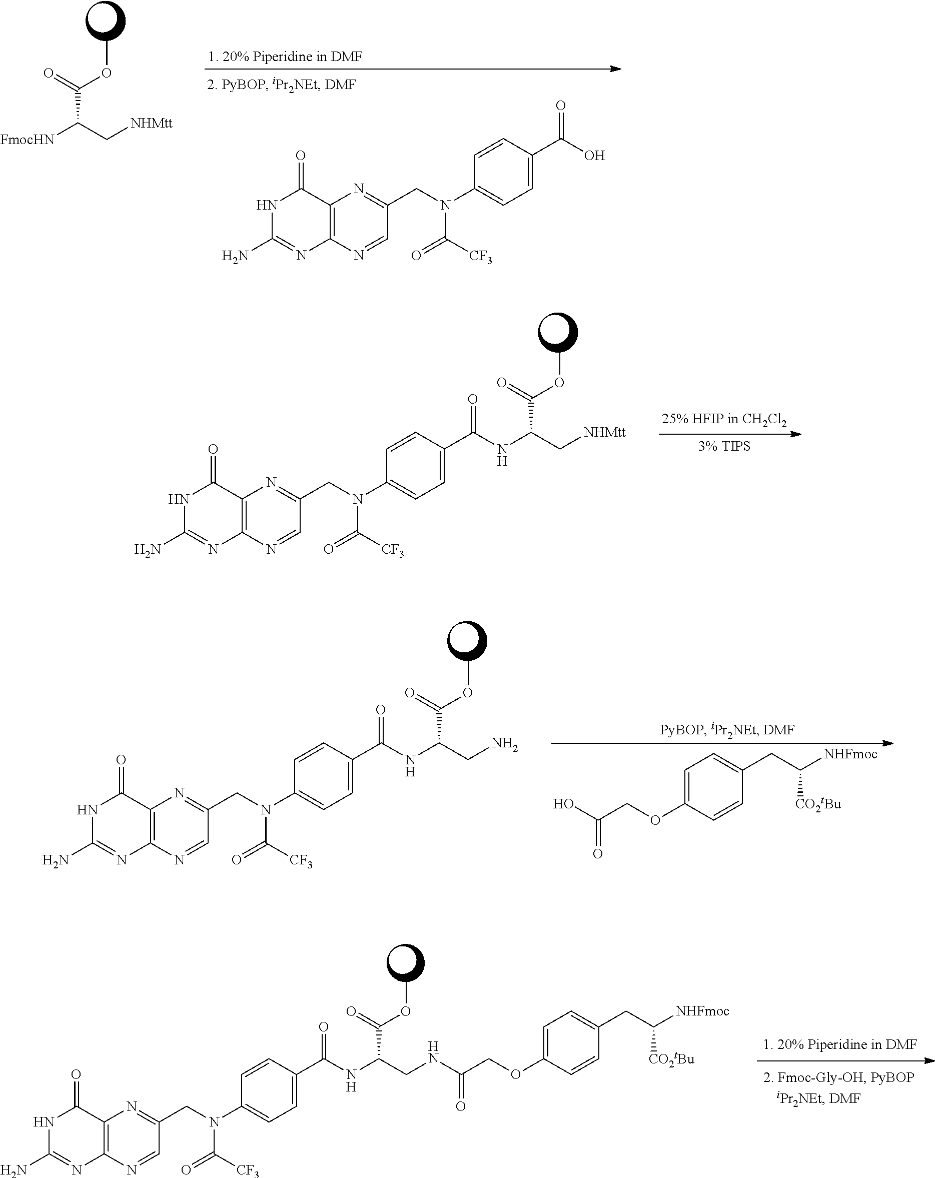

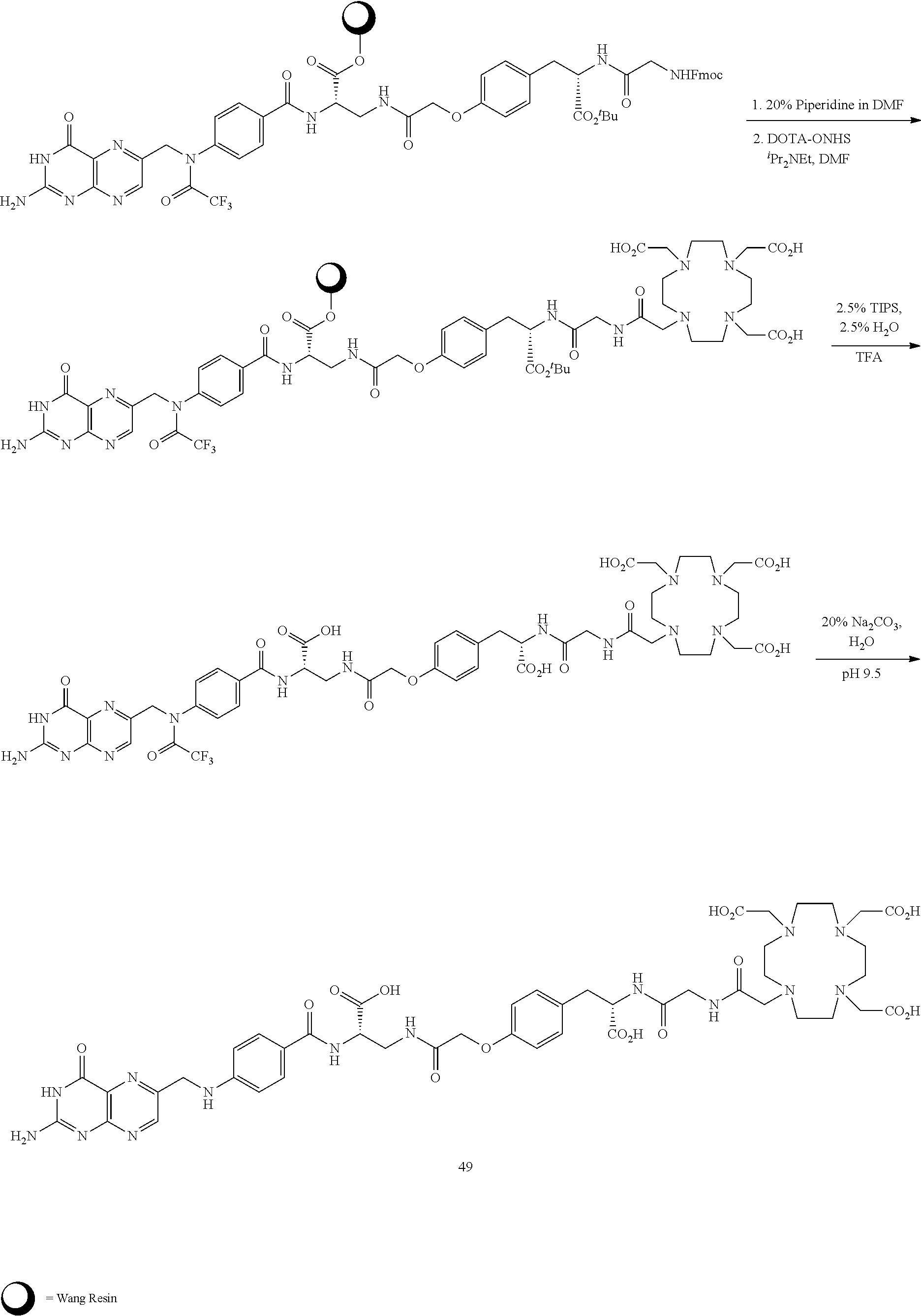

Compound 49 was synthesized by solid phase in six steps starting from Fmoc-Dap(N-4-methoxytrityl)-Wang-Resin (Table 10).

TABLE 10
Solid phase reaction steps:

| | | | MW | | |
|---|---|---|---|---|---|
| | mmol. | equiv. | (g/mol) | amount | |
| Fmoc-Dap(N-4-methoxytrityl)- | 0.188 | — | — | 1.0 | g |
| Wang Resin | | | | | |
| (loading 0.188 mmol/g.) | | | | | |
| (commercially obtained) | | | | | |

Procedure A: Fmoc Deprotection - 20% Piperidine in DMF (3X)
Procedure C: N¹⁰-TFA-Pteroic Acid Coupling

| | | | | | |
|---|---|---|---|---|---|
| N¹⁰-TFA-Pteroic Acid | 0.38 | 2 | 408 | 154 | mg |
| ⁱPr₂NEt | 0.75 | 4 | 129 | 0.131 | mL |
| PyBOP | 0.38 | 2 | 520 | 196 | mg |

Procedure B: Mtt Deprotection - 25% HFIP in CH₂Cl₂ (6X)
Procedure C: Fmoc-Tyr(OCH₂CO₂H)-O^{t}Bu Coupling

| | | | | | |
|---|---|---|---|---|---|
| Fmoc-Tyr(OCH₂CO₂H) | 0.38 | 2 | 517 | 194 | mg |
| ⁱPr₂NEt | 0.75 | 4 | 129 | 0.131 | mL |
| PyBOP | 0.38 | 2 | 520 | 196 | mg |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.38 | 2 | 297 | 112 | mg |
| (commercially obtained) | | | | | |
| ⁱPr₂NEt | 0.75 | 4 | 129 | 0.131 | mL |
| PyBOP | 0.38 | 2 | 520 | 196 | mg |

Procedure A: Fmoc Deprotection - 20% Piperidine in DMF (3X)
Procedure C: N-Hydroxysuccinamidyl 1,4,7,10-tetraazacyclododecane-
1,4,7,10-tetraacetate {DOTA-ONHS} Coupling

| | | | | | |
|---|---|---|---|---|---|
| DOTA-ONHS | 0.38 | 2 | 762 | 286 | mg |
| (commercially obtained) | | | | | |
| ⁱPr₂NEt | 0.75 | 4 | 129 | 0.131 | mL |

Procedure D: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

### Procedure E: Deprotection of N¹⁰-TFA Group in Pteroic Acid and Purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

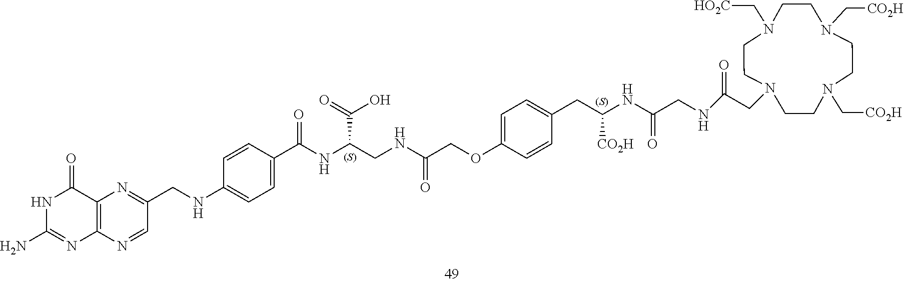

LCMS (ESI): [M+H]⁺=Calculated for C₄₆H₅₉N₁₄O₁₆, 1063.42; found 1063.30.

Selected data ¹H NMR (500 MHz, DMSO-d6, D₂O): δ 8.63 (s, 1H), 7.55 (d, J=9.0 Hz, 2H), 7.06 (d, J=9.0 Hz, 2H), 6.77 (d, J=9.0 Hz, 2H), 6.62 (d, J=8.5 Hz, 2H), 4.48 (s, 2H), 4.38 (s, 2H), 4.34 (dd, J₁=7.5 Hz, J₂=5.0 Hz, 1H), 4.24 (dd, J₁=7.0 Hz, J₂=6.5 Hz, 1H).

Utilizing above SPPS procedures, the following DOTA conjugate was prepared (see Example 37):

### Example 37: Pte-Dap(Val-Gly-DOTA)-OH (Compound 50)

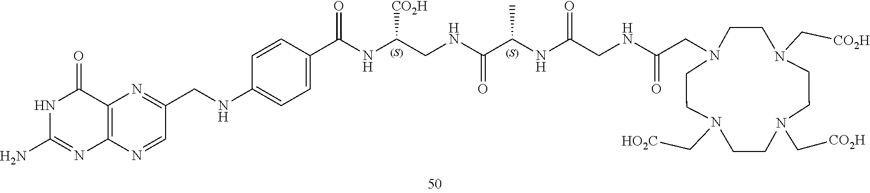

LCMS (ESI): [M+H]⁺=Calculated for C₃₈H₅₃N₁₄O₁₃, 913.38; found 913.30.

Selected data ¹H NMR (500 MHz, DMSO-d6, D₂O): δ 8.66 (s, 1H), 7.61 (d, J=9.0 Hz, 2H), 6.66 (d, J=8.0 Hz, 2H), 4.50 (s, 2H), 4.46 (m, 1H), 4.26 (ABq, J₁=13.75 Hz, J₂=6.5 Hz, 1H).

### Pteroyl-Aspartic Acid Based DOTA Conjugates

### Example 38: Synthesis of Pte-Asp(DOTA-Gly-ε-Lys-OH)—OH (Compound 51)

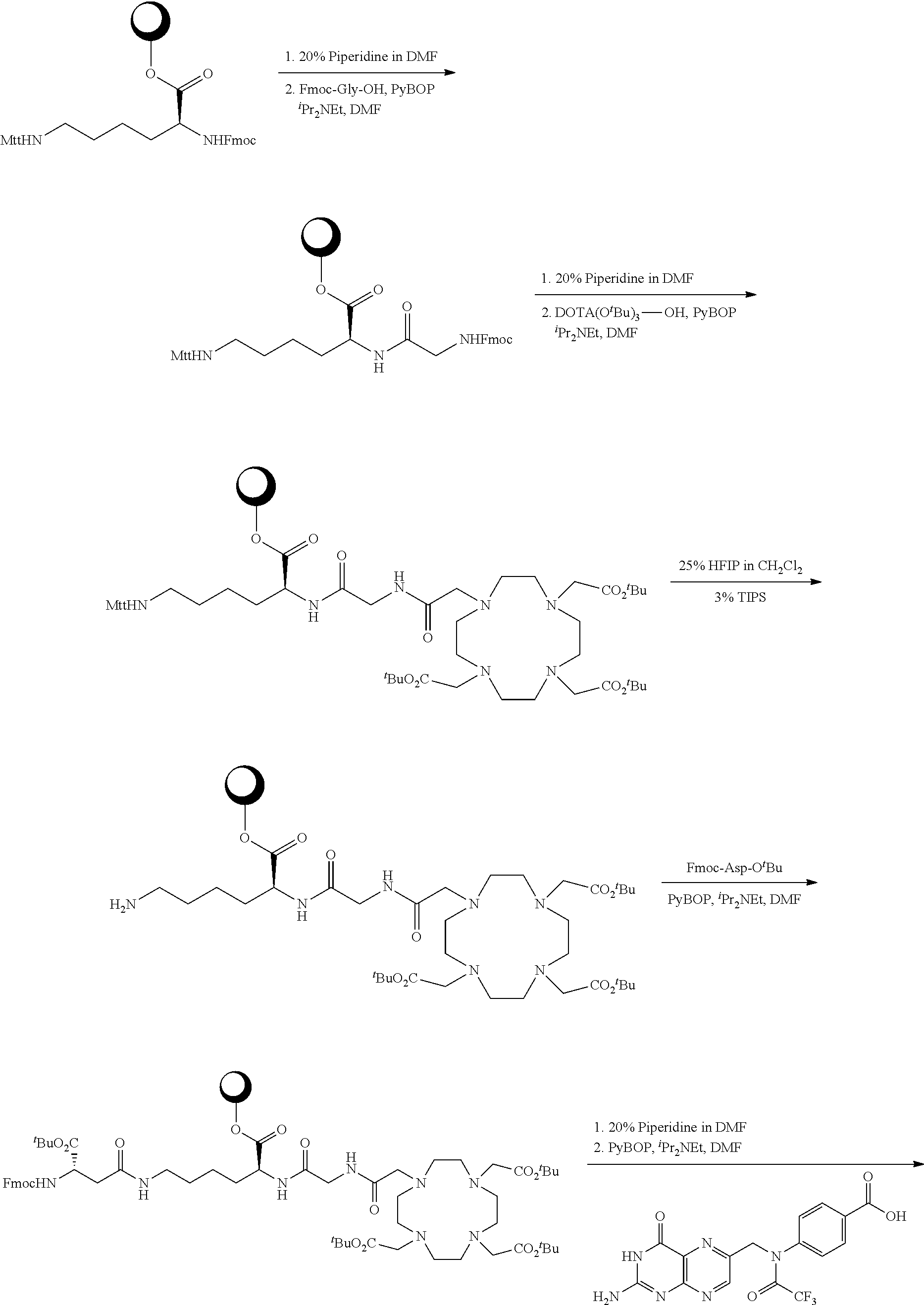

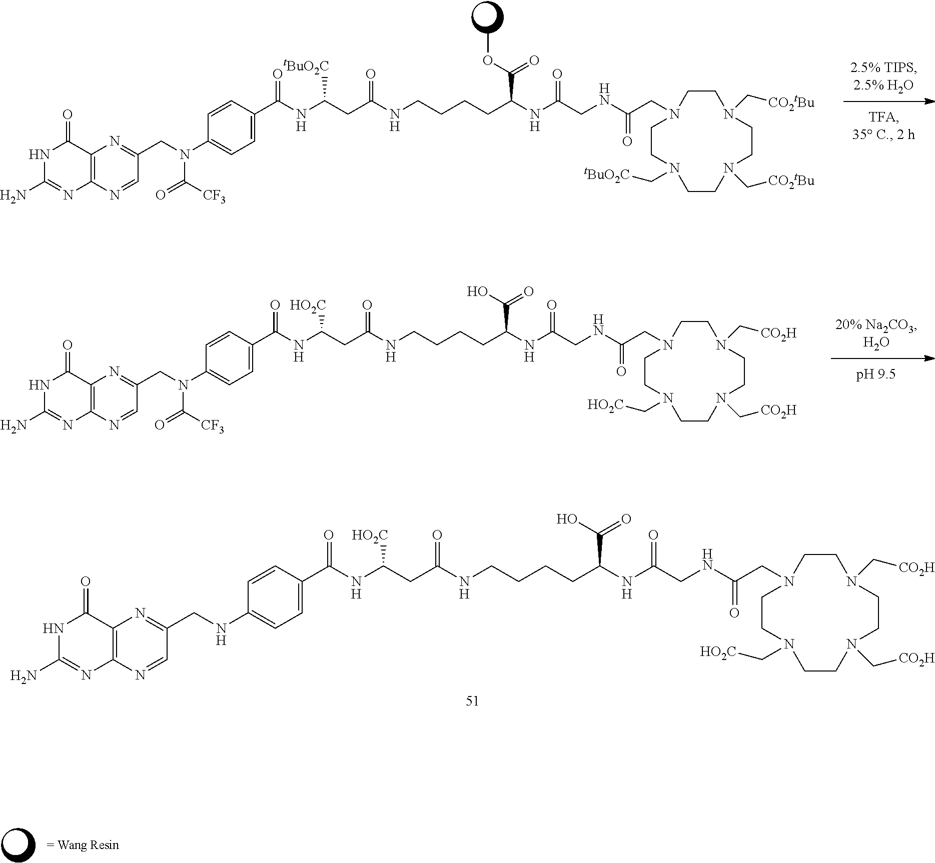 [SMILES: COC(=O)[C@H](CCCCNC(=O)C[C@H](NC(=O)c1ccc(N(Cc2cnc3nc(N)[nH]c(=O)c3n2)C(=O)C(F)(F)F)cc1)C(=O)OC(C)(C)C)NC(=O)CNC(=O)CN1CCN(CC(=O)OC(C)(C)C)CCN(CC(=O)OC(C)(C)C)CCN(CC(=O)OC(C)(C)C)CC1 ; Nc1nc2ncc(CN(C(=O)C(F)(F)F)c3ccc(C(=O)N[C@@H](CC(=O)NCCCC[C@H](NC(=O)CNC(=O)CN4CCN(CC(=O)O)CCN(CC(=O)O)CCN(CC(=O)O)CC4)C(=O)O)C(=O)O)cc3)nc2c(=O)[nH]1 ; Nc1nc2ncc(CNc3ccc(C(=O)N[C@@H](CC(=O)NCCCC[C@H](NC(=O)CNC(=O)CN4CCN(CC(=O)O)CCN(CC(=O)O)CCN(CC(=O)O)CC4)C(=O)O)C(=O)O)cc3)nc2c(=O)[nH]1]

Compound 51 was synthesized by solid phase in six steps starting from Fmoc-Lys(N-4-methoxytrityl)-Wang-Resin (Table 11).

TABLE 11
Solid phase reaction steps:

| | | | MW | | |
|---|---|---|---|---|---|
| | mmol. | equiv. | (g/mol) | amount | |
| Fmoc-Lys(N-4-methoxytrityl)- | 0.27 | — | — | 0.5 | g |
| Wang Resin | | | | | |
| (loading 0.538 mmol/g.) | | | | | |
| (commercially obtained) | | | | | |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.54 | 2 | 297 | 160 | mg |
| (commercially obtained) | | | | | |
| ⁱPr₂NEt | 1.08 | 4 | 129 | 0.187 | mL |
| PyBOP | 0.54 | 2 | 520 | 280 | mg |

Procedure A: Fmoc Deprotection - 20% Piperidine in DMF (3X)
Procedure C: Tri-tert-butyl 1,4,7,10-tetraazacyclododecane-
1,4,7,10-tetraacetate DOTA(O^{t}Bu)₃-OH} Coupling

| | | | | | |
|---|---|---|---|---|---|
| DOTA(O^{t}Bu)₃-OH | 0.54 | 2 | 573 | 308 | mg |
| (commercially obtained) | | | | | |
| ⁱPr₂NEt | 1.08 | 4 | 129 | 0.187 | mL |
| PyBOP | 0.54 | 2 | 520 | 280 | mg |

Procedure B: Mtt Deprotection - 25% HFIP in CH₂Cl₂ (6X)
Procedure C: Fmoc-L-Asp-O^{t}Bu Coupling

| | | | | | |
|---|---|---|---|---|---|
| Fmoc-Asp-O^{t}Bu | 0.54 | 2 | 412 | 221 | mg |
| (commercially obtained) | | | | | |
| ⁱPr₂NEt | 1.08 | 4 | 129 | 0.187 | mL |
| PyBOP | 0.54 | 2 | 520 | 280 | mg |

Procedure A: Fmoc Deprotection - 20% Piperidine in DMF (3X)
Procedure C: N¹⁰-TFA-Pteroic Acid Coupling

| | | | | | |
|---|---|---|---|---|---|
| N¹⁰-TFA-Pteroic Acid | 0.40 | 1.5 | 408 | 165 | mg |
| ⁱPr₂NEt | 1.08 | 4 | 129 | 0.187 | mL |
| PyBOP | 0.40 | 1.5 | 520 | 210 | mg |

Procedure D₂: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D₂: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The filtrate was stirred at 35° C. under argon for 2 h. The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

### Procedure E: Deprotection of N¹⁰-TFA Group in Pteroic Acid and Purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

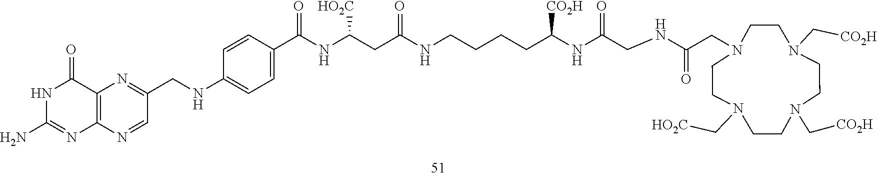 [SMILES: Nc1nc2ncc(CNc3ccc(C(=O)N[C@@H](CC(=O)NCCCC[C@H](NC(=O)CNC(=O)CN4CCN(CC(=O)O)CCN(CC(=O)O)CCN(CC(=O)O)CC4)C(=O)O)C(=O)O)cc3)nc2c(=O)[nH]1]

LCMS (ESI): [M+H]⁺=Calculated for C₄₂H₅₉N₁₄O₁₅, 999.42; found 999.46.

Selected data ¹H NMR (500 MHz, DMSO-d6, D₂O): δ 8.61 (s, 1H), 7.55 (d, J=8.0 Hz, 2H), 6.62 (d, J=8.5 Hz, 2H), 4.64 (dd, J₁=7.5 Hz, J₂=5.5 Hz, 1H), 4.47 (s, 2H), 4.12 (dd, J₁=8.5 Hz, J₂=5.5 Hz, 1H).

Utilizing the above SPPS procedures, the following DOTA conjugate was prepared (see Example 39):

### Example 39: Pte-Asp(DOTA-Ala-ε-Lys-OH)—OH (Compound 52)

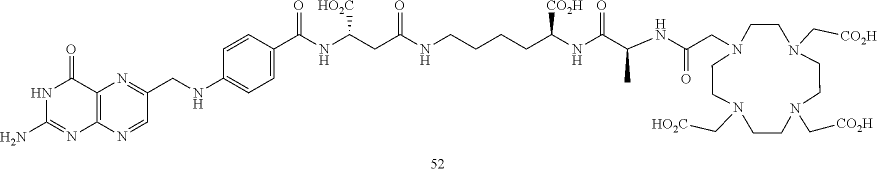 [SMILES: C[C@H](NC(=O)CN1CCN(CC(=O)O)CCN(CC(=O)O)CCN(CC(=O)O)CC1)C(=O)N[C@@H](CCCCNC(=O)C[C@H](NC(=O)c1ccc(NCc2cnc3nc(N)[nH]c(=O)c3n2)cc1)C(=O)O)C(=O)O]

LCMS (ESI): [M+H]⁺=Calculated for C₄₃H₆₁N₁₄O₁₅, 1013.44; found 1013.40.

Selected data ¹H NMR (500 MHz, DMSO-d6, D₂O): δ 8.62 (s, 1H), 7.55 (d, J=8.5 Hz, 2H), 6.63 (d, J=9.5 Hz, 2H), 4.52-4.42 (m, 1H), 4.48 (s, 2H), 4.12 (ABq, J₁=14.5 Hz, J₂=7.0, 7.5 Hz, 1H).

### Pteroyl-Aspartic Acid Based DOTA Conjugate with Hippuryl Lysine-Motif

### Example 40: Synthesis of Pte-Asp(DOTA-3-amino-Benzoyl-Gly-ε-Lys-OH)—OH (Compound 53)

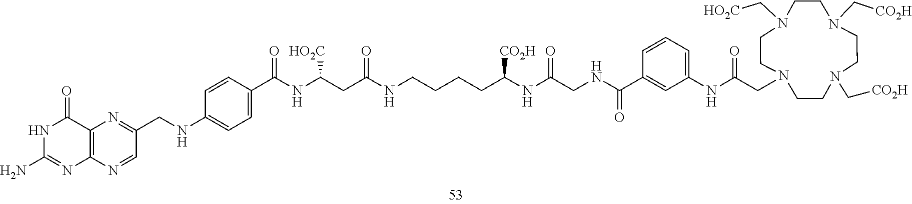 [SMILES: Nc1nc2ncc(CNc3ccc(C(=O)N[C@@H](CC(=O)NCCCC[C@H](NC(=O)CNC(=O)c4cccc(NC(=O)CN5CCN(CC(=O)O)CCN(CC(=O)O)CCN(CC(=O)O)CC5)c4)C(=O)O)C(=O)O)cc3)nc2c(=O)[nH]1]

LCMS (ESI): [M+H]⁺=Calculated for C₄₉H₆₄N₁₅O₁₆, 1118.46; found 1118.50.

Selected data ¹H NMR (500 MHz, DMSO-d6, D₂O): δ 8.60 (s, 1H), 8.26 (s, 1H), 7.90 (d, J=7.5 Hz, 1H), 7.55 (d, J=9.0 Hz, 2H), 7.44 (d, J=7.0 Hz, 1H), 7.26 (dd, J₁=7.5 Hz, J₂=8.0 Hz, 1H), 6.63 (d, J=9.0 Hz, 2H), 4.49 (dd, J₁=8.0 Hz, J₂=5.5, 6.0 Hz, 1H), 4.46 (s, 2H), 4.02 (dd, J₁=5.0 Hz, J₂=5.5 Hz, 1H).

### Pteroyl-Aspartic ACID based DOTA Conjugate with MVK-Motif

### Example 41: Synthesis of Pte-Asp(DOTA-Met-Val-ε-Lys-OH)—OH (Compound 54)

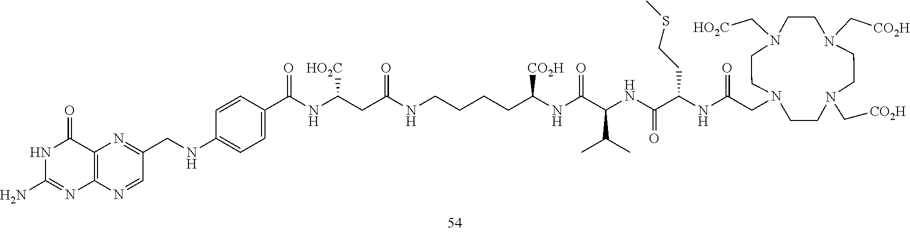 [SMILES: CSCC[C@H](NC(=O)CN1CCN(CC(=O)O)CCN(CC(=O)O)CCN(CC(=O)O)CC1)C(=O)N[C@H](C(=O)N[C@@H](CCCCNC(=O)C[C@H](NC(=O)c1ccc(NCc2cnc3nc(N)[nH]c(=O)c3n2)cc1)C(=O)O)C(=O)O)C(C)C]

LCMS (ESI): [M+H]⁺=Calculated for C₅₀H₇₄N₁₅O₁₆S, 1172.51; found 1172.30.

Selected data ¹H NMR (500 MHz, DMSO-d6, D₂O): δ 8.61 (s, 1H), 7.54 (d, J=8.5 Hz, 2H), 6.62 (d, J=8.5 Hz, 2H), 4.51-4.41 (m, 1H), 4.46 (s, 2H), 4.28 (m, 1H), 4.05 (d, J=7.0 Hz, 1H), 4.0 (m, 1H).

### Folate Based DOTA Conjugates with MVK-Motif

### Example 42: Synthesis of Boc-Met-Val-Lys-OH (Compound 55)

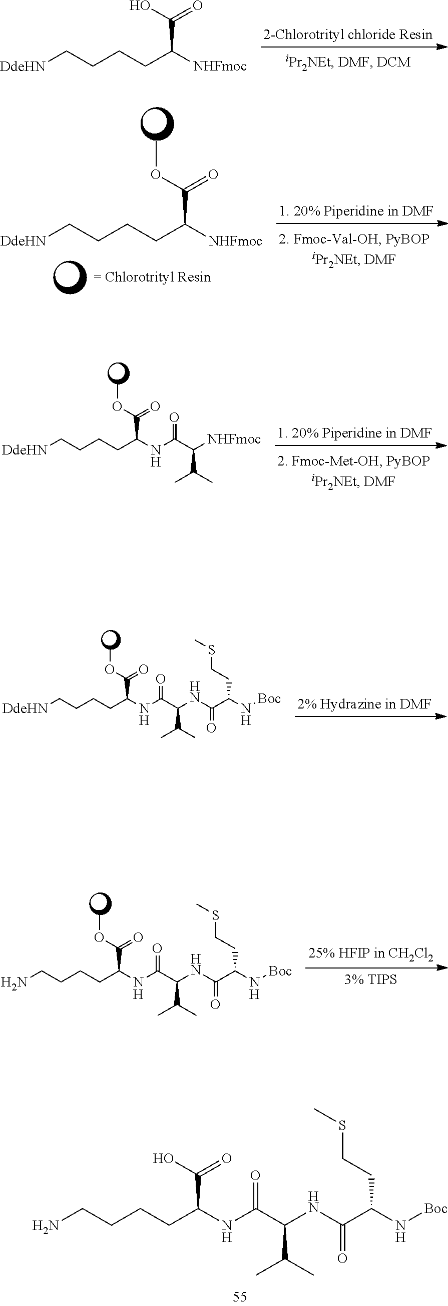

Compound 55 was synthesized by solid phase in four steps starting from Fmoc-Lys(N-(1-(4,4-dimethyl-2,6-dioxocyclohexylidine)ethyl))-cholrotrityl-Resin (Table 12).

TABLE 12
Solid phase reaction steps:

| | | | MW | | |
|---|---|---|---|---|---|
| | mmol. | equiv. | (g/mol) | amount | |
| Fmoc-Lys(N-(1- | 0.40 | — | — | 1.0 | g |
| (4,4-dimethyl-2,6- | | | | | |
| dioxocyclohexylidine)ethyl))- | | | | | |
| Chlorotrityl Resin | | | | | |
| (loading 0.40 mmol/g.) | | | | | |

Procedure A: Fmoc Deprotection - 20% Piperidine in DMF (3X)
Procedure C: Fmoc-Val-OH Coupling

| | | | | | |
|---|---|---|---|---|---|
| Fmoc-Val-OH | 0.80 | 2 | 340 | 272 | mg |
| (commercially obtained) | | | | | |
| ⁱPr₂NEt | 1.60 | 4 | 129 | 0.279 | mL |
| PyBOP | 0.80 | 2 | 520 | 416 | mg |

Procedure A: Fmoc Deprotection - 20% Piperidine in DMF (3X)
Procedure C: Boc-Met-OH Coupling

| | | | | | |
|---|---|---|---|---|---|
| Boc-Met-OH | 0.80 | 2 | 249 | 199 | mg |
| (commercially obtained) | | | | | |
| ⁱPr₂NEt | 1.60 | 4 | 129 | 0.279 | mL |
| PyBOP | 0.80 | 2 | 520 | 416 | mg |

Procedure B₂: Dde Deprotection - 2% hydrazine in DMF (3X)
Procedure D₃: Resin Cleavage - 25% HFIP, 75% DCM, 2.5% TIPS

### Procedure A: Fmoc Deprotection

The deprotection step was performed before each amino acid coupling steps. A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B₂: Dde Deprotection

A solution of 2% hydrazine in DMF (˜20 mL) for Dde deprotection was added. Argon was bubbled through the solution for 20 min and then drained. 2% hydrazine in DMF (˜20 mL) was added and bubbling continued for 20 min before draining (2×). The resin was washed with DMF (˜20 mL×3) followed by IPA (˜20 mL×3) and dried.

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D₃: Resin Cleavage

Resin was cleaved using 25% HFIP in CH₂Cl₂ (˜20 mL) and 2.5% TIPS. Argon was bubbled through the solution for 1 h and drained into clean flask. Washed the resin with cleavage solution for 10 min (2×) and drained. Combined cleaved solution was concentrated to smaller volume and precipitated with ether. Solid was washed with ether (3×) and dried under high vacuum.

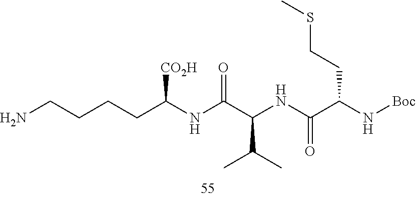 [SMILES: CSCC[C@H](NC(=O)OC(C)(C)C)C(=O)N[C@H](C(=O)N[C@@H](CCCCN)C(=O)O)C(C)C]

LCMS (ESI): [M+H]⁺=Calculated for C₂₁H₄₁N₄O₆S, 477.27; found 477.09.

Selected data ¹H NMR (500 MHz, CD₃OD): δ 4.24 (dd, J₁=7.0 Hz, J₂=5.0 Hz, 1H), 4.20 (dd, J₁=9.0 Hz, J₂=5.5 Hz, 1H), 4.18 (d, J=7.5 Hz, 1H), 2.92 (t, J₁=7.5 Hz, J₂=7.0 Hz, 2H), 2.46-2.62 (m, 2H), 2.10 (s, 3H), 1.98-2.07 (m, 2H), 1.81-1.92 (m, 2H), 1.60-1.76 (m, 3H), 1.46 (s, 9H), 1.36-1.46 (m, 2H), 0.98 (d, J=7.0 Hz, 3H), 0.97 (d, J=6.0 Hz, 3H).

### Example 43: Synthesis of Boc-Met-Val-Lys(Maleimido)-OH (Compound 56)

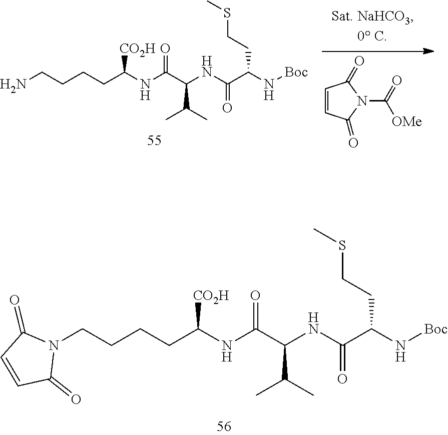 [SMILES: CSCC[C@H](NC(=O)OC(C)(C)C)C(=O)N[C@H](C(=O)N[C@@H](CCCCN)C(=O)O)C(C)C ; COC(=O)N1C(=O)C=CC1=O ; CSCC[C@H](NC(=O)OC(C)(C)C)C(=O)N[C@H](C(=O)N[C@@H](CCCCN1C(=O)C=CC1=O)C(=O)O)C(C)C]

To a solution of Compound 55 (0.0086 g, 0.018 mM) in water (0.3 mL) was added sat. NaHCO₃ (0.13 mL). Reaction was cooled to 0° C., and added N-methoxycarbonyl-Maleimide (commercially obtained, 0.004 g, 0.026 mM). The reaction was allowed to stir for 2 h, LCMS analysis (20 mM NH₄HCO₃, pH 7.4) indicated that the reaction was complete. The reaction mixture was treated with 5% citric acid at 0° C. until pH reaches to 3.0, extracted with dichloromethane (3×), dried over Na₂SO₄, concentrated and dried. Crude Compound 56 is confirmed by LCMS and used for next reaction without further purification.

LCMS (ESI): [M+Na]⁺=Calculated for C₂₅H₄₀N₄O₈SNa, 579.26; found 579.29.

### Example 44: Synthesis of Met-Val-Lys(Maleimido)-OH (Compound 57)

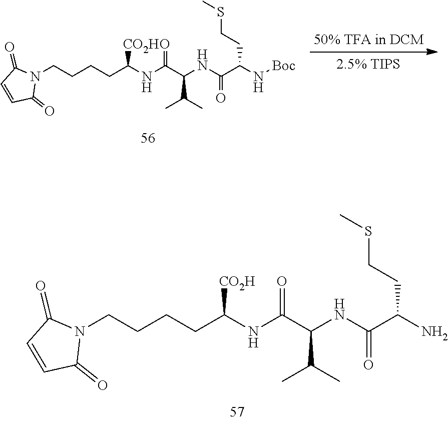 [SMILES: CSCC[C@H](NC(=O)OC(C)(C)C)C(=O)N[C@H](C(=O)N[C@@H](CCCCN1C(=O)C=CC1=O)C(=O)O)C(C)C ; CSCC[C@H](N)C(=O)N[C@H](C(=O)N[C@@H](CCCCN1C(=O)C=CC1=O)C(=O)O)C(C)C]

To a solution of Compound 56 (0.007 g, 0.013 mM) in dichloromethane (0.5 mL) was added trifluoroacetic acid (0.5 mL) and triisopropyl silane (0.025 mL). The reaction was allowed to stir at RT for 30 min, LCMS analysis (20 mM NH₄HCO₃, pH 7.4) indicated that the reaction was complete. The reaction mixture was concentrated, co-evaporated with dichloromethane (3×), and dried under high vacuum. Crude Compound 57 is confirmed by LCMS and used for next reaction without further purification.

LCMS (ESI): [M+H]⁺=Calculated for C₂₀H₃₃N₄O₆S, 457.20; found 457.17 0.

### Example 45: Synthesis of DOTA-Bn-NHC(S)NH-Met-Val-Lys(Mal-S-Cys-Asp-Asp-Arg-Asp-Folate)-OH (Compound 58)

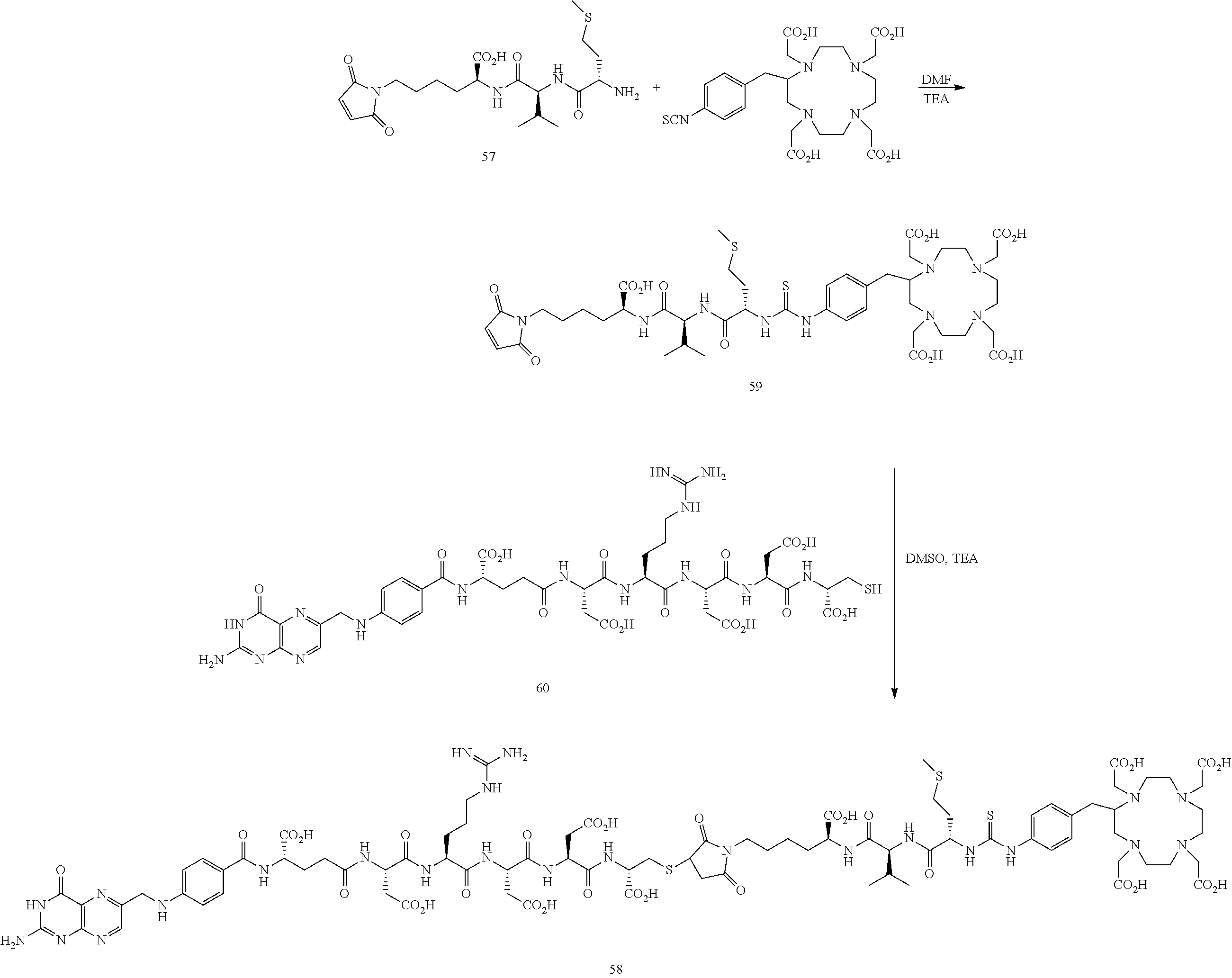

To a solution of Compound 57 (0.007 g, 0.013 mM) in DMF (0.5 mL) was added DOTA-benzyl iso-thiocyanate (commercially obtained, 0.035 g, 0.063 mM) and triethylamine (0.017 mL, 0.13 mM). The resulting homogeneous solution was stirred at ambient temperature under argon for 2 h. LCMS analysis confirmed the product (Compound 59) formation. Compound 60, synthesized according to Vlahov et al, Bioorg. & Med. Chem. Letters 16(2006), 5093-5096, (0.014 g, 0.014 mM) in DMSO (0.5 mL) and triethylamine (0.017 mL, 0.13 mM) was added, stirred at ambient temperature under argon for 1 h. LCMS analysis confirmed the product formation. Reaction mixture was diluted with DMSO, and loaded onto a preparatory HPLC (Mobile phase A=50 mM Ammonium bicarbonate, pH=7.0. B=ACN. Method: 5-50% B in 25 min.) for purification. Fractions containing the desired product were collected, combined, ACN was removed and freeze-dried to afford the conjugate Compound 58 as a yellow solid.

LCMS (ESI): [M+2H]²⁺=Calculated for C₈₄H₁₁₇N₂₄O₃₁S₃, 1027.37; found 1027.50.

Selected data ¹H NMR (500 MHz, DMSO-d6, D₂O): δ 8.63 (s, 1H), 7.60 (d, J=8.5 Hz, 2H), 7.62-7.35 (m, 2H), 7.35-7.00 (m, 2H), 6.63 (d, J=8.5 Hz, 2H), 5.00-4.84 (m, 1H), 4.67-4.54 (m, 1H), 4.54-4.44 (m, 3H), 4.44-4.36 (m, 1H), 4.26 (br s, 1H), 4.19 (br s, 1H), 4.17-3.99 (m, 4H).

### Example 46: Synthesis of Pte-Tyr(OCH₂CO-EDA-DOTA)-OH (Compound 61)

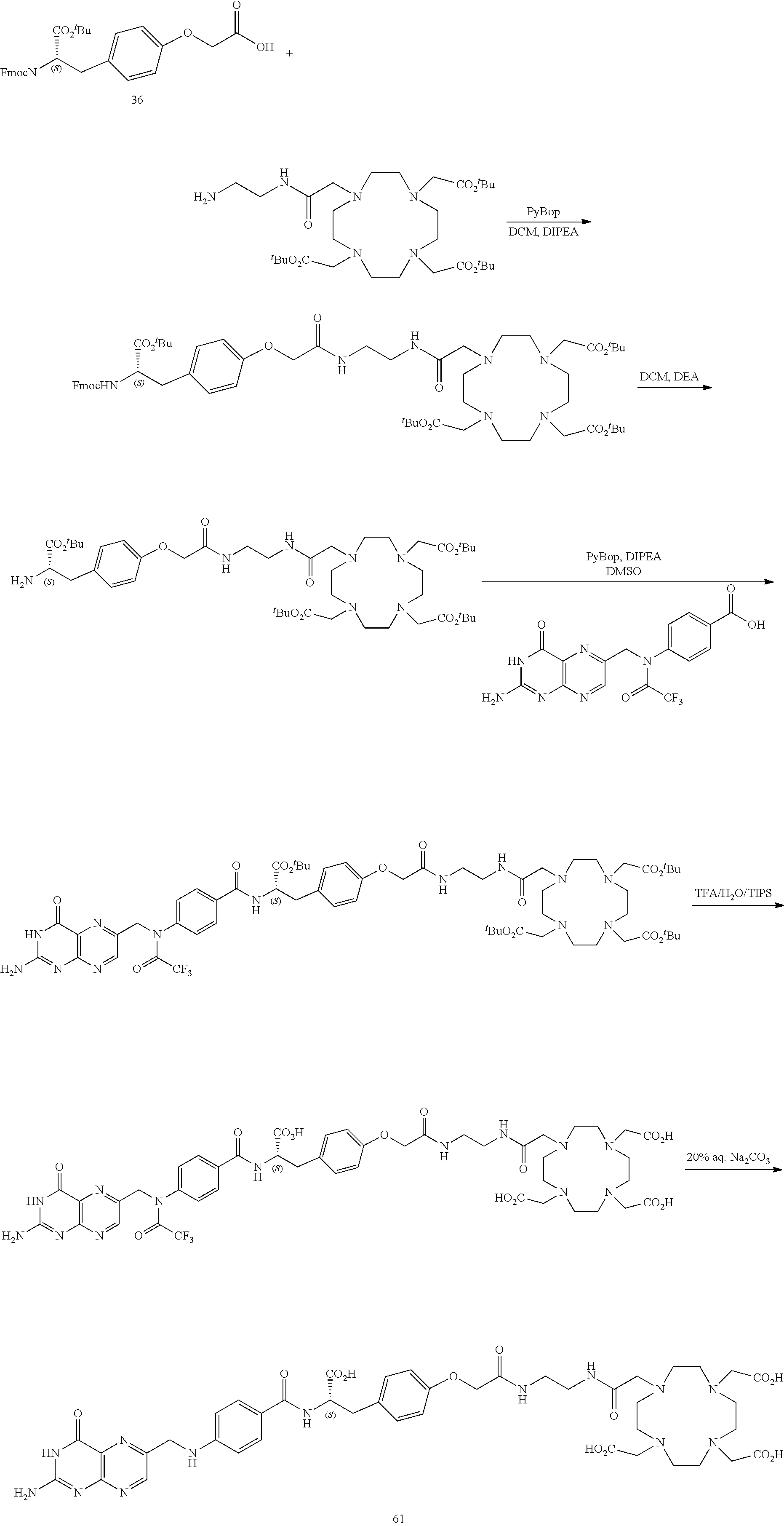

To a solution of Compound 36 (0.043 g, 0.083 mM) in DCM (4.0 mL) was added EDA-DOTA(O^{t}Bu)₃ (commercially obtained, 0.058 g, 0.083 mM), PyBop (0.048 g, 0.091 mM), and diisopropylethylamine (0.145 mL, 0.83 mM) respectively. The resulting homogeneous solution was stirred at ambient temperature under argon for 2 h. LCMS analysis confirmed the coupled product formation. Diethylamine (1.4 mL) was added, stirred at ambient temperature under argon for 3 h. LCMS analysis confirmed the de-Fmoc product formation. DCM and diethylamine were evaporated and the residue was co-evaporated with DCM (3×) and dried. Residue was dissolved in DMSO (1.0 mL), N¹⁰-TFA-pteroic acid (0.034 g, 0.083 mM), PyBop (0.048 g, 0.091 mM), and diisopropylethylamine (0.145 mL, 0.83 mM) were added. The resulting homogeneous solution was stirred at ambient temperature under argon for 2 h. LCMS analysis confirmed the coupling reaction is complete. Reaction mixture was precipitated with ether. Solid was washed with ether (3×) and dried under high vacuum to yield crude protected 61. Crude material was treated with 95% TFA, 2.5% H₂0, 2.5% Triisopropylsaline (25 mL) and stirred at 35° C. under argon for 2 h. LCMS analysis confirmed the product, N¹⁰-TFA-61, formation. The reaction mixture was concentrated under reduced pressure until 5 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum. The crude precipitate was suspended in water. 20% Na₂CO₃ was added until pH of the solution reached to 9.5. The clear solution was stirred for 1 h, LCMS analysis confirmed the N¹⁰-TFA deprotection. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization to yield Compound 61.

LCMS (ESI): [M+H]⁺=Calculated for C₄₃H₅₆N₁₃O₁₃, 962.40; found 962.50.

Selected data ¹H NMR (500 MHz, DMSO-d6, D₂O): δ 8.61 (s, 1H), 7.48 (d, J=9.0 Hz, 2H), 7.08 (d, J=8.5 Hz, 2H), 6.77 (d, J=9.0 Hz, 2H), 6.58 (d, J=9.0 Hz, 2H), 4.45 (s, 2H), 4.41 (s, 2H), 4.36 (dd, J₁=9.0 Hz, J₂=5.0 Hz, 1H).

### Example 47: Synthesis of Cbz-Tyr(O-CH₂CH₂NHBoc)-O^{t}Bu (Compound 62)

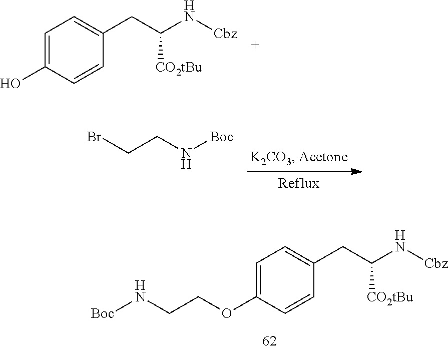 [SMILES: CC(C)(C)OC(=O)[C@H](Cc1ccc(O)cc1)NC(=O)OCc1ccccc1 ; CC(C)(C)OC(=O)NCCBr ; CC(C)(C)OC(=O)NCCOc1ccc(C[C@H](NC(=O)OCc2ccccc2)C(=O)OC(C)(C)C)cc1]

To a solution of Cbz-Tyr-O^{t}Bu (commercially obtained, 1.11 g, 3.0 mM) in dry acetone (10 mL) was added potassium carbonate (1.24 g, 9.0 mM) and stirred for 5 min. Boc-aminoethyl bromide (commercially obtained, 0.74 g, 3.3 mM) was added. The reaction was allowed to reflux for 24 h, LCMS analysis (20 mM NH₄HCO₃, pH 7.4) indicated the product formation. The reaction mixture was cooled to ambient temperature, filtered and concentrated. Residue was dissolved in dichloromethane, and purified by combi-flash chromatography (0-100% ethyl acetate in petroleum ether) to yield Compound 62.

LCMS (ESI): [M+Na]⁺=Calculated for C₂₈H₃₈N₂O₇Na, 537.27; found 537.40.

¹H NMR (500 MHz, CDCl₃): δδ 7.29-7.39 (m, 5H), 7.06 (d, J=9.0 Hz, 2H), 6.79 (d, J=8.5 Hz, 2H), 5.22 (d, J=8.5 Hz, 1H), 5.10 (ABq, J₁=19.5 Hz, J₂=12.5 Hz, 2H), 5.0 (br s, 1H), 4.50 (dd, J₁=13.75 Hz, J₂=6.0 Hz, 5.5 Hz, 1H), 3.99 (t, J₁=5.5 Hz, J₂=5.0 Hz, 2H), 3.53 (d, J=5.0 Hz, 2H), 2.96-3.09 (m, 2H), 1.46 (s, 9H), 1.42 (s, 9H).

### Example 48: Synthesis of Tyr(O-CH₂CH₂NHBoc)-O^{t}Bu (Compound 63)

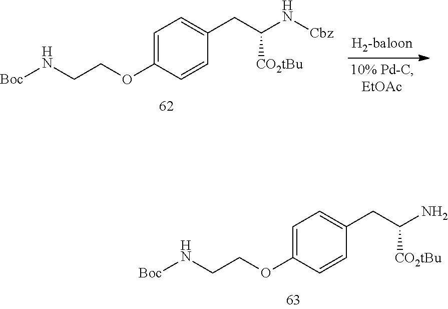 [SMILES: CC(C)(C)OC(=O)NCCOc1ccc(C[C@H](NC(=O)OCc2ccccc2)C(=O)OC(C)(C)C)cc1 ; CC(C)(C)OC(=O)NCCOc1ccc(C[C@H](N)C(=O)OC(C)(C)C)cc1]

To a solution of Compound 62 (0.38 g, 0.74 mM) in ethyl acetate (12 mL) was added 10% Pd/C (0.13 g) and stirred for 3 h under H₂ atmosphere (balloon). LCMS analysis (20 mM NH₄HCO₃, pH 7.4) indicated that the reaction was complete. The reaction mixture was filtered, concentrated and dried to yield Compound 63. Crude material was directly used for next coupling reaction.

LCMS (ESI): [M+H]⁺=Calculated for C₂₀H₃₃N₂O₅, 381.23; found 381.49.

### Example 49: Synthesis of Pte-Tyr(OCH₂CH₂—NH-DOTA)-OH (Compound 64)

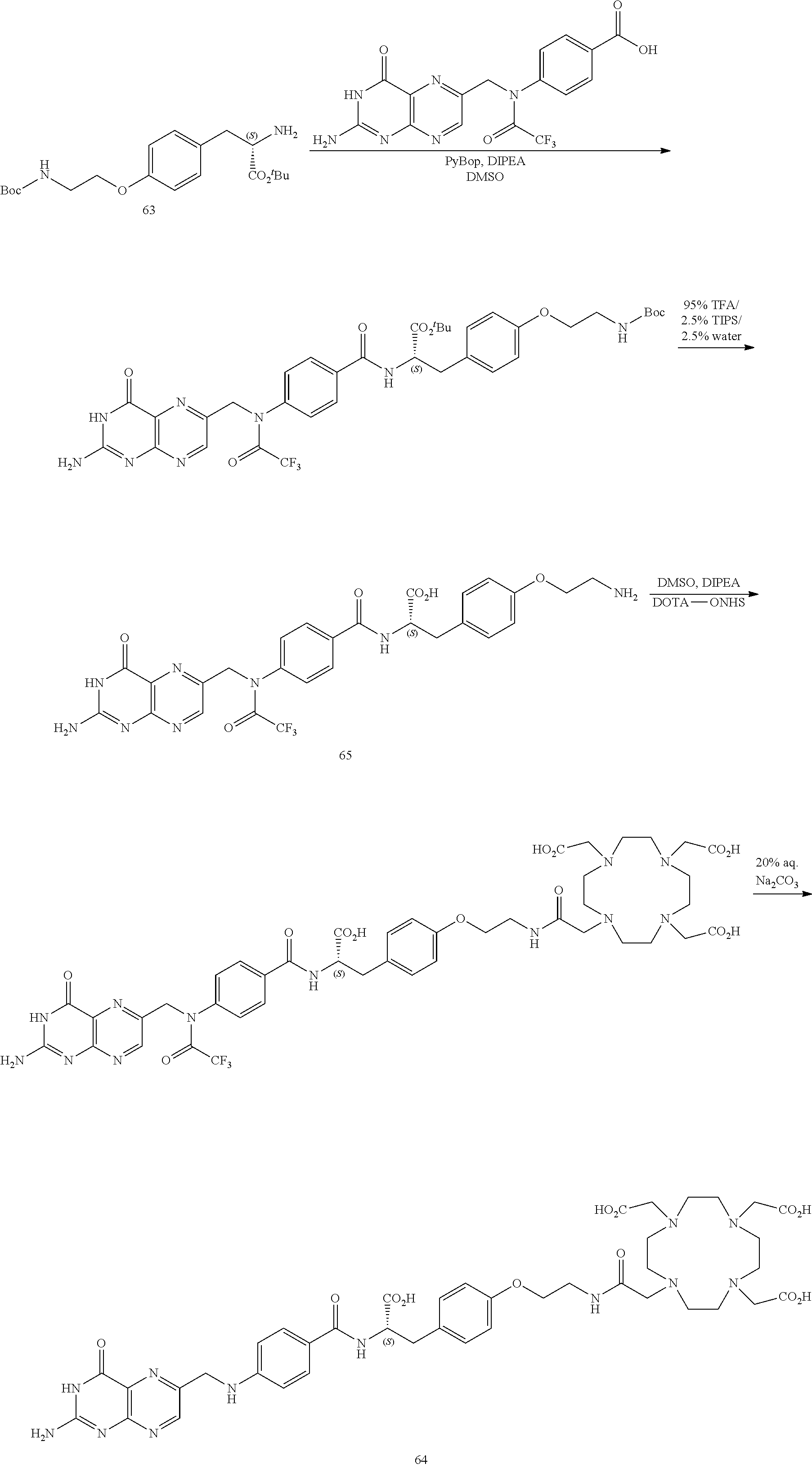

To a solution of Compound 63 (0.075 g, 0.197 mM) in DCM (1.0 mL) was added N¹⁰-TFA-pteroic acid (0.089 g, 0.217 mM) in DMSO (1.6 mL). PyBop (0.113 g, 0.217 mM), and diisopropylethylamine (0.189 mL, 1.09 mM) were added. The resulting homogeneous solution was stirred at ambient temperature under argon for 2 h. LCMS analysis confirmed the coupling reaction is complete. DCM was removed under reduced pressure, diluted with water and freeze dried for 16 h. Crude material was treated with 95% TFA, 2.5% H₂0, 2.5% Triisopropylsaline (25 mL) and stirred at RT under argon for 1 h. LCMS analysis confirmed the product formation. The reaction mixture was concentrated under reduced pressure until 5 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum for 18 h to yield Compound 65. Crude product was directly used for next coupling reaction.

LCMS (ESI): [M+H]⁺=Calculated for C₂₇H₂₆F₃N₈O₆, 615.18; found 614.88.

To a solution of Compound 65 (0.055 g, 0.090 mM) in DMSO (1.5 mL) was added DOTA-ONHS (commercially obtained, 0.068 g, 0.090 mM). Diisopropylethylamine (0.156 mL, 0.895 mM) was added. The resulting homogeneous solution was stirred at ambient temperature under argon for 1 h. LCMS analysis confirmed the coupling reaction is complete. Triturated in 10 mL of diethyl ether to separate oil out. Centrifuged and washed with ether (3×10 mL). The gummy product was dried over a stream of argon and then high vacuum for 18 h. The crude material was suspended in water. 20% Na₂CO₃ was added until pH of the solution reached to 9.5. The clear solution was stirred for 1 h, LCMS analysis confirmed the N¹⁰-TFA deprotection. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization to yield Compound 64.

LCMS (ESI): [M+H]⁺=Calculated for C₄₁H₅₃N₁₂O₁₂, 905.38; found 905.50.

Selected data ¹H NMR (500 MHz, DMSO-d6, D₂O): δ 8.60 (s, 1H), 7.43 (d, J=9.0 Hz, 2H), 7.04 (d, J=8.5 Hz, 2H), 6.71 (d, J=8.5 Hz, 2H), 6.57 (d, J=8.5 Hz, 2H), 4.45 (s, 2H), 4.34 (dd, J₁=8.5 Hz, J₂=5.0 Hz, 1H).

### Benzoyl-Aspartic Acid Based DOTA Conjugates

### Example 50: Synthesis of Benzoyl-Asp(DOTA-Gly-ε-Lys-OH)—OH (Compound 66)

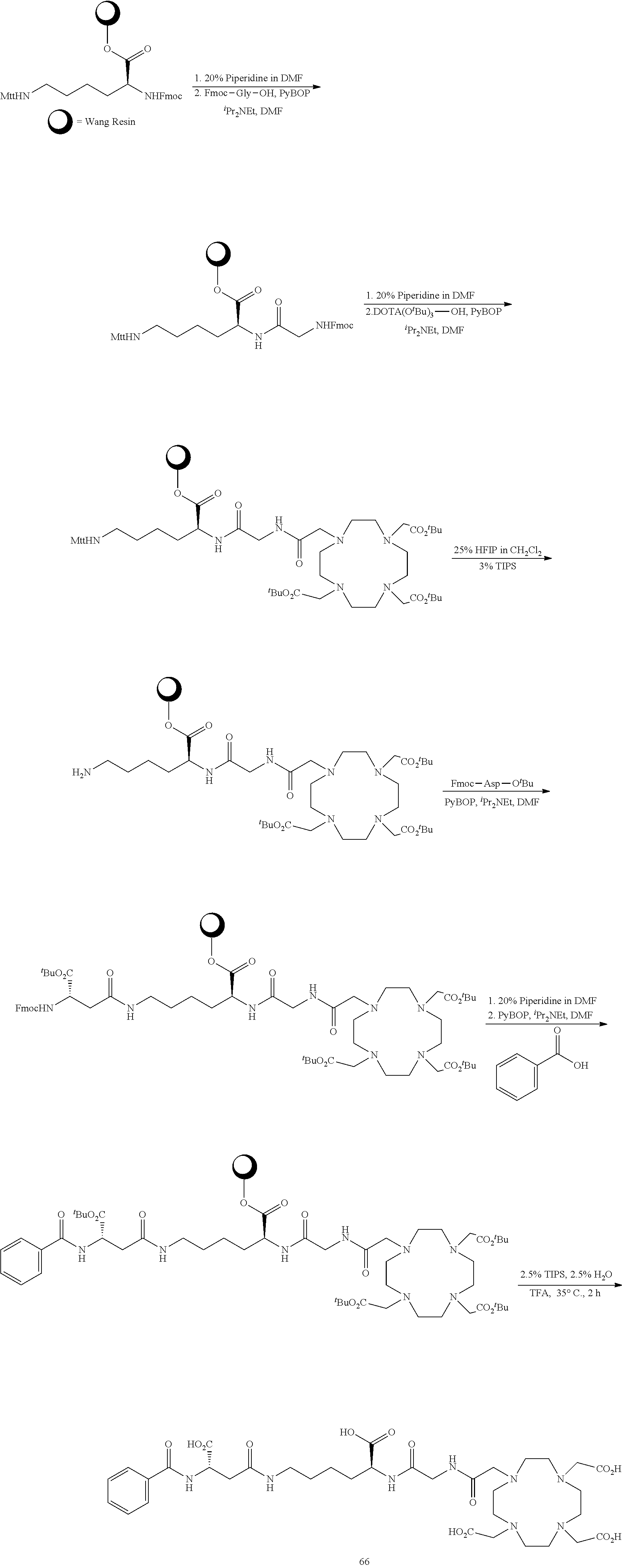

Compound 66 was synthesized by solid phase in six steps starting from Fmoc-Lys(N-4-methoxytrityl)-Wang-Resin (Table 13).

TABLE 13
Solid phase reaction steps:

| | | | MW | | |
|---|---|---|---|---|---|
| | mmol. | equiv. | (g/mol) | Amount | |
| Fmoc-Lys(N-4-methoxytrityl)- | 0.22 | — | — | 0.333 | g |
| Wang Resin | | | | | |
| (loading 0.538 mmol/g.) | | | | | |
| (commercially obtained) | | | | | |

Procedure A: Fmoc Deprotection - 20% Piperidine in DMF (3X)
Procedure C: Fmoc-Gly-OH Coupling

| | | | | | |
|---|---|---|---|---|---|
| Fmoc-Gly-OH | 0.44 | 2 | 297 | 131 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Tri-tert-butyl 1,4,7,10-tetraazacyclododecane-
1,4,7,10-tetraacetate {DOTA(O^{t}Bu)₃-OH} Coupling

| | | | | | |
|---|---|---|---|---|---|
| DOTA(O^{t}Bu)₃-OH | 0.33 | 1.5 | 573 | 189 | mg |
| (commercially obtained) | | | | | |
| ⁱPr₂NEt | 0.66 | 3 | 129 | 0.115 | mL |
| PyBOP | 0.33 | 1.5 | 520 | 172 | mg |

Procedure B: Mtt Deprotection - 25% HFIP in CH₂Cl₂ (6X)
Procedure C: Fmoc-L-Asp-O^{t}Bu Coupling

| | | | | | |
|---|---|---|---|---|---|
| Fmoc-Asp-O^{t}Bu | 0.44 | 2 | 412 | 181 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure A: Fmoc Deprotection - 20% Piperidine in DMF (3X)
Procedure C: Benzoic Acid Coupling

| | | | | | |
|---|---|---|---|---|---|
| Benzoic Acid | 0.44 | 2 | 122 | 54 | mg |
| (commercially obtained) | | | | | |
| ⁱPr₂NEt | 0.88 | 4 | 129 | 0.153 | mL |
| PyBOP | 0.44 | 2 | 520 | 229 | mg |

Procedure D₂: Cleavage - 2.5% TIPS, 2.5% H₂O, TFA

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D₂: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The filtrate was stirred at 35° C. under argon for 2 h. The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

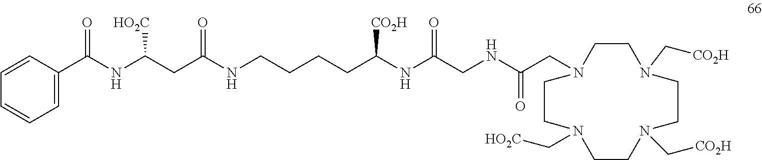 [SMILES: O=C(O)CN1CCN(CC(=O)O)CCN(CC(=O)NCC(=O)N[C@@H](CCCCNC(=O)C[C@H](NC(=O)c2ccccc2)C(=O)O)C(=O)O)CCN(CC(=O)O)CC1]

LCMS (ESI): [M+H]⁺=Calculated for C₃₅H₅₃N₈O₁₄, 809.36; found 809.40.

Selected data ¹H NMR (500 MHz, DMSO-d6, D₂O): δ 7.79 (d, J=8.5 Hz, 2H), 7.54 (dt, J₁=7.50 Hz, J₂=1.5 Hz, 1H), 7.47 (dt, J₁=7.50 Hz, J₂=1.0 Hz, 2H), 4.74 (dt, J₁=7.00 Hz, J₂=1.5 Hz, 1H), 4.13 (dd, J₁=7.250 Hz, J₂=5.0 Hz, 1H).

The compound of Example 51 is synthesized using similar procedures described in the Examples, above, using appropriate starting materials:

### Example 51: Synthesis of 4-((Naphthalen-2-ylmethyl)amino)benzoyl-Lys(DOTA)-OH (Compound 67)

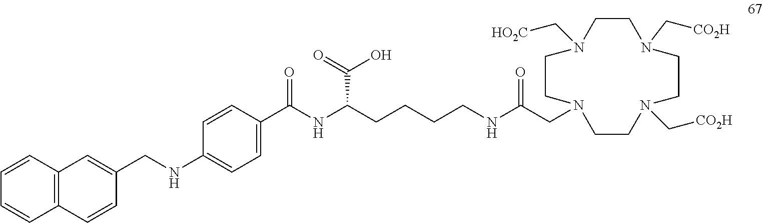 [SMILES: O=C(O)CN1CCN(CC(=O)O)CCN(CC(=O)NCCCC[C@H](NC(=O)c2ccc(NCc3ccc4ccccc4c3)cc2)C(=O)O)CCN(CC(=O)O)CC1]

LCMS (ESI): [M+H]⁺=Calculated for C₄₀H₅₄N₇O₁₀, 792.39; found 792.18.

Selected data ¹H NMR (500 MHz, DMSO-d6, D₂O): δ 7.85 (d, J=8.5 Hz, 2H), 7.83-7.77 (m, 2H), 7.58 (d, J=8.5 Hz, 2H), 7.51-7.41 (m, 3H), 6.61 (d, J=8.5 Hz, 2H), 4.47 (s, 2H), 4.20 (m, 1H).

### Example 52: Synthesis of Pte-Dap(Gly-DOTA)-OH (Compound 68)

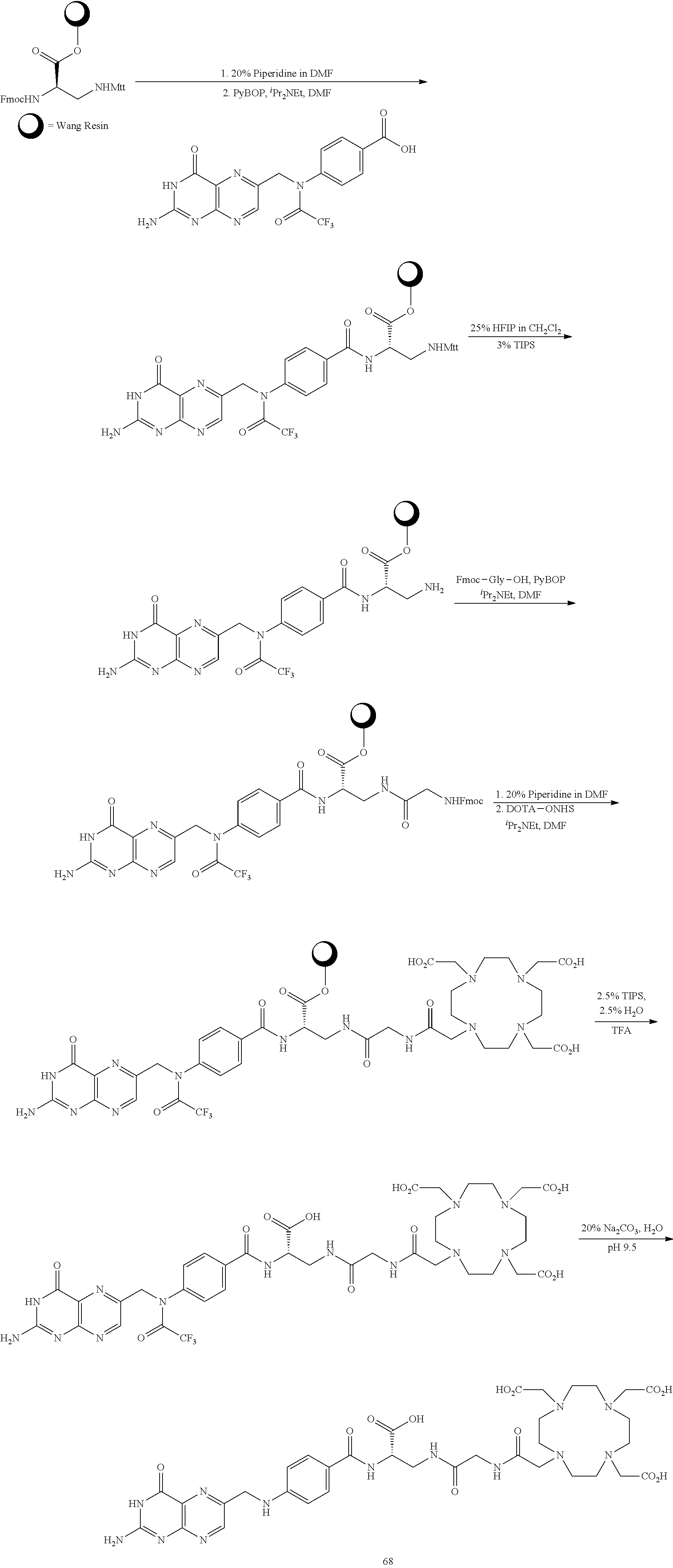

Compound 68 was synthesized by solid phase in five steps starting from Fmoc-Dap(N-4-methoxytrityl)-Wang-Resin.

Solid phase reaction steps:

| | | | MW | | |
|---|---|---|---|---|---|
| | mmol. | equiv. | (g/mol) | amount | |
| Fmoc-Dap(N-4-methoxytrityl)- | 0.188 | — | — | 1.0 | g |
| Wang Resin | | | | | |
| (loading 0.188 mmol/g.) | | | | | |
| (commercially obtained) | | | | | |

Procedure A: Fmoc Deprotection - 20% Piperidine in DMF (3X)
Procedure C: N¹⁰-TFA-Pteroic Acid Coupling

| | | | | | |
|---|---|---|---|---|---|
| N¹⁰-TFA-Pteroic Acid | 0.38 | 2 | 408 | 154 | mg |
| ⁱPr₂NEt | 0.75 | 4 | 129 | 0.131 | mL |
| PyBOP | 0.38 | 2 | 520 | 196 | mg |

Procedure B: Mtt Deprotection - 25% HFIP in CH₂Cl₂ (6X)
Procedure C: Fmoc-Gly-OH Coupling

| | | | | | |
|---|---|---|---|---|---|
| Fmoc-Gly-OH | 0.38 | 2 | 297 | 112 | mg |
| (commercially obtained) | | | | | |
| ⁱPr₂NEt | 0.75 | 4 | 129 | 0.131 | mL |
| PyBOP | 0.38 | 2 | 520 | 196 | mg |

Procedure A: Fmoc Deprotection - 20% Piperidine in DMF (3X)
Procedure C: N-Hydroxysuccinamidyl 1,4,7,10-tetraazacyclododecane-
1,4,7,10-tetraacetate {DOTA-ONHS} Coupling

| | | | | | |
|---|---|---|---|---|---|
| DOTA-ONHS | 0.38 | 2 | 762 | 286 | mg |
| (commercially obtained) | | | | | |
| ⁱPr₂Net | 0.75 | 4 | 129 | 0.131 | mL |

Procedure D: Cleavage - 2.5% TIPS, 2.5% H₂O, TFA
Procedure E: Deprotection of N¹⁰-TFA group in pteroic acid -
20% Na₂CO₃ and purification

### Procedure A: Fmoc Deprotection

The deprotection step was performed before each amino acid coupling steps (besides the Mtt deprotection which used 25% HFIP in CH₂Cl₂). A solution of 20% piperidine in DMF (˜20 mL) for Fmoc deprotection was added. Argon was bubbled through the solution for 10 min and then drained. 20% piperidine in DMF (˜20 mL) was added and bubbling continued for 10 min before draining (2×). The resin was washed with DMF (˜20 mL×3) followed by IPA (˜20 mL×3) and with DMF again (˜20 mL×3).

### Procedure B: Mtt Cleavage

25% HFIP in CH₂Cl₂ (˜20 mL) was added and argon was bubbled through the solution for 10 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. The resin was washed with DMF (˜20 mL×3) followed by IPA (˜20 mL×3) and with DMF again (˜20 mL×3).

### Procedure C: Amino Acid Coupling

An amino acid solution in DMF (˜20 mL), ⁱPr₂NEt, and PyBOP were added to a peptide synthesis vessel. Argon was bubbled through the solution for 2 h and then drained. The resin was washed with DMF (˜20 mL×3) followed by IPA (˜20 mL×3) and with DMF (˜20 mL×3) again.

### Procedure D: Resin Cleavage

The resin was washed with MeOH (˜20 mL×3) and dried over stream of argon. 25 mL of cleavage reagent (95% TFA, 2.5% H₂0, 2.5% Triisopropylsaline) was added to the peptide synthesis vessel and Argon was bubbled for 1 h, drain, and repeated with cleavage reagent (10 mL for 5 min (×2)). The reaction mixture was concentrated under reduced pressure until 10 ml remained. The product was triturated in 25 mL of diethyl ether and centrifuged. The solution was decanted from the resulting pellet. The previous step was repeated twice by resuspending the pellet in 25 mL of diethyl ether and centrifuging. The pellet was dried over a stream of argon and then high vacuum.

### Procedure E: Deprotection of N¹⁰-TFA Group in Pteroic Acid and Purification

The crude precipitate was suspended in water. 20% Na₂CO₃ was added until pH of the solution reached to 9.5. The clear solution was stirred for 1 h, LCMS analysis confirmed the product formation. pH of the solution was adjusted to 6.5 using 1N HCl, and loaded onto a C18 column. The desired product was purified by reverse phase chromatography (5-50% acetonitrile in 50 mM ammonium bicarbonate buffer at pH 7.0). Acetonitrile was evaporated under reduced pressure, and the remaining aqueous buffer solution was frozen and removed by lyophilization.

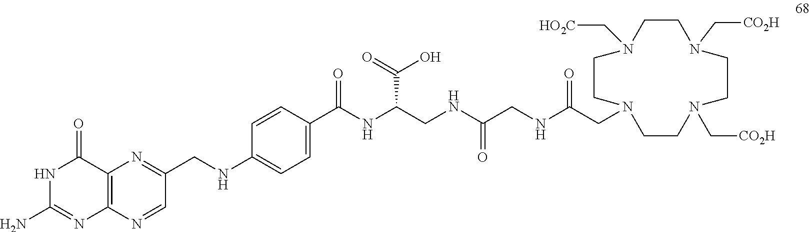 [SMILES: Nc1nc2ncc(CNc3ccc(C(=O)N[C@@H](CNC(=O)CNC(=O)CN4CCN(CC(=O)O)CCN(CC(=O)O)CCN(CC(=O)O)CC4)C(=O)O)cc3)nc2c(=O)[nH]1]

LCMS (ESI): [M+H]⁺=Calculated for C₃₅H₄₈N₁₃O₁₂, 842.35; found 842.52.

### Example 53: Preparation of [¹⁷⁷Lu]-Compound 34 Materials

| Name | Supplier |
|---|---|
| Lu-177 Chloride, n.c.a. in 0.04M HCl. | ITM |
| Sodium Acetate, Anhydrous 99.99% | Sigma-Aldrich |
| Suprapur | |
| Gentisic Acid (2,5 dihydroxybezoic acid), | Sigma-Aldrich |
| 99.0% matrix substance for MALDI-MS | |
| Hydrochloric Acid, TraceMetal Grade | Fisher |
| Sodium Hydroxide, | Fluka |
| Tracesclect grade >30% | |
| DTPA, disodium salt, for | Sigma-Aldrich |
| complexometry >99.0% | |
| Trizma Base, Bioxtra >99.9% | Sigma-Aldrich |
| Sodium Ascorbate, USP | Spectrum |
| Water for Injection, USP | RMBI |

Preparation of sodium acetate buffer solution (0.3M, pH 5.5): Sodium acetate (12.3 g) was dissolved in 300 mL of water for injection. The pH was adjusted to 5.5 using hydrochloric acid. Water for injection was added to the 500 mL mark. The solution was stored in a refrigerator.

Preparation of gentisic acid solution (10 mg/mL): Gentisic acid (250 mg) was dissolved in 20 mL of 0.3M sodium acetate pH 5.5 solution. The pH was adjusted to 5.5 using 30% NaOH solution. Sodium acetate buffer pH 5.5 was added to 25 mL mark. The solution (1 mL) was dispensed to 10 mL glass vials, stoppered and sealed under nitrogen and stored in a freezer at −20° C.

Preparation of DTPA/Sodium Ascorbate/Tris buffer solution: DTPA (22 mg), sodium ascorbate (5.0 g) and trizama base (2.42 g) were added to a 100 mL bottle. Water For Injection (80 mL) was added to dissolve the solids. The solution was sparged with nitrogen and the pH was adjusted to 7.4 using hydrochloric acid. Water for injection was added to 100 mL mark. Final concentration: DTPA 0.22 mg/mL; Sodium Ascorbate: 50 mg/mL, Tris Buffer: 0.2M, pH 7.4. The solution (5 mL) was dispensed to 10 mL glass vials, stoppered and sealed under nitrogen. The vials were stored in a refrigerator.

Preparation of Compound 34 solution (2 mM): Compound 34 (1.2 mg) was dissolved in 1.0 mL of water for injection. The vial was stored in a freezer at −20° C.

Preparation of [¹⁷⁷Lu]-Compound 34: Compound 34 solution (250 μL, 2 mM) was added to a vial. Gentisic acid/acetate buffer pH 5.5 (800 μL) and ¹⁷⁷LuCl₃ solution (170 μL, 184 mCi) were added to the vial. The vial was placed in a shielded heating block and heated at 95° C. for 15 min. After cooling to room temperature, 7 mL of DTPA/sodium ascorbate solution pH 7.4 was added to the labeling mixture. The final solution contains 184 mCi of ¹⁷⁷Lu, 0.6 mg of Compound 34, 8 mg of gentisic acid, 1.5 mg of DTPA, 350 mg of sodium ascorbate.

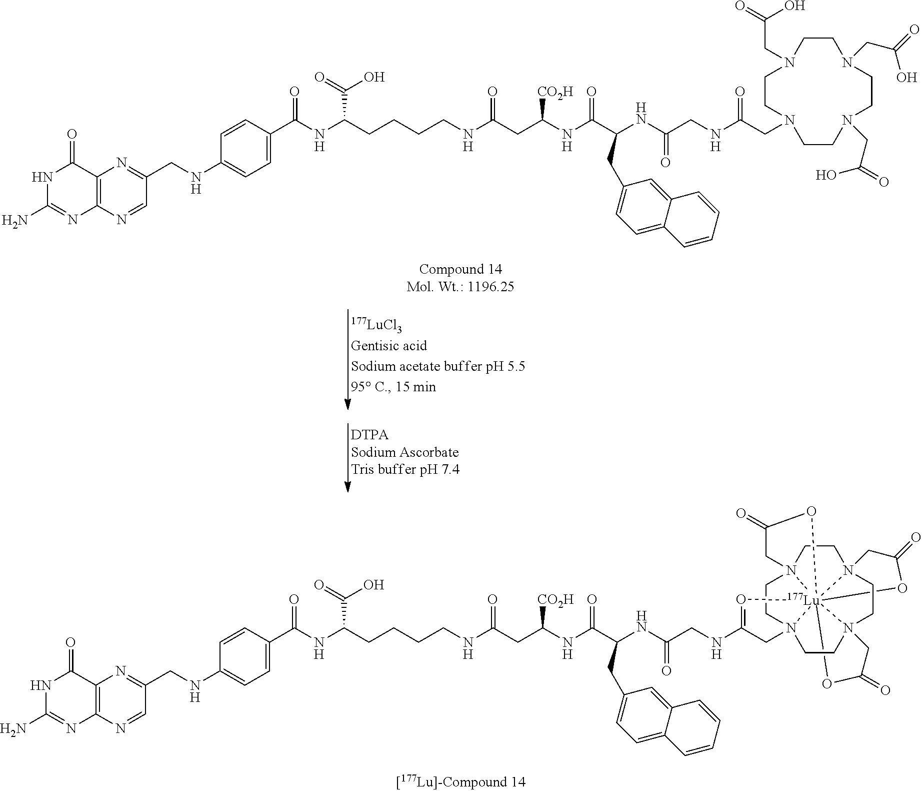

### Stability of [¹⁷⁷Lu]-Compound 34

[¹⁷⁷Lu]-Compound 34 solution was stored at room temperature and in a refrigerator. The radiochemical purities were monitored using radio-HPLC. [¹⁷⁷Lu]-Compound 34 was stable up to 6 days.

| | Radiochemical purity (%) | | |
|---|---|---|---|
| Storage conditions | initial | 3 days | 6 days |
| Room temperature (24° C. ) | 97.14 | 96.77 | 95.89 |
| 0° C. | 97.14 | 97.19 | 96.58 |

### Example 54: Preparation of [¹⁷⁵Lu]-Compound 34]

| | | | | |
|---|---|---|---|---|
| ¹⁷⁵Lutetium (III) | Sigma- | 450960-1G | 281.33 | MKCB0960v |
| chloride | Aldrich | | | |

Preparation of Compound 34 solution: Dissolved 49.8 mg (0.04 mmol) of Compound 34 in 5 mL of 1 M NaOAc buffer pH 5.5.

Preparation of ¹⁷⁵LuCl₃ solution: Dissolved 100 mg (0.36 mmol) of ¹⁷⁵LuCl₃ in 2 mL of 0.1 M HCl.

Preparation of [¹⁷⁵Lu]-Compound 34: Lutetium chloride solution (1 mL, 0.18 mmol) was added to vial containing 49.8 mg (0.04 mmol) of Compound 34. The mixture was heated at 95° C. for 15 min. LC-MS confirmed that Compound 34 completely converted to [¹⁷⁵Lu]-Compound 34.

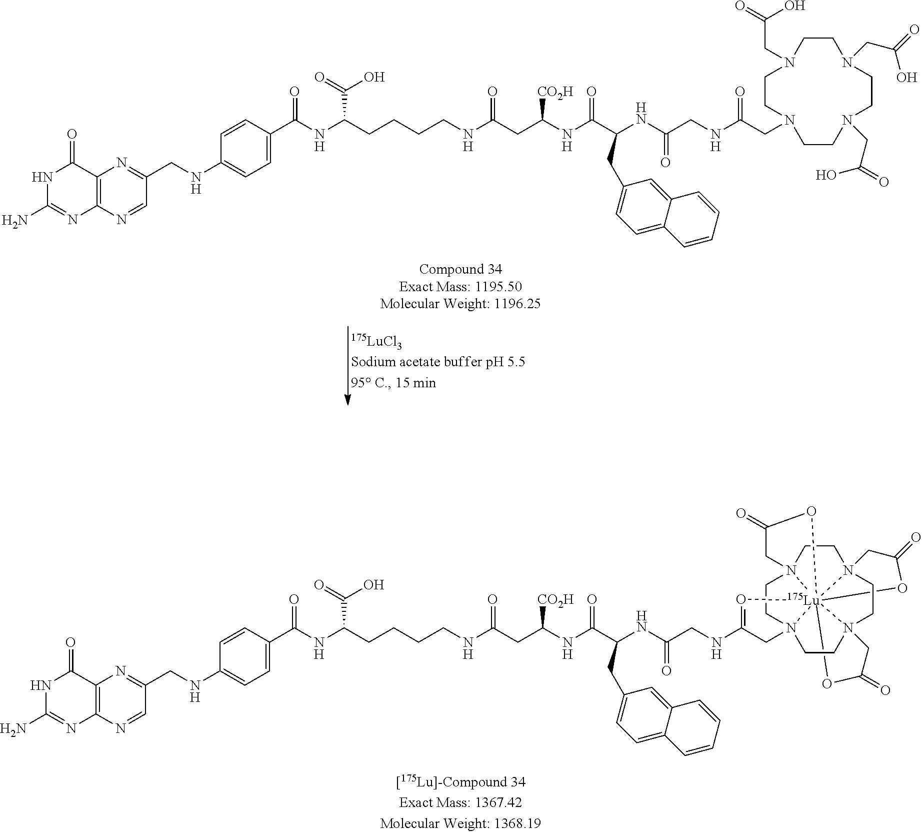

Purification: The material was purified using a Biotage SNAP ultra C18 30G cartridge. Mobile phase A: 10 mM NH₄HCO₃, B: acetonitrile, Gradient: 0%₀B in 2 CV, 0%₀B to 50%₀B in 10CV. Flow rate: 25 mL/min. UV 280 nm. The fractions containing [¹¹⁵Lu]-Compound 34 were combined and lyophilized. 37 mg of [¹⁷⁵Lu]-Compound 34 was obtained.

### Example 55: Preparation of [¹⁷⁷Lu]-Compound 37

### Materials

| Name | Supplier |
|---|---|
| Lu-177 Chloride, n.c.a. in 0.04M HCl. | ITM |
| Sodium Acetate, Anhydrous 99.99% | Sigma-Aldrich |
| Suprapur | |
| Gentisic Acid (2,5 dihydroxybezoic acid), | Sigma-Aldrich |
| 99.0% matrix substance for MALDI-MS | |
| Hydrochloric Acid, TraceMetal Grade | Fisher |
| Sodium Hydroxide, | Fluka |
| Tracesclect grade >30% | |
| DTPA, disodium salt, for | Sigma-Aldrich |
| complexometry >99.0% | |
| Trizma Base, Bioxtra >99.9% | Sigma-Aldrich |
| Sodium Ascorbate, USP | Spectrum |
| Water for Injection, USP | RMBI |

Preparation of sodium acetate buffer solution (0.3M, pH 5.5): Sodium acetate (12.3 g) was dissolved in 300 mL of water for injection. The pH was adjusted to 5.5 using hydrochloric acid. Water for injection was added to the 500 mL mark. The solution was stored in a refrigerator.

Preparation of gentisic acid solution (10 mg/mL): Gentisic acid (250 mg) was dissolved in 20 mL of 0.3M sodium acetate pH 5.5 solution. The pH was adjusted to 5.5 using 30% NaOH solution. Sodium acetate buffer pH 5.5 was added to 25 mL mark. The solution (1 mL) was dispensed to 10 mL glass vials, stoppered and sealed under nitrogen and stored in a freezer at −20° C.

Preparation of DTPA/Sodium Ascorbate/Tris buffer solution: DTPA (22 mg), sodium ascorbate (5.0 g) and trizama base (2.42 g) were added to a 100 mL bottle. Water For Injection (80 mL) was added to dissolve the solids. The solution was sparged with nitrogen and the pH was adjusted to 7.4 using hydrochloric acid. Water for injection was added to 100 mL mark. Final concentration: DTPA 0.22 mg/mL; Sodium Ascorbate: 50 mg/mL, Tris Buffer: 0.2M, pH 7.4. The solution (5 mL) was dispensed to 10 mL glass vials, stoppered and sealed under nitrogen. The vials were stored in a refrigerator.

Preparation of Compound 37 solution (2 mM): Compound 37 (2.7 mg) was dissolved in 1.2 mL of water for injection. The solution was stored in a freezer at −20° C.

Preparation of [¹⁷⁷Lu]-Compound 37: Compound 37 solution (5 μL, 2 mM) was added to a vial. Gentisic acid/acetate buffer pH 5.5 (300 μL) and ¹⁷⁷LuCl₃ solution (15p, 16 mCi) were added to the vial. The vial was placed in a shielded heating block and heated at 95° C. for 15 min. After cooling to room temperature, 2 mL of DTPA/sodium ascorbate solution pH 7.4 was added to the labeling mixture.

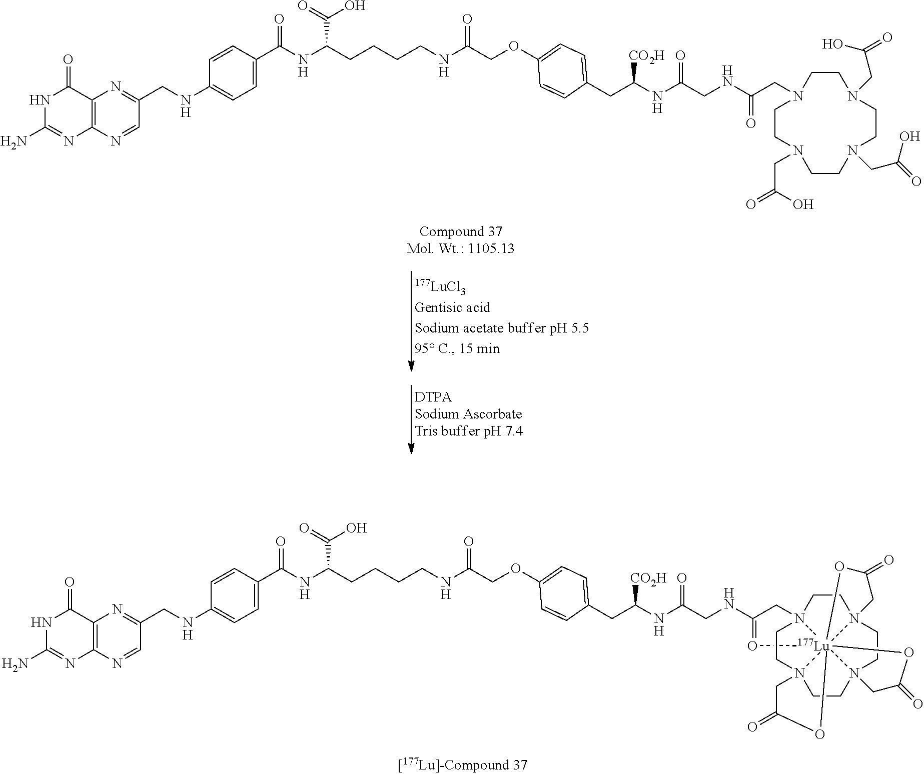

The invention further includes any variant of the present processes (including those provided in Examples 1-55), in which an intermediate obtainable at any stage thereof is used as starting material and the remaining steps are carried out, or in which the starting materials are formed in situ under the reaction conditions, or in which the reaction components are used in the form of their salts or optically pure material. Compounds of the present disclosure and intermediates can also be converted into each other according to methods generally known to those skilled in the art.

The compounds of the present disclosure exhibit valuable pharmacological properties as FR targeting compounds, e.g. as indicated in vitro and in vivo tests as provided in the next sections, and are therefore indicated for therapy, for diagnosis, for imaging, or for use as research chemicals, e.g. as tool compounds.

### BIOLOGICAL EXAMPLES

The activity of a compound according to the present disclosure can be assessed by the following in vitro and in vivo methods. The radiolabeled compounds used in the following Biological Compounds were prepared using the radiolabeling methods described in Examples 53-55 above, or methods analogous to these methods.

### Biological Example 1: Relative Affinity Assay

FR-positive KB cells were seeded in 24-well Falcon plates and allowed to form adherent monolayers (>90% confluent) overnight in in FDRPMI/10% FCS media. Spent incubation medium was replaced with FFRPMI supplemented with 10% HIFCS and containing 100 nmol/L of [³H]FA in the absence and presence of increasing concentrations of unlabeled folic acid (FA), Compound 34, Compound 37, or non-targeted control (Table 14). Cells were incubated for 1 h at 37° C. and then rinsed three times with 0.5 mL PBS (phosphate-buffered saline). Five hundred microliters of 1% SDS (sodium dodecyl sulfate) in PBS were added to each well; after 5 min, cell lysates were collected, transferred to individual vials containing 5 mL of scintillation cocktail, and then counted for radioactivity. Cells exposed to only the [³H]FA in FFRPMI (no competitor) were designated as negative controls, whereas cells exposed to the [³H]FA plus 1 mmol/L unlabeled FA served as positive controls. Disintegrations per minute (DPM) measured in the latter samples (representing nonspecific binding of label) were subtracted from the DPM values from all samples. Relative affinities were defined as the inverse molar ratio of compound required to displace 50% of [³H]FA bound to FR on KB cells, and the relative affinity of FA for the FR was set to 1.

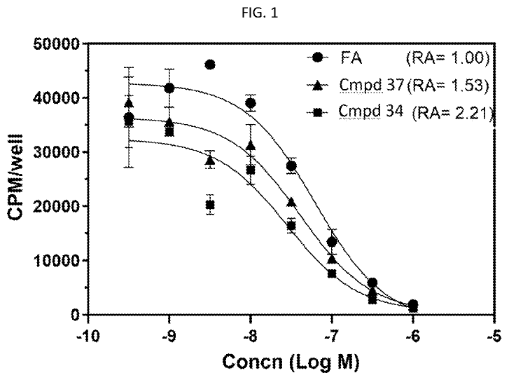

Table 14 details the relative binding affinities of the positive/negative controls and compounds 34 and 37. As shown in Table 14, Compound 34 and Compound 37 were shown to bind folate receptors (FRs) with a higher affinity than folic acid, with relative affinities (RA's) of 1.53 and 2.21, respectively (see also FIG. 1).

### Biological Example 2: Binding Affinity Assay

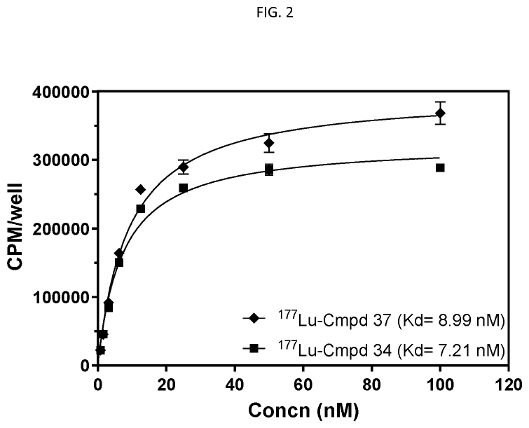

FR-positive KB cells and FR-negative A549 cells were seeded in 24-well Falcon plates and allowed to form adherent monolayers (>90% confluent) overnight in FFRPMI/HIFCS. Spent incubation medium was replaced with FFRPMI supplemented with 10% HIFCS containing increasing concentrations (0.78 to 100 nmol/L) of [¹⁷⁷Lu]-Compound 34, [¹⁷⁷Lu]-Compound 37, or [¹⁷⁷Lu]-(non-targeted control) in the absence and presence of 10 μM FA. Cells were incubated for 1 h at 37° C. and then rinsed three times with 0.5 mL PBS. Five hundred microliters of 1% NaOH in PBS were added to each well; after 5 min, cell lysates were collected, transferred to individual tubes, and then counted for radioactivity on a gamma counter. Counts per minute (CPM) values were measured in all samples and plotted against concentration of [¹⁷⁷Lu]-Compound 34, [1⁷⁷Lu]-Compound 37, or [¹⁷⁷Lu]-(non-targeted control) using GraphPad Prism 8 program. Dissociation constants (Kd) was calculated using a GraphPad's nonlinear regression one site binding method.

[¹⁷⁷Lu]-Compound 34 and [¹⁷⁷Lu]-Compound 37 were shown to bind folate receptors with high affinity with dissociation constants (Kd) values of 7.21 nM and 8.99 nM, respectively (see FIG. 2). Results are further provided in Table 14.

TABLE 14
Results for Biological Example 1 and Biological Example 2

| Test Article | | Relative | Direct |
|---|---|---|---|
| Com- | | Binding | Binding |
| pound | Structural Formula | Affinity* | (Kd, nM) |
| Folic Acid | 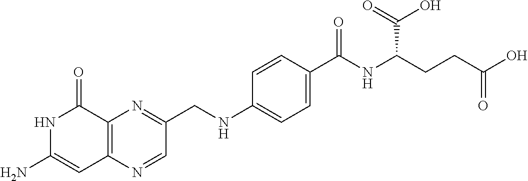 [SMILES: Nc1cc2ncc(CNc3ccc(C(=O)N[C@@H](CCC(=O)O)C(=O)O)cc3)nc2c(=O)[nH]1] | 1.00 | 2.6 |
| 37 | 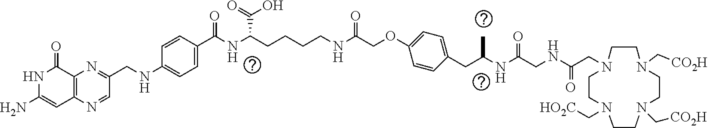 [SMILES: C[C@H](Cc1ccc(OCC(=O)NCCCC[C@H](NC(=O)c2ccc(NCc3cnc4cc(N)[nH]c(=O)c4n3)cc2)C(=O)O)cc1)NC(=O)CNC(=O)CN1CCN(CC(=O)O)CCN(CC(=O)O)CCN(CC(=O)O)CC1] | 1.53 | 8.99 |
| 34 | 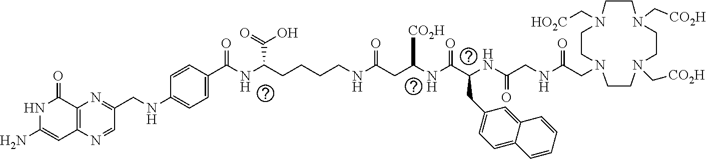 [SMILES: Nc1cc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1] | 2.21 | 7.21 |
| (non- targeted control) | 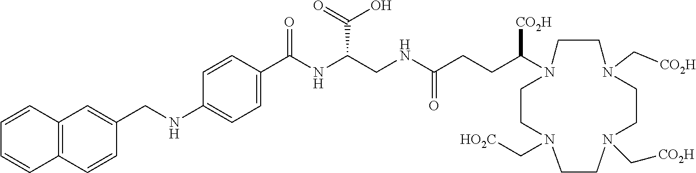 [SMILES: O=C(O)CN1CCN(CC(=O)O)CCN([C@@H](CCC(=O)NC[C@H](NC(=O)c2ccc(NCc3ccc4ccccc4c3)cc2)C(=O)O)C(=O)O)CCN(CC(=O)O)CC1] | Non- binder | Non- binder |

*Affinity of folic acid is set to unity

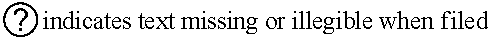

### Biological Example 3: In Vivo Biodistribution Experiments

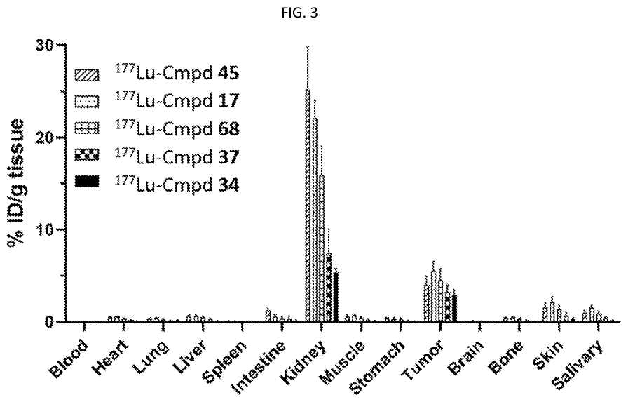

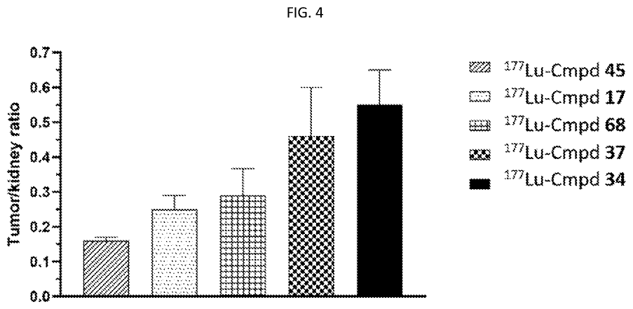

Four- to eight-week-old female nu/nu mice or NSG mice (Harlan Sprague-Dawley, Inc.) were maintained on a standard 12-h light-dark cycle and fed ad libitum with Folate deficient purified rodent diet (TestDiet #AIN-93G) for the duration of the experiment. FR-positive M109 or FR-negative HT29 tumor cells were inoculated in the subcutis dorsal medial area of mice. The biodistribution studies were typically performed when tumors were approximately 400-800 mm³ in volume. Mice were divided into groups of three, and freshly prepared test articles and competitors were injected through the lateral tail vein in a volume of 100 μL/10 g of PBS. Four h to six days post radioactive-agent dose administration, mice were euthanized and organs (blood, heart, lungs, liver, spleen, and kidneys, intestine, stomach, muscle, brain and tumor) were collected, weighed and placed inside counting vials. Each tissue sample was counted for the activities of radioelement using a gamma-counter. Samples of the injectate were used as decay correction standards. Final bar graphs are expressed as % injected dose per gram of tissue or tumor to kidney ratio, or % tumor to (kidneys+liver+spleen) ratio. Results are shown in Table 15 and in FIGS. 3-4.

TABLE 15
Biodistribution Studies

| | Tested | | Dose | | | | | |
|---|---|---|---|---|---|---|---|---|
| CPD | Chelate | Tumor | (nmol/kg) | n | T/Kid | T²/kid | T/Liv | T/spl |
| 2 | ²²⁵Ac | KB | 30 | 3 | 0.07 +/− 0.03 | n.d. | 1.2 +/− 0.5 | 7.3 +/− 2.3 |
| 3 | ¹⁷⁷Lu | M109 | 100 | 9 | 0.30 +/− 0.04 | 1.6 +/− 0.4 | 0.93 +/− 0.15 | 10.6 +/− 1.8 |
| | ²²⁵Ac | KB | 30 | 3 | 0.04 +/− 0.01 | n.d. | 0.5 +/− 0.3 | 6.8 +/− 2.9 |
| 5 | ¹⁷⁷Lu | M109 | 100 | 6 | 0.25 +/− 0.07 | 2.0 +/− 1.2 | 5.2 +/− 1.7 | 24.4 +/− 8.8 |
| | ¹⁷⁷Lu | M109 | 100 | 18 | 0.22 +/− 0.08 | 1.6 +/− 1.0 | 5.3 +/− 3.0 | 25.3 +/− 11.4 |
| | ¹⁷⁷Lu | M109 | 100 | 3 | 0.16 +/− 0.01 | 1.1 +/− 0.3 | 2.2 +/− 0.2 | 14.6 +/− 3.0 |
| | ²²⁵Ac | KB | 30 | 6 | 0.09 +/− 0.02 | n.d. | 3.5 +/− 1.9 | 19.9 +/− 10.9 |
| 11 | ¹¹¹In | M109 | 100 | 3 | 0.19 +/− 0.01 | 1.0 +/− 0.14 | 5.7 +/− 1.5 | 28.0 +/− 31 |
| 13 | ¹¹¹In | M109 | 100 | 3 | 0.22 +/− 0.06 | 1.7 +/− 1.1 | 6.4 +/− 1.5 | 31.9 +/− 7.6 |
| 17 | ¹⁷⁷Lu | M109 | 100 | 3 | 0.20 +/− 0.03 | 1.7 +/− 0.5 | 4.9 +/− 1.3 | 19.6 +/− 3.9 |
| 20 | ¹⁷⁷Lu | M109 | 100 | 3 | 0.21 +/− 0.01 | 1.5 +/− 0.3 | 4.6 +/− 2.1 | 16.4 +/− 1.9 |
| 23 | ²²⁵Ac | KB | 30 | 3 | 0.08 +/− 0.03 | n.d. | 1.8 +/− 0.7 | 10.1 +/− 4.7 |
| 25 | ²²⁵Ac | KB | 30 | 6 | 0.07 +/− 0.03 | n.d. | 3.3 +/− 1.8 | 15.8 +/− 5.9 |
| 26 | ²²⁵Ac | KB | 30 | 3 | 0.08 +/− 0.02 | n.d. | 3.3 +/− 1.1 | 20.4 +/− 5.6 |
| 28 | ¹¹¹IN | M109 | 100 | 3 | 0.098 +/− 0.003 | 0.03 +/− 0.001 | 1.2 +/− 0.1 | 2.1 +/− 0.3 |
| 68 | ¹⁷⁷Lu | M109 | 200 | 13 | 0.29 +/− 0.08 | 1.37 +/− 0.73 | 10.1 +/− 3.2 | 55.6 +/− 17.1 |
| 46 | ¹⁷⁷Lu | KB | 200 | 3 | 0.19 +/− 0.03 | 0.84 +/− 0.32 | 6.3 +/− 1.9 | 66.9 +/− 30.8 |
| 45 | ¹⁷⁷Lu | KB | 200 | 3 | 0.16 +/− 0.01 | 0.64 +/− 0.19 | 6.8 +/− 1.1 | 50.7 +/− 24.0 |
| 51 | ¹⁷⁷Lu | KB | 200 | 3 | 0.32 +/− 0.09 | 1.35 +/− 0.54 | 11.0 +/− 2.3 | 54.8 +/− 11.3 |
| 54 | ¹⁷⁷Lu | M109 | 200 | 3 | 0.23 +/− 0.03 | 0.76 +/− 0.12 | 9.0 +/− 0.9 | 56.4 +/− 6.2 |
| 37 | ¹⁷⁷Lu | M109 | 300 | 11 | 0.46 +/− 0.14 | 1.51 +/− 0.57 | 13.2 +/− 4.5 | 61.9 +/− 14.4 |
| 58 | ¹⁷⁷Lu | M109 | 300 | 3 | 0.59 +/− 0.12 | 0.59 +/− 0.16 | 28.8 +/− 2.9 | 83.1 +/− 17.3 |
| 38 | ¹⁷⁷Lu | M109 | 300 | 3 | 0.46 +/− 0.03 | 1.28 +/− 0.11 | 10.5 +/− 2.9 | 69.9 +/− 7.9 |
| 39 | ¹⁷⁷Lu | M109 | 300 | 6 | 0.57 +/− 0.22 | 1.68 +/− 0.59 | 17.1 +/− 6.4 | 83.8 +/− 29.7 |
| 34 | ¹⁷⁷Lu | M109 | 300 | 12 | 0.55 +/− 0.1 | 1.7 +/− 0.9 | 25.3 +/− 6.6 | 98.3 +/− 25.1 |
| | | | 600 | 6 | 0.80 +/− 0.18 | 1.97 +/− 0.7 | 29.1 +/− 5.4 | 92.8 +/− 5.4 |
| 61 | ¹⁷⁷Lu | M109 | 300 | 3 | 0.27 +/− 0.03 | 1.05 +/− 0.28 | 8.6 +/− 3.9 | 40.3 +/− 18.7 |
| 64 | ¹⁷⁷Lu | M109 | 300 | 3 | 0.38 +/− 0.07 | 1.48 +/− 0.51 | 14.6 +/− 2.0 | 85.5 +/− 7.7 |
| 42 | ¹⁷⁷Lu | M109 | 300 | 3 | 0.49 +/− 0.16 | 1.64 +/− 0.62 | 17.3 +/− 4.9 | 84.4 +/− 10.4 |
| 43 | ¹⁷⁷Lu | M109 | 300 | 3 | 0.58 +/− 0.10 | 1.57 +/− 0.53 | 13.0 +/− 3.7 | 70.2 +/− 6.7 |
| 23 | ¹⁷⁷Lu | M109 | 300 | 3 | 0.37 +/− 0.11 | 1.47 +/− 0.66 | 25.8 +/− 2.4 | 108 +/− 12.7 |

Biological Example 4: In Vivo Anti-Tumor Activity Experiments

Four- to eight-week-old female nu nu mice or NSG mice (Harlan Sprague-Dawley, Inc.) were maintained on a standard 12-h light-dark cycle and fed ad libitum with folate deficient purified rodent diet (TestDiet #AIN-93G). FR-positive (human breast adenocarcinoma) or IGROV (human ovarian adenocarcinoma) or KB (human cervical adenocarcinoma) tumor cells were inoculated subcutaneously at the right flank of each mouse. Mice were dosed with [¹⁷⁷Lu]-Compound 34, [¹⁷⁷Lu]-Compound 37, or [²²⁵Ac]-Compound 5 through the lateral tail vein under sterile conditions in a volume of 100 μL/10 g of phosphate-buffered saline (PBS). Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. A stable disease (SD) was defined as volume reduction of <50% and increase of <50% in two weeks. A partial response (PR) was defined as volume regression >50% but with measurable tumor (>2 mm³) remaining at all times. Complete response (CR) was defined as a disappearance of measurable tumor mass (<2 mm³) at some point within the study. Cures were defined as CRs without tumor regrowth within the study time frame. As a general measure of gross toxicity, changes in body weights were determined on the same schedule as tumor volume measurements.

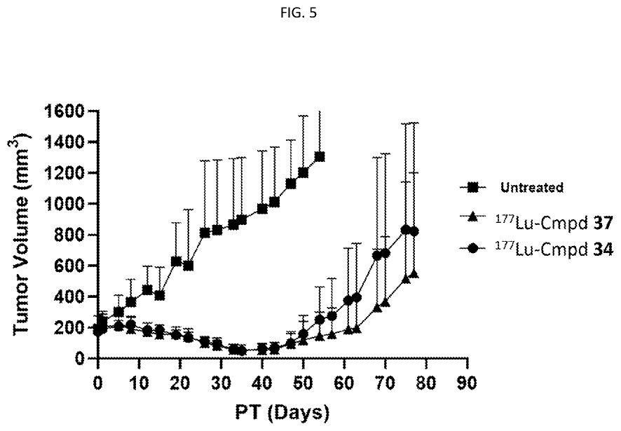

Results are shown in FIGS. 5-8. FIG. 5 is a chart showing the anti-tumor activity of [¹⁷⁷Lu]-Compound 37 and [¹⁷⁷Lu]-Compound 34 at 300 nmol/3.7 GBq/kg in female nu nu mice bearing MDA-MB-231 tumors (n=5); four partial responses (PRs) and one complete response (CR) were found for [¹⁷⁷Lu]-Compound 37 and five PRs were found for [¹⁷⁷Lu]-Compound 34; (▪) control; (▴) [¹⁷⁷Lu]-Compound 37; (●) [¹⁷⁷Lu]-Compound 34.

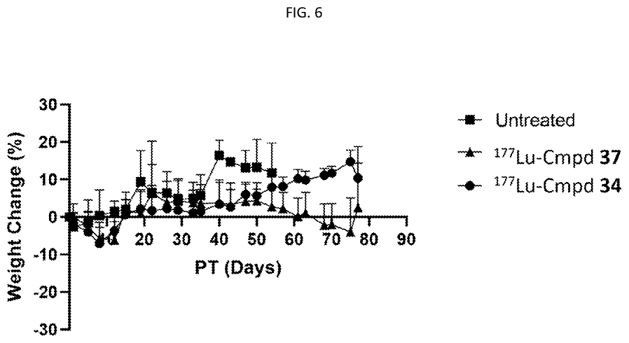

FIG. 6 is a chart showing the average weight of mice from the study in FIG. 5. The results show treatment was well tolerated; mice in both of the treated groups did not lose any significant weight immediately after dosing and beyond; (▪) control; (▴) [¹⁷⁷Lu]-Compound 37; (●) [¹⁷⁷Lu]-Compound 34.

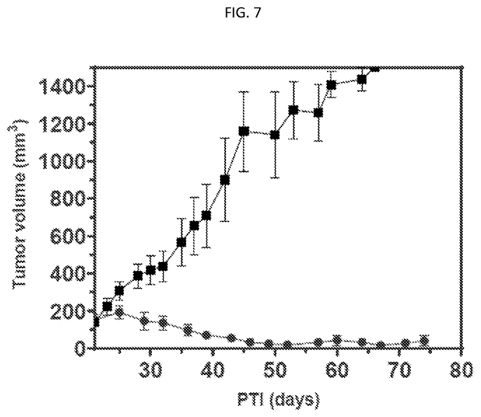

FIG. 7 is a chart showing the anti-tumor activity of [²²⁵Ac]-Compound 5 at 100 nmol/30 mCi/kg in mice bearing MDA-MB-231 tumors. The results show treatment with [²²⁵Ac]-Compound 5 provided 50% complete response and 50% partial response. (▪) control; (●) [²²⁵Ac]-Compound 5.

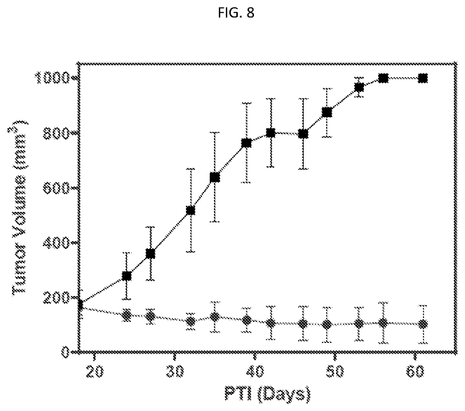

FIG. 8 is a chart showing the anti-tumor activity of [²²⁵Ac]-Compound 5 at 100 nmol/30 mCi/kg in mice bearing KB tumors. The results show treatment with [²²⁵Ac]-Compound 5 provided 80% partial response and 20% stable disease. (▪) control; (●) [²²⁵Ac]-Compound 5.

### Biological Example 5: In Vivo Biodistribution Experiments

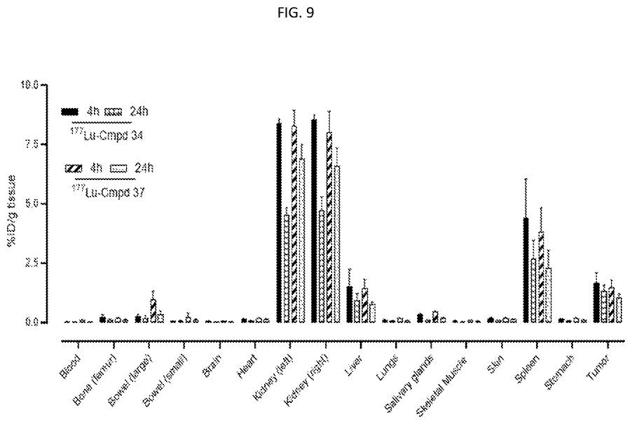

Six week old female Athymic Nude-Foxn1nu mice (Envigo) were maintained on a standard 12-h light-dark cycle and fed ad libitum with folate deficient purified rodent dies (SSniff #E15321-147) for the duration of the experiment. FR-positive IGROV-1 tumor cells were inoculated in the subcutis dorsal medial area of mice. The biodistribution studies were 147±60 mm3 in volume. Mice were divided into groups of four, and freshly prepared test articles were injected through the lateral tail vein in a volume of 100 μL/10 g of PBS. Four h to 24 h post radioactive agent dose administration, mice were euthanized and organs (blood, bone, bowel (large and small), brain, heart, kidneys, liver, lungs, salivary glands, skeletal muscle, skin, spleen, stomach and tumor) were collected, weight and placed inside counting vials. Each tissue sample was counted for the activities of radioelement using a gamma-counter. Samples of the injective were used as decay correction standards. Final bar graph is expressed as % injected dose per gram of tissue FIG. 9. Results of tumor to (kidneys+liver+spleen) ratios are shown in Table 16.

TABLE 16
Tumor to kidney, liver, spleen ratio at 24 h post injection (mean ± SD)

| | Tested | | Dose | | | | | |
|---|---|---|---|---|---|---|---|---|
| CPD | Chelate | Tumor | (nmol/kg) | n | T/Kid | T²/kid | T/Liv | T/spl |
| 34 | ¹⁷⁷Lu | IGROV-1 | 600 | 4 | 0.29 +/− 0.05 | n.d. | 1.53 +/− 0.57 | 0.49 +/− 0.07 |
| 37 | ¹⁷⁷Lu | IGROV-1 | 600 | 4 | 0.15 +/− 0.03 | n.d. | 1.37 +/− 0.37 | 0.49 +/− 0.17 |

### Biological Example 6: In Vivo Biodistribution Experiments

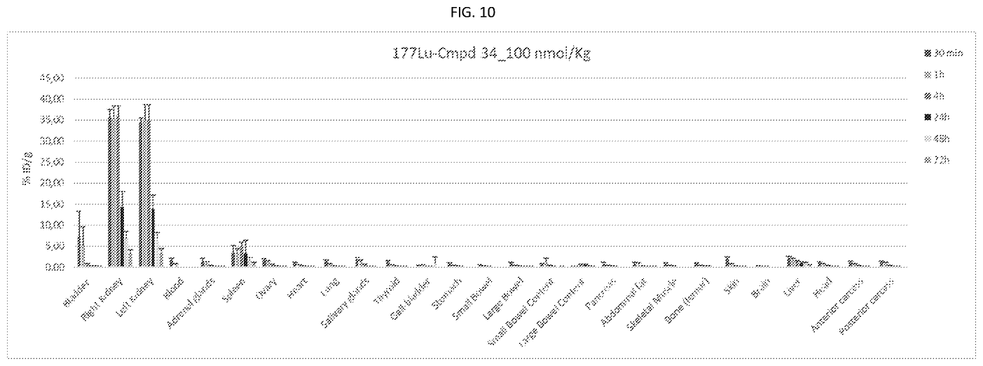

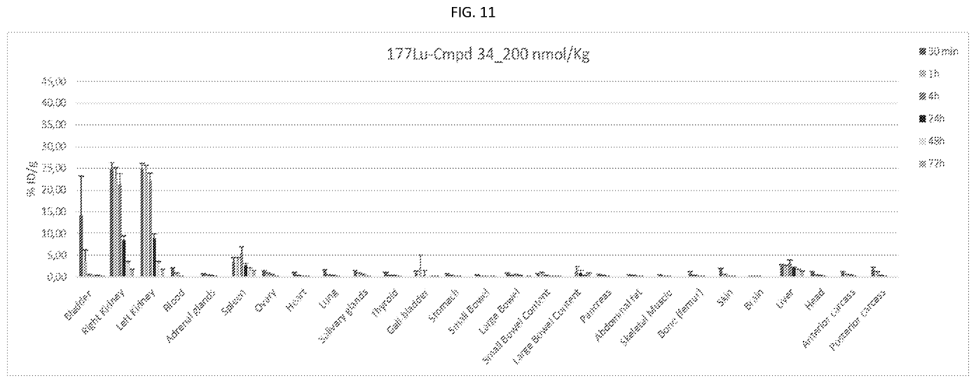

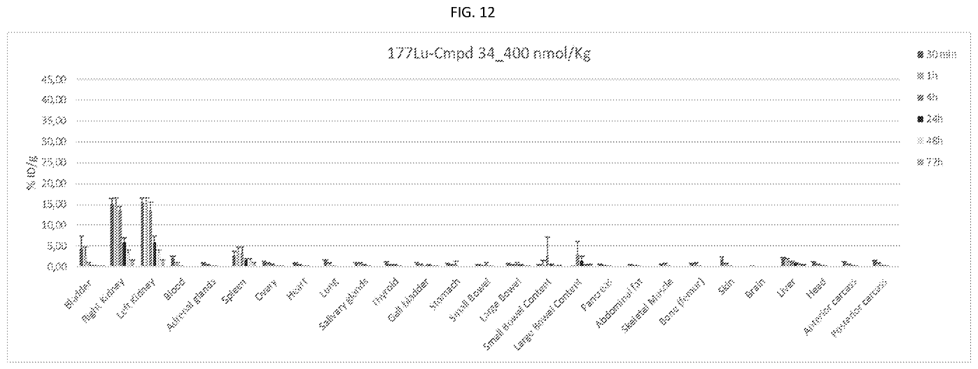

Five week old female Athymic Nude-Foxn1nu mice (Envigo) were maintained on a standard 12-h light-dark cycle and fed ad libitum with folate deficient purified rodent diet (Ssniff #E15321-147) for the duration of the experiment. Mice were divided into groups of three (corresponding to same radioactive dose with three different cold precursor molar amounts) and test articles were injected through the tail vein in a volume of ca. 100 μL/mouse. Thirty minutes to 72 hours post radioactive agent dose administration, mice were euthanized and organs (abdominal fat, adrenals, bladder, blood, bone (femur), brain, gallbladder, heart, large bowel, liver, lungs, ovary, pancreas, right and left kidney, salivary gland, skeletal muscle, skin, small bowel, spleen, stomach, tail, thyroid, and the animal carcass) were collected, weighed and placed inside counting vials. Each tissue sample was counted for the activities of radioelement using a gamma-counter. The calibration factor was calculated in order to transform cpm to organ activity and it was determined based on a standard calibration curve. Final bar graph is expressed as % injected dose per gram of tissue (see FIGS. 10-12).

### INCORPORATION BY REFERENCE

All publications, patents, and Accession numbers mentioned herein are hereby incorporated herein by reference in their entirety as if each individual publication or patent was specifically and individually indicated to be incorporated herein by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of formula (I) 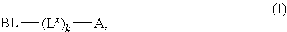
or a pharmaceutically acceptable salt thereof, wherein
BL is a folate receptor binding ligand,
A is a chelating group Ch which can comprise a metal, a radioelement, Si-¹⁸F, B-¹⁸F, or Al-¹⁸F, or A is a radiolabeled prosthetic group PG,
k is 1, 2, 3, 4, 5, 6, 7, 8, 9, or 10, and
each L^{X} is independently AA, L¹, L² or L³, wherein
each AA is independently an amino acid residue;
each L¹ is independently of the formula 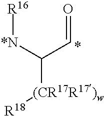
wherein
R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′} —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl, or a sugar;
w is 1, 2, 3, 4 or 5;
p is 1, 2, 3, 4 or 5;
q is 1, 2, 3, 4 or 5;
each L² is independently of the formula 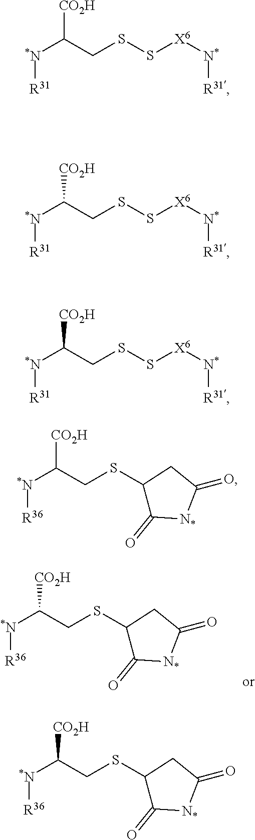
wherein
each R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
X⁶ is C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{5′} are independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
R³⁶ is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each L³ is independently C₁-C₆ alkylene, —OC₁-C₆ alkylene, —SC₁-C₆ alkylene, C₃-C₆ cycloalkylene, —C(O)C₃-C₆ cycloalkylene-, —C(O)C₃-C₆ cycloalkylene-(CR³⁹R^{39′})ᵣ—, —C(O)C₃-C₆ cycloalkylene-(CR³⁹R^{39′})ᵣNR³⁷—, 3- to 7-membered heterocycloalkylene, C₆-C₁₀ aryl, 5- to 7-membered heteroaryl, —NR³⁶(CR^{36′}R^{36″})ᵣ—S-(succinimid-1-yl)-, —(CR^{36′}R^{36″})ᵣC(O)NR³⁷—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ—, —C(O)O(CR³⁹R^{39′})ᵣ—, —NR³⁷C(O)(CR³⁹R^{39′})ᵣ—, —(CR³⁹R^{39′})ᵣC(O)NR³⁷—, —NR³⁷C(O)(CR^{39′}R^{39″})ᵣS—, —NR³⁷(CR³⁹R^{39′})ᵣ—, —(CR³⁹R^{39′})ᵣNR³⁸—, —NR³⁷(CR³⁹R^{39′})ᵣNR³⁸—, —NR³⁷(CR³⁹R^{39′})ᵣS—, —NR³⁷(CR³⁹R^{39′}CR³⁹R^{39′}O)ᵣ—, —NR³⁷(CR³⁹R^{39′}CR³⁹R^{39′}O)ᵣₚ—(CR³⁶R^{36′})ₜC(O)—, —C(O)(CR³⁶R^{36′})ₜ—(OCR³⁹R^{39′}CR³⁹R^{39′})ₚ-NR³⁷—, —(CR³⁹R^{39′}CR³⁹R^{39′}O)ᵣ—(CR³⁶R^{36′})ₜC(O)—, —C(O)(CR³⁶R^{36′})ₜ(OCR³⁹R^{39′}CR³⁹R^{39′}CR³⁹R^{39′})ᵣ—, —C(O)(CR³⁶R^{36′})ₜ(OCR³⁹R^{39′}CR³⁹R^{39′}CR³⁹R^{39′})ᵣNR³⁷—, —C(O)(CR³⁶R^{36′})ᵣ—O—(C₆-C₁₀ aryl)- (CR^{36″}R^{36′″})ₜNR³⁷—, —NR³⁷(CR³⁶R^{36′})ᵣ—(C₆-C₁₀ aryl)-O—(CR^{36″}R^{36′″})ₜC(O)—, —C(O)—(CR³⁶R^{36′})ᵣ—, —NR³⁷—C(O)—(C₆-C₁₀ aryl)-NR^{37′}—, —NR³⁷—(C₆-C₁₀ aryl)-C(O)— NR^{37′}—(CR³⁶R^{36′})ᵣ—C(O)—, —NR³⁷(CR³⁶R^{36′})ᵣ—(C₆-C₁₀ aryl)-O—(CR^{36″}R^{36′″})ₜ, —(CR^{36″}R^{36′″})ₜ—O—(C₆-C₁₀ aryl)-(CR³⁶R^{36′})ᵣ—NR³⁷—, —NR³⁷(CR³⁶R^{36′})ᵣ—(C₆-C₁₀ aryl)-O—(CR^{36″}R^{36′″})—NR^{37′}—, or —NR^{37′}—(CR^{36″}R^{36′″})ₜ—O—(C₆-C₁₀ aryl)-(CR³⁶R^{36′})ᵣ—NR³⁷—, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
wherein
each R³⁶, R^{36′}, R^{36″} and R^{36′″} is independently selected from the group consisting of H, C₁-C₆ alkyl, C₂-C₆ alkenyl, C₂-C₆ alkynyl, C₃-C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰R^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR⁴⁰ and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each r independently is 1, 2, 3, 4, 5, 6, 7, 8, 9, or 10;
each rp independently is an integer from 1 to 80;
each t independently is 1, 2, 3, 4, 5, 6, 7, 8, 9, or 10; and
each * represents a covalent bond;
wherein when k is larger than 3, at least 2 of the L^{x} in formula (I) are independently selected from 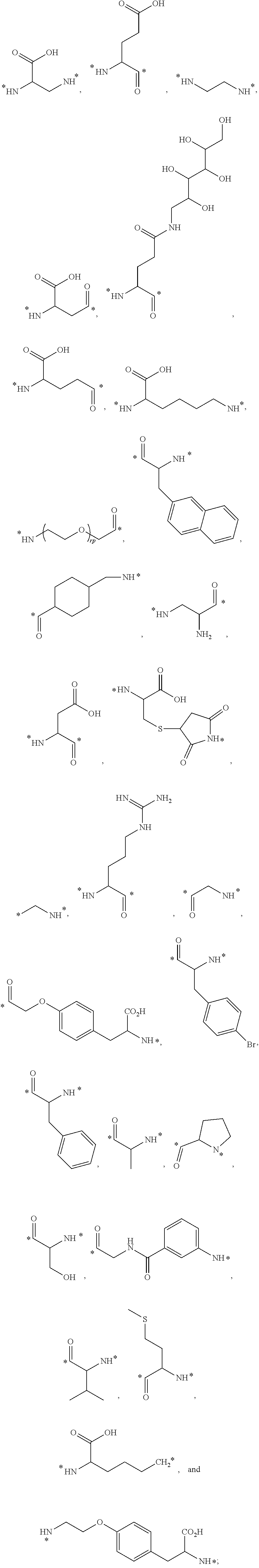
and
provided that the compound is not 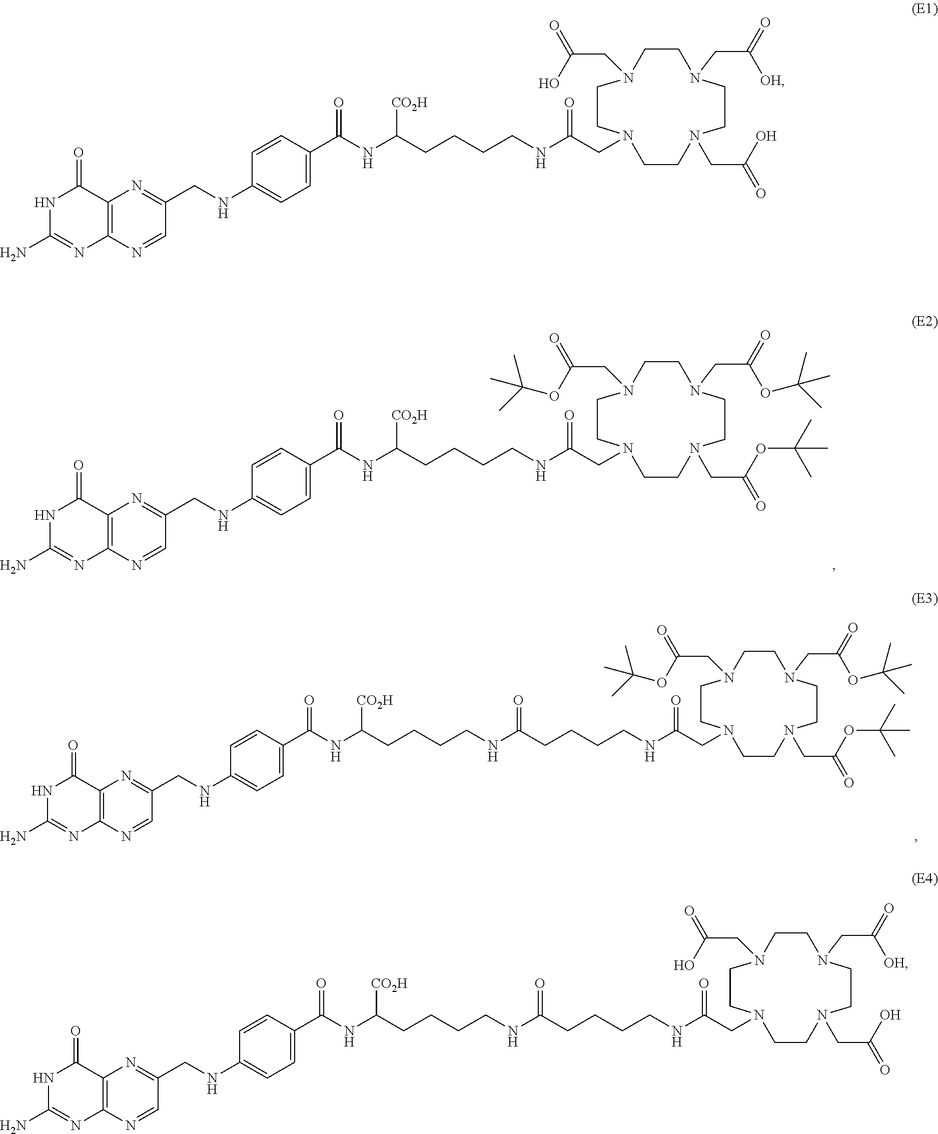 [SMILES: Nc1nc2ncc(CNc3ccc(C(=O)NC(CCCCNC(=O)CN4CCN(CC(=O)O)CCN(CC(=O)O)CCN(CC(=O)O)CC4)C(=O)O)cc3)nc2c(=O)[nH]1 ; CC(C)(C)OC(=O)CN1CCN(CC(=O)NCCCCC(NC(=O)c2ccc(NCc3cnc4nc(N)[nH]c(=O)c4n3)cc2)C(=O)O)CCN(CC(=O)OC(C)(C)C)CCN(CC(=O)OC(C)(C)C)CC1 ; CC(C)(C)OC(=O)CN1CCN(CC(=O)NCCCCC(=O)NCCCCC(NC(=O)c2ccc(NCc3cnc4nc(N)[nH]c(=O)c4n3)cc2)C(=O)O)CCN(CC(=O)OC(C)(C)C)CCN(CC(=O)OC(C)(C)C)CC1 ; Nc1nc2ncc(CNc3ccc(C(=O)NC(CCCCNC(=O)CCCCNC(=O)CN4CCN(CC(=O)O)CCN(CC(=O)O)CCN(CC(=O)O)CC4)C(=O)O)cc3)nc2c(=O)[nH]1] 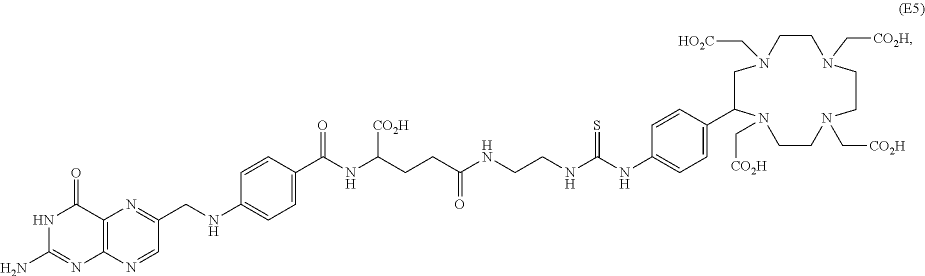 [SMILES: Nc1nc2ncc(CNc3ccc(C(=O)NC(CCC(=O)NCCNC(=S)Nc4ccc(C5CN(CC(=O)O)CCN(CC(=O)O)CCN(CC(=O)O)CCN5CC(=O)O)cc4)C(=O)O)cc3)nc2c(=O)[nH]1]
a tautomer of (E1)-(E5), a compound of (E1)-(E5) in which a metal or radioelement is chelated, or a pharmaceutical salt thereof.

2. The compound of claim 1, or a pharmaceutically acceptable salt thereof, wherein each L¹ is independently of the formula 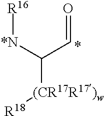
wherein
R¹⁶ is H,
each R¹⁷ and R^{17′} is independently H, C₁-C₆ alkyl, or —C(O)OH,
R¹⁸ is independently H, C₆-C₁₀ aryl, —OH—SH, —NHC(═NH′)NH₂, or —C(O)OH, wherein each hydrogen atom in C₆-C₁₀ aryl is independently optionally substituted by halogen;
each L² is independently of the formula 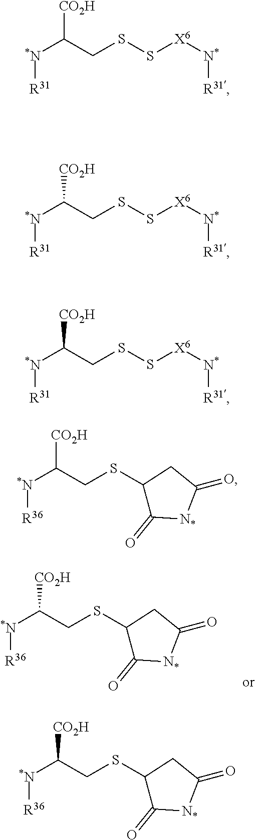
wherein
R³¹ and R^{31′} are H,
R³⁶ is H; and
each L³ is independently —C(O)C₃-C₆ cycloalkylene-(CH₂)ᵣNH—, —(CR³⁹R^{39′})ᵣC(O)—, —C(O)(CR³⁹R^{39′})ᵣ—, —NH(CR³⁹R^{39′})ᵣ—, —(CR³⁹R^{39′})ᵣNH—, —NH(CR³⁹R^{39′})ᵣNH—, —NH(CH₂CH₂O)ᵣₚ—(CR³⁶R^{36′})ₜC(O)—, —C(O)(CR³⁶R^{36′})ₜ-(OCR³⁹R^{39′}CR³⁹R^{39′})ᵣₚ—NH—, —C(O)(CR³⁶R^{36′})ᵣ—O—(C₆-C₁₀ aryl)-(CR^{36″}R^{36′″})ₜNH—, —NH(CR³⁶R^{36′})ᵣ—(C₆-C₁₀ aryl)-O—(CR^{36′}R^{36′″})ₜC(O)—, —C(O)—(CR³⁶R^{36′})ᵣ-NH—C(O)—(C₆-C₁₀ aryl)-NH—, —NR³⁷—(C₆-C₁₀ aryl)-C(O)—NH—(CR³⁶R^{36′})ᵣ—C(O)—, —NH(CR³⁶R^{36′})ᵣ—(C₆-C₁₀ aryl)-O—(CR^{36″}R^{36″})ₜ—, —(CR^{36″}R^{36′″})ₜ—O—(C₆-C₁₀ aryl)-(CR³⁶R^{36′})ᵣ—NH—, —NH(CR³⁶R^{36′})ᵣ—(C₆-C₁₀ aryl)-O—(CR³⁶R^{36′″})ₜ—NH—, or —NH—(CR^{36″}R^{36′″})ₜ—O—(C₆-C₁₀ aryl)-(CR³⁶R^{36′})ᵣ—NH—;
wherein
each R³⁶, R^{36′}, R^{36′}, R^{36′″}, R³⁹ and R^{39′} is independently H or —COOH;
each r independently is 1, 2, 3, 4, 5; and
each t independently is 1, 2, 3, 4, 5.

3. The compound of claim 1 or 2, or a pharmaceutically acceptable salt thereof, wherein BL-(L^{x})ₖ-Ch is BL-L³-Ch, BL-L¹-L³-Ch, BL-L³-L³-L¹-L¹-L¹-L³-Ch, BL-L³-L¹-Ch, BL-L³-L³-L³-Ch, BL-L³-L³-L¹-L³-L³-Ch, BL-L³-L¹-L³-Ch, BL-L³-L³-AA-L¹-L²-L³-Ch, BL-L³-L³-L¹-L¹-L¹-L²-Ch, BL-L³-L³-L³-L¹-AA-Ch, BL-L³-L³-AA-Ch, BL-L³-L³-Ch, BL-L³-L¹-AA-Ch, BL-L³-L³-L³-L¹-Ch, BL-L³-L³-L³-L¹-L¹-Ch, BL-L³-L¹-L¹-L-L¹-AA-AA-AA-AA-Ch, BL-L³-AA-Ch, BL-L³-L¹-L¹-L¹-AA-AA-AA-AA-Ch, BL-L³-L³-L³-AA-Ch, or BL-L³-L³-L³-L³-Ch, wherein each AA independently is an amino acid residue.

4. The compound of any one of claims 1 to 3, or a pharmaceutically acceptable salt thereof, wherein BL comprises one amino acid residue covalently attached to a pteryl group or derivative thereof and BL-(L^{x})ₖ-Ch is BL-L³-Ch, BL-L¹-L¹-L¹-L³-Ch, BL-L¹-Ch, BL-L³-L³-Ch, BL-L¹-L³-L³-Ch, BL-L¹-L³-Ch, BL-L³-L³-AA-L¹-L²-L³-Ch, BL-L¹-L¹-L¹-L²-Ch, BL-L³-L³-L¹-AA-Ch, BL-L³-AA-Ch, BL-L¹-AA-Ch, BL-L³-L³-L¹-Ch, BL-L³-L¹-Ch, BL-L³-L³-L¹-L¹-Ch, BL-L³-L¹-L¹-Ch, BL-L¹-L¹-L¹-L¹-AA-AA-AA-AA-Ch, BL-AA-Ch, BL-L¹-L¹-L¹-AA-AA-AA-AA-Ch, or BL-L¹-L¹-L¹-L¹-L²-L³-L¹-L¹-Ch, wherein each AA independently is an amino acid residue.

5. The compound of any one of claims 1 to 4, or a pharmaceutically acceptable salt thereof, wherein when k is larger than 4, at least 3 of the L^{x} in formula (I) are independently selected from

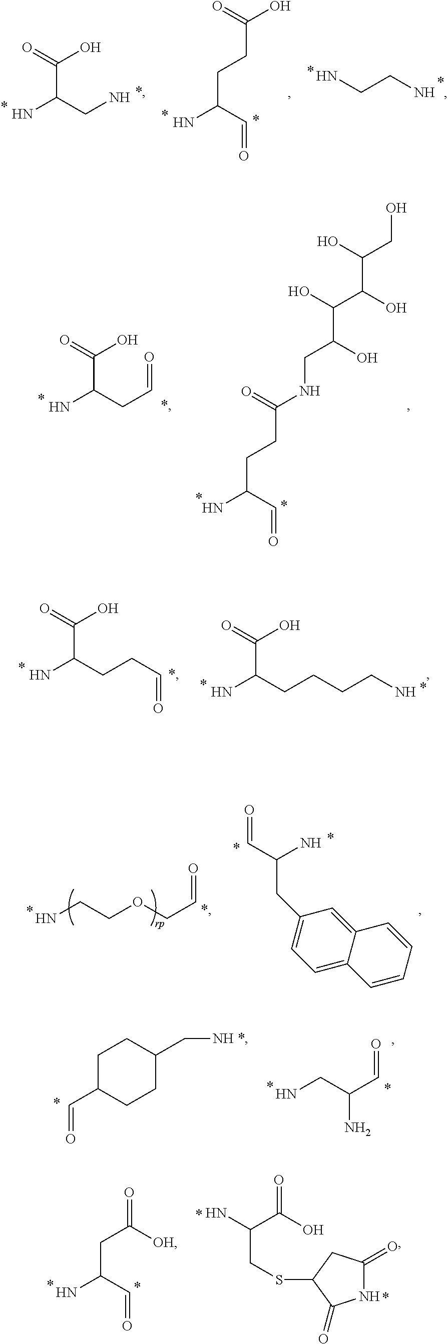

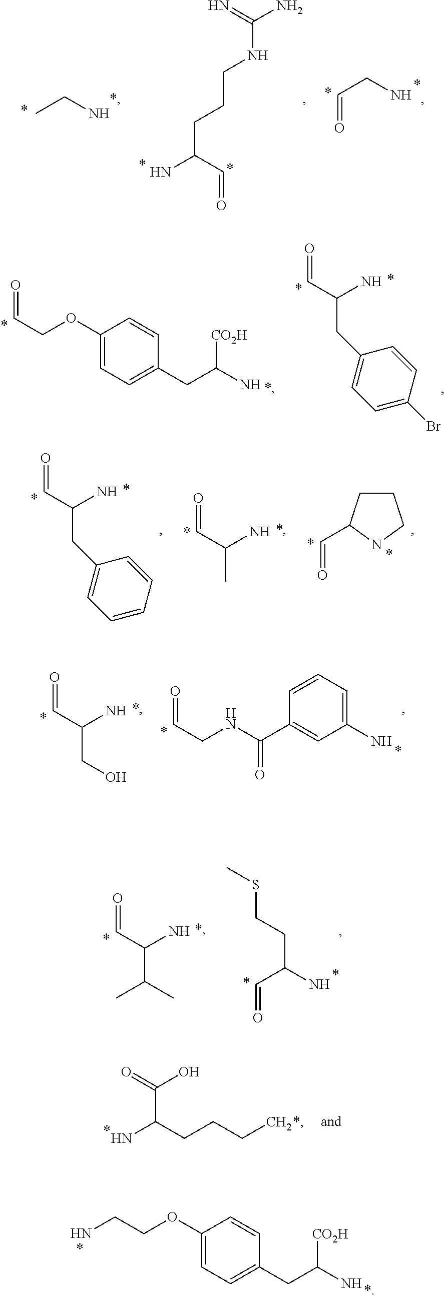

6. The compound of any one of claims 1 to 4, or a pharmaceutically acceptable salt thereof, wherein when k is larger than 4, at least 3 of the L^{x} in formula (I) are independently

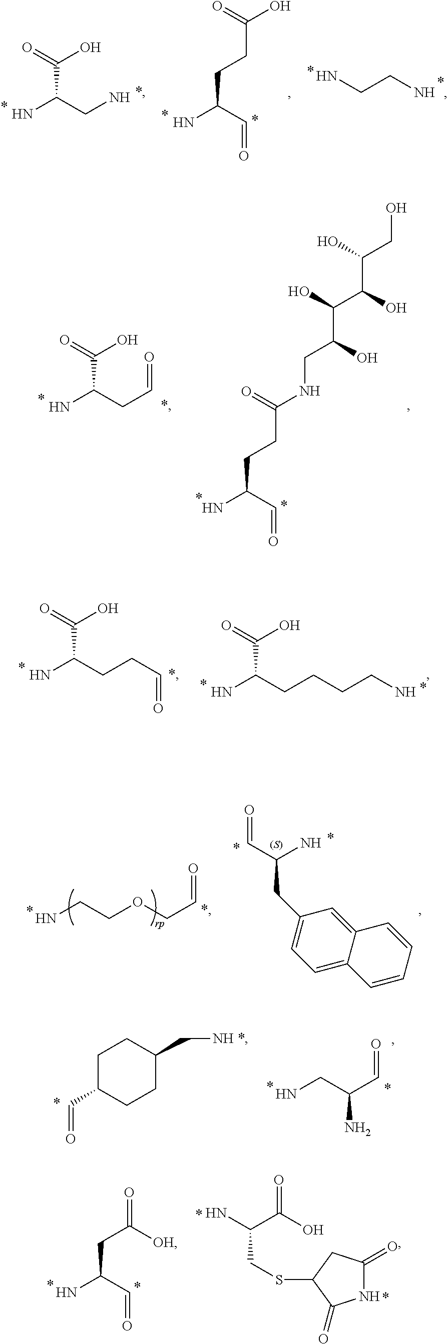

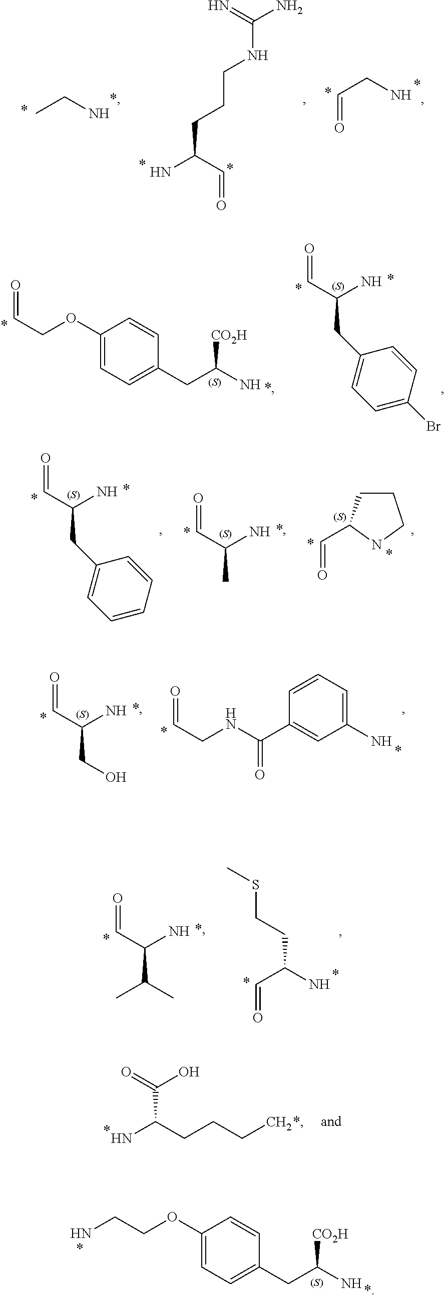

7. The compound of any one of claims 1 to 6, or a pharmaceutically acceptable salt thereof, wherein at least one L^{x} is

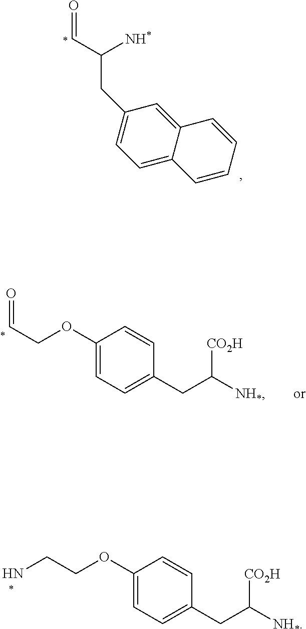

8. The compound of any one of claims 1 to 6, or a pharmaceutically acceptable salt thereof, wherein BL-(L^{x})ₖ-Ch is of the formula BL-L^{x}-L^{a}-L^{x}-Ch, BL-L^{x}-L^{x}-L^{a}-L^{x}-Ch, BL-L^{x}-L^{x}-L^{a}-Ch, or BL-L^{x}-L^{x}-L^{a}-La-Ch, wherein L^{a} is 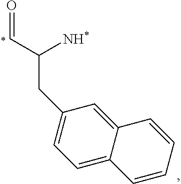
and each L^{x} independently is AA, L¹, or L³.

9. The compound of any one of claims 1 to 6, or a pharmaceutically acceptable salt thereof, wherein BL-(L^{x})ₖ-Ch is of the formula BL-L^{a}-L^{x}-Ch, BL-L^{x}-L^{a}-L^{x}-Ch, BL-L^{x}-L^{a}-Ch, or BL-L^{x}-L^{a}-La-Ch, wherein L^{a} is 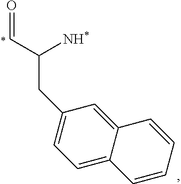
and each L^{x} independently is AA, L¹, or L³.

10. The compound of claim 8 or 9, or pharmaceutically acceptable salt thereof, wherein L^{a} is

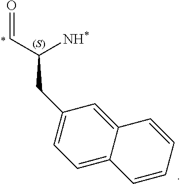

11. The compound of any one of claims 1 to 6, or a pharmaceutically acceptable salt thereof, wherein at least one L^{x} is

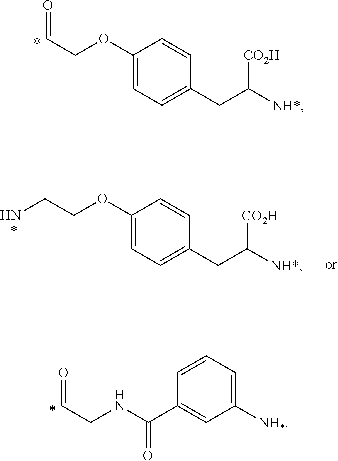

12. The compound of any one of claims 1 to 6, or a pharmaceutically acceptable salt thereof, wherein at least one L^{x} is

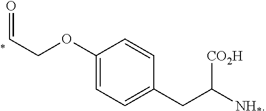

13. The compound of any one of claims 1 to 6, or a pharmaceutically acceptable salt thereof, wherein BL-(L^{x})ₖ-Ch is of the formula BL-L^{x}-L^{b}-L^{x}-Ch, BL-L^{x}-L^{b}-Ch, or BL-L^{x}-L^{b}-L^{b}-Ch, wherein L^{b} is 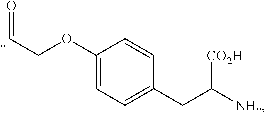
and each L^{x} independently is AA, L¹, or L³.

14. The compound of any one of claims 1 to 6, or a pharmaceutically acceptable salt thereof, wherein BL-(L^{x})ₖ-Ch is of the formula BL-L^{b}-L^{x}-Ch, BL-L^{b}-Ch, or BL-L^{b}-L^{b}-Ch, wherein L^{b} is 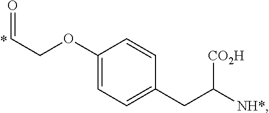
and each L^{x} independently is AA, L¹, or L³.

15. The compound of claim 13 or 14, or pharmaceutically acceptable salt thereof, wherein L^{b} is

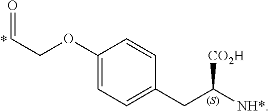

16. The compound of any one of the preceding claims, or a pharmaceutically acceptable salt thereof, wherein BL comprises a pteryl group or a derivative thereof.

17. The compound of any one of the preceding claims, or a pharmaceutically acceptable salt thereof, wherein BL is of the formula 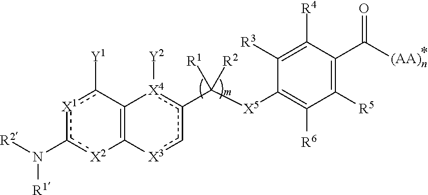
wherein
AA is an amino acid residue;
R¹ and R² in each instance are independently selected from the group consisting of H, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂-C₆ alkynyl;
X¹ is —N(R¹¹)—, ═N—, —N═, —C(R¹¹)═ or ═C(R¹¹)—;
X² is —N(R^{11′})— or ═N—;
X³ is —N(R^{11′})—, —N═ or —C(R^{11′})═;
X⁴ is —N═ or —C═;
X⁵ is —N(R¹²)— or —C(R¹²)(R^{12′})—;
Y¹ is H, —OR¹³ or —SR¹³ when X¹ is —N═ or —C(R¹¹)═, or Y¹ is ═O when X¹ is —NR¹¹—, ═N— or ═C(R¹¹)—;
Y² is H, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;
R^{1′}, R^{2′}, R¹¹, R^{11′}, R^{11″}, R¹², R^{12′}, R¹³, R¹⁴ and R^{14′} are each independently selected from the group consisting of H, C₁-C₆ alkyl, C₂-C₆ alkenyl, C₂-C₆ alkynyl, —C(O)R¹⁵, —C(O)OR¹⁵ and —C(O)NR¹⁵R^{15′};
R¹⁵ and R^{15′} are each independently H, or C₁-C₆ alkyl;
m is 1, 2, 3 or 4; and
n is 0 or 1;
wherein * represents a covalent bond to the rest of the compound.

18. The compound of any one of claims 1 to 17, or a pharmaceutically acceptable salt thereof, wherein m is 1.

19. The compound of any one of claims 1 to 18, or a pharmaceutically acceptable salt thereof, wherein X¹ is —NR¹¹—.

20. The compound of any one of claims 1 to 19, or a pharmaceutically acceptable salt thereof, wherein X² is ═N—.

21. The compound of any one of claims 1 to 20, or a pharmaceutically acceptable salt thereof, wherein Y¹ is ═O.

22. The compound of any one of claims 1 to 21 or a pharmaceutically acceptable salt thereof, wherein X¹ is —NR¹¹—, and R¹¹ is H.

23. The compound of any one of claims 1 to 22, or a pharmaceutically acceptable salt thereof, wherein X³ is —C(R^{11′})═.

24. The compound of claim 23, or a pharmaceutically acceptable salt thereof, wherein R^{11′} is H.

25. The compound of any one of claims 1 to 22, or a pharmaceutically acceptable salt thereof, wherein X⁴ is —C═.

26. The compound of any one of claims 1 to 23, or a pharmaceutically acceptable salt thereof, wherein Y² is H.

27. The compound of any one of claims 1 to 20, or a pharmaceutically acceptable salt thereof, wherein X³ is —N═.

28. The compound of any one of claims 1 to 22 or 27, or a pharmaceutically acceptable salt thereof, wherein X⁴ is —N═.

29. The compound of any one of claims 1 to 28, or a pharmaceutically acceptable salt thereof, wherein X⁵ is —NR¹²—.

30. The compound of any one of claims 1 to 29, or a pharmaceutically acceptable salt thereof, wherein R¹² is H.

31. The compound of any one of claims 1 to 30, or a pharmaceutically acceptable salt thereof, wherein R^{1′} and R^{2′} are H.

32. The compound of any one of claims 1 to 31, or a pharmaceutically acceptable salt thereof, wherein each R¹ and R² is H.

33. The compound of any one of claims 1 to 32, or a pharmaceutically acceptable salt thereof, wherein R³, R⁴, R⁵ and R⁶ are H.

34. The compound of any one of claims 1 to 33, or a pharmaceutically acceptable salt thereof, wherein n is 1.

35. The compound of any one of claims 1 to 16, or a pharmaceutically acceptable salt thereof, wherein BL is of the formula 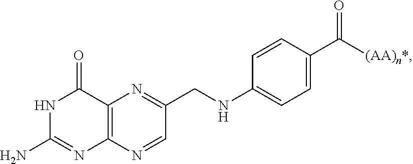
wherein n is 0 or 1, and AA is an amino acid residue.

36. The compound of any one of claims 1 to 17, or pharmaceutically acceptable salt thereof, wherein BL is of formula

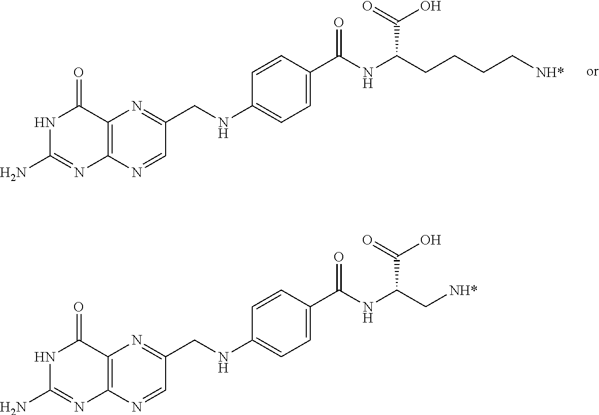

37. The compound of any one of claims 1 to 36, or a pharmaceutically acceptable salt thereof, wherein Ch comprises a radioelement selected from the group consisting of ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

38. The compound of any one of claims 1 to 36, or a pharmaceutically acceptable salt thereof, wherein Ch comprises a radioelement selected from the group consisting of ⁶⁶Ga, ⁶⁷Ga, ⁶⁸Ga, ¹⁷⁷Lu, and ²²⁵Ac.

39. The compound of any one of the preceding claims, or a salt thereof, wherein Ch is selected from the group consisting of 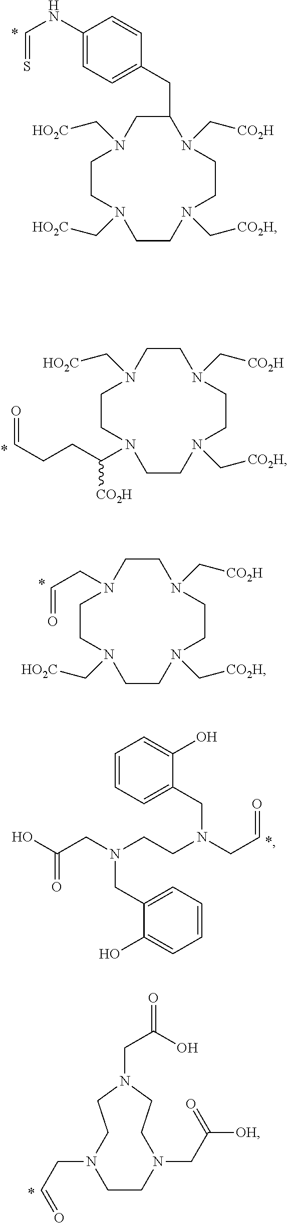 [SMILES: O=C(O)CN1CCN(CC(=O)O)CCN(CC(=O)O)C(Cc2ccc(NC=S)cc2)CN(CC(=O)O)CC1 ; O=CCCC(C(=O)O)N1CCN(CC(=O)O)CCN(CC(=O)O)CCN(CC(=O)O)CC1 ; O=CCN1CCN(CC(=O)O)CCN(CC(=O)O)CCN(CC(=O)O)CC1 ; O=CCN(CCN(CC(=O)O)Cc1ccccc1O)Cc1ccccc1O ; O=CCN1CCN(CC(=O)O)CCN(CC(=O)O)CC1] 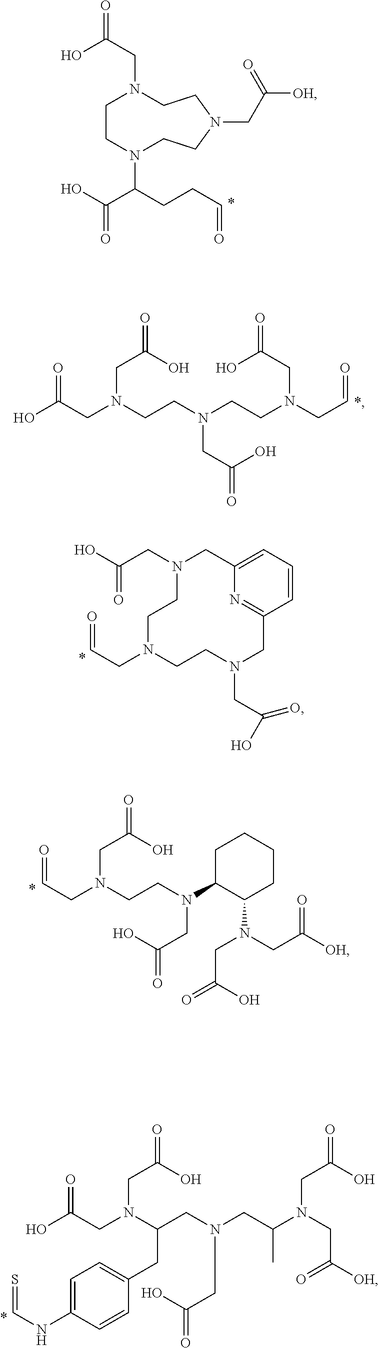 [SMILES: O=CCCC(C(=O)O)N1CCN(CC(=O)O)CCN(CC(=O)O)CC1 ; O=CCN(CCN(CCN(CC(=O)O)CC(=O)O)CC(=O)O)CC(=O)O ; O=CCN1CCN(CC(=O)O)Cc2cccc(n2)CN(CC(=O)O)CC1 ; O=CCN(CCN(CC(=O)O)[C@H]1CCCC[C@@H]1N(CC(=O)O)CC(=O)O)CC(=O)O ; CC(CN(CC(=O)O)CC(Cc1ccc(NC=S)cc1)N(CC(=O)O)CC(=O)O)N(CC(=O)O)CC(=O)O] 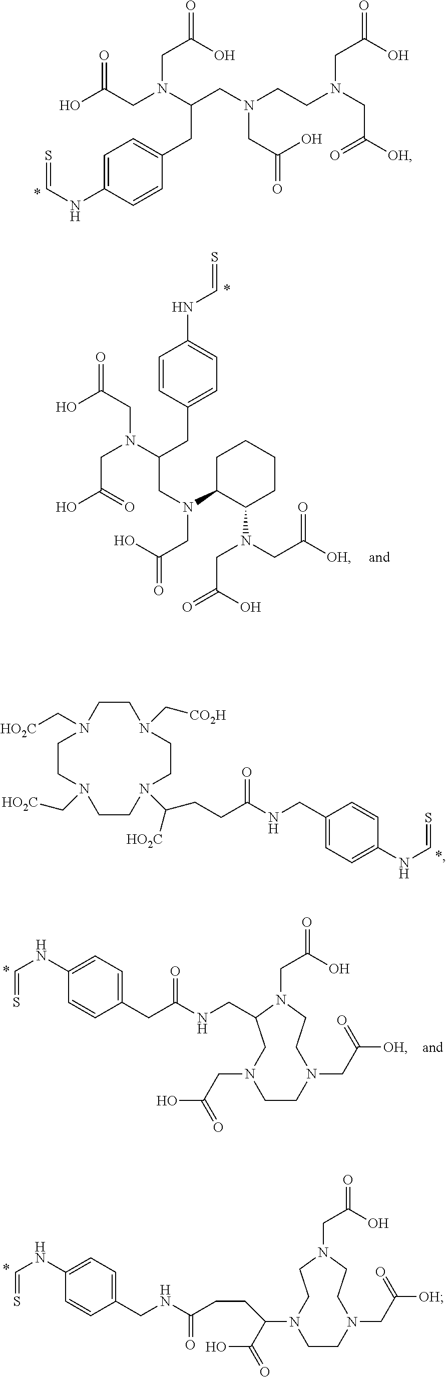 [SMILES: O=C(O)CN(CCN(CC(=O)O)CC(Cc1ccc(NC=S)cc1)N(CC(=O)O)CC(=O)O)CC(=O)O ; O=C(O)CN(CC(=O)O)C(Cc1ccc(NC=S)cc1)CN(CC(=O)O)[C@H]1CCCC[C@@H]1N(CC(=O)O)CC(=O)O ; O=C(O)CN1CCN(CC(=O)O)CCN(C(CCC(=O)NCc2ccc(NC=S)cc2)C(=O)O)CCN(CC(=O)O)CC1 ; O=C(O)CN1CCN(CC(=O)O)CC(CNC(=O)Cc2ccc(NC=S)cc2)N(CC(=O)O)CC1 ; O=C(O)CN1CCN(CC(=O)O)CCN(C(CCC(=O)NCc2ccc(NC=S)cc2)C(=O)O)CC1]
and Ch can comprise a radioelement, Si-¹⁸F, B-¹⁸F, or Al-¹⁸F.

40. The compound of any one of the preceding claims, or a pharmaceutically acceptable salt thereof, wherein Ch is 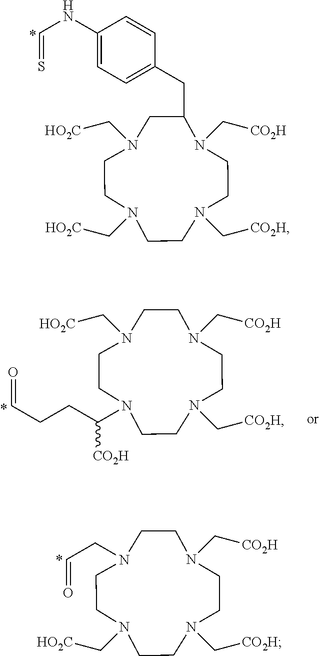 [SMILES: O=C(O)CN1CCN(CC(=O)O)CCN(CC(=O)O)C(Cc2ccc(NC=S)cc2)CN(CC(=O)O)CC1 ; O=CCCC(C(=O)O)N1CCN(CC(=O)O)CCN(CC(=O)O)CCN(CC(=O)O)CC1 ; "or" ; O=CCN1CCN(CC(=O)O)CCN(CC(=O)O)CCN(CC(=O)O)CC1]
and Ch can comprise a radioelement, Si-¹⁸F, B-¹⁸F, or Al-¹⁸F.

41. The compound of any one of claims 1 to 40, wherein BL comprises a pteryl group or a derivative thereof, and the pteryl group or derivative thereof is covalently bonded to a group selected from

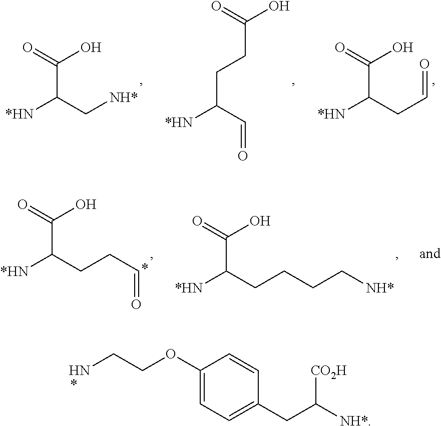

42. The compound of any one of claims 1 to 41, wherein one, two or three L^{x} independently are L¹ in which independently w is 1 or 2, and R¹⁸ is C₆-C₁₀ aryl wherein each hydrogen is optionally substituted by halogen or C₁-C₆ alkyl.

43. The compound of claim 42, wherein one, two or three L^{x} independently are of formula

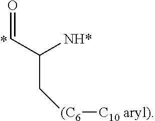

44. The compound of claim 1, or a pharmaceutically acceptable salt thereof, wherein the compound is of any one of formula (C1) to (C32):

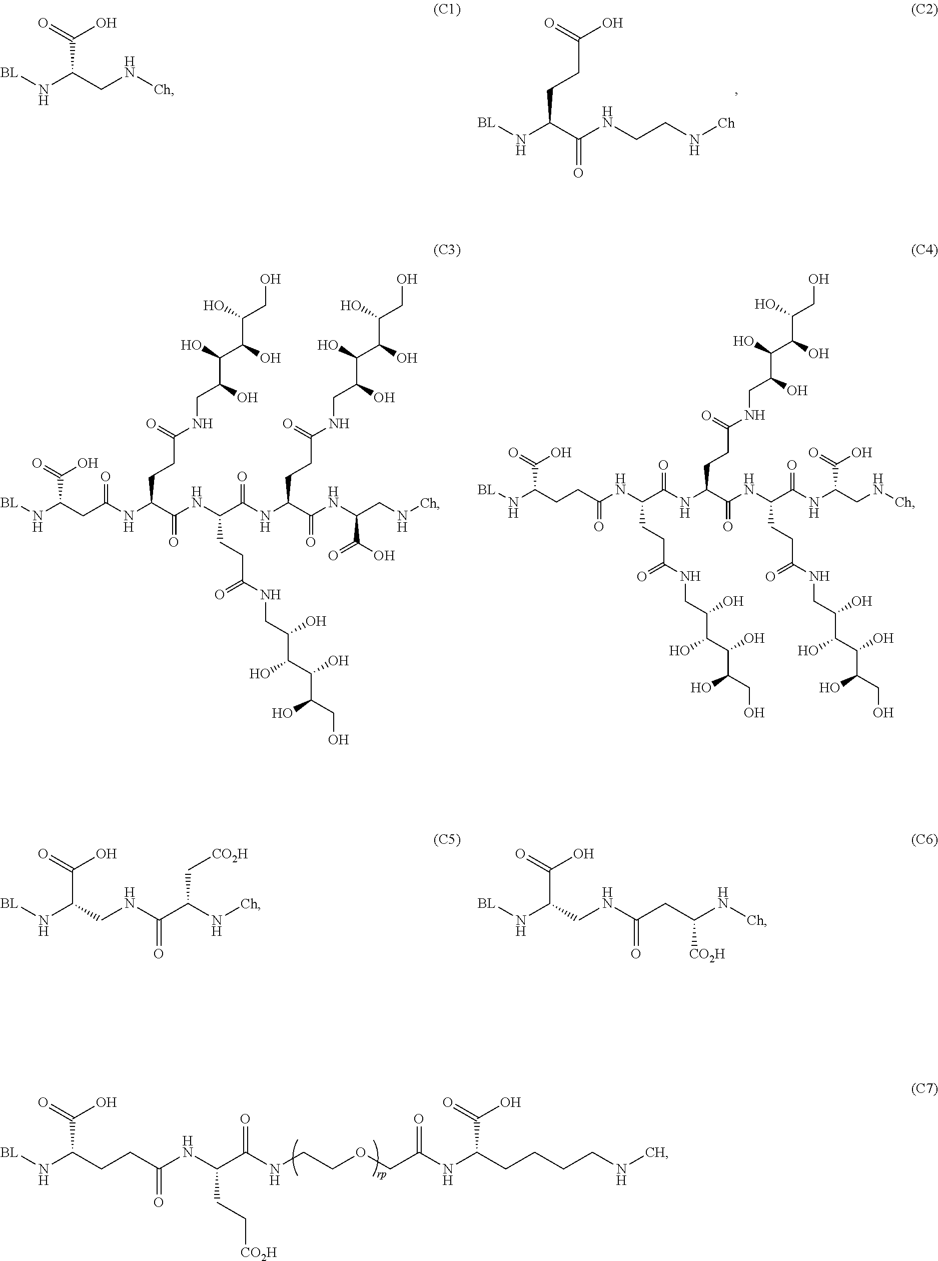

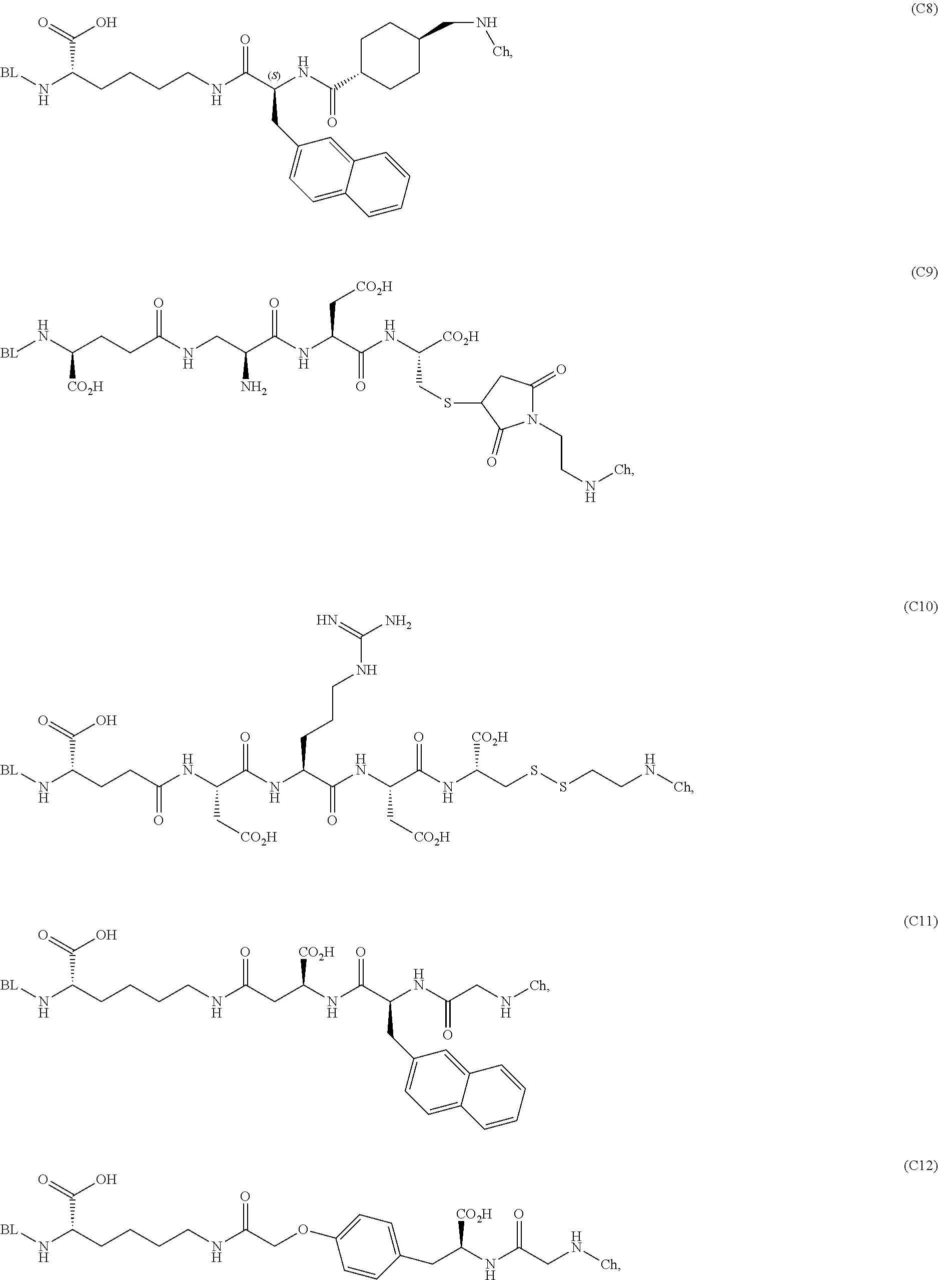

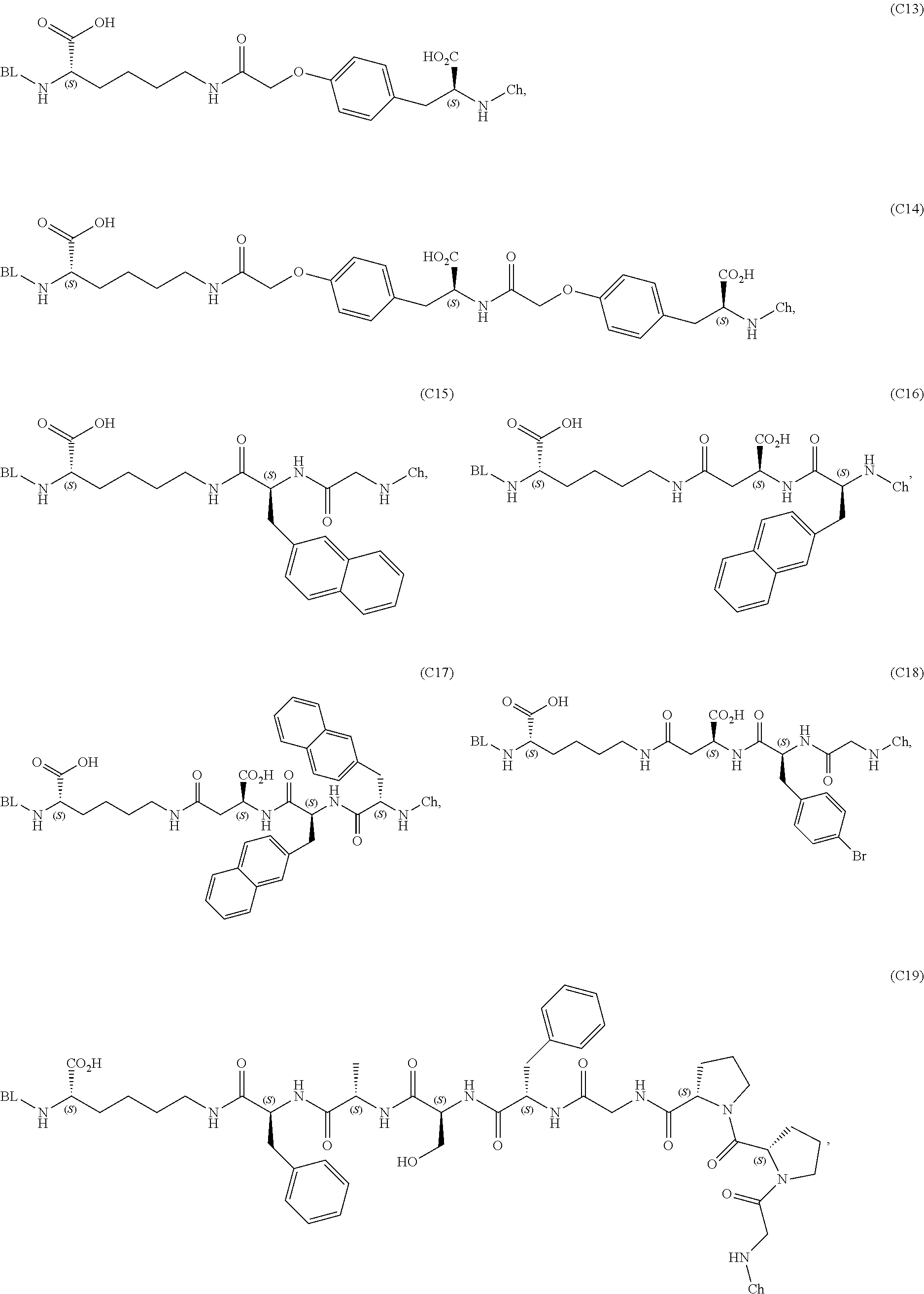

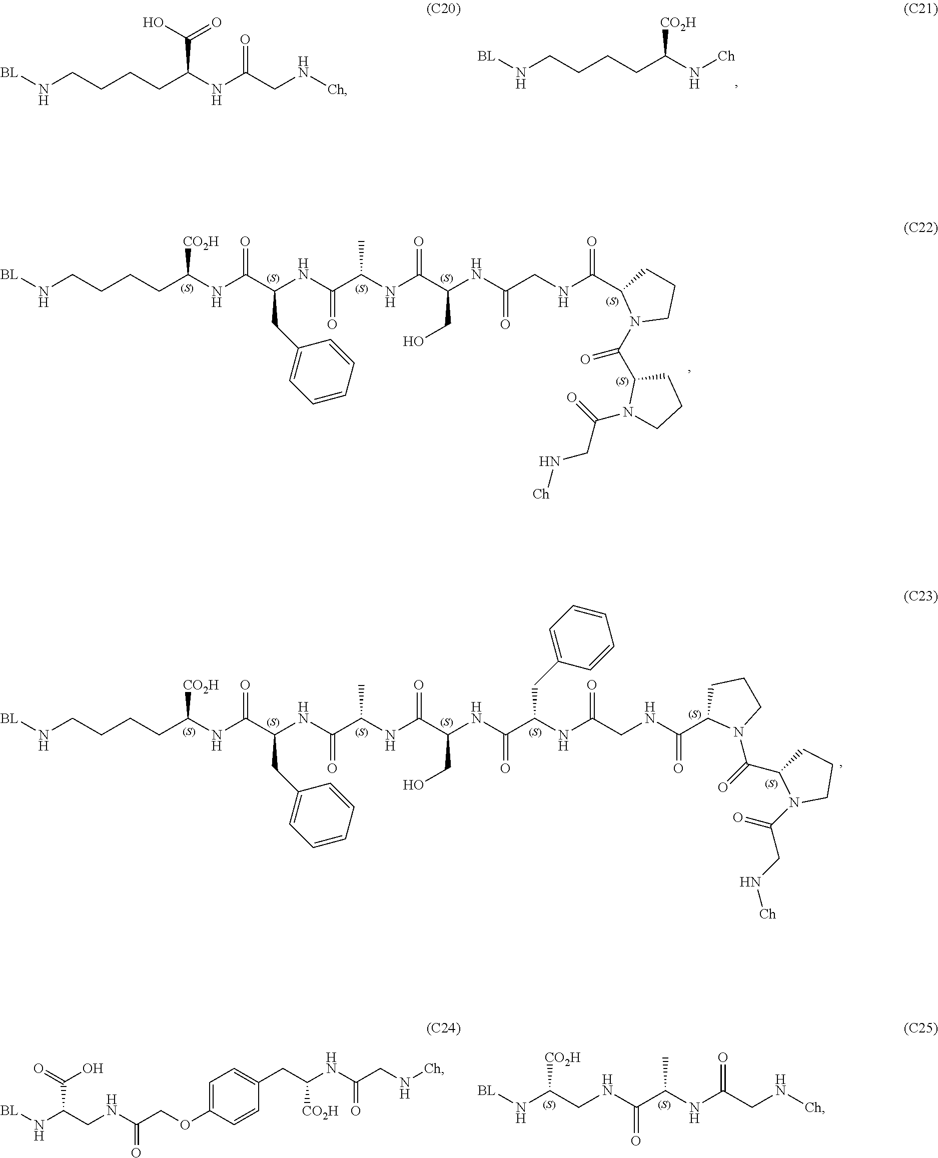

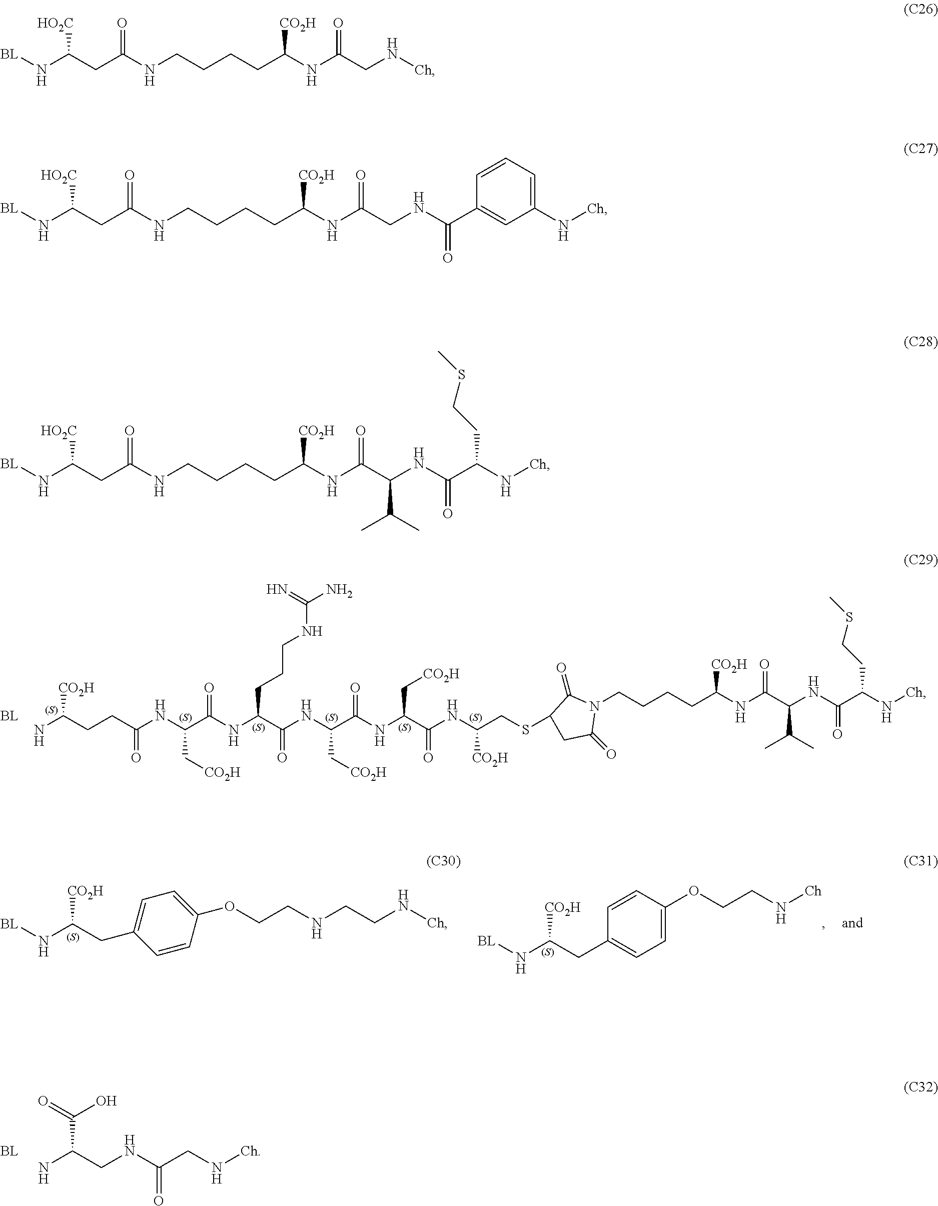

45. The compound of claim 1, wherein the compound is a compound of any one of formula (C1) to (C32), 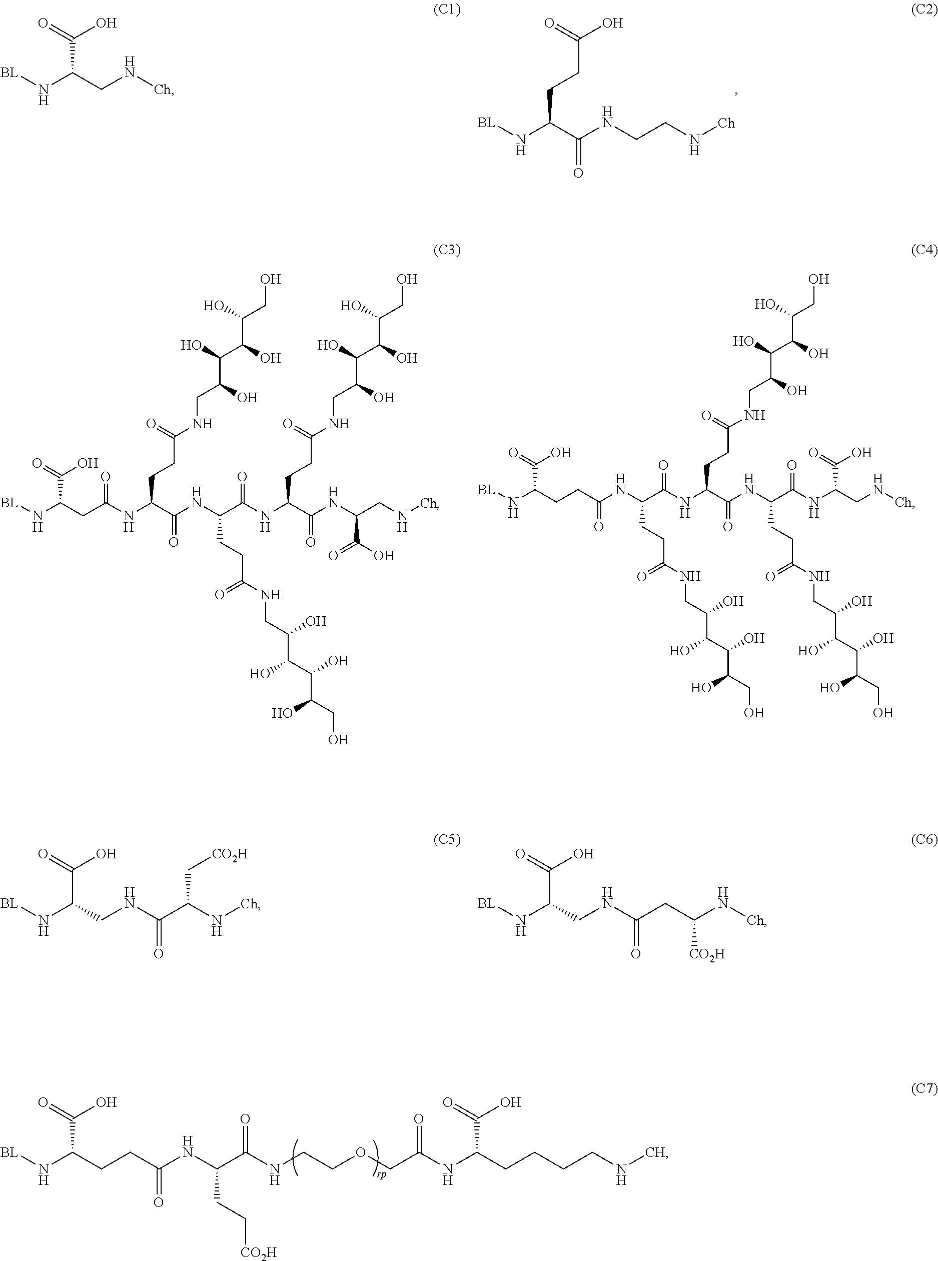 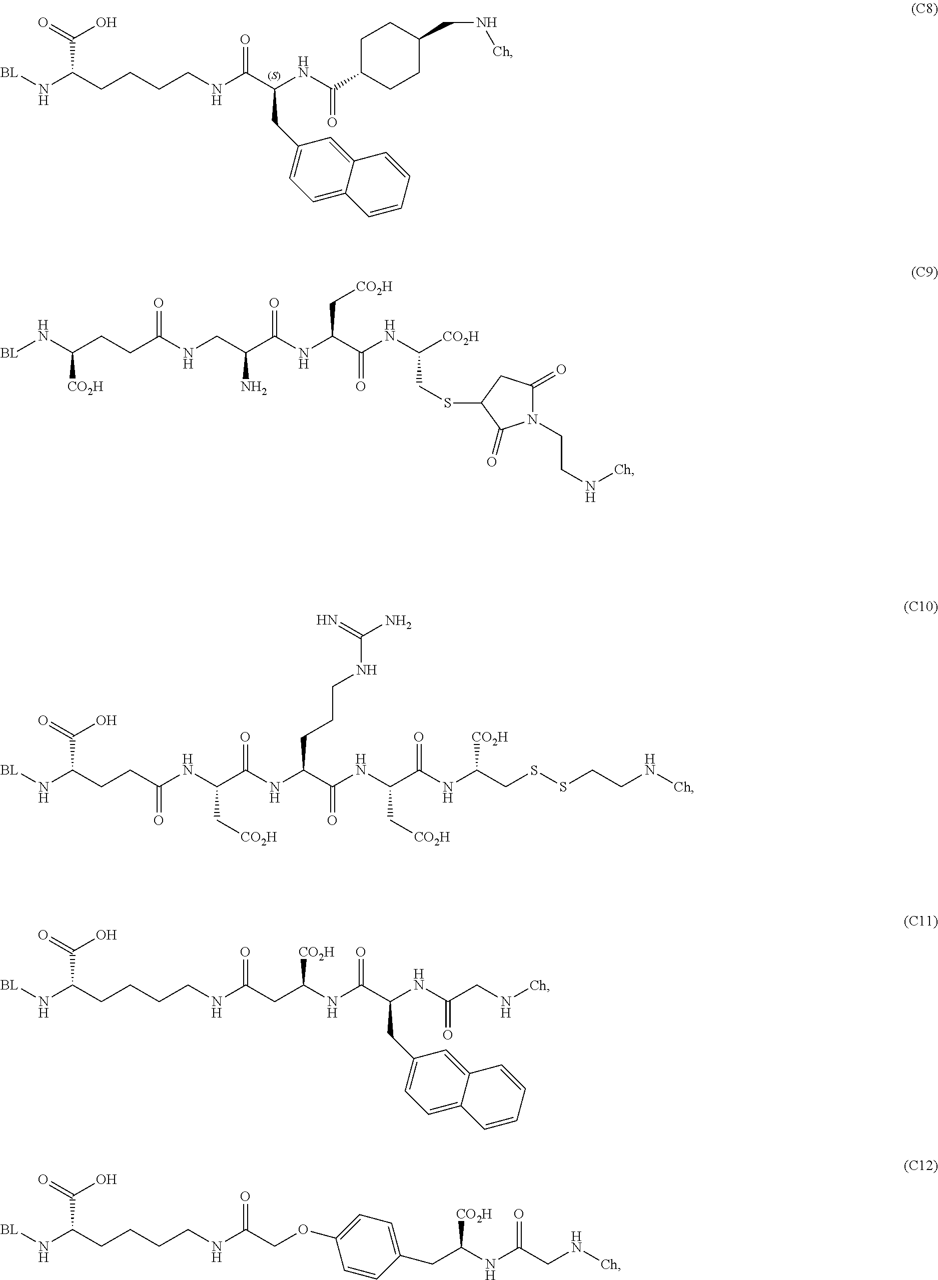 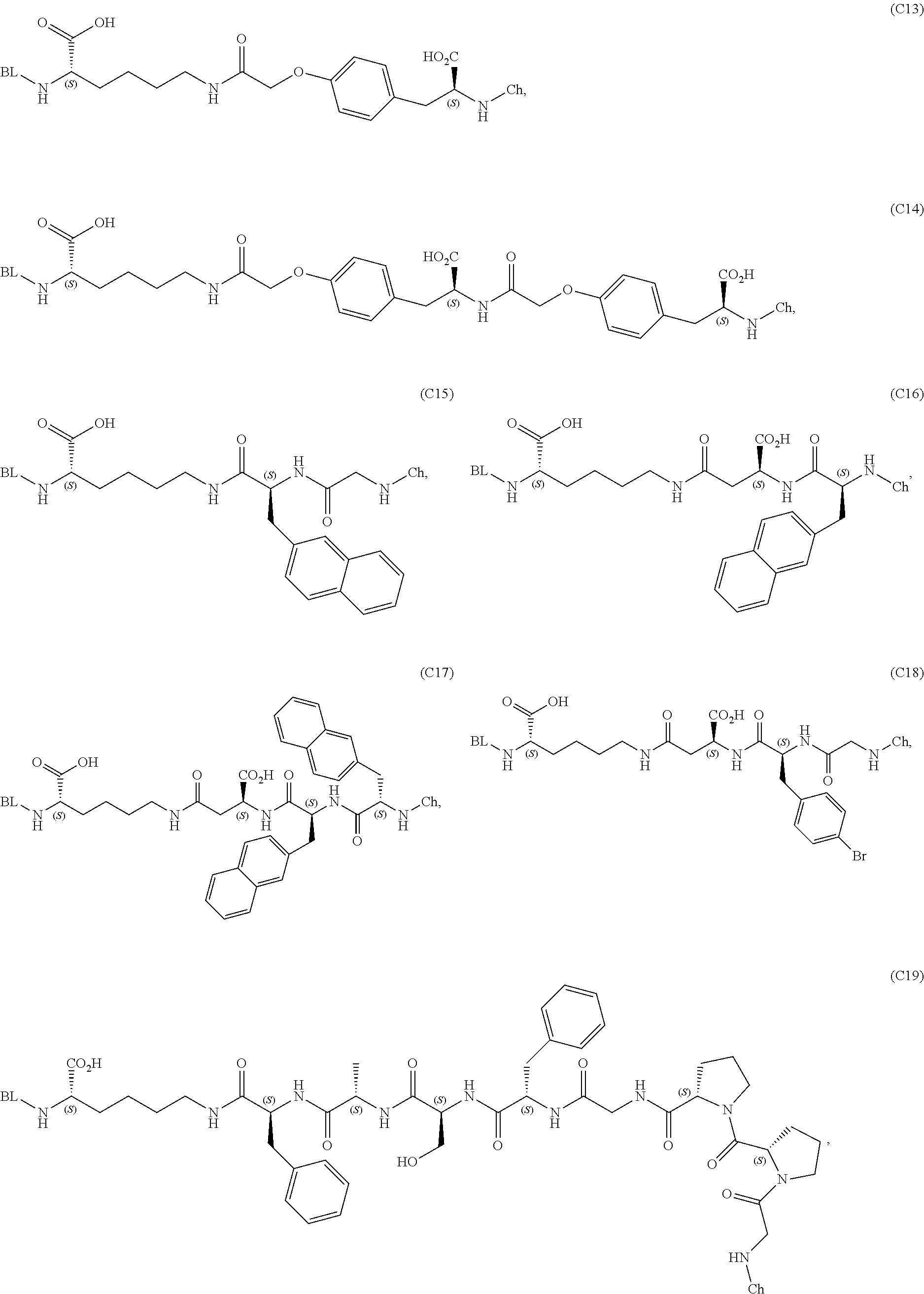 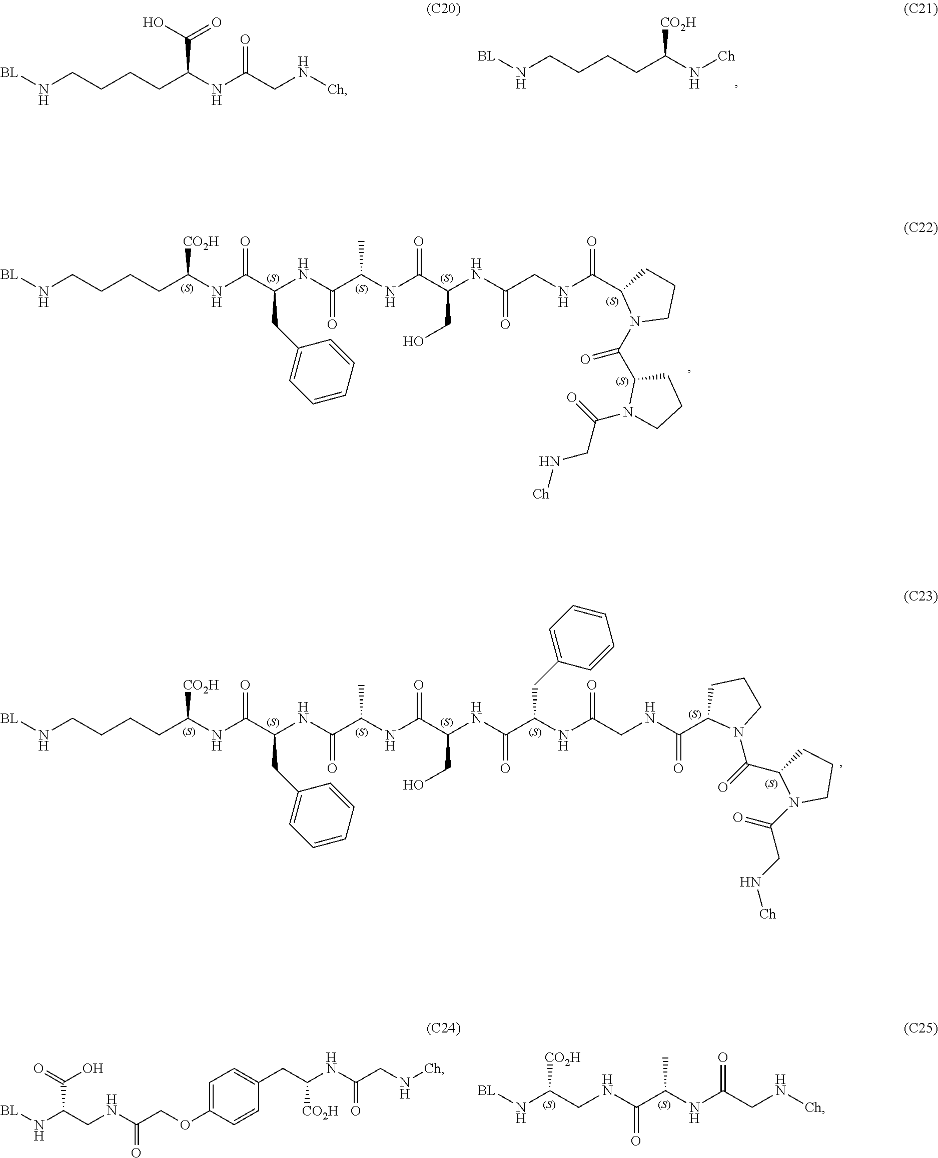 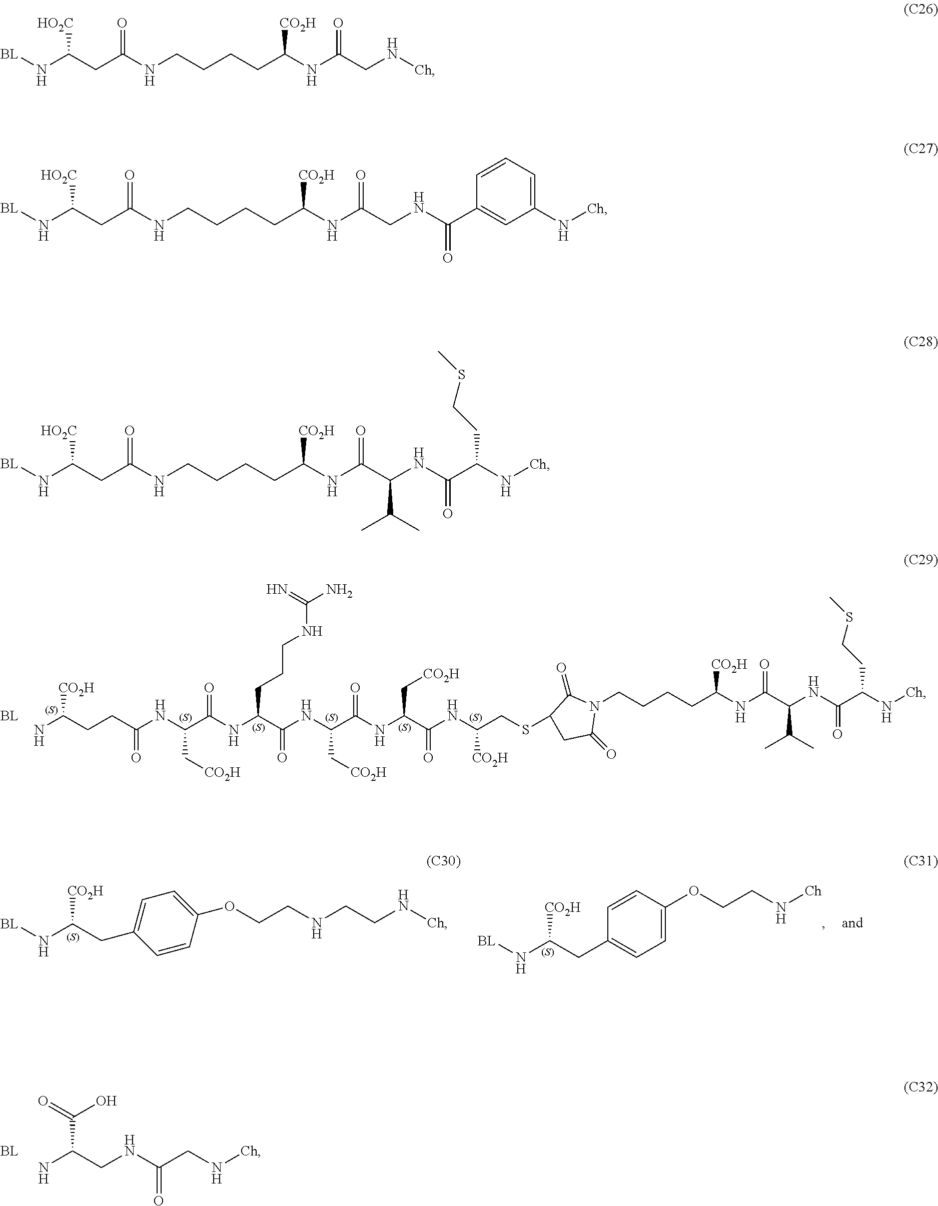
except that one group, corresponding to L^{x}, within said any one of formula (C1) to (C32) is replaced by a different L^{x}.

46. The compound of claim 45, or a pharmaceutically acceptable salt thereof, wherein the one group, which is replaced by a different L^{x}, is an AA group, the different L^{x} is a different AA group, and the different AA group is a conservative amino acid substitution of the AA group.

47. The compound of any one of claims 1 to 43, or a pharmaceutically acceptable salt thereof, wherein -(L^{x})ₖ- comprises a group of formula (III)

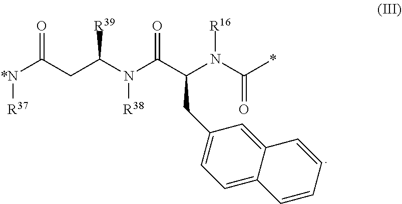

48. The compound of claim 47, wherein R¹⁶, R³⁷ and R³⁸ are H.

49. The compound of claim 47 or 48, wherein R³⁹ is —COOH.

50. The compound of claim 1, wherein the compound is selected from 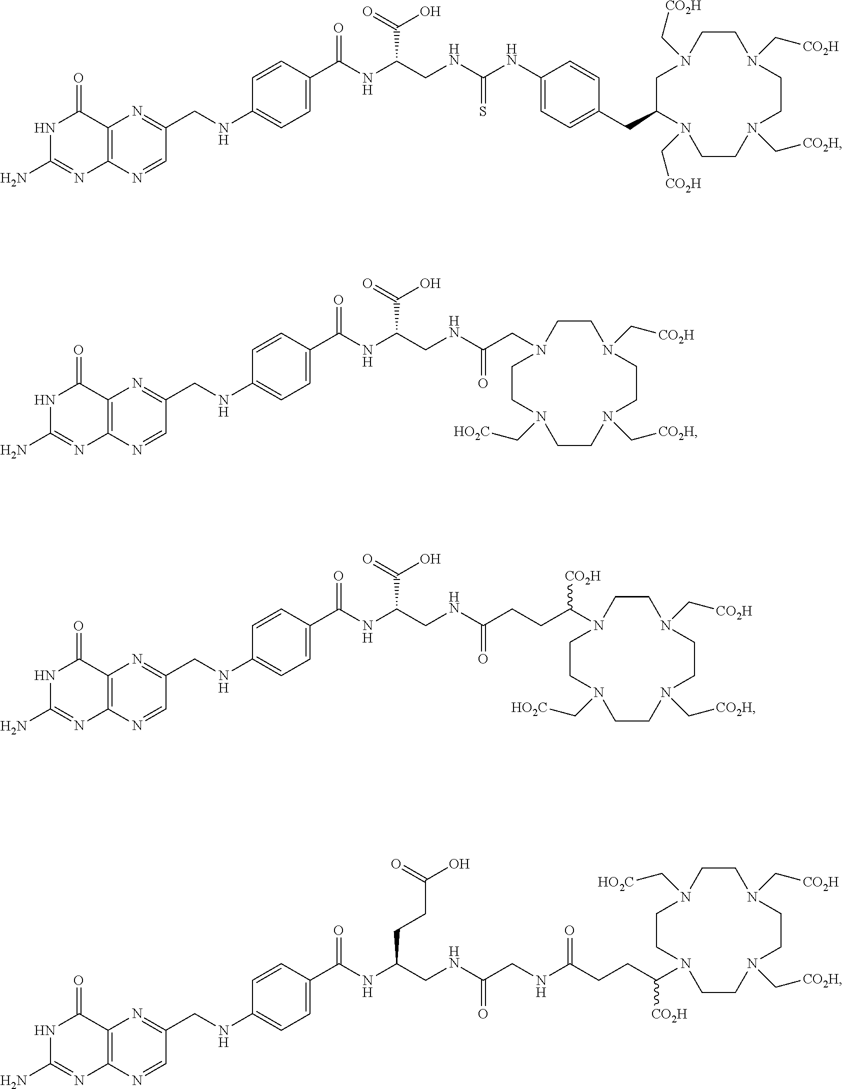 [SMILES: Nc1nc2ncc(CNc3ccc(C(=O)N[C@@H](CNC(=S)Nc4ccc(C[C@H]5CN(CC(=O)O)CCN(CC(=O)O)CCN(CC(=O)O)CCN5CC(=O)O)cc4)C(=O)O)cc3)nc2c(=O)[nH]1 ; Nc1nc2ncc(CNc3ccc(C(=O)N[C@@H](CNC(=O)CN4CCN(CC(=O)O)CCN(CC(=O)O)CCN(CC(=O)O)CC4)C(=O)O)cc3)nc2c(=O)[nH]1 ; Nc1nc2ncc(CNc3ccc(C(=O)N[C@@H](CNC(=O)CCC(C(=O)O)N4CCN(CC(=O)O)CCN(CC(=O)O)CCN(CC(=O)O)CC4)C(=O)O)cc3)nc2c(=O)[nH]1 ; Nc1nc2ncc(CNc3ccc(C(=O)N[C@@H](CCC(=O)O)CNC(=O)CNC(=O)CCC(C(=O)O)N4CCN(CC(=O)O)CCN(CC(=O)O)CCN(CC(=O)O)CC4)cc3)nc2c(=O)[nH]1] 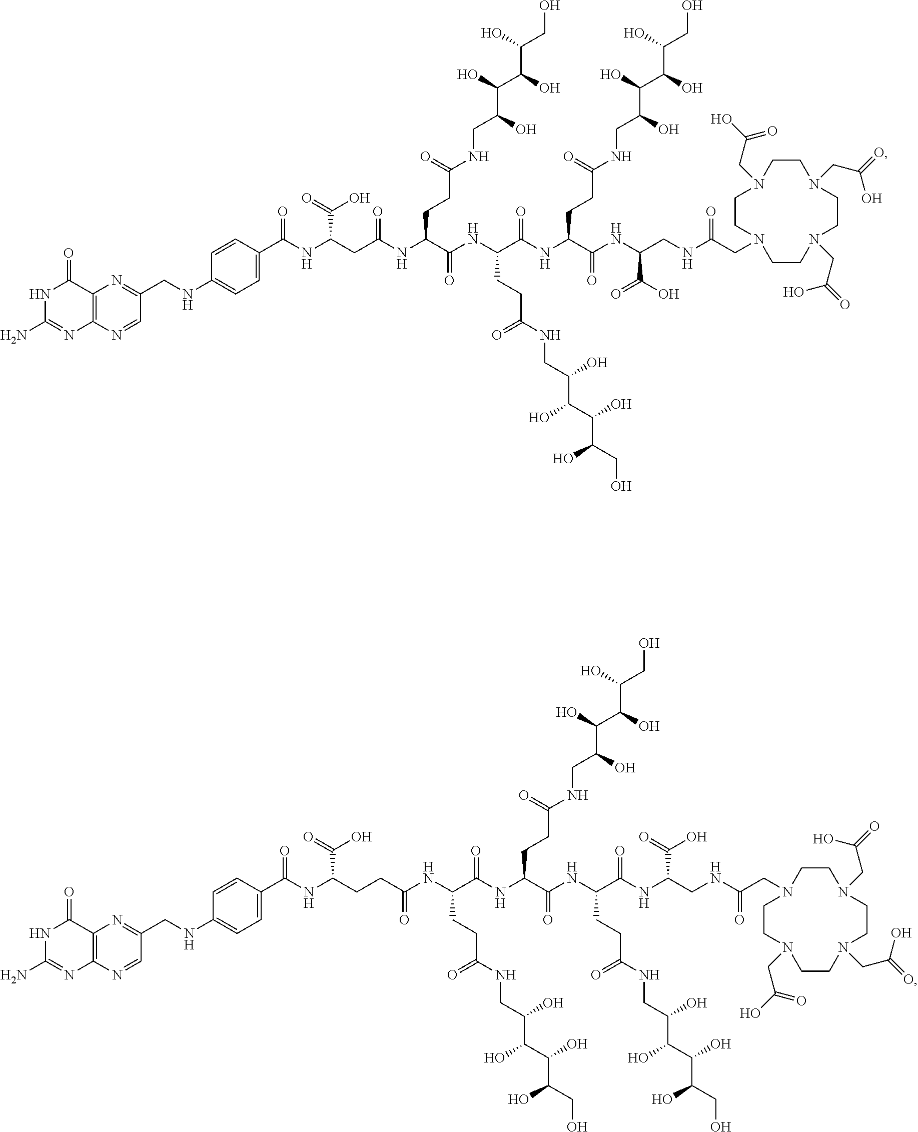 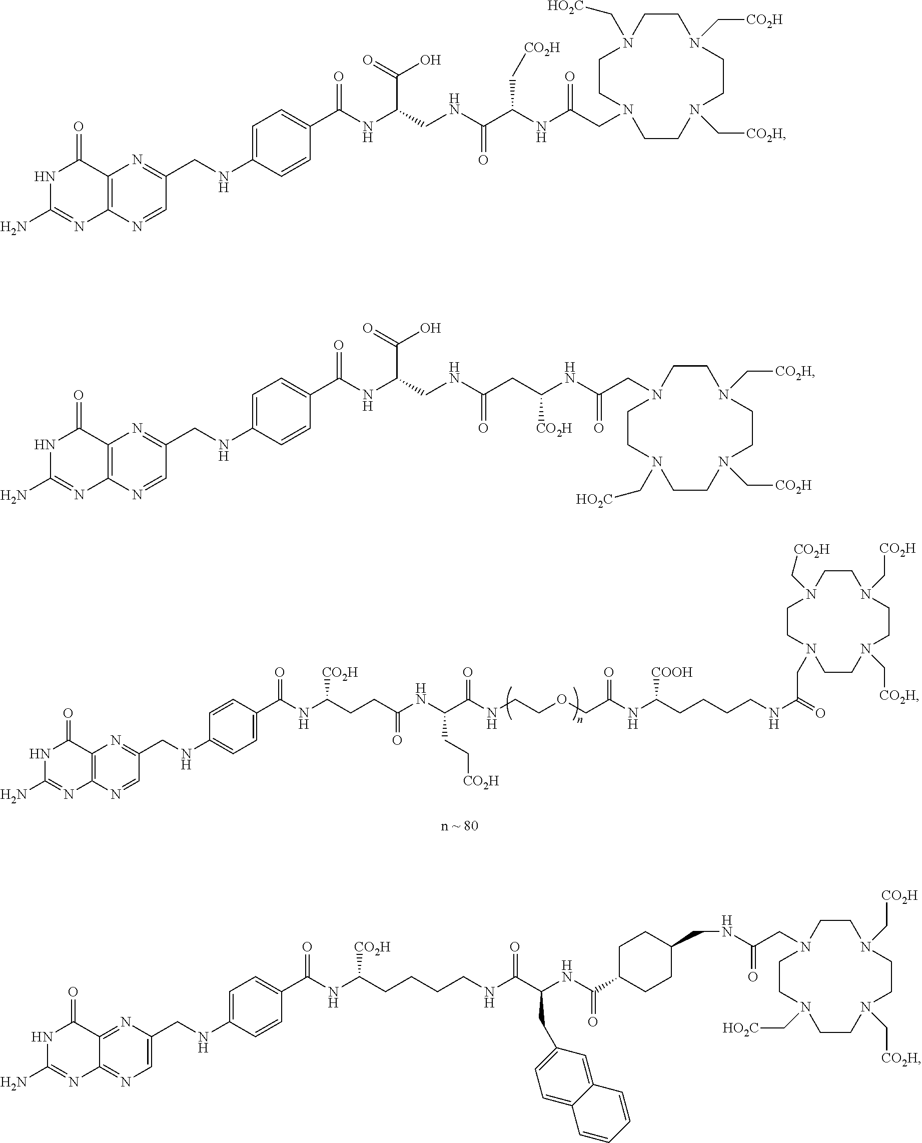 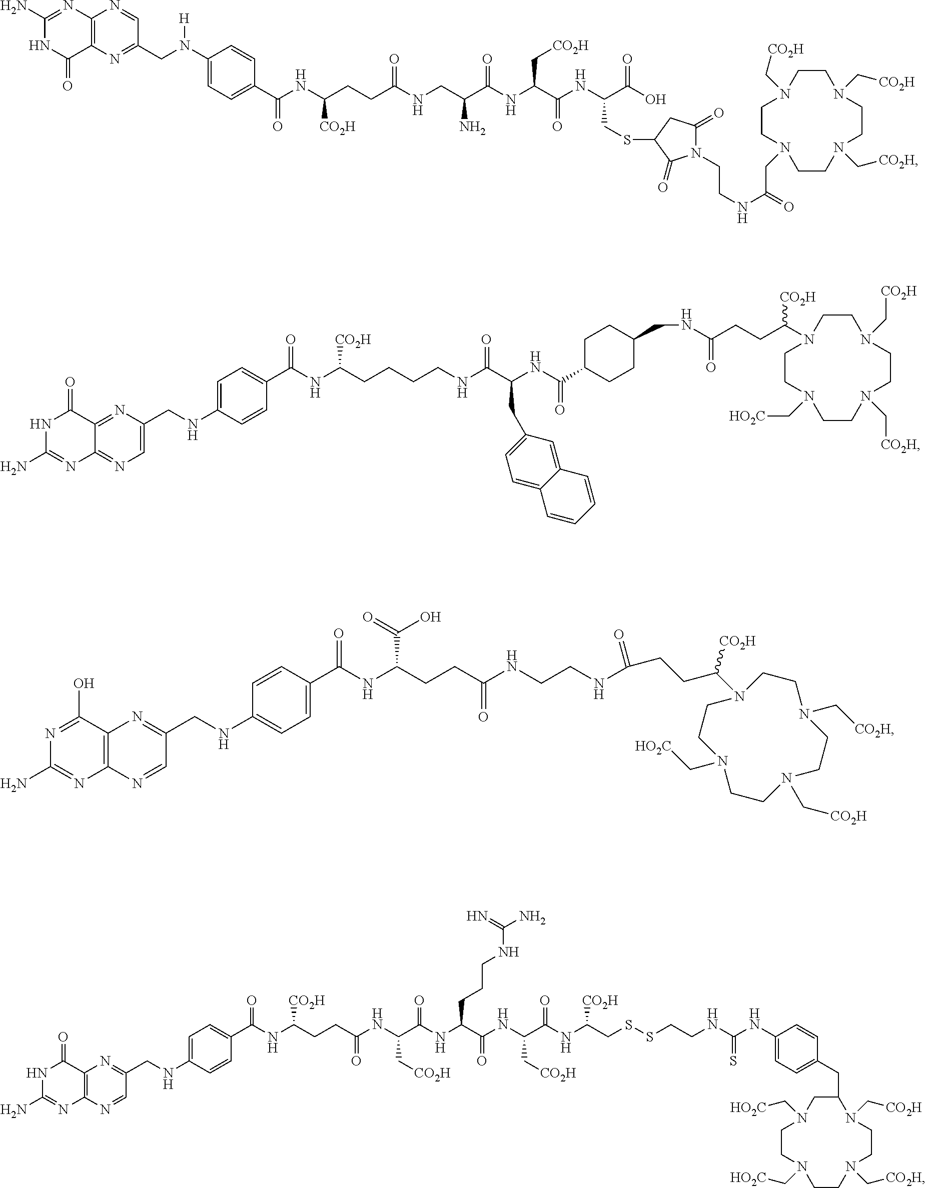 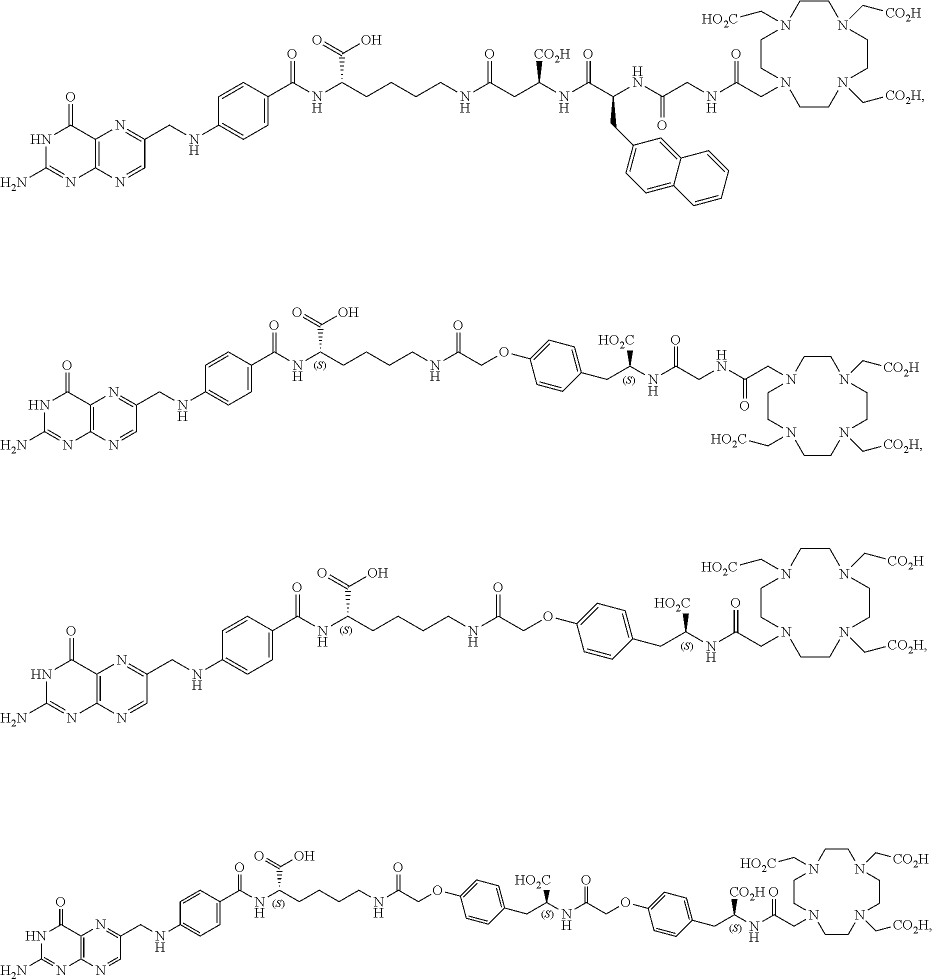 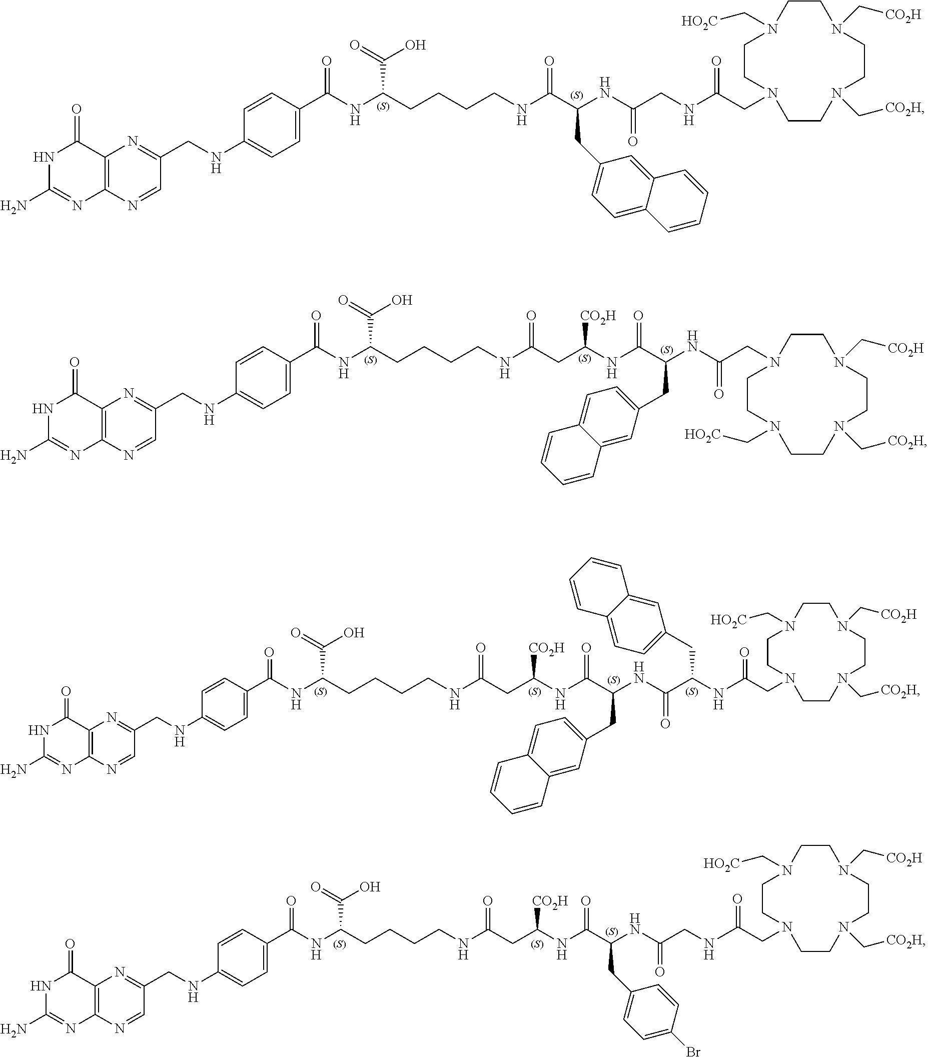 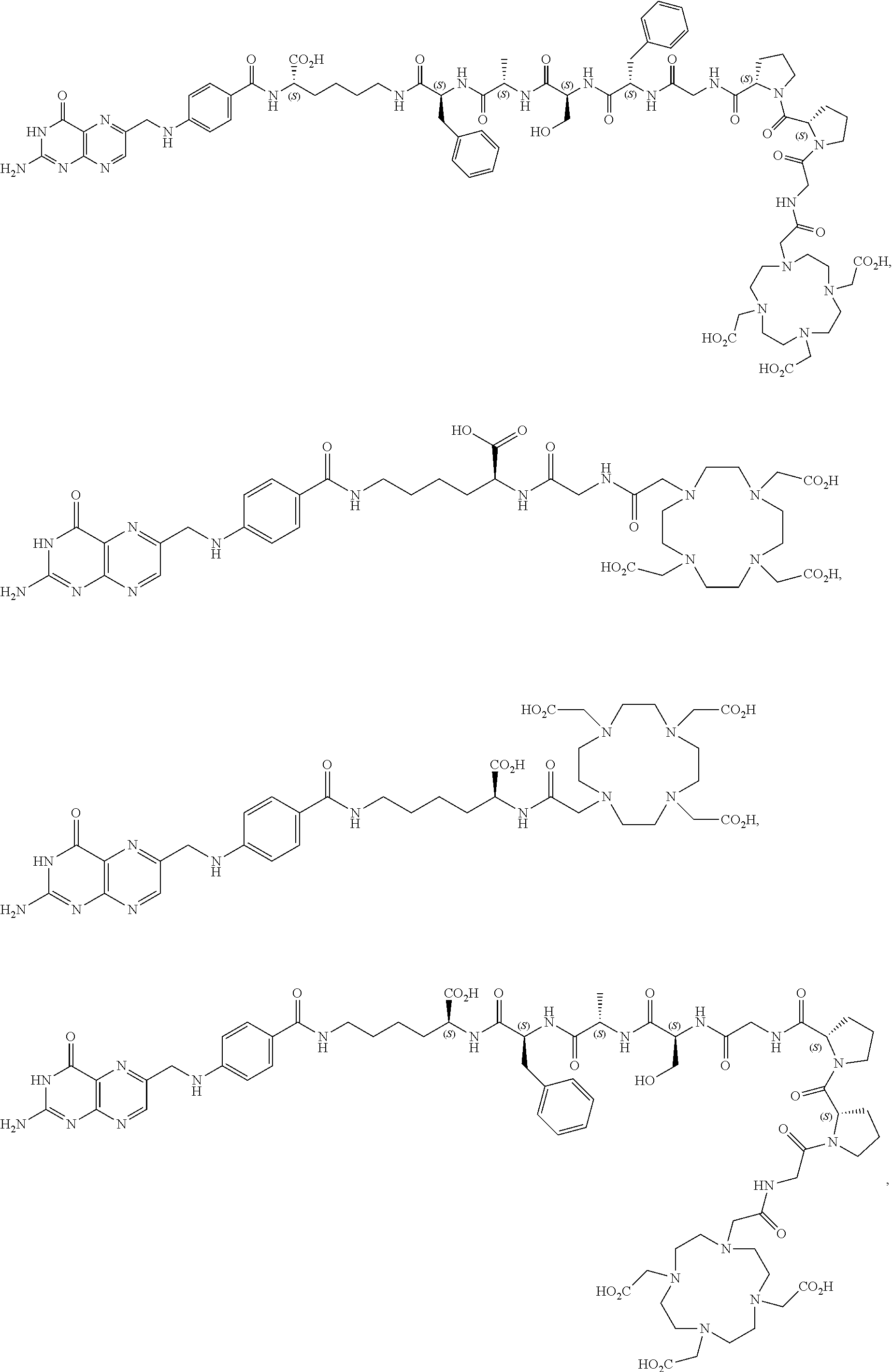 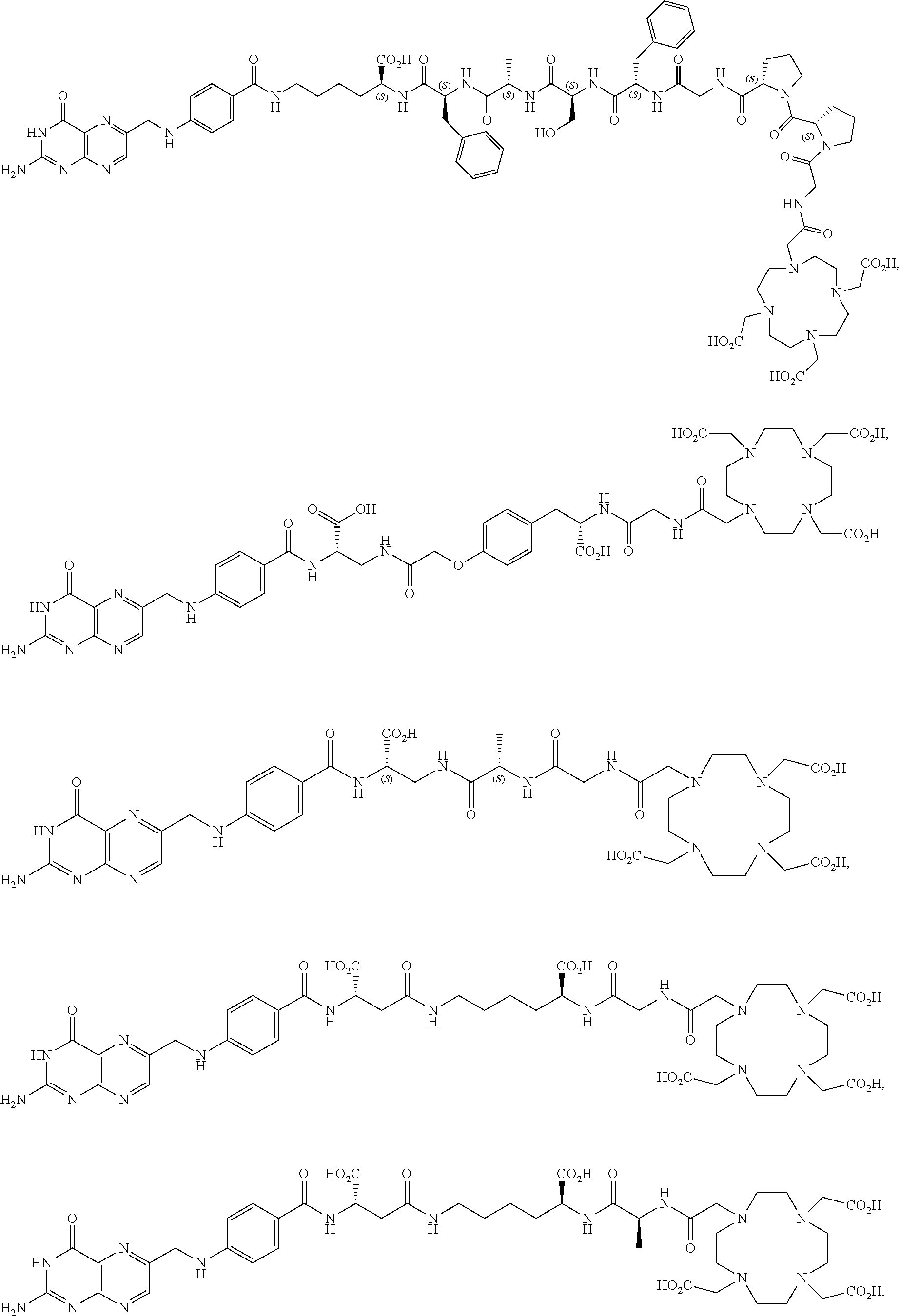 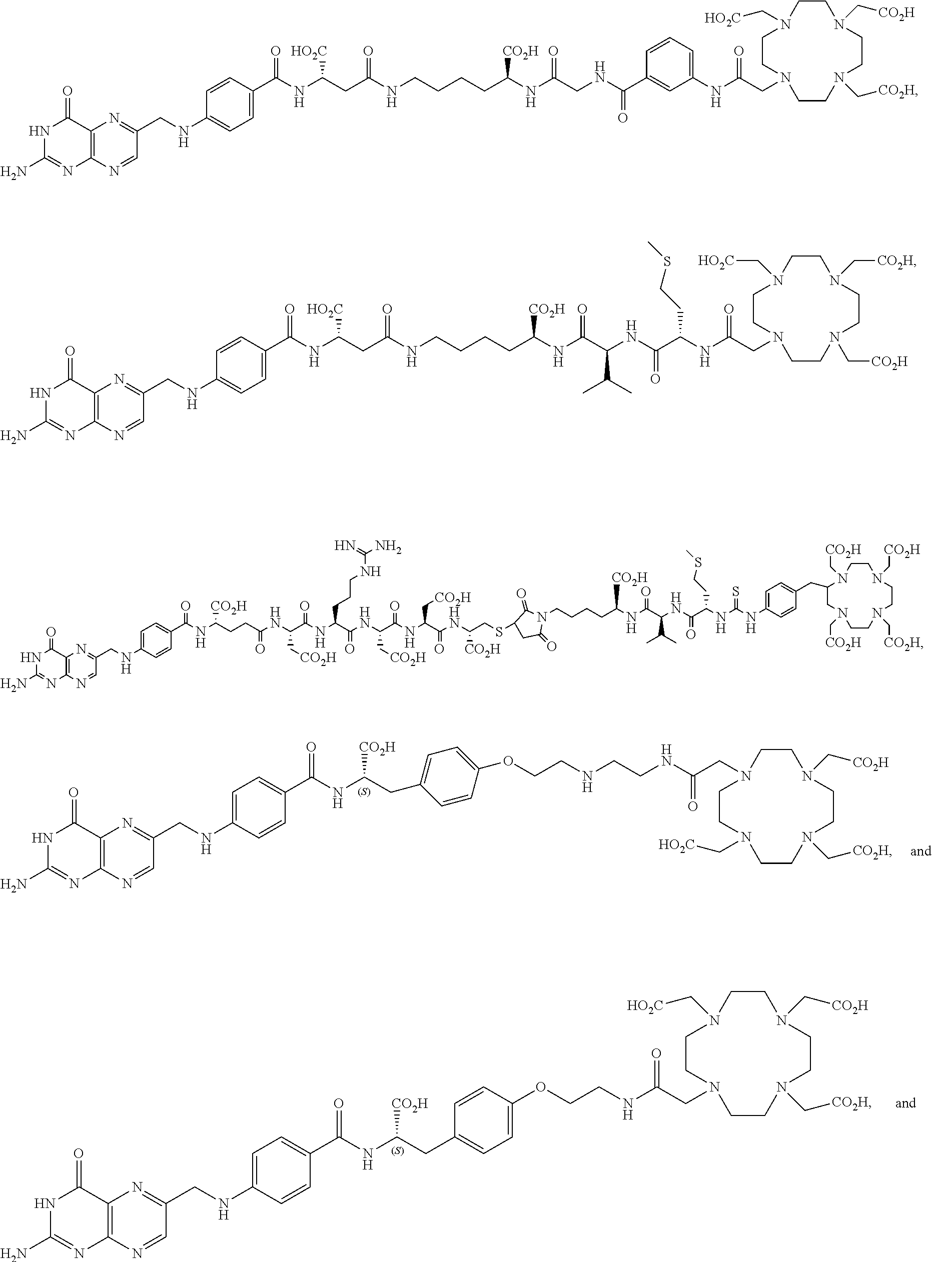 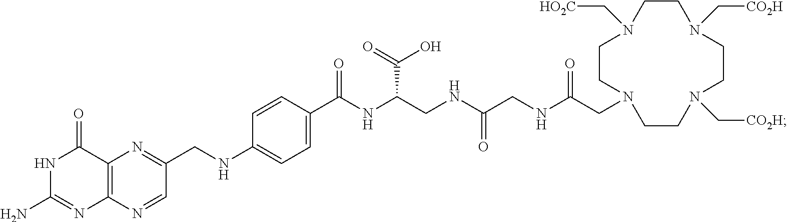 [SMILES: Nc1nc2ncc(CNc3ccc(C(=O)N[C@@H](CNC(=O)CNC(=O)CN4CCN(CC(=O)O)CCN(CC(=O)O)CCN(CC(=O)O)CC4)C(=O)O)cc3)nc2c(=O)[nH]1]
or a pharmaceutically acceptable salt thereof; wherein the chelating group exhibited in the above structural formulas can comprise a radioelement, Si-¹⁸F, B-¹⁸F, or Al-¹⁸F.

51. The compound of claim 1, wherein the compound is of formula 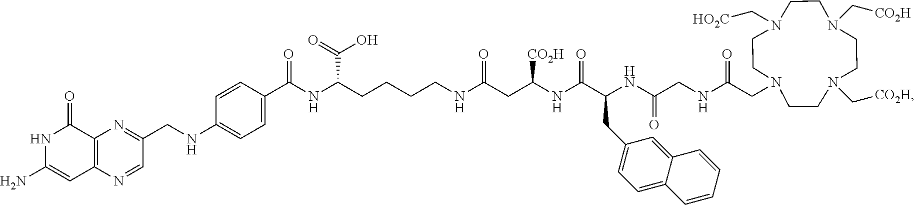 [SMILES: Nc1cc2ncc(CNc3ccc(C(=O)N[C@@H](CCCCNC(=O)C[C@H](NC(=O)[C@H](Cc4ccc5ccccc5c4)NC(=O)CNC(=O)CN4CCN(CC(=O)O)CCN(CC(=O)O)CCN(CC(=O)O)CC4)C(=O)O)C(=O)O)cc3)nc2c(=O)[nH]1]
or a pharmaceutically acceptable salt thereof.

52. The compound of claim 1, wherein the compound is of formula 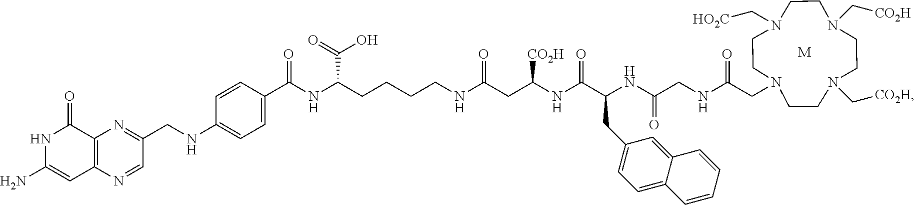
wherein M is a bound radioelement and M is ¹⁷⁷Lu or ²²⁵Ac; or a pharmaceutically acceptable salt thereof.

53. The compound of claim 1, wherein the compound is of formula 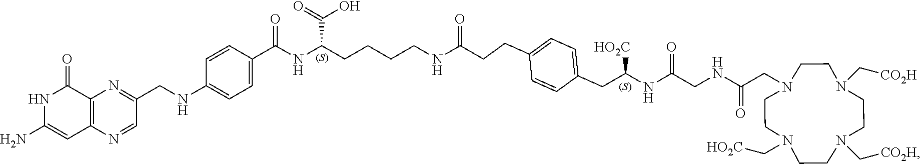
or a pharmaceutically acceptable salt thereof.

54. The compound of claim 1, wherein the compound is of formula, 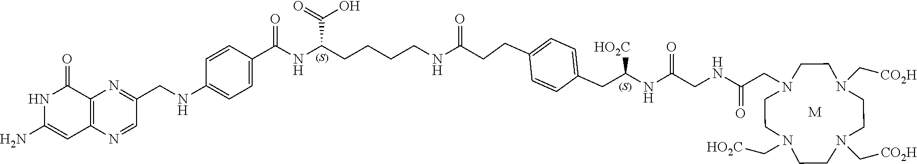
wherein M is a bound radioelement and M is ¹⁷⁷Lu or ²²⁵Ac; or a pharmaceutically acceptable salt thereof.

55. The compound of any one of the preceding claims, wherein the compound comprises a group of formula 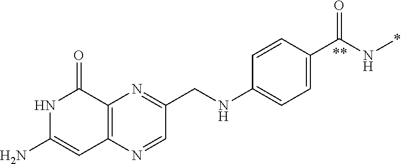 [SMILES: *NC(=O)c1ccc(NCc2cnc3cc(N)[nH]c(=O)c3n2)cc1]
and a carboxyl group in β, γ, δ, ε, or ζ position relative to the carbonyl indicated with “**” in above formula; or a pharmaceutically acceptable salt thereof.

56. The compound of any one of claims 1, 2, 5, 6, 7, 11, 12, 16-36, 41-43, 47-49, and 50, or a pharmaceutically acceptable salt thereof, wherein PG is labeled with a radiohalogen selected from the group consisting of ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I and ²¹¹At.

57. A pharmaceutical composition comprising a compound according to any one of the preceding claims, or a pharmaceutically acceptable salt thereof, and a pharmaceutically acceptable carrier.

58. A method of treating an FR expressing tumor or cell, the method comprising contacting the one or more FR expressing tumor or cell with an effective amount of a compound, or pharmaceutically acceptable salt thereof, according to any one of claims 1 to 55 or with an effective amount of the pharmaceutical composition of claim 57, wherein the compound comprises a chelating group which chelates a radioelement.

59. The method of claim 58, wherein the FR expressing tumor or cell is in vitro, in-vivo, or ex vivo.

60. A method of treating a proliferative disease in a subject in need thereof, comprising administering to the subject a therapeutically effective amount of a compound, or a pharmaceutically acceptable salt thereof, according to any one of claims 1 to 55, or a therapeutically effective amount of a pharmaceutical composition of claim 57, wherein the compound comprises a chelating group which chelates a radioelement.

61. The method of claim 60, wherein the proliferative disease is cancer.

62. The method of claim 61, wherein the cancer is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

63. The method of any one of claims 60 to 62, further comprising administering to the subject an effective amount of folic acid.

64. The method of any one of claims 60 to 63, further comprising administering to the subject an effective amount of an antifolate.

65. The method of any one of claims 60 to 64, further comprising administering to the subject and effective amount of a radio-sensitizer.

66. The method of any one of claims 60 to 65, wherein the subject is a human.

67. Use of a compound according to any one of claims 1 to 55, or a pharmaceutically acceptable salt thereof, in the preparation of a medicament for the treatment of cancer.

68. A compound according to any one of claims 1 to 55, or a pharmaceutically acceptable salt thereof, for use in a method of treating cancer in a subject.

69. A method for imaging FR expressing cells in a subject, comprising administering to the subject an effective amount of a compound, or a pharmaceutically acceptable salt thereof, according to any one of claims 1 to 56, or an effective amount of a pharmaceutical composition of claim 57, wherein the compound comprises a metal, a radioelement or radiohalogen.
